(19) United States
(12) Patent Application Publication
(10) Pub. No.: US 2025/0154463 A1
(43) Pub. Date: May 15, 2025

(54) COMPOSITIONS INCLUDING CYTOTOXIC INNATE LYMPHOID CELLS AND USES THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ming Li, New York, NY (US); Briana G. Nixon, New York, NY (US); Chun Chou, New York, NY (US); Emily R. Kansler, New York, NY (US); Saida Dadi, New York, NY (US); Jing Zhang, New York, NY (US)

(21) Appl. No.: 18/838,567

(22) PCT Filed: Feb. 14, 2023
(86) PCT No.: PCT/US2023/062550

§ 371 Date: Aug. 14, 2024

Related U.S. Application Data
(60) Provisional application No. 63/310,236, filed on Feb. 15, 2022.

(51) Int. Cl. C12N 5/0783; A61K 40/31; A61K 45/06; A61P 35/00; C12N 15/85

(52) U.S. Cl. CPC C12N 5/0646; A61K 40/31; A61K 45/06; A61P 35/00; C12N 15/85; C12N 2510/00

(57) ABSTRACT

Disclosed herein are compositions including cytotoxic innate lymphoid cells (ILCs), methods for preparing ILCs for adoptive cell therapy, and methods of using ILCs to treat cancer.

### CROSS-REFERENCE TO RELATED APPLICATIONS

This application is the U.S. National Stage Application of International Application No. PCT/US2023/062550, filed Feb. 14, 2023, which claims the benefit of and priority to U.S. Provisional Patent Application No. 63/310,236, filed Feb. 15, 2022, the contents of each of which is incorporated herein by reference in its entirety.

### STATEMENT OF GOVERNMENT SUPPORT

This invention was made with government support under CA008748 awarded by the National Institutes of Health. The government has certain rights in the invention.

### SEQUENCE LISTING

The instant application contains a Sequence Listing which has been submitted electronically in. xml format and is hereby incorporated by reference in its entirety. Said, xml copy, created on Aug. 13, 2024, is named 115872-3064_SL.xml and is 90,112 bytes in size.

### TECHNICAL FIELD

The present disclosure provides compositions including cytotoxic innate lymphoid cells (ILCs), methods for preparing cytotoxic ILCs for adoptive cell therapy, and methods of using cytotoxic ILCs to treat cancer.

### BACKGROUND

The following description of the background of the present technology is provided simply as an aid in understanding the present technology and is not admitted to describe or constitute prior art to the present technology.

Innate lymphocytes are integral components of the cellular immune system that coordinates host defense against a multitude of challenges and can trigger immunopathology when dysregulated. Natural killer (NK) cells and innate lymphoid cells (ILCs) are innate immune effectors postulated to functionally mirror conventional cytotoxic T lymphocytes and helper T cells, respectively.

### SUMMARY OF THE PRESENT TECHNOLOGY

In one aspect, the present disclosure provides an engineered cytotoxic innate lymphoid cell (ILC) comprising a non-endogenous expression vector including a mammalian IL-15 nucleic acid sequence or a mammalian STAT5B nucleic acid sequence, wherein the IL-15 nucleic acid sequence or the STAT5B nucleic acid sequence is operably linked to an expression control sequence. The expression control sequence may comprise an inducible promoter, a constitutive promoter, a native IL-15 or STAT5B promoter, or a heterologous promoter. In certain embodiments, the IL-15 nucleic acid sequence encodes the amino acid sequence of SEQ ID NO: 19 or SEQ ID NO: 20. In other embodiments, the STAT5B nucleic acid sequence encodes the amino acid sequence of SEQ ID NO: 9 or SEQ ID NO: 23. Additionally or alternatively, in some embodiments, the non-endogenous expression vector is a plasmid, a cosmid, a bacmid, a bacterial artificial chromosome (BAC), a yeast artificial chromosome (YAC), a viral vector, or a retroviral vector. The engineered ILCs may be derived from an autologous donor or an allogenic donor.

Additionally or alternatively, in some embodiments, the engineered cytotoxic ILC further comprises a chimeric antigen receptor (CAR) that binds to a tumor antigen and/or a nucleic acid encoding the CAR. In some embodiments, the CAR comprises (i) an extracellular antigen binding domain; (ii) a transmembrane domain; and (iii) an intracellular domain comprising one or more co-stimulatory domains, wherein the extracellular antigen binding domain binds to the tumor antigen. In any of the preceding embodiments, the heterologous promoter is induced by binding of the CAR to the tumor antigen, optionally wherein binding of the CAR to the tumor antigen results in antigen-dependent JAK-STAT5 pathway activation.

In another aspect, the present disclosure provides an engineered cytotoxic innate lymphoid cell (ILC) comprising a chimeric antigen receptor (CAR) that binds to a tumor antigen, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains. In some embodiments, the truncated cytoplasmic domain of IL-2RβΔ comprises the amino acid sequence of SEQ ID NO: 7.

In any and all embodiments of the engineered cytotoxic ILCs disclosed herein, the extracellular antigen binding fragment of the CAR comprises a single-chain variable fragment (scFv), preferably a human scFv. Examples of tumor antigens include, but are not limited to, 5T4, alpha 5β1-integrin, 707-AP, AFP, ART-4, B7H4, BCMA, Bcr-abl, CA125, CA19-9, CDH1, CDH17, CAMEL, CAP-1, CASP-8, CD5, CD25, CDC27/m, CD37, CD52, CDK4/m, c-Met, CS-1, CT, Cyp-B, cyclin B1, DAGE, DAM, EBNA, ErbB3, ELF2M, EMMPRIN, ephrinB2, estrogen receptor, ETV6-AML1, FAP, ferritin, folate-binding protein, G250, GM2, HAGE, HLA-A*0201-R170I, HPV E6, HPV E7, HSP70-2M, HST-2, hTERT (or hTRT), iCE, IL-2R, IL-5, KIAA0205, LAGE, LDLR/FUT, MART-1/melan-A, MART-2/Ski, MC1R, mesothelin, MUC16, myc, MUM-2, MUM-3, NA88-A, NYESO-1, NY-Eso-B, proteinase-3, p190 minor bcr-abl, Pm1/RARα, progesterone receptor, PSCA, RU1 or RU2, RORI, SART-1 or SART-3, survivin, TEL/AML1, TGFβ, TPI/m, TRP-1, TRP-2, TRP-2/INT2, tenascin, TSTA tyrosinase, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PIGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Ley) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, and peptide MHC complexes (with peptides derived from TP53, KRAS, MYC, EBNA1-6, PRAME, tyronsinase, MAGEA1-A6, pmel17, LMP2, or WT1).

Additionally or alternatively, in some embodiments of the engineered cytotoxic ILCs disclosed herein, the CAR transmembrane domain comprises a CD8 transmembrane domain, a CD28 transmembrane domain, a NKG2D transmembrane domain, a CD35 transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, or a BTLA transmembrane domain.

Additionally or alternatively, in certain embodiments of the engineered cytotoxic ILCs disclosed herein, the one or more CAR co-stimulatory domains may be selected from among a CD28 co-stimulatory domain, a 4-1BB co-stimulatory domain, an OX40 co-stimulatory domain, an ICOS co-stimulatory domain, a DAP-10 co-stimulatory domain, a PD-1 co-stimulatory domain, a CTLA-4 co-stimulatory domain, a LAG-3 co-stimulatory domain, a 2B4 co-stimulatory domain, a BTLA co-stimulatory domain, a NKG2C co-stimulatory domain, a NKG2D co-stimulatory domain, or any combination thereof. In some embodiments, the one or more co-stimulatory domains comprise a DAP-10 co-stimulatory domain and a 2B4 co-stimulatory domain

In one aspect, the present disclosure provides a composition comprising an effective amount of any and all embodiments of the engineered cytotoxic ILCs described herein and a pharmaceutically acceptable carrier.

In another aspect, the present disclosure provides a method of preparing immune cells for adoptive cell therapy (ACT) comprising: (a) isolating cytotoxic innate lymphoid cells (ILCs) from a donor subject, (b) transducing the cytotoxic ILCs with a nucleic acid encoding IL-15 or STAT5B or an expression vector comprising said nucleic acid, and (c) administering the transduced cytotoxic ILCs to a recipient subject. In certain embodiments, the nucleic acid encodes the amino acid sequence of SEQ ID NO: 19, SEQ ID NO: 20, SEQ ID NO: 9 or SEQ ID NO: 23. Additionally or alternatively, in some embodiments, the method further comprises transducing the cytotoxic ILCs with a nucleic acid encoding a chimeric antigen receptor (CAR) that binds to a tumor antigen. Also disclosed herein is a method of preparing immune cells for adoptive cell therapy (ACT) comprising: (a) isolating cytotoxic innate lymphoid cells (ILCs) from a donor subject, (b) transducing the cytotoxic ILCs with a nucleic acid encoding a chimeric antigen receptor (CAR) that binds to a tumor antigen or an expression vector comprising said nucleic acid, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains, and (c) administering the transduced cytotoxic ILCs to a recipient subject. In some embodiments of the ACT methods described herein, the donor subject and the recipient subject are the same or different.

In yet another aspect, the present disclosure provides a method for treating cancer or inhibiting tumor growth in a subject in need thereof comprising administering to the subject an effective amount of any and all embodiments of the engineered cytotoxic ILCs described herein or the pharmaceutical compositions described herein. The cancer or tumor may be selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Additionally or alternatively, in some embodiments of the methods disclosed herein, the engineered cytotoxic ILC is administered pleurally, intravenously, subcutaneously, intranodally, intratumorally, intrathecally, intrapleurally or intraperitoneally. In certain embodiments, the methods of the present technology further comprise sequentially, separately, or simultaneously administering to the subject an additional cancer therapy. Examples of additional cancer therapy include, but are not limited to, chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

Also disclosed herein are kits comprising an expression vector that includes a nucleic acid sequence encoding a mammalian IL-15 or STAT5B amino acid sequence, such as SEQ ID NO: 19, SEQ ID NO: 20, SEQ ID NO: 9 or SEQ ID NO: 23, and instructions for transducing cytotoxic ILCs with the expression vector. Additionally or alternatively, the kits of the present technology may comprising a vector encoding any and all embodiments of CAR constructs described herein or another cell-surface ligand that binds to a tumor antigen.

### BRIEF DESCRIPTION OF THE DRAWINGS

FIGS. 1A-1C: S1pr5 and granzyme C mark subsets of circulating and tissue-resident group 1 innate lymphocytes, respectively. FIG. 1A: Fold change versus fold change plot showing log fold change (LFC) of gene expression from microarray of liver (Liv) ILC1s versus Liv NK cells (y-axis) versus mean accessibility LFC for Liv ILC1s versus Liv NK cells. Genes with significant differentially accessible peaks are included; genes with significant differential expression are shown in black, red (enriched in ILC1) or green (enriched in NK cell), and genes without significant differential expression are shown in gray. FIG. 1B: Gene accessibility tracks for S1pr5 and Gzmc, displaying average peaks for splenic (Spl) NK cell, Liv NK cell, Liv ILC1, and salivary gland (SG) ILC1, which had differential accessibility between overall ILC1 and NK cell as well as differential gene expression between Liv ILC1 and Liv NK cell. Differentially accessible peaks are highlighted in red box. FIG. 1C: Representative histograms (left) and quantification (right) of CD49b, CD11b, KLRG1, CD49a, CD103, CXCR6, CD127, CD200R1, CD69, Eomes, T-bet, and IFN-γ expression in Spl, Liv, and SG S1pr5^{eGFP}-positive or granzyme C (GzmC)-positive CD3 NK1.1⁺NKp46⁺ cells of S1pr5^{eGFP-iCre} mice, or fluorescence minus one (FMO) staining. Each dot represents one mouse, 3-12 mice per group. All data are combined from three or more independent experiments and shown as mean+/−SEM (one-way ANOVA with Tukey's multiple comparisons test, “ns”=not significant, *=p<0.05, **=p<0.01, ***=p<0.001, ****=p<0.0001).

FIGS. 2A-2F: Granzyme C-expressing innate lymphocytes differentiate from ILCps, but not from NK cells, ILC2s, or ILC3s. FIG. 2A: Experimental design for S1pr5 fate-mapper (S1pr5^{FM}) chimeric mice. Expression of S1pr5^{FM} and granzyme C (GzmC) in splenic (Spl), liver (Liv), and salivary gland (SG) CD3⁻NK1.1⁺ cells. FIG. 2B: Expression of S1pr5^{FM} in Liv and SG CXCR6⁺CD49a⁺, SG Eomes⁺CD49a⁺, and Liv CD49a⁻CD49b⁺ NK cells from S1pr5^{FM} chimeric mice. FIG. 2C: Experimental design for ILCp (Lin⁻CD127⁺α4β7⁺Flt3⁻CD25⁻PD-1⁺) transfer. Expression of CD49a and GzmC in Liv ILCp-derived CD3⁻NK1.1⁺NKp46⁺ cells. FIG. 2D: Experimental design for IL-5 fate-mapper (Il5^{FM}) mice. Expression of Il5^{FM} and GzmC in Liv, SG, and small intestine lamina propria (SI LP) Lin⁻Thy1.2⁺ and/or NK1.1⁺ innate lymphocytes. FIG. 2E: Experimental design for IL-17A fate-mapper (Il17a^{FM}) mice. Expression of Il17a^{FM} and GzmC in Liv, SG, and SI LP Lin Thy1.2⁺ and/or NK1.1⁺ innate lymphocytes. FIG. 2F: Experimental design for IL-22 fate-mapper (Il22^{FM}) mice. Expression of Il22^{FM} and GzmC in Liv, SG, and SI LP Lin⁻Thy1.2⁺ and/or NK1.1⁺ innate lymphocytes. Each dot represents one mouse, n=3-6 mice per group. All data are combined from three or more independent experiments and shown as mean+/−SEM (one-way ANOVA with Tukey's multiple comparisons test, “ns”=not significant, *=p<0.05, **=p<0.01, ***=p<0.001, ****=p<0.0001).

FIGS. 3A-3G. Granzyme C-expressing cells are not precursors for NK cells, ILC2s, or ILC3s. FIG. 3A: Representative flow cytometric analysis (left) and quantification (right) of granzyme C (GzmC) and S1pr5^{eGFP} expression in NK1.1⁺NKp46⁺ innate lymphocytes in livers of 0.5 day-, 7 day-, 14 day-old and adult (8-12 weeks of age) mice. FIG. 3B: Representative plots of Gzmc^{td-Tomato} and CD49a expression in splenic (Spl) and liver (Liv) NK phenotype and Liv and salivary gland (SG) ILC1 phenotype cells of Gzmc^{tdT-T2A-iCre} mice. FIG. 3C: Experimental design of granzyme C fate-mapper (Gzmc^{FM}) mice. FIG. 3D: Quantification of Gzmc^{FM} expression in Spl and Liv NK phenotype as well as Liv and SG ILC1 phenotype cells. FIG. 3E: Expression of Gzmc^{FM} and IL-5 in Liv, SG, and small intestine lamina propria (SI LP) Lin⁻Thy1.2⁺ and/or NK1.1⁺ innate lymphocytes after four-hour PMA/Ionomycin/Golgi stop treatment. FIG. 3F: Expression of Gzmc^{FM} and IL-17A in Liv, SG, and SI LP Lin⁻Thy1.2⁺ and/or NK1.1⁺ innate lymphocytes after four-hour PMA/Ionomycin/Golgi stop treatment. FIG. 3G: Expression of Gzmc^{FM} and IL-22 in Liv, SG, and SI LP Lin⁻Thy1.2⁺ and/or NK1.1⁺ innate lymphocytes after four-hour PMA/Ionomycin/Golgi stop treatment. Each dot represents one mouse, n=3-10 mice per group. All data are combined from three or more independent experiments and shown as mean+/−SEM (one-way ANOVA with Tukey's multiple comparisons test, “ns”=not significant, *=p<0.05, **=p<0.01, ***=p<0.001, ****=p<0.0001).

FIGS. 4A-4B. Differential regulation of ILC1 populations across tissues by T-bet, Eomes and TGF-β. FIG. 4A: Representative (left) flow cytometric analysis of granzyme C (GzmC) and CD49a expression among NK1.1⁺CD3⁻ cells in the liver (upper) and salivary gland (lower) of wild-type (WT), GzmC^{iCre}Tbx21^{fl/fl} (Gzmc^{ΔTbx21}) (yellow), GzmC^{iCre}Eomes^{fl/fl} (Gzmc^{ΔEomes}) (green), and GzmC^{iCre}Tbx21^{fl/fl}Eomes^{fl/fl} (Gzmc^{ΔTbx21Δeomes}) (blue) mice. (Right) abundance of GzmC⁺NK1.1⁺CD3⁻ (GzmC⁺) and CD49a⁺NK1.1⁺CD3⁻ (ILC1) cells out of total CD45⁺ cells in liver and salivary gland. FIG. 4B: Representative (left) and quantification (right) of flow cytometric analysis of group 1 innate lymphocytes in liver (upper) and salivary gland (lower) of WT (white bar) and GzmC^{iCre}Tgfbr2^{fl/fl} (Gzmc^{ΔTgfbr2}) (gray bar) mice, quantifying CD49a⁺ cells among NK1.1⁺CD3⁻ cells and CD103⁺ and GzmC⁺ cells among CD49a⁺NK1.1⁺CD3⁻ cells in each organ. Each dot represents one mouse (n=4-10 mice per group). All data are combined from three or more independent experiments and shown as mean+/−SEM (unpaired student's t-test for two groups, one-way ANOVA with Tukey's multiple comparisons test with more than two groups, “ns”=not significant, *=p<0.05, **=p<0.01, ***=p<0.001, ****=p<0.0001).

FIGS. 5A-5I: Granzyme C-expressing ILC1s mediate cancer immunosurveillance. FIG. 5A: Abundance of CXCR6⁺CD49a⁺ NK1.1⁺CD3⁻ (CXCR6⁺CD49a⁺), Eomes⁺CD49a⁺ NK1.1⁺CD3⁻ (Eomes⁺CD49a⁺) and CD49a⁻ CD49b⁺NK1.1⁺CD3⁻ (NK) cells out of total CD45⁺ cells in PyMT tumors. Expression of S1pr5^{eGFP} FP (FIG. 5B), granzyme C (GzmC) (FIG. 5C), S1pr5^{FM} (FIG. 5D), and Gzmc^{FM} (FIG. 5E) in CXCR6⁺CD49a⁺ ILC1 (red), Eomes⁺CD49a⁺ILC1 (blue), and CD49a⁻ CD49b⁺ NK cell (green) populations in PyMT tumors of S1pr5^{eGFP}PyMT (FIG. 5B), PyMT (FIG. 5C), S1pr5^{FM}PyMT chimera (FIG. 5D), and Gzmc^{FM}PyMT (FIG. 5E) mice. FIG. 5F: Abundance of ILC1s in tumors of PyMT (white bar) and GzmC^{iCre}Tgfbr2_{fl/fl}PyMT (Gzmc^{ΔTgfbr2}PyMT) (gray bar) mice, quantifying CD49a⁺ cells among NK1.1⁺CD3⁻ cells and CD103⁺ and GzmC⁺ cells among CD49a⁺NK1.1⁺CD3⁻ cells in the tumor. FIG. 5G: Tumor volume at 12 and 16 weeks of age from PyMT (white bar) and Gzmc^{ΔTgfbr2}PyMT (gray bar) mice. FIG. 5H: Abundance of ILC1s in tumors of PyMT (white bar) and GzmC^{iCre}Rosa26^{LSL-DTA/+} PyMT (Gzmc^{DTA}PyMT) (black bar) mice, quantifying CD49a⁺ cells among NK1.1+CD3⁻ cells and CD103⁺ and GzmC⁺ cells among CD49a⁺NK1.1⁺CD3⁻ cells in the tumor. FIG. 5I: Tumor volume at 12 and 16 weeks of age from PyMT (white bar) and Gzmc^{DTA}PyMT (black bar) mice. Each dot represents one mouse (n=5-9 mice per group). All data are combined from three or more independent experiments and shown as mean+/−SEM (unpaired student's t-test for two groups, one-way ANOVA with Tukey's multiple comparisons test with more than two groups, “ns”=not significant, *=p<0.05, **=p<0.01, ***=p<0.001, ****=p<0.0001).

FIGS. 6A-6D: ILC1s broadly express cytotoxic molecules and granzyme C expression marks a mature effector state. FIG. 6A: Representative (upper) and quantification (lower) of granzyme B (GzmB) and perforin expression among splenic (Spl) and liver (Liv) NK cells as well as Liv and salivary gland (SG) ILC1s. FIG. 6B: Representative and quantification of GzmB and perforin protein expression among subsets of ILC1s in Liv (upper) and SG (lower) based on current and history of granzyme C expression (GzmC⁻Gzmc^{FM-}, double-negative [DN], blue; GzmC⁻Gzmc^{FM+}, fate-mapped single positive [FMSP], purple; GzmC⁺Gzmc^{FM+}, double positive [DP], red). FIG. 6C: Number of differentially expressed genes in each of six pairwise comparisons conducted between the four sequenced populations. Color indicates direction of higher expression.

FIG. 6D: 62 of 74 genes significantly differentially expressed between DP and DN ILC1s with higher expression in DP, grouped by function and localization. 12 genes could not be grouped (data not shown). Each dot represents one mouse (n=4-5 mice per group). Flow cytometry data are combined from three or more independent experiments and shown as mean+/−SEM (one-way ANOVA with Tukey's multiple comparisons test, “ns”=not significant, *=p<0.05, **=p<0.01, ***=p<0.001, ****=p<0.0001).

FIGS. 7A-7B: CXCR6⁺ ILC1s from liver can mediate perforin-dependent cytotoxicity regardless of initial granzyme C expression. FIG. 7A: Expression of granzyme C (GzmC) in group 1 innate lymphocyte subsets sorted based on current or history of GzmC expression and cultured in 100 ng/ml IL-15/IL-15Rα for 24 hours (h). Sorted populations included DP (CD49a⁺CXCR6⁺Gzmc^{tdT+}Gzmc^{FM+}) (red), FMSP (CD49a⁺CXCR6⁺Gzmc^{tdT-}Gzmc^{FM+}) (purple), DN (CD49a⁺CXCR6⁺Gzmc^{tdT-}Gzmc^{FM-}) (blue), and NK (CD49a⁻CD49b⁺CD11b⁺) (green) cell subsets. Data are combined from four independent experiments. FIG. 7B: Killing assay, displaying death rate of target cells after coincubation with subsets of CXCR6⁺ILC1s from the liver. Effector cells were either Gzmc^{tdT+} or Gzmc^{tdT-} ILC1s (CXCR6⁺CD49a⁺NK1.1⁺CD3⁻) and were sorted from the livers of Gzmc^{tdT-T2A-iCre/+}Prf1^{+/+} or Gzmc^{tdT-T2A-iCre/+}Prf1^{-/-} mice. Effectors were expanded in 100 ng/ml IL-15/IL-15Rα and cocultured with CTV-labeled RMA-S target cells for 16 h at a 10:1 effector: target ratio in media supplemented with 100 ng/ml IL-15/IL-15Rα. Data are representative from one of three independent experiments. Each dot represents one mouse (n=3-6 mice per group). All data are shown as mean+/−SEM (one-way ANOVA with Tukey's multiple comparisons test for more than two groups, “ns”=not significant, *=p<0.05, **=p<0.01, ***=p<0.001, ****=p<0.0001).

FIGS. 8A-8F: IL-15 promotes generation of granzyme C-expressing ILC1s with enhanced signaling causing perforin-dependent cytotoxicity and lethal immunopathology. FIG. 8A: Granzyme C (GzmC) expression in liver (Liv) and salivary gland (SG) CD3⁻NK1.1⁺NKp46⁺ cells of wild-type (WT) and IL-15^{-/-} mice. FIG. 8B: Experimental design of Gzmc^{Stat5b-CA} mice. FIG. 8C: Survival curves for Gzmc^{Stat5b-CA/+}, Gzmc^{Stat5b-CA/+}Rag1^{-/-}, and littermate control mice. FIG. 8D: GzmC⁺NK1.1⁺NKp46⁺ cells per gram of Liv tissue from seven-day-old WT and Gzmc^{Stat5b-CA/+} mice. FIG. 8E: Representative images of hematoxylin and eosin staining (H&E, first row) and NKp46 (second row) and cleaved caspase 3 (CC3, third row) immunoreactivities (indicated by arrows) of Liv sections from 14-day-old WT, Gzmc^{Stat5b-CA/+}, and Gzmc^{Stat5b-CA/+}Prf1^{-/-} mice. Images and inserts are 200× and 400×, respectively. Arrowheads indicate immunopositive cells. NKp46 and CC3 immunoreactivity was also quantified on serial sections as percent of total tissue using digital pathology software (Halo). Scale bar indicates 100 μm. FIG. 8F: Survival curves for Gzmc^{Stat5b-CA/+} and Gzmc^{Stat5b-CA/+}Prf1^{-/-} littermate control mice (n=15-18 per group). Each dot represents one mouse; FIG. 8A, FIG. 8D, FIG. 8E: n=3-4 mice per group, FIG. 8C: n=7-18 per group, FIG. 8F: n=15-18 mice per group. All data are combined from three or more independent experiments and shown as mean+/−SEM (FIGS. 8A, 8D, 8E: one-way ANOVA with Tukey's multiple comparisons test, FIGS. 8C, 8F: Log-rank [Mantel Cox] test; “ns”=not significant, *=p<0.05, **=p<0.01, ***=p<0.001, ****=p<0.0001)

FIGS. 9A-9D: Chromatin accessibility of circulating and resident group 1 innate lymphocytes reveals differentially accessible peaks. (Related to FIG. 1) FIG. 9A: Sorting strategy for NK cell and ILC1 samples submitted for ATACsequencing from spleen (Spl), liver (Liv), and salivary gland (SG). All samples were gated as live, single cell, CD45⁺NK1.1⁺NKp46⁺Lineage⁻ (Lineage=CD19⁺CD3⁺CD5⁺Ly6G⁺TCRβ⁺) . NK cells were CD49b⁺CD49a⁻, while ILC1s were CD49a⁺. FIG. 9B: Heatmap of significantly differently accessible peaks shared after following pairwise comparisons: splenic NK cell versus Liv ILC1, versus SG ILC1; liver NK cell versus liver ILC1, versus SG ILC1 (blue decreased, orange increased accessibility). FIG. 9C: Comparing accessible chromatin to current gene expression between NK cell and ILC1: using peaks from comparisons performed in FIG. 9B, CDF plot showing enrichment of genes with more accessible peaks in NK cell (green), ILC1 (red), or background genes (black) in liver NK cell or liver ILC1 microarray-assessed gene fold change (x-axis). FIG. 9D: Gene accessibility tracks for Sel1, Slpr1, Itga1, and Cxcr6, displaying average peaks for Spl NK cell, Liv NK cell, Liv ILC1, and SG ILC1, which had differential accessibility between overall ILC1 and NK cell as well as differential gene expression between Liv ILC1 and Liv NK cell. Differentially accessible peaks are highlighted in red box.

FIGS. 10A-10E: S1pr5^{eGFP-T2A-iCre} mice reveal single-cell resolution of S1pr5 expression that marks mature NK cells. (Related to FIG. 1) FIG. 10A: Schematic of S1pr5^{eGFP-FP-T2A-iCre} knock-in mouse, generated employing CRISPR-Cas9 nickase in pX335 vector to introduce insertion into 3′ UTR of the S1pr5 gene. FIG. 10B: Southern Blot analysis of untargeted (7.6 kb) and successfully targeted (11 kb) mouse embryonic stem cells. FIG. 10C: PCR reaction on DNA from wild-type (WT) and S1pr5^{eGFP-T2A-iCre/+} mouse pups (WT=234 bp, Cre insertion=350 bp). FIG. 10D: Representative flow cytometric analysis (left) and quantification (right) of S1pr5^{eGFP} and granzyme C (GzmC) expression in splenic (Spl) and liver (Liv) NK cells (CD3⁻NK1.1⁺NKp46⁺CD49a⁻CD49b⁺) as well as Liv and salivary gland (SG) ILC1s (CD3⁻NK1.1⁺NKp46⁺CD49a⁺) from S1pr5^{eGFP-T2A-iCre} mice. FIG. 10E: Sample flow plots (left) and quantification (right) of S1pr5^{eGFP} expression positivity and geometric mean fluorescence intensity (MFI) in Spl NK cells with different maturation states defined by CD27 and CD11b expression. MFI was calculated as fold change of the CD27⁺CD11b NK cell population. Each dot represents one mouse (n=7-10 mice per group). All data is shown as mean+/−SEM (one-way ANOVA with Tukey's multiple comparisons test, “ns”=not significant, ****=p<0.0001).

FIGS. 11A-11G: Granzyme C-expressing innate lymphocytes are not derived from S1pr5⁺ NK cells, but can be differentiated from ILCps. (Related to FIG. 2) FIG. 11A: Schematic outlining whether S1pr5⁺ NK cells or ILCps can give rise to granzyme C (GzmC)-positive innate lymphocytes. FIG. 11B: S1pr5 fate-mapping (S1pr5^{eGFP-T2A-iCre/+}R26^{LSL-YFP} [S1pr5^{FM}]) in bone marrow LSK (Lin [B220, CD11b, CD19, CD3e, Gr1, Ly6D, TER119]⁻Scal⁺cKit [CD117]⁺) cells, which include hematopoietic stem cells. Varying degree of S1pr5^{FM} expression among LSK in multiple S1pr5^{FM} mice is shown. FIG. 11C: Expression of S1pr5^{FM} in T cells (CD3+), group 1 innate lymphocytes (CD3⁻NK1.1⁺), classical monocytes (F4/80⁺Ly6C⁺), and patrolling monocytes (F4/80⁺Ly6C⁻) from blood of chimeric mice (irradiated mice that received sorted fate map-negative LSK from bone marrow of S1pr5^{eGFP-T2A-iCre/+}R26^{LSL-YFP} mice, see FIG. 3A), and splenocytes from wild-type (WT) mice shown in gray. FIG. 11D: Related to FIG. 2A, representative flow cytometry analysis of expression of S1pr5^{FM} versus GzmC in CD3⁻NK1.1⁺NKp46⁺ cells from spleen, liver, and salivary gland of S1pr5^{FM} chimeric mice. FIG. 11E: Quantification of CXCR6 and Eomes expression among CD49a⁺NK1.1⁺CD3⁻ cells in the salivary gland (SG) (left) and representative flow cytometric analysis and quantification (right) of granzyme C expression among these two subsets in the SG. FIG. 11F: Gating strategy for ILC precursor (ILCp, Lin[B220, CD11b, CD19, CD3e, Gr1, Ly6D, TER119]⁻ CD127⁺α4β7 Flt3⁻CD25 PD-1⁺) in bone marrow of WT mice. FIG. 11G: Related to FIG. 2C, representative flow cytometry analysis of expression of CD49a versus GzmC in ILCp-derived CD3⁻NK1.1⁺NKp46⁺ cells from the liver. Each dot represents one mouse (n=3-5 mice per group). All data is shown as mean+/−SEM (unpaired student's t-test for two groups, one-way ANOVA with Tukey's multiple comparisons test for more than two groups, “ns”=not significant, ***=p<0.01, ***=p<0.0001).

FIGS. 12A-12E: Cytokine-producing effector ILC2s and ILC3s do not give rise to granzyme C-positive innate lymphocytes. (Related to FIG. 2) FIG. 12A: Schematic proposing whether IL-5-, IL-17A- or IL-22-producing ILCs can give rise to granzyme C (GzmC)-positive innate lymphocytes. FIG. 12B: Gating strategy for innate lymphocytes from Il5^{FM}, Il17a^{FM} or Il22^{FM} mice: Lin(CD3, CD19)⁺NK1.1⁺ and/or Thy 1.2⁺ in liver, salivary gland, and small intestine lamina propria (SI LP). Representative flow cytometric analysis of expression of GzmC versus Il5^{FM} (FIG. 12C), Il17a^{FM} (FIG. 12D), or Il22^{FM} (FIG. 12E), quantified in FIGS. 2C, 2D, and 2E, respectively.

FIGS. 13A-13G: Gzmc^{tdT-T2A-iCre/+} mice allow for lineage tracing of cells with a history of granzyme C expression. (Related to FIG. 3) FIG. 13A: Schematic of Gzmc^{tdT-T2A-iCre} knock-in mouse, generated employing CRISPR-Cas9 nickase in pX335 vector to introduce insertion into 3′ UTR of the Gzmc gene. FIG. 13B: Southern Blot analysis of untargeted (5.9 kb) and successfully targeted (10.7 kb) mouse embryonic stem cells. FIG. 13C: PCR reaction on DNA from wild-type (WT) and Gzmc^{tdT-T2A-iCre/+} mouse pups (WT GzmC=175 bp, iCre insertion=317 bp). FIG. 13D: Expression of GzmC protein versus CD49a in spleen, liver, and salivary gland group 1 innate lymphocytes (NK1.1⁺CD3⁻). FIG. 13E: Expression of Gzmc^{td-Tomato} versus GzmC protein in spleen and liver NK and liver and salivary gland ILC1 phenotype cells after fixation with 4% PFA and further fixation/permeabilization for intracellular protein staining. FIG. 13F: Schematic proposing whether GzmC-positive innate lymphocytes can give rise to circulating NK cells. FIG. 13G. Related to FIG. 3D, representative flow cytometric analysis of Gzmc^{FM} in Spl and Liv NK and Liv and SG ILC1.

FIGS. 14A-14D: Cells with a history of granzyme C expression do not give rise to IL-5-, IL-17A-, or IL-22-producing ILC2s or ILC3s. (Related to FIG. 3). FIG. 14A: Schematic proposing whether granzyme C (GzmC)-positive innate lymphocytes can give rise to IL-5-, IL-17A-, or IL22-producing ILCs. Representative flow cytometric analysis of expression of Gzmc^{FM} versus IL-5 (FIG. 14B), IL-17A (FIG. 14C), or IL-22 (FIG. 14D) in innate lymphocytes (Lin[CD3, CD19]⁺NK1.1⁺ and/or Thy1.2⁺) from liver, salivary gland, and small intestine lamina propria (SI LP) of Gzmc^{FM} mice after 4-hour incubation with PMA/Ionomycin and Golgi stop, quantified in FIGS. 3E, 3F and 3G respectively.

FIGS. 15A-15D: Regulation of ILC1 by T-bet, Eomes and TGF-β. (Related to FIG. 4) FIG. 15A: Abundance of NK cells out of total CD45+ cells in liver of WT, GzmC^{iCre}Tbx21^{fl/fl} (Gzmc^{ΔTbx21}) (yellow), GzmC^{iCre}Eomes^{fl/fl} (Gzmc^{ΔEomes}) (green), and GzmC^{iCre}Tbx21^{fl/fl}Eomes^{fl/fl} (Gzmc^{ΔTbx21ΔEomes}) (blue) mice. FIG. 15B: Cell number per gram of tissue of GzmC⁺CD49a⁺NK1.1⁺CD3⁺ (GzmC), CD49a⁺NK1.1⁺CD3⁺ (CD49a⁺), and NK (CD49a⁺CD49b⁺CD11b⁺) cells in liver and salivary gland of WT, Gzmc^{ΔTbx21} (yellow), Gzmc^{ΔEomes} (green), and Gzmc^{ΔTbx21ΔEomes} (blue) mice. FIG. 15C: Expression of Eomes versus GzmC among group 1 innate lymphocytes (NK1.1⁺CD3⁻) in salivary gland of WT, Gzmc^{ΔTbx21} (yellow), Gzmc^{ΔEomes} (green), and Gzmc^{ΔTbx21ΔEomes} (blue) mice. FIG. 15D: Representative plot and quantification of expression of CD103 versus GzmC in salivary gland CD49a⁺NK1.1⁺CD3⁻ cells.

FIGS. 16A-16E: Granzyme C-expressing innate lymphocytes in the tumor require TGF-β signaling and mediate cancer immunosurveillance. (Related to FIG. 5). FIG. 16A: Representative (left) and quantification (right) of GzmC expression within NK1.1⁺CD3⁻ population in mammary gland of WT mice and breast tumor of PyMT mice. FIG. 16B: Related to FIG. 5F, representative flow cytometry plots of CD49a expression among NK1.1+CD3⁻ cells and CD103 and GzmC expression among CD49a⁺NK1.1⁺CD3⁻ cells in tumors of PyMT and Gzmc^{ΔTgfbr2}PyMT mice. FIG. 16C: Schematic of Gzmc^{DTA} mice. FIG. 16D: Related to FIG. 5H, representative flow cytometry plots of CD49a expression among NK1.1⁺CD3⁻ cells and CD103 and GzmC expression among CD49a⁺NK1.1⁺CD3⁻ cells in tumors of PyMT and Gzmc^{DTA}PyMT mice. FIG. 16E. Representative (left) and quantification (right) of abundance of CXCR6⁺CD49a⁺NK1.1⁺CD3⁻, Eomes⁺CD49a⁺NK1.1⁺CD3⁻, and CD49a⁻CD49b⁺ (NK) populations in tumors of PyMT (white bar) and Gzmc^{DTA}PyMT (black bar) mice.

FIGS. 17A-17C: Potential heterogeneity among ILC1 subsets with differential history of granzyme C expression. (Related to FIG. 6) FIG. 17A: Quantification of abundance of ILC1 populations based on expression of GzmC protein and Gzmc^{FM} in liver and salivary gland of Gzmc^{FM} mice. FIG. 17B: Gating strategy for sorted populations for RNAsequencing experiment. All populations: CD45⁺NK1.1⁺CD3⁻, then Double-positive (DP): CD49a⁺CXCR6⁺Gzmc^{tdT+}Gzmc^{FM+} (red), fate-mapped single positive (FMSP): CD49a⁺CXCR6⁺Gzmc^{tdT-}Gzmc^{FM+} (purple), double negative (DN): CD49a⁺CXCR6⁺Gzmc^{tdT-}Gzmc^{FM-}, and NK: CD49a⁻CD49b⁺CD11b⁺S1pr5^{eGFP+} (green). FIG. 17C: Heat map showing genes differentially expressed and enriched either all in NK or all in ILC1s across three pairwise comparisons between NK and each of the ILC1 populations.

FIG. 18: Genes enriched in DN relative to DP ILC1 are enriched for NK-related and proliferation-related genes. (Related to FIG. 6) 228 of 245 genes significantly differentially expressed between GzmC⁺Gzmc^{FM+} (double-positive, DP) and GzmC⁻Gzmc^{FM-} (double-negative, DN) ILC1 with higher expression in DN, grouped by gene function and localization. 17 genes could not be grouped (data not shown).

FIGS. 19A-19B: Liver ILC1s can mediate perforin-dependent cytotoxicity. (Related to FIG. 7) FIG. 19A: Gating strategy for cell sorting of effector cells used in FIG. 7 killing assay, sorting from Gzmc^{tdT-T2A-iCre/+}Prf1^{+/+} and Gzmc^{tdT-T2A-iCre/+}Prf1^{-/-} mice. FIG. 19B: Representative flow plot of death rate in condition with target cells alone, no effector cells, in killing assay.

FIGS. 20A-20B. Granzyme C-expressing innate lymphocytes are lost in the absence of IL-15 and expanded upon gain-of-function in Stat5 signaling. (Related to FIG. 8). FIG. 20A: Representative flow cytometric analysis of granzyme C (GzmC) expression among NK1.1⁺NKp46⁺CD3⁻ cells in the liver and salivary gland of wild-type and IL-15^{-/-} mice. FIG. 20B: Representative flow cytometric gating strategy for quantification of GzmC⁺NK1.1⁺NKp46⁺ cell number per gram of tissue in liver of 7-day-old wild-type (WT) and Gzmc^{Stat5b-CA/+} mice.

FIGS. 21A-21C: Human type 1 innate lymphocytes armed with IL-2Rβ chain STAT5 chimeric antigen receptor (CAR) signaling show enhanced cytotoxicity to target cells. FIG. 21A: Schematic diagram of anti-CD19 CAR constructs with or without an IL-2Rβ chain STAT5 signaling domain. CAR constructs encoding a truncated cytoplasmic domain of IL-2Rβ together with co-stimulatory signaling domains show antigen-dependent JAK-STAT pathway activation (Kagoya et al., Nat Med. March; 24(3): 352-359 (2018). FIG. 21B: CD107a expression on NK-92MI cells transduced with indicated CARs in the presence or absence of target Raji cells. FIG. 21C: Specific cytotoxicity of NK-92MI cells transduced with indicated CARs to target Raji cells at 10:1 effector to target ratio. The mean percentages of specific target cell lysis±SD were shown. *=p<0.05.

FIGS. 22A-22B: Validation of chimeric antigen receptor (CAR) constructs to temporarily drive expression of a constitutively active form of STAT5. FIG. 22A: Schematic diagram of a synthetic Notch-based CAR system for inducible expression of a constitutively active form of STAT5b (STAT5bCA). Following EpCAM recognition by an anti-EpCAM synthetic Notch CAR results in cleavage and translocation of a TetRVP64 transcription factor complex to the nucleus to drive expression of STAT5bCA in innate lymphoid cells (ILCs). Red color denotes expression of CAR, and surface expression of a truncated form of EGF receptor (hEGFRt) denotes integration of the STAT5bCA construct. FIG. 22B: A 58α-β-cell line was transduced with retrovirus expressing the Myc-EpCAM-scFV-Notch-TetRVP64 and TetRE-Stat5bCA-mNeonGreen-PGK-hEGFRt constructs and co-cultured with 293T cells for 2 days. Engagement of EpCAM-scFV with EpCAM triggers cleavage of Notch intracellular domain, releasing TetVP64 to localize into the nucleus, transactivating transcription of Stat5bCA. mNeonGreen as a reporter of Stat5bCA expression was shown. These results demonstrate that induction of JAK-STAT activation can be successfully extrapolated to ILCs to achieve anti-tumor effects.

FIGS. 23A-23F: chRCC and ccRCC tumor exhibit differential immune cell infiltration and CD8⁺ T cell phenotypes. FIG. 23A: t-distributed Stochastic Neighbor Embedding (tSNE) embedding of transcriptional profiles from two renal cell carcinoma (RCC) patient tumors, one chromophobe RCC (chRCC) and one clear cell RCC (ccRCC). Each dot represents a single CD45+ cell, and colors represent clusters denoted by cell type inferred from lineage markers and differential gene expression. FIG. 23B: tSNE plot as in FIG. 23A, colored by histology (chromophobe or clear cell). FIG. 23C: For each CD8⁺ cluster, the frequency out of all CD8⁺ clusters at which it was found in chRCC and ccRCC tumors. FIG. 23D: Violin plots showing log-normalized expression of selected differentially expressed genes between the three CD8⁺ clusters. FIG. 23E: Representative plots of flow cytometric analysis of the percentage of CD3⁺CD8⁺ T cells out of the lymphocyte gate (CD45⁺SSC^{Low}) in blood, adjacent normal kidney and tumor samples from one patient of the indicated histology. Quantification is CD3⁺CD8⁺ T cells out of total CD45⁺ immune cells. FIG. 23F: Representative histograms of PD-1 expression in CD8⁺ T cells from blood, adjacent normal kidney, and tumor tissues from a single patient of the indicated histology. Quantification of flow cytometric analysis of percentage of total PD-1⁺ CD8⁺ T cells in blood, adjacent normal kidney, and tumor samples from the indicated histology. FIGS. 23E-23F: Each pair of symbols connected by a line denotes an individual patient (chRCC n=5-10, ccRCC n=14-16). One-way ANOVA with Tukey's multiple comparison test was used for statistical analysis, NS=non-significant, **p<0.01, ****p<0.0001.

FIGS. 24A-24D: chRCC tumors are highly infiltrated by CD56⁺CD49a⁺CD103⁺ ILC1s. FIG. 24A: Violin plots showing log-normalized expression of differentially expressed genes between the two innate lymphocyte clusters. FIG. 24B: For each innate lymphocyte (KLRB1⁺) cluster, the frequency out of all KLRB1⁺ clusters at which it was found in chromophobe renal cell carcinoma (chRCC) and clear cell RCC (ccRCC) tumors. FIG. 24C: Representative plots of flow cytometric analysis of percentage of CD3⁻CD56⁺ innate lymphocytes out of the lymphocyte gate (CD45⁺SSC^{Low}) in blood, adjacent normal kidney, and tumor samples from one patient of the indicated histology. Quantification is CD3⁻CD56⁺ innate lymphocytes out of total CD45⁺ immune cells. FIG. 24D: Representative plots and quantification of CD49a and CD103 expression in CD3-CD56⁺ innate lymphocytes from blood, adjacent normal kidney, and tumor tissue from one patient of the indicated histology. FIGS. 24C-24D: Each pair of symbols connected by a line denotes an individual patient (chRCC n=5-9, ccRCC n=14-15). One-way ANOVA with Tukey's multiple comparison test was used for statistical analysis. Two-tailed unpaired t test was used to analyze significance of CD49a⁺CD103⁺ abundance in chRCC versus ccRCC tumors. NS=non-significant, **p<0.01, ***p<0.001, ****p<0.0001.

FIGS. 25A-25D: High expression of the ILC1 signature predicts better survival of chRCC patients. FIG. 25A: Heatmap showing enrichment of the CD8_2 signature genes in the indicated clusters. FIG. 25B: Survival analysis demonstrating association of the CD8_2 signature across the TCGA clear cell renal cell carcinoma (ccRCC, top, 535 patients) and chromophobe RCC (chRCC, bottom, 66 patients) cohorts. FIG. 25C: Heatmap showing enrichment of the ILC1 signature genes in the indicated clusters. FIG. 25D: Survival analysis demonstrating association of the ILC1 signature across the TCGA ccRCC (top, 535 patients) and chRCC (bottom, 66 patients) cohorts. FIGS. 25B, 25D: High represents the top quartile of the distribution of signature scores, low represents the bottom 3 quartiles. Statistical p values calculated using a Cox regression and log-rank test.

FIGS. 26A-26J: ILC1s are induced in human and murine breast cancers in association with IL-15 expression in tumor. FIG. 26A: Survival analysis demonstrating association of the ILC1 signature across the TCGA BRCA (1102 patients) cohort. FIG. 26B: Survival analysis demonstrating association of the ILC1 signature across a subset of the TCGA BRCA patient cohort with hotspot activating mutations (p.H1047R, p.E545K, p.E542K) in PIK3CA (231 patients). FIGS. 26A-26B: High represents the top quartile of the distribution of signature scores, low represents the bottom 3 quartiles. Statistical p values calculated using a Cox regression and log-rank test. FIG. 26C: Correlation between level of IL-15 expression and ILC1 signature in a subset of the TCGA BRCA patient cohort with hotspot activating mutations (p.H1047R, p.E545K, p.E542K) in PIK3CA (231 patients). Statistical analyses calculated using Spearman's correlation. FIG. 26D: tSNE embedding of transcriptional profiles from pooled tumors of a PyMT mouse. Each dot represents a single CD3 NK1.1+ cell, and colors represent clusters denoted by cell type inferred from lineage markers and differential gene expression. FIG. 26E: GSEA of the list of DEGs between clusters mNK and mILC1 against the list of DEGs between human clusters NK and ILC1. FIGS. 26F-26G: Violin plots showing log-normalized expression of selected differentially expressed genes between the two CD3⁻NK1.1⁺ clusters. FIG. 26H: Representative plot of CD49a and CD103 expression in CD3⁻NK1.1⁺ innate lymphocytes with representative plots and quantification of granzyme B (GzmB) and granzyme C (GzmC) expression within the CD49a⁻CD103⁻, CD49a⁺CD103⁻, and CD49a⁺CD103⁺ subsets. Data are pooled from 3 or more independent experiments. FIG. 26I: Representative plots and quantification of CD3⁻NK1.1⁺ CD49a⁺CD103⁺ ILC1s in wild-type mammary glands (MG) and transformed PyMT tumors. Data are pooled from 3 or more independent experiments (MG n=4, tumor n=5). FIG. 26J: ELISA was performed using lysates prepared from pooled control mammary glands (MG) and transformed PyMT tumors. IL-15 quantity in ng was calculated for 1 mg of tissue. FIGS. 26H, 26I: All error bars represent the mean±SEM. One-way ANOVA with Tukey's multiple comparison test was used for statistical analysis, NS=non-significant, *p<0.05, **p<0.01. FIG. 26J: Error bars represent the mean±SEM. Two-tailed unpaired t test was used for statistical analysis, NS=non-significant, *p<0.05.

FIGS. 27A-27B: ILC1s expand in transformed tissue where they are stationary but active. FIG. 27A: Representative immunofluorescence images of E-cadherin^{CFP} (white), PyMT (green), GzmC^{tdT} (red), and DAPI (blue) from a tumor section of a 13-week old Gzmc^{tdT-T2A-iCre}CdhI^{mCFP}PyMT mouse. Scale bar, 150 μm. Green outline denotes transformed (PyMT⁺) area and white outline denotes non-transformed (PyMT⁻) area. Quantification is of total number of individual GzmC^{tdT}_positive cells in non-transformed or transformed areas, taken from tumor sections of two mice, each with 4-5 fields of view and a range of 1-8 distinct regions of non-transformed or transformed areas. Each dot represents discrete, individual E-Cadherin+ areas. Error bars represent the mean+SEM. Two-tailed unpaired t test was used for statistical analysis, ***p<0.001. FIG. 27B: Still images of live imaging time lapse of a mammary gland from a 13-week-old PyMT mouse with E-cadherin^{CFP} (teal), Ca²⁺ (green), and GzmC^{tdT} (red). Scale bar, 10 μm. Circles denote cells in which we observe a calcium flux, indicated by a yellow arrow.

FIGS. 28A-28F: Cancer cell-expressed IL-15 dictates ILC1 responses in tumor. FIG. 28A: Flow cytometric analysis of eGFP expression in CD24⁺CD29⁺EpCAM⁺ epithelial cells from mammary tissue of 20-week-old IL-15^{2a-eGFP}PyMT (green solid), IL-15^{2a-eGFP} (green empty), PyMT (gray filled), or control mouse (gray empty) (n=2 mice for each group). Numbers represent mean fluorescence intensity (MFI). Plot is representative of 2 independent experiments. Paired ratio t tests were used for statistical analysis, NS=non-significant, *p<0.05. FIG. 28B: Representative plot and quantification of percentage of NK1.1⁺ cells out of total CD3⁻ cells isolated from pooled tumors of 20-24-week-old IL-15^{fl/fl}PyMT (n=6) or Mrp8-CreIL-15^{fl/fl}PyMT (n=7) mice. FIG. 28C: Representative plot and quantification of percentage of CD49a⁺CD103⁺ ILC1s out of total CD45⁺CD3⁻NK1.1⁺ cells isolated from pooled tumors of 20-24-week-old IL-15^{fl/fl}PyMT (n=6) or Mrp8-Crell15^{fl/fl}PyMT mice (n=7). FIG. 28D: Representative histogram and quantification of granzyme B (GzmB) expression in CD49a⁺CD103⁺ ILCs from pooled tumors of 20-24-week-old IL-15^{fl/fl}PyMT (n=8) or Mrp8-CreIL-15^{fl/fl}PyMT (n=6) mice. FIG. 28E: Representative histogram and quantification of granzyme C (GzmC) expression in CD49a⁺CD103⁺ ILC1s from pooled tumors of 20-24-week-old IL-15^{fl/fl}PyMT (n=6) or Mrp8-CreIL-15^{fl/fl}PyMT (n=6) mice. FIG. 28F: Total tumor burden of IL-15^{fl/fl}PyMT and Mrp8-CreIL-15^{fl/fl}PyMT mice monitored between 11 and 20 weeks of age (n=11-17). Error bars represent the mean±SEM. Two-tailed unpaired t test was used for statistical analyses, *p<0.05. FIGS. 28B-28E: Data are pooled from 3 or more independent experiments. Each dot represents an individual mouse. All error bars represent the mean±SEM. Two-tailed unpaired t test was used for all statistical analyses, **p<0.01, ****p<0.0001.

FIGS. 29A-29G: IL-15 governs the cytolytic effector function of ILC1s in RCC. FIG. 29A: (Left) Representative histograms of granzyme A (GzmA) expression in CD49a⁻CD103⁻ NK cells (green) and CD49a⁺CD103⁺ILC1s (orange) from the same chromophobe renal cell carcinoma (chRCC, n=5) or clear cell RCC (ccRCC, n=14) patient. FMO=fluorescence minus one control. (Right) Mean fluorescence intensity (MFI) of GzmA expression in CD49a⁺CD103⁺ ILC1s and CD49a⁻CD103⁻ NK cells in tumor samples. FIG. 29B: (Left) Representative histograms of GzmA expression in CD49a⁺CD103⁺ ILC1s in adjacent normal kidney (black) and tumor (green) of the same patient of the indicated histology. (Right) MFI of GzmA in CD49a⁺CD103⁺ ILC1s in tumor (green) and adjacent normal kidney (black) of the same patient of the indicated histology. chRCC n=6, ccRCC n=14. FIG. 29C: (Left) CD49a⁺CD103⁺ ILC1s were sorted from ccRCC tumors and cultured in the indicated concentrations of IL-15/IL-15Rα complex and analyzed by flow cytometry for GzmA expression. Plot is representative of 4 independent experiments using cells isolated from 4 different ccRCC patients. (Right) GzmA MFI of CD49a⁺CD103⁺ ILCs cultured with 10 ng/ml or 100 ng/ml of IL-15/IL-15Rα complex. Each pair of symbols denotes cells isolated from an individual patient (n=4). FIG. 29D: CD49a⁺CD103⁺ ILC1s were sorted and cultured either with 10 ng/ml or 100 ng/ml of IL-15/IL-15Rα complex and subject to single-cell killing assays. (Left) Representative still images from the imaging time course. Times are in format of hour: minute. Blue cells are cell trace violet (CTV)-stained K562 target cells. Red arrows indicate CD49a⁺CD103⁺ ILC1 effector cells and white arrows indicate a dead (PI⁺) K562 target cell. (Right) The plot shows the percentage of wells with target cell killing events out of total wells containing both target and ILC1 effector cells over time. Plot is representative of 3 independent experiments using cells isolated from 3 different RCC patients. FIG. 29E: CD49a⁺CD103⁺ ILC1s were sorted from RCC tumors and cultured in the indicated concentrations of IL-15/IL-15Rα complex and analyzed by flow cytometry for Ki67 expression. Plot is representative of 4 independent experiments using cells isolated from 4 different patients. (Right) Percent Ki67⁺ CD49a⁺CD103⁺ ILC1s cultured with 100 ng/ml versus 10 ng/ml of IL-15/IL-15Rα complex. Each pair of symbols denotes cells isolated from an individual patient (n=4). FIG. 29F: Correlation between level of IL-15 expression and ILC1 signature in chRCC cases from the TCGA database. Statistical analyses calculated using Spearman's correlation. FIG. 29G: Association of IL-15 expression and overall survival across the TCGA chRCC cohort. High represents the top quartile and low represents the bottom 3 quartiles of IL-15 expression level. P values calculated using a Cox regression and log-rank test. FIGS. 29A-29B: Each pair of symbols connected by a line denotes an individual patient (chRCC n=6, ccRCC n=14). Paired ratio t test was used for statistical analysis. *p<0.05. FIG. 29C: Each pair of symbols represents cells isolated from the same individual patient (n=4). Paired ratio t test was used for statistical analysis, *p<0.05. FIG. 29E: Each pair of symbols represents cells isolated from the same individual patient (n=4). Two-tailed unpaired t test was used for statistical analysis, *p<0.05.

FIGS. 30A-30C: Cluster-defining marker plots for all clusters and heatmap of differential gene expression analysis among the three CD8⁺ T cell clusters. FIG. 30A: Marker plots showing normalized expression of selected common markers for lymphoid and myeloid populations (CD3D—T cells, CD4—CD4⁺ T cells, CD8A—CD8⁺ T cells, KLRB1—innate lymphocytes, CD14-monocytes and tumor-associated macrophages [TAM]). FIG. 30B: Heatmap of expression of the top 30 differentially expressed (FDR P<0.05) genes by log fold change across the three CD8⁺ T cell clusters within the chromophobe renal cell carcinoma (chRCC) and clear cell RCC (ccRCC) patients. Each column represents an individual cell. FIG. 30C: Plots showing the back gating strategy for plots shown in FIG. 23E.

FIGS. 31A-31C: Heatmap of differential gene expression analysis between clusters NK and ILC1 and comparison of CD56 expression between CD49a⁺CD103⁺ ILC1s and CD49a⁻CD103⁻ NK cells. FIG. 31A: Heatmap of expression of the top 30 differentially expressed (FDR P<0.05) genes by log fold change across the two innate lymphocyte clusters in the chromophobe renal cell carcinoma (chRCC) and clear cell RCC (ccRCC) patients. Each column represents an individual cell. FIG. 31B: Plots showing the back gating strategy for plots shown in FIG. 24C. FIG. 31C: CD56 MFI in CD49a⁺CD103⁺ ILC1s (orange) compared to CD49a⁻CD103⁻ NK cells (green) in the indicated histology. Each pair of symbols connected by a line denotes an individual patient. Paired ratio t test was used for statistical analysis, NS=non-significant, ****p<0.0001.

FIGS. 32A-32C: Validation of the ILC1 signature. FIG. 32A: Table outlining the cell surface markers used to define each immune cell population sorted for bulk RNA sequencing. FIG. 32B: Area under the receiver operating characteristic (ROC) curve and FIG. 32C: precision recall (PR) curve, when using the ILC1 signature to discriminate resident ILC1 populations (n=12) vs all others (n=57). The areas under the ROC and PR curves were calculated using the PRROC package in R.

FIGS. 33A-33E. CD49a⁺CD103⁺ ILC1s and CD49a⁺CD103-NK cells are phenotypically distinct in terms of NKG2A expression, but HLA-E expression does not track with ILC1 response in chRCC patients. FIG. 33A: Violin plot showing KLRC1 expression in the indicated clusters. FIG. 33B: (Left) Representative histograms of NKG2A expression in CD49a⁻CD103⁻ NK cells (green) and CD49a⁺CD103⁺ILC1s (orange) from the same patient of the indicated histology. (Center) Quantification of NKG2A⁺ cells within the indicated cell type and histology. (Right) MFI of NKG2A in NKG2A⁺CD49a⁺CD103⁺ ILC1s compared to NKG2A⁺CD49a⁻CD103⁻ NK cells in tumor samples. chRCC (n=6), ccRCC (n=9). Each pair of symbols connected by a line denotes an individual patient. Two-tailed unpaired t test was used for statistical analysis of the percent NKG2A positive, and paired ratio t test was used for statistical analysis of MFI, ***p<0.001, ****p<0.0001. FIG. 33C: Violin plot showing log-normalized expression of the HLA-E gene in the TCGA ccRCC and chRCC cohorts. Two-sided Wilcoxon test was used for statistical analysis ****p<0.0001. FIG. 33D: Correlation between level of HLA-E expression and ILC1 signature in chRCC cases from the TCGA database. Statistical analyses calculated using Spearman's correlation. FIG. 33E: Association of HLA-E expression and overall survival across the TCGA chRCC cohort. High represents the top quartile and low represents the bottom 3 quartiles of IL-15 expression level. P value calculated using a Cox regression and log-rank test.

FIGS. 34A-34D: IL2RB expression in clusters NK and ILC1, IL-15 expression in chRCC and ccRCC tumors from the TCGA database, IL-15-dependent regulation of CD56 expression in CD49a⁺CD103⁺ ILCs, and localization of ILC1s in chRCC tumor tissue. FIG. 34A: Violin plot showing log-normalized expression of the IL2RB gene in the indicated innate lymphocyte clusters. FIG. 34B: Violin plot showing level of IL-15 expression across chromophobe renal cell carcinoma (chRCC) and clear cell RCC (ccRCC) patients in the TCGA cohort. One-sided Wilcoxon test was used for statistical analysis, *p<0.05. FIG. 34C: (Left) Representative histograms of CD56 expression in CD49a⁺CD103⁺ ILC1s treated with the indicated concentration of IL-15/IL-15Rα complex. (Right) MFI of CD56 in CD49a⁺CD103⁺ innate lymphocytes isolated from tumors treated with 100 ng/ml IL-15/IL-15Rα complex compared to 10 ng/ml IL-15/IL-15Rα complex. Each pair of symbols connected by a line denotes cells isolated from an individual patient (n=5, 1 chRCC and 4 ccRCC), in 5 independent experiments. Paired ratio t test was used for statistical analysis, *p<0.05. FIG. 34D: chRCC tumor tissue was stained with anti-CD103 (red), anti-E-Cadherin (white), anti-CD3 (green), and DAPI (blue). White arrows denote CD3 CD103⁺ ILC1s. Scale bar=20 μM. Quantification is representative of three chRCC patient tumor tissues. Error bar represents mean±SEM.

FIGS. 35A-350: ILCs function independently of dendritic cell and macrophage sources of IL-15. FIG. 35A: Schematic describing the IL-15^{2a-eGFP} reporter mouse strain. FIG. 35B: Types of cancer cells and stromal cells with the potential for IL-15 expression in PyMT tumors. FIG. 35C: Table listing the Cre recombinase lines used to delete IL-15 in the listed target cell populations. FIG. 35D: Schematic of an IL-15 floxed allele. FIG. 35E: Gating strategy for determining eGFP expression in the indicated myeloid cell populations isolated from pooled tumors of a 20-week-old IL-15^{2a-eGFP}PyMT mouse. FIG. 35F: Flow cytometric analysis of eGFP expression in the indicated myeloid cell populations from pooled tumors of a 20-week-old IL-15^{2a-eGFP}PyMT (colored) or PyMT mouse (gray). FIG. 35G: qPCR analysis of IL-15 mRNA expression in sorted dendritic cells (DC) from pooled tumors of 20-24-week-old IL-15^{fl/fl}PyMT or CD11c-CreIL-15^{fl/fl}PyMT mice. FIG. 35H: qPCR analysis of IL-15 mRNA expression in sorted tumor-associated macrophages (TAM) from pooled tumors of 20-24-week-old IL-15^{fl/fl}PyMT or CD11c-CreIL-15^{fl/fl}PyMT mice. FIG. 35I: Representative plot and quantification of NK1.1⁺ cells out of total CD45⁺CD3⁻ cells isolated from pooled tumors of 20-24-week-old IL-15^{fl/fl}PyMT or CD11c-CreIL-15^{fl/fl}PyMT mice. FIG. 35J: Representative plot and quantification of percentage of CD49a⁺CD103⁺ ILC1s out of total CD45⁺CD3⁻NK1.1⁺ cells isolated from pooled tumors of 20-24-week-old IL-15^{fl/fl}PyMT or CD11c-CreIL-15^{fl/fl}PyMT mice. FIG. 35K: Representative histogram and quantification of granzyme B (GzmB) expression in CD49a⁺CD103⁺ ILC1s from pooled tumors of 20-24-week-old IL-15^{fl/fl}PyMT or CD11c-CreIL-15^{fl/fl}PyMT mice. FIG. 35L: (Left) Representative histogram and quantification of granzyme C (GzmC) expression in CD49a⁺CD103⁺ ILC1s from pooled tumors of 20-24-week-old IL-15^{fl/fl}PyMT or CD11c-CreIL-15^{fl/fl}PyMT mice. FIG. 35M: Total tumor burden of IL-15^{fl/fl}PyMT and CD11c-CreIL-15^{fl/fl}PyMT mice monitored between 11 and 20 weeks of age (n=6-13). FIG. 35N: Representative histograms of CD49b (DX5) expression among total CD3⁻NK1.1⁺ cells in spleens of the indicated mouse genotype. (Right) Percentage of DX5⁺ NK cells quantified out of total splenic CD45⁺ immune cells. FIG. 35O: (Left) Representative plots of CD27 and CD11b expression among total CD3⁻NK1.1⁺DX5⁺ NK cells in spleens of the indicated mouse genotype. (Right) Percentage of DX5⁺CD11b⁺CD27⁻ cells quantified out of total splenic CD45+ cells. FIGS. 35G-35O: Each dot represents an individual mouse. Data are pooled from 3 or more independent experiments. All error bars represent the mean±SEM. Two-tailed unpaired t test was used for statistical analysis, NS=non-significant, *p<0.05, **p<0.01, ***p<0.001.

FIGS. 36A-36K: ILC1s function independently of hematopoietic and stromal cell sources of IL-15. FIG. 36A: Gating strategy and YFP expression in the indicated populations from pooled tumors of a 20-week-old FSP1-CreRosa26^{LSL-YFP}PyMT mouse. FIG. 36B: Flow cytometric analysis of eGFP expression in the indicated populations from pooled tumors of 20-week-old IL-15^{2a-eGFP}PyMT (colored) or control PyMT (gray) mice. FIG. 36C: qPCR analysis of IL-15 mRNA expression in CD45⁺ immune cells isolated from 20-24-week-old IL-15^{fl/fl}PyMT or FSP1-CreIL-15^{fl/fl}PyMT mice. FIG. 36D: qPCR analysis of IL-15 mRNA expression in CD45⁻CD31⁻Ter119⁻CD24⁻ CD29⁺EpCAM-stromal cells isolated from 20-24-week-old IL-15^{fl/fl}PyMT or FSP1-CreIL-15^{fl/fl}PyMT mice. FIG. 36E: Representative plot and quantification of percentage of NK1.1⁺ cells out of total CD3⁻ cells isolated from pooled tumors of 20-24-week-old IL-15^{fl/fl}PyMT or FSP1-CreIL-15^{fl/fl}PyMT mice. FIG. 36F: Representative plot and quantification of percentage of CD49a⁺CD103⁺ ILC1s out of total CD45⁺CD3⁻NK1.1⁺ cells isolated from pooled tumors of 20-24-week-old IL-15^{fl/fl}PyMT or FSP1-Crell15^{fl/fl}PyMT mice. FIG. 36G: Representative histogram and quantification of granzyme B (GzmB) expression in CD49a⁺CD103⁺ ILC1s isolated from pooled tumors of 20-24-week-old IL-15^{fl/fl}PyMT or FSP1-Crell15^{fl/fl}PyMT mice. FIG. 36H: Representative histogram and quantification of granzyme C (GzmC) expression in CD49a⁺CD103⁺ ILC1s isolated from pooled tumors of 20-24-week-old IL-15^{fl/fl}PyMT or FSP1-Crell15^{fl/fl}PyMT mice. FIG. 36I: Total tumor burden of IL-15^{fl/fl}PyMT and FSP1-CreIL-15^{fl/fl}PyMT mice monitored between 11 and 20 weeks of age (n=5-9). FIG. 36J: Representative histograms of CD49b (DX5) expression among total CD3⁻NK1.1⁺ cells in spleens of the indicated mouse genotype. (Right) Percentage of DX5⁺ NK cells quantified out of total splenic CD45⁺ cells. FIG. 36K: (Left) Representative plots of CD27 and CD11b expression among total CD3⁻ NK1.1⁺DX5⁺ NK cells in spleens of the indicated mouse genotype. (Right) Percentage of DX5⁺CD11b⁺CD27 cells quantified out of total splenic CD45⁺ cells. FIGS. 36C-36K: Each dot represents an individual mouse. Data are pooled from 3 or more independent experiments. All error bars represent the mean±SEM. Two-tailed unpaired t test was used for statistical analysis, NS=non-significant, **p<0.01, ***p<0.001, ****p<0.0001.

FIGS. 37A-37E. Mouse models utilized for characterization of tissue-resident ILC1 responses in PyMT tumors. FIG. 37A: Diagram denoting the Gzmc gene locus of Gzmc^{tdT-T2A-iCre} mice. FIG. 37B: Flow cytometric analysis of GzmC^{tdT} reporter expression and granzyme C protein expression among the indicated CD3 NK1.1⁺ innate lymphocyte populations in PyMT tumors. FIG. 37C: Diagram denoting the Cdh1 gene locus of Cdh1^{mCFP} mice. FIG. 37D: Diagram denoting the Polr2a gene locus harboring an expression cassette for a GCaMP5 calcium indicator and a tdT reporter. FIG. 37E: Expected fluorescent phenotype that results when calcium signaling is sensed in GCaMP5-expressing granzyme C-expressing ILC1s.

FIGS. 38A-38E: Mrp8-Cre targets cancer cells, and splenic NK cells are unaffected in Mrp8-CreIL-150^{fl/fl}PyMT mice. FIG. 38A: Gating strategy for determining eGFP expression in CD24⁺CD29 EpCAM⁺ epithelial cells from non-reporter mammary gland, IL-15^{2a-eGFP} mammary gland, PyMT tumors, and IL-15^{2a-eGFP}PyMT tumors. FIG. 38B: Gating strategy and YFP expression in CD24⁺CD29⁺ cancer cells from pooled tumors of a 20-week-old Mrp8-CreRosa26^{LSL-YFP}PyMT mouse. FIG. 38C: qPCR analysis of IL-15 mRNA expression in CD45⁻CD24⁺CD29⁺EpCAM⁺ cancer cells isolated from 20-24-week-old IL-15^{fl/fl}PyMT or Mrp8-CreIL-15^{fl/fl}PyMT mice. FIG. 38D: (Left) Representative histograms of CD49b (DX5) expression among total CD3⁻NK1.1⁺ cells in spleens of the indicated mouse genotype. (Right) Percentage of DX5⁺ NK cells quantified out of total splenic CD45⁺ cells. FIG. 38E: (Left) Representative plots of CD27 and CD11b expression among total CD3⁻NK1.1⁺DX5⁺ cells in spleens of the indicated mouse genotype. (Right) Percentage of DX5⁺CD11b⁺CD27⁻ cells quantified out of total splenic CD45⁺ cells. FIGS. 38C-38E: Each dot represents an individual mouse (n=3-4). Data are pooled from 3 independent experiments. All error bars represent the mean±SEM. Two-tailed unpaired t test was used for statistical analysis, NS=non-significant, ****p<0.0001.

FIGS. 39A-39C: Human type 1 innate lymphoid cells armed with IL-2Rβ chain chimeric antigen receptor (CAR) exhibit enhanced cytotoxicity towards CD19-expressing leukemia cells. FIG. 39A: A schematic diagram of anti-CD19 CAR constructs with or without an IL-2Rβ chain signaling domain that drives the IL-15-induced signaling. FIG. 39B: CD107a expression as a marker of degranulation on a human type 1 innate lymphoid cell line NK-92MI transduced with the indicated CARs in the absence or presence of target Raji leukemia cells. FIG. 39C: Specific cytotoxicity of NK-92MI cells transduced with the indicated CARs to target Raji cells at a 5:1 effector to target ratio. The mean percentages of specific target cell lysis+SD were shown. *P<0.05.

FIGS. 40A-40C: Human type 1 innate lymphoid cells armed with IL-2Rβ chain chimeric antigen receptor (CAR) exhibit enhanced cytotoxicity towards GPC3-expressing hepatocellular carcinoma cells. FIG. 40A: A schematic diagram of anti-GPC3 CAR constructs with or without an IL-2Rβ chain signaling domain that drives the IL-15-induced signaling. FIG. 40B: CD107a expression as a marker of degranulation on a human type 1 innate lymphoid cell line NK-92MI transduced with the indicated CARs in the absence or presence of target HepG2 hepatocellular carcinoma cells. FIG. 40C: Specific cytotoxicity of NK-92MI cells transduced with the indicated CARs to target HepG2 cells at a 5:1 effector to target ratio. The mean percentages of specific target cell lysis±SD were shown. *P<0.05.

FIGS. 41A-41C: Human type 1 innate lymphoid cells armed with IL-2Rβ chain chimeric antigen receptor (CAR) exhibit enhanced cytotoxicity towards HER2-expressing breast cancer cells. FIG. 41A: A schematic diagram of anti-HER2 CAR constructs with or without an IL-2Rβ chain signaling domain that drives the IL-15-induced signaling. FIG. 41B: CD107a expression as a marker of degranulation on a human type 1 innate lymphoid cell line NK-92MI transduced with the indicated CARs in the absence or presence of target HER2-overexpressing K562 (K562-HER2) breast cancer cells. FIG. 41C: Specific cytotoxicity of NK-92MI cells transduced with the indicated CARs to target K562-HER2 cells at a 5:1 effector to target ratio. The mean percentages of specific target cell lysis±SD were shown. *P<0.05.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

While conventional T cell-based adoptive cellular transfer therapy has seen tremendous success in treating liquid cancers, its therapeutic use in solid cancers was hampered in part by conventional CD8⁺ T cell's propensity to exhaustion as well as poor tissue infiltration and retention. Additionally, conventional T cell-based adoptive transfer therapies require a priori knowledge of the target antigen, rendering the approach highly personalized and time-consuming.

The present disclosure demonstrates that while clear cell renal cell carcinoma (ccRCC) was infiltrated by exhaustion phenotype CD8⁺ T cells which negatively correlated with patient prognosis, chromophobe RCC had abundant infiltration of granzyme A-expressing tissue-resident innate lymphocytes that positively associated with patient survival. Notably, interleukin-15 (IL-15) promoted granzyme A expression and natural killer (NK) cell-like cytotoxicity, with IL-15 expressed by tumor tissue controlling the magnitude of the innate lymphocyte response. These findings demonstrate that induction of tissue resident cytotoxic innate lymphocytes represents an evolutionarily conserved anti-tumor immune response with its immunosurveillance outcome tuned towards the sensing of cancer cell expressed IL-15. The present disclosure demonstrates that ILC-based cellular transfer therapy is an appealing alternative to conventional T cell-based adoptive transfer therapies.

### Definitions

As it would be understood, the section or subsection headings as used herein is for organizational purposes only and are not to be construed as limiting and/or separating the subject matter described.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1.0 or 0.1, as appropriate or alternatively by a variation of +/−20% or +/−15%, or alternatively 10% or alternatively 5% or alternatively 2%. As will be understood by one skilled in the art, for any and all purposes, all ranges disclosed herein also encompass any and all possible subranges and combinations of subranges thereof. Furthermore, as will be understood by one skilled in the art, a range includes each individual member.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another. “Administration” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

As used herein “adoptive cell therapeutic composition” refers to any composition comprising cells suitable for adoptive cell transfer. In exemplary embodiments, the adoptive cell therapeutic composition comprises cytotoxic innate lymphoid cells (ILCs), genetically engineered cytotoxic ILCs (e.g., comprising non-endogenous expression vectors encoding IL-15 or STAT5B), CAR (i.e. chimeric antigen receptor) modified cytotoxic ILCs (e.g., CAR ILCs). In one embodiment, the adoptive cell therapeutic composition comprises cytotoxic ILCs.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system. As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen. It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and CHI domains; a Fv fragment consisting of the V_{L} and V_{H} domains of a single arm of an antibody; a dAb fragment (Ward et al., Nature 341:544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85:5879-5883 (1988). These antibody fragments are obtained using conventional techniques known to those of ordinary skill in the art, and the fragments are screened for utility in the same manner as are intact antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having GGGGSGGGGSGGGGS (SEQ ID NO: 37). In certain embodiments, the nucleic acid sequence encoding the amino acid sequence of SEQ ID NO: 37 is ggcggcggcggatctggaggtggtggctcaggtggcggaggctcc (SEQ ID NO: 38).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife eta., J Clin Invst 116(8):2252-61 (2006); Brocks et al., Immunotechnology 3(3):173-84 (1997); Moosmayer et al., Ther Immunol 2(10):31-40 (1995). Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed in a cancer cell or tumor cell (such as on the cell surface) or tissue, and the term “cancer or tumor targeting antibody” refers to an antibody that targets such an antigen. In some embodiments, the cancer or tumor antigen is not expressed in a non-cancer cell or tissue. In some embodiments, the cancer or tumor antigen is expressed in a non-cancer cell or tissue at a level significantly lower compared to a cancer cell or tissue. In some embodiments, the cancer is a solid tumor. In other embodiments, the cancer is not a solid tumor. In some embodiments, the cancer is from a carcinoma, a sarcoma, a myeloma, a leukemia, or a lymphoma. In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

In some embodiments, the cancer is selected from: circulatory system, for example, heart (sarcoma [angiosarcoma, fibrosarcoma, rhabdomyosarcoma, liposarcoma], myxoma, rhabdomyoma, fibroma, and lipoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), placenta, vagina (clear cell carcinoma, squamous cell carcinoma, botryoid sarcoma (embryonal rhabdomyosarcoma), fallopian tubes (carcinoma) and other sites associated with female genital organs; penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Kaposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. In some embodiments, the cancer is a colon cancer, colorectal cancer or rectal cancer. In some embodiments, the cancer is a lung cancer. In some embodiments, the cancer is a pancreatic cancer. In some embodiments, the cancer is an adenocarcinoma, an adenocarcinoma, an adenoma, a leukemia, a lymphoma, a carcinoma, a melanoma, an angiosarcoma, or a seminoma.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “chimeric co-stimulatory receptor” or “CCR” refers to a chimeric receptor that binds to an antigen and provides co-stimulatory signals, but does not provide a T-cell activation signal.

As used herein, a “cleavable peptide”, which is also referred to as a “cleavable linker,” means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. Sci Rep. 2015; 5:16273. Published 2015 Nov. 5. As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 39), wherein X refers to any amino acid generally thought to be self-cleaving.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “composition” is intended to mean a combination of active agent and another compound or composition, inert (for example, a nanoparticle, detectable agent or label) or active, such as an adjuvant, diluent, binder, stabilizer, buffers, salts, lipophilic solvents, preservative, adjuvant or the like and include carriers, such as pharmaceutically acceptable carriers. In some embodiments, the carrier (such as the pharmaceutically acceptable carrier) comprises, or consists essentially of, or yet further consists of a nanoparticle, such as an polymeric nanoparticle carrier or an lipid nanoparticle that can be used alone or in combination with another carrier, such as an adjuvant or solvent. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol. A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include 2B4, CD27, CD28, 4-1BB (CD137), OX40 (CD134), CD30, CD40, ICOS (CD278), LFA-1, CD2, CD7, LIGHT, NKG2C, NKG2D, B7-H2 and a ligand that specifically binds CD83. Accordingly, while the present disclosure provides exemplary co-stimulatory domains derived from CD28 and 4-1BB, other co-stimulatory domains are contemplated for use with the CARs described herein. The intracellular signaling and co-stimulatory signaling domains can be linked in any order in tandem to the carboxyl terminus of the transmembrane domain.

As used herein, the phrase “derived” means isolated, purified, mutated, or engineered, or any combination thereof. For example, an ILC derived from a donor refers to the ILC isolated from a biological sample of the donor and optionally engineered.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “heterologous nucleic acid molecule or polypeptide” refers to a nucleic acid molecule (e.g., a cDNA, DNA or RNA molecule) or polypeptide that is either not normally expressed or is expressed at an aberrant level in a cell or sample obtained from a cell. This nucleic acid can be from another organism, or it can be, for example, an mRNA molecule that is not normally expressed in a cell or sample.

As used herein, a “host cell” is a cell that is used to receive, maintain, reproduce and amplify an expression vector. A host cell also can be used to express the polypeptide encoded by the expression vector. The nucleic acid contained in the expression vector is replicated when the host cell divides, thereby amplifying the nucleic acids. In some embodiments, the host cell as disclosed herein is a eukaryotic cell or a prokaryotic cell. In some embodiments, the host cell is a human cell. In some embodiments, the host cell is a cell line, such as a human embryonic kidney 293 cell (HEK 293 cell or 293 cell), or a 293T cell. These cells are commercially available, for example, from the American Type Culture Collection (ATCC).

As used herein “cytotoxic ILC/ILC1” or “cytotoxic innate lymphoid cells” refer to innate lymphocytes that are derived from innate lymphoid cell (ILC) progenitors, express the integrin molecule ITGA1 (CD49a), the IL-2/IL-15 receptor beta chain IL2RB (CD122), and the transcription factor ZNF683 (HOBIT), exhibit epithelial tissue-residency properties, and can exert lytic granule-mediated cytotoxicity against cancer cells. Cytotoxic ILCs are distinct from circulating NK cells, ILC2 or ILC3 subsets. Innate lymphocytes are characterized by their lack of functionally re-arranged antigen receptors. See Chou & Li, Front. Immunol., 9 (2018). As used herein, the term “engineered cytotoxic ILC” refers to a cytotoxic ILC that is genetically modified. As used herein, the term “native cytotoxic ILC” refers to a cytotoxic ILC that naturally occurs in the immune system.

As used herein, the term “linker” refers to any amino acid sequence comprising from a total of 1 to 200 amino acid residues; or about 1 to 10 amino acid residues, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (G4S)n (SEQ ID NO: 40), wherein n is 1, or 2, or 3, or 4, or 5, or 6, or 7, or 8, or 9, or 10, or 11, or 12, or 13, or 14, or 15.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the “percent homology” between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

“Pharmaceutically acceptable carriers” refers to any diluents, excipients, or carriers that may be used in the compositions disclosed herein. In some embodiments, a pharmaceutically acceptable carrier comprises, or consists essentially of, or yet further consists of a nanoparticle, such as an polymeric nanoparticle carrier or an lipid nanoparticle (LNP). Additionally or alternatively, pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They can be selected with respect to the intended form of administration, that is, oral tablets, capsules, elixirs, syrups and the like, and consistent with conventional pharmaceutical practices.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, “regulatory sequence” or “regulatory region” or “expression control sequence” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Polymerase II and III are examples of promoters. A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors. In some embodiments, the promoter is a constitutive promoter. As used herein, the term “constitutive promoter” refers to a promoter that allows for continual transcription of the coding sequence or gene under its control in all or most tissues of a subject at all or most developing stages. Non-limiting examples of the constitutive promoters include a CMV promoter, a simian virus 40 (SV40) promoter, a polyubiquitin C (UBC) promoter, an EF1-alpha promoter, a PGK promoter and a CAG promoter. In some embodiments, the promoter is a conditional promoter, which allows for continual transcription of the coding sequence or gene under certain conditions. In further embodiments, the conditional promoter is an immune cell specific promoter, which allows for continual transcription of the coding sequence or gene in an immune cell. Non-limiting examples of the immune cell specific promoters include a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, a “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

### IL-15

As used herein, the terms “interleukin-15,” “IL-15,” “interleukin 15” and “IL-5” refer to a cytokine that regulates T cell, natural killer cell activation and innate lymphoid cell proliferation. The encoded protein induces the activation of JAK kinases, as well as the phosphorylation and activation of transcription activator STAT5B, and mTORC1 signaling. Studies of the mouse counterpart suggested that this cytokine may increase the expression of apoptosis inhibitor BCL2L1/BCL-x (L), possibly through the transcription activation activity of STAT5B, and thus prevent apoptosis. Non-limiting exemplary sequences of this protein or the underlying gene may be found under NCBI Entrez Gene: 3600 (retrieved from www.ncbi.nlm.nih.gov/gene/3600), or UniProtKB/Swiss-Prot: P40933 (retrieved from www.uniprot.org/uniprot/P40933), which are incorporated by reference herein.

Exemplary amino acid sequences of IL-15 are set forth below:

>NP_000576.1 interleukin-15 isoform 1 pre-
proprotein [Homo sapiens]
(SEQ ID NO: 19)
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS;
or
>NP_751915.1 interleukin-15 isoform 2 pre-
proprotein [Homo sapiens]
(SEQ ID NO: 20)
MVLGTIDLCSCFSAGLPKTEANWVNVISDLKKIEDLIQSMHIDATLYTE
SDVHPSCKVTAMKCFLLELQVISLESGDASIHDTVENLIILANNSLSSN
GNVTESGCKECEELEEKNIKEFLQSFVHIVQMFINTS

In some embodiments, the engineered cytotoxic ILCs express a heterologous amino acid sequence that is at least 80%, 85%, 90%, 95%, 96%, 97%, 98%, or 99% identical to SEQ ID NO: 19, SEQ ID NO: 20, or a biological equivalent thereof. In further embodiments, the biological equivalent of SEQ ID NO: 19 or SEQ ID NO: 20 comprises one or more conservative amino acid substitutions relative to SEQ ID NO: 19 or SEQ ID NO: 20, respectively. Additionally or alternatively, in some embodiments, the cytokine function of the biological equivalent is substantially similar to or is significantly more efficient compared to the protein of SEQ ID NO: 19 or SEQ ID NO: 20.

Exemplary nucleic acid sequences of human IL-15 are set forth below:

>NM_000585.5 Homo sapiens interleukin 15 (IL15), transcript variant 3, mRNA
(SEQ ID NO: 21)
CTTTTCGCCAGGGGTTGGGACTCCGGGTGGCAGGCGCCCGGGGGAATCCCAGCTGACTCGCTCACTGCCT
TCGAAGTCCGGCGCCCCCCGGGAGGGAACTGGGTGGCCGCACCCTCCCGGCTGCGGTGGCTGTCGCCCCC
CACCCTGCAGCCAGGACTCGATGGAGAATCCATTCCAATATATGGCCATGTGGCTCTTTGGAGCAATGTT
CCATCATGTTCCATGCTGCTGACGTCACATGGAGCACAGAAATCAATGTTAGCAGATAGCCAGCCCATAC
AAGATCGTATTGTATTGTAGGAGGCATTGTGGATGGATGGCTGCTGGAAACCCCTTGCCATAGCCAGCTC
TTCTTCAATACTTAAGGATTTACCGTGGCTTTGAGTAATGAGAATTTCGAAACCACATTTGAGAAGTATT
TCCATCCAGTGCTACTTGTGTTTACTTCTAAACAGTCATTTTCTAACTGAAGCTGGCATTCATGTCTTCA
TTTTGGGCTGTTTCAGTGCAGGGCTTCCTAAAACAGAAGCCAACTGGGTGAATGTAATAAGTGATTTGAA
AAAAATTGAAGATCTTATTCAATCTATGCATATTGATGCTACTTTATATACGGAAAGTGATGTTCACCCC
AGTTGCAAAGTAACAGCAATGAAGTGCTTTCTCTTGGAGTTACAAGTTATTTCACTTGAGTCCGGAGATG
CAAGTATTCATGATACAGTAGAAAATCTGATCATCCTAGCAAACAACAGTTTGTCTTCTAATGGGAATGT
AACAGAATCTGGATGCAAAGAATGTGAGGAACTGGAGGAAAAAAATATTAAAGAATTTTTGCAGAGTTTT
GTACATATTGTCCAAATGTTCATCAACACTTCTTGATTGCAATTGATTCTTTTTAAAGTGTTTCTGTTAT
TAACAAACATCACTCTGCTGCTTAGACATAACAAAACACTCGGCATTTCAAATGTGCTGTCAAAACAAGT
TTTTCTGTCAAGAAGATGATCAGACCTTGGATCAGATGAACTCTTAGAAATGAAGGCAGAAAAATGTCAT
TGAGTAATATAGTGACTATGAACTTCTCTCAGACTTACTTTACTCATTTTTTTAATTTATTATTGAAATT
GTACATATTTGTGGAATAATGTAAAATGTTGAATAAAAATATGTACAAGTGTTGTTTTTTAAGTTGCACT
GATATTTTACCTCTTATTGCAAAATAGCATTTGTTTAAGGGTGATAGTCAAATTATGTATTGGTGGGGCT
GGGTACCAATGCTGCAGGTCAACAGCTATGCTGGTAGGCTCCTGCCAGTGTGGAACCACTGACTACTGGC
TCTCATTGACTTCCTTACTAAGCATAGCAAACAGAGGAAGAATTTGTTATCAGTAAGAAAAAGAAGAACT
ATATGTGAATCCTCTTCTTTATACTGTAATTTAGTTATTGATGTATAAAGCAACTGTTATGAAATAAAGA
AATTGCAATAACTGGCATATAATGTCCATCAGTAAATCTTGGTGGTGGTGGCAATAATAAACTTCTACTG
ATAGGTAGAATGGTGTGCAAGCTTGTCCAATCACGGATTGCAGGCCACATGCGGCCCAGGACAACTTTGA
ATGTGGCCCAACACAAATTCATAAACTTTCATACATCTCGTTTTTAGCTCATCAGCTATCATTAGCGGTA
GTGTATTTAAAGTGTGGCCCAAGACAATTCTTCTTATTCCAATGTGGCCCAGGGAAATCAAAAGATTGGA
TGCCCCTGGTATAGAAAACTAATAGTGACAGTGTTCATATTTCATGCTTTCCCAAATACAGGTATTTTAT
TTTCACATTCTTTTTGCCATGTTTATATAATAATAAAGAAAAACCCTGTTGATTTGTTGGAGCCATTGTT
ATCTGACAGAAAATAATTGTTTATATTTTTTGCACTACACTGTCTAAAATTAGCAAGCTCTCTTCTAATG
GAACTGTAAGAAAGATGAAATATTTTTGTTTTATTATAAATTTATTTCACCTTAA
>NM_172175.3 Homo sapiens interleukin 15 (IL15), transcript variant 2, mRNA
(SEQ ID NO: 22)
CTTTTCGCCAGGGGTTGGGACTCCGGGTGGCAGGCGCCCGGGGGAATCCCAGCTGACTCGCTCACTGCCT
TCGAAGTCCGGCGCCCCCCGGGAGGGAACTGGGTGGCCGCACCCTCCCGGCTGCGGTGGCTGTCGCCCCC
CACCCTGCAGCCAGGACTCGATGGAGAATCCATTCCAATATATGGCCATGTGGCTCTTTGGAGCAATGTT
CCATCATGTTCCATGCTGCTGACGTCACATGGAGCACAGAAATCAATGTTAGCAGATAGCCAGCCCATAC
AAGATCGTTTTCAACTAGTGGCCCCACTGTGTCCGGAATTGATGGGTTCTTGGTCTCACTGACTTCAAGA
ATGAAGCCGCGGACCCTCGCGGTGAGTGTTACAGCTCTTAAGGTGGCGCATCTGGAGTTTGTTCCTTCTG
ATGTTCGGATGTGTTCGGAGTTTCTTCCTTCTGGTGGGTTCGTGGTCTCGCTGGCTCAGGAGTGAAGCTA
CAGACCTTCGCGGAGGCATTGTGGATGGATGGCTGCTGGAAACCCCTTGCCATAGCCAGCTCTTCTTCAA
TACTTAAGGATTTACCGTGGCTTTGAGTAATGAGAATTTCGAAACCACATTTGAGAAGTATTTCCATCCA
GTGCTACTTGTGTTTACTTCTAAACAGTCATTTTCTAACTGAAGCTGGCATTCATGTCTTCATTTTGGGA
TGCAGCTAATATACCCAGTTGGCCCAAAGCACCTAACCTATAGTTATATAATCTGACTCTCAGTTCAGTT
TTACTCTACTAATGCCTTCATGGTATTGGGAACCATAGATTTGTGCAGCTGTTTCAGTGCAGGGCTTCCT
AAAACAGAAGCCAACTGGGTGAATGTAATAAGTGATTTGAAAAAAATTGAAGATCTTATTCAATCTATGC
ATATTGATGCTACTTTATATACGGAAAGTGATGTTCACCCCAGTTGCAAAGTAACAGCAATGAAGTGCTT
TCTCTTGGAGTTACAAGTTATTTCACTTGAGTCCGGAGATGCAAGTATTCATGATACAGTAGAAAATCTG
ATCATCCTAGCAAACAACAGTTTGTCTTCTAATGGGAATGTAACAGAATCTGGATGCAAAGAATGTGAGG
AACTGGAGGAAAAAAATATTAAAGAATTTTTGCAGAGTTTTGTACATATTGTCCAAATGTTCATCAACAC
TTCTTGATTGCAATTGATTCTTTTTAAAGTGTTTCTGTTATTAACAAACATCACTCTGCTGCTTAGACAT
AACAAAACACTCGGCATTTCAAATGTGCTGTCAAAACAAGTTTTTCTGTCAAGAAGATGATCAGACCTTG
GATCAGATGAACTCTTAGAAATGAAGGCAGAAAAATGTCATTGAGTAATATAGTGACTATGAACTTCTCT
CAGACTTACTTTACTCATTTTTTTAATTTATTATTGAAATTGTACATATTTGTGGAATAATGTAAAATGT
TGAATAAAAATATGTACAAGTGTTGTTTTTTAAGTTGCACTGATATTTTACCTCTTATTGCAAAATAGCA
TTTGTTTAAGGGTGATAGTCAAATTATGTATTGGTGGGGCTGGGTACCAATGCTGCAGGTCAACAGCTAT
GCTGGTAGGCTCCTGCCAGTGTGGAACCACTGACTACTGGCTCTCATTGACTTCCTTACTAAGCATAGCA
AACAGAGGAAGAATTTGTTATCAGTAAGAAAAAGAAGAACTATATGTGAATCCTCTTCTTTATACTGTAA
TTTAGTTATTGATGTATAAAGCAACTGTTATGAAATAAAGAAATTGCAATAACTGGCATATAATGTCCAT
CAGTAAATCTTGGTGGTGGTGGCAATAATAAACTTCTACTGATAGGTAGAATGGTGTGCAAGCTTGTCCA
ATCACGGATTGCAGGCCACATGCGGCCCAGGACAACTTTGAATGTGGCCCAACACAAATTCATAAACTTT
CATACATCTCGTTTTTAGCTCATCAGCTATCATTAGCGGTAGTGTATTTAAAGTGTGGCCCAAGACAATT
CTTCTTATTCCAATGTGGCCCAGGGAAATCAAAAGATTGGATGCCCCTGGTATAGAAAACTAATAGTGAC
AGTGTTCATATTTCATGCTTTCCCAAATACAGGTATTTTATTTTCACATTCTTTTTGCCATGTTTATATA
ATAATAAAGAAAAACCCTGTTGATTTGTTGGAGCCATTGTTATCTGACAGAAAATAATTGTTTATATTTT
TTGCACTACACTGTCTAAAATTAGCAAGCTCTCTTCTAATGGAACTGTAAGAAAGATGAAATATTTTTGT
TTTATTATAAATTTATTTCACCTTAA

In some embodiments, the engineered cytotoxic ILCs comprise a heterologous nucleic acid sequence that is at least 80%, 85%, 90%, 95%, 96%, 97%, 98%, or 99% identical to SEQ ID NO: 21, or SEQ ID NO: 22. Additionally or alternatively, in some embodiments, the expression levels and/or activity of IL-15 in the engineered cytotoxic ILC is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native cytotoxic ILC.

In some embodiments, the engineered cytotoxic ILC further comprises a first regulatory sequence operably linked to the nucleic acid encoding the IL-15. In further embodiments, the first regulatory sequence directs the expression of the IL-15. Additionally or alternatively, in some embodiments, the first regulatory sequence comprises, or consists essentially of, or yet further consists of a promoter, for example a constitutive promoter or a conditional promoter. In further embodiments, the conditional promoter is an immune cell specific promoter.

In one aspect, the engineered cytotoxic ILCs provided herein overexpress IL-15 and/or comprise a heterologous nucleic acid encoding the IL-15 gene. In certain embodiments, the engineered cytotoxic ILCs of the present disclosure target and kill a cancer cell expressing a target antigen more efficiently at a tissue site. The engineered cytotoxic ILCs disclosed herein can be generated by in vitro transduction of cytotoxic ILCs with a nucleic acid as disclosed herein.

### STAT5B

As used herein, the terms “STAT5B,” and “signal transducer and activator of transcription 5B” refer to a member of the STAT family of transcription factors. In response to cytokines and growth factors, STAT family members are phosphorylated by the receptor associated kinases, and then form homo- or heterodimers that translocate to the cell nucleus where they act as transcription activators.

STAT5B mediates the signal transduction triggered by various cell ligands, such as IL-2, IL-15, and different growth hormones. It has been shown to be involved in diverse biological processes, such as apoptosis, adult mammary gland development, and sexual dimorphism of liver gene expression. Non-limiting exemplary sequences of STAT5B protein or the underlying gene may be found under NCBI Entrez Gene: 6777 (retrieved from www.ncbi.nlm.nih.gov/gene/6777), or UniProtKB/Swiss-Prot: P42229 (retrieved from www.uniprot.org/uniprot/P42229), which are incorporated by reference herein.

Exemplary amino acid sequences of STAT5B are set forth below:

Constitutively active STAT5B (Stat5bCA), [Homo sapiens]
(SEQ ID NO: 9)
MAMWIQAQQPQGDALHQMQALYGQHFPIEVRHYLSQWIESQAWDSID
LDNPQENIKATQLLEGLVQELQKKAEHQVGEDGFLLKIKLGHYATQLQSTYDRCP
MELVRCIRHILYNEQRLVREANNGSSPAGSLADAMSQKHLQINQTFEELRLITQDTE
NELKKLQQTQEYFIIQYQESLRIQAQFAQLGQLNPQERMSRETALQQKQVSLETWL
QREAQTLQQYRVELAEKHQKTLQLLRKQQTIILDDELIQWKRRQQLAGNGGPPEGS
LDVLQSWCEKLAEIIWQNRQQIRRAERLCQQLPIPGPVEEMLAEVNATITDIISALVT
STFIIEKQPPQVLKTQTKFAATVRLLVGGKLNVHMNPPQVKATIISEQQAKSLLKNE
NTRNDYSGEILNNCCVMEYHQATGTLSAHFRNMSLKRIKRSDRRGAESVTEEKFTI
LFDSQFSVGGNELVFQVKTLSLPVVVIVHGSQDNNATATVLWDNAFAEPGRVPFA
VPDKVLWPQLCEALNMKFKAEVQSNRGLTKENLVFLAQKLFNISSNHLEDYNSMS
VSWSQFNRENLPGRNYTFWQWFDGVMEVLKKHLKPHWNDGAILGFVNKQQAHD
LLINKPDGTFLLRFSDSEIGGITIAWKFDSQERMFWNLMPFTTRDFSIRSLADRLGDL
NYLIYVFPDRPKDEVYSKYYTPVPCEPATAKAADGYVKPQIKQVVPEFANAFTDAG
SGATYMDQAPSPVVCPQAHYNMYPPNPDSVLDTDGDFDLEDMMDVARRVEELLG
RPMDSQWIPHAQS;
or
>NP_036580.2 signal transducer and activator of transcription 5B
[Homo sapiens]
(SEQ ID NO: 23)
MAVWIQAQQLQGEALHQMQALYGQHFPIEVRHYLSQWIESQAWDSVD
LDNPQENIKATQLLEGLVQELQKKAEHQVGEDGFLLKIKLGHYATQLQNTYDRCP
MELVRCIRHILYNEQRLVREANNGSSPAGSLADAMSQKHLQINQTFEELRLVTQDT
ENELKKLQQTQEYFIIQYQESLRIQAQFGPLAQLSPQERLSRETALQQKQVSLEAWL
QREAQTLQQYRVELAEKHQKTLQLLRKQQTIILDDELIQWKRRQQLAGNGGPPEGS
LDVLQSWCEKLAEIIWQNRQQIRRAEHLCQQLPIPGPVEEMLAEVNATITDIISALVT
STFIIEKQPPQVLKTQTKFAATVRLLVGGKLNVHMNPPQVKATIISEQQAKSLLKNE
NTRNDYSGEILNNCCVMEYHQATGTLSAHFRNMSLKRIKRSDRRGAESVTEEKFTI
LFESQFSVGGNELVFQVKTLSLPVVVIVHGSQDNNATATVLWDNAFAEPGRVPFAV
PDKVLWPQLCEALNMKFKAEVQSNRGLTKENLVFLAQKLFNNSSSHLEDYSGLSV
SWSQFNRENLPGRNYTFWQWFDGVMEVLKKHLKPHWNDGAILGFVNKQQAHDL
LINKPDGTFLLRFSDSEIGGITIAWKFDSQERMFWNLMPFTTRDFSIRSLADRLGDLN
YLIYVFPDRPKDEVYSKYYTPVPCESATAKAVDGYVKPQIKQVVPEFVNASADAG
GGSATYMDQAPSPAVCPQAHYNMYPQNPDSVLDTDGDFDLEDTMDVARRVEELL
GRPMDSQWIPHAQS

In some embodiments, the engineered cytotoxic ILCs express a heterologous amino acid sequence that is at least 80%, 85%, 90%, 95%, 96%, 97%, 98%, or 99% identical to SEQ ID NO: 9, SEQ ID NO: 23, or a biological equivalent thereof. In further embodiments, the biological equivalent of SEQ ID NO: 9 or SEQ ID NO: 23 comprises one or more conservative amino acid substitutions relative to SEQ ID NO: 9 or SEQ ID NO: 23, respectively. Additionally or alternatively, in some embodiments, the function of the biological equivalent is substantially similar to or is significantly more efficient compared to the protein of SEQ ID NO: 9 or SEQ ID NO: 23.

Exemplary nucleic acid sequences of human STAT5B are set forth below:

Constitutively active STAT5B (Stat5bCA), mRNA
(SEQ ID NO: 18)
ATGGCTATGTGGATACAGGCTCAGCAGCCCCAGGGCGATGCCCTTCACCAGATG
CAGGCCTTGTACGGCCAGCATTTCCCCATCGAGGTGCGACATTATTTATCACAG
TGGATCGAAAGCCAAGCCTGGGACTCAATAGATCTTGATAATCCACAGGAGAA
CATTAAGGCCACCCAGCTCCTGGAGGGCCTGGTGCAGGAGCTGCAGAAGAAGG
CGGAGCACCAGGTGGGGGAAGATGGGTTTTTGCTGAAGATCAAGCTGGGGCAC
TATGCCACACAGCTCCAGAGCACGTACGACCGCTGCCCCATGGAGCTGGTTCGC
TGTATCCGGCACATTCTGTACAACGAACAGAGGCTGGTTCGCGAAGCCAACAA
CGGCAGCTCTCCAGCTGGAAGTCTTGCTGACGCCATGTCCCAGAAGCACCTTCA
GATCAACCAAACGTTTGAGGAGCTGCGCCTGATCACACAGGACACGGAGAACG
AGCTGAAGAAGCTGCAGCAGACCCAAGAGTACTTCATCATCCAGTACCAGGAG
AGCCTGCGGATCCAAGCTCAGTTTGCCCAGCTGGGACAGCTGAACCCCCAGGA
GCGCATGAGCAGGGAGACGGCCCTCCAGCAGAAGCAAGTGTCCCTGGAGACCT
GGCTGCAGCGAGAGGCACAGACACTGCAGCAGTACCGAGTGGAGCTGGCTGAG
AAGCACCAGAAGACCCTGCAGCTGCTGCGGAAGCAGCAGACCATCATCCTGGA
CGACGAGCTGATCCAGTGGAAGCGGAGACAGCAGCTGGCCGGGAACGGGGGTC
CCCCCGAGGGCAGCCTGGACGTGCTGCAGTCCTGGTGTGAGAAGCTGGCCGAG
ATCATCTGGCAGAATCGGCAGCAGATCCGCAGGGCTGAGCGCCTGTGCCAGCA
GCTGCCCATCCCAGGCCCCGTGGAGGAGATGCTGGCTGAGGTCAACGCCACCA
TCACGGACATCATCTCAGCCCTGGTCACCAGCACGTTCATCATCGAGAAGCAGC
CTCCTCAGGTCCTGAAGACCCAGACCAAGTTTGCGGCCACTGTGCGCCTGCTGG
TGGGGGGGAAGCTGAATGTGCACATGAACCCCCCGCAGGTGAAGGCGACCATC
ATCAGCGAGCAGCAGGCCAAGTCCCTGCTCAAGAATGAGAACACCCGCAATGA
TTACAGCGGCGAGATCCTGAACAACTGTTGCGTCATGGAGTACCACCAGGCCAC
TGGCACGCTCAGCGCCCACTTCAGAAACATGTCCCTGAAACGAATCAAGAGAT
CTGACCGCCGTGGTGCAGAGTCAGTAACGGAAGAGAAGTTCACGATCCTGTTTG
ACTCACAGTTCAGCGTCGGTGGAAACGAGCTGGTCTTTCAAGTCAAGACCTTGT
CGCTCCCGGTGGTGGTGATTGTTCACGGCAGCCAGGACAACAATGCCACAGCC
ACTGTCCTCTGGGACAACGCCTTTGCAGAGCCTGGCAGGGTGCCATTTGCCGTG
CCTGACAAGGTGCTGTGGCCGCAGCTGTGTGAAGCGCTCAACATGAAATTCAA
GGCTGAAGTACAGAGCAACCGGGGCTTGACCAAGGAGAACCTCGTGTTCCTGG
CACAGAAACTGTTCAACATCAGCAGCAACCACCTCGAGGACTACAACAGCATG
TCCGTGTCCTGGTCCCAGTTCAACCGGGAGAATTTGCCAGGACGGAATTACACT
TTCTGGCAGTGGTTTGATGGCGTGATGGAAGTATTGAAAAAACATCTCAAGCCT
CACTGGAATGATGGGGCTATCCTGGGTTTCGTGAACAAGCAACAGGCCCACGA
CCTGCTCATCAACAAGCCGGACGGGACCTTCCTGCTGCGCTTCAGCGACTCGGA
AATCGGGGGCATCACCATTGCTTGGAAGTTTGACTCTCAGGAGAGAATGTTTTG
GAATCTGATGCCTTTTACCACTAGAGACTTCTCTATCCGGTCCCTCGCTGACCGC
CTGGGGGACCTGAATTACCTCATATACGTGTTTCCTGATCGGCCAAAGGATGAA
GTATATTCTAAGTACTACACACCGGTCCCCTGTGAGCCCGCAACTGCGAAAGCA
GCTGACGGATACGTGAAGCCACAGATCAAGCAGGTGGTCCCCGAGTTTGCAAA
TGCATTCACAGATGCTGGGAGTGGCGCCACCTACATGGATCAGGCTCCTTCCCC
AGTCGTGTGCCCTCAGGCTCACTACAACATGTACCCACCCAACCCGGACTCCGT
CCTTGATACCGATGGGGACTTCGATCTGGAAGACATGATGGACGTGGCGCGGC
GCGTGGAAGAGCTCTTAGGCCGGCCCATGGACAGTCAGTGGATCCCTCACGCA
CAGTCA
>NM_012448.4 Homo sapiens signal transducer and activator of
transcription 5B (STAT5B), mRNA
(SEQ ID NO: 24)
GGCGGCCGGAGCCGTCACCCCGGGCGGGGACCCAGCGCAGGCAACTCCGCGCG
GCGGCCCGGCCGAGGGAGGGAGCGAGCGGGCGGGCGGGCAAGCCAGACAGCT
GGGCCGGAGCAGCCGCGGGCGCCCGAGGGGCCGAGCGAGATTGTAAACCATGG
CTGTGTGGATACAAGCTCAGCAGCTCCAAGGAGAAGCCCTTCATCAGATGCAA
GCGTTATATGGCCAGCATTTTCCCATTGAGGTGCGGCATTATTTATCCCAGTGG
ATTGAAAGCCAAGCATGGGACTCAGTAGATCTTGATAATCCACAGGAGAACAT
TAAGGCCACCCAGCTCCTGGAGGGCCTGGTGCAGGAGCTGCAGAAGAAGGCAG
AGCACCAGGTGGGGGAAGATGGGTTTTTACTGAAGATCAAGCTGGGGCACTAT
GCCACACAGCTCCAGAACACGTATGACCGCTGCCCCATGGAGCTGGTCCGCTGC
ATCCGCCATATATTGTACAATGAACAGAGGTTGGTCCGAGAAGCCAACAATGG
TAGCTCTCCAGCTGGAAGCCTTGCTGATGCCATGTCCCAGAAACACCTCCAGAT
CAACCAGACGTTTGAGGAGCTGCGACTGGTCACGCAGGACACAGAGAATGAGT
TAAAAAAGCTGCAGCAGACTCAGGAGTACTTCATCATCCAGTACCAGGAGAGC
CTGAGGATCCAAGCTCAGTTTGGCCCGCTGGCCCAGCTGAGCCCCCAGGAGCGT
CTGAGCCGGGAGACGGCCCTCCAGCAGAAGCAGGTGTCTCTGGAGGCCTGGTT
GCAGCGTGAGGCACAGACACTGCAGCAGTACCGCGTGGAGCTGGCCGAGAAGC
ACCAGAAGACCCTGCAGCTGCTGCGGAAGCAGCAGACCATCATCCTGGATGAC
GAGCTGATCCAGTGGAAGCGGCGGCAGCAGCTGGCCGGGAACGGCGGGCCCCC
CGAGGGCAGCCTGGACGTGCTACAGTCCTGGTGTGAGAAGTTGGCCGAGATCA
TCTGGCAGAACCGGCAGCAGATCCGCAGGGCTGAGCACCTCTGCCAGCAGCTG
CCCATCCCCGGCCCAGTGGAGGAGATGCTGGCCGAGGTCAACGCCACCATCAC
GGACATTATCTCAGCCCTGGTGACCAGCACGTTCATCATTGAGAAGCAGCCTCC
TCAGGTCCTGAAGACCCAGACCAAGTTTGCAGCCACTGTGCGCCTGCTGGTGGG
CGGGAAGCTGAACGTGCACATGAACCCCCCCCAGGTGAAGGCCACCATCATCA
GTGAGCAGCAGGCCAAGTCTCTGCTCAAGAACGAGAACACCCGCAATGATTAC
AGTGGCGAGATCTTGAACAACTGCTGCGTCATGGAGTACCACCAAGCCACAGG
CACCCTTAGTGCCCACTTCAGGAATATGTCCCTGAAACGAATTAAGAGGTCAGA
CCGTCGTGGGGCAGAGTCGGTGACAGAAGAAAAATTTACAATCCTGTTTGAATC
CCAGTTCAGTGTTGGTGGAAATGAGCTGGTTTTTCAAGTCAAGACCCTGTCCCT
GCCAGTGGTGGTGATCGTTCATGGCAGCCAGGACAACAATGCGACGGCCACTG
TTCTCTGGGACAATGCTTTTGCAGAGCCTGGCAGGGTGCCATTTGCCGTGCCTG
ACAAAGTGCTGTGGCCACAGCTGTGTGAGGCGCTCAACATGAAATTCAAGGCC
GAAGTGCAGAGCAACCGGGGCCTGACCAAGGAGAACCTCGTGTTCCTGGCGCA
GAAACTGTTCAACAACAGCAGCAGCCACCTGGAGGACTACAGTGGCCTGTCTG
TGTCCTGGTCCCAGTTCAACAGGGAGAATTTACCAGGACGGAATTACACTTTCT
GGCAATGGTTTGACGGTGTGATGGAAGTGTTAAAAAAACATCTCAAGCCTCATT
GGAATGATGGGGCCATTTTGGGGTTTGTAAACAAGCAACAGGCCCATGACCTA
CTCATTAACAAGCCAGATGGGACCTTCCTCCTGAGATTCAGTGACTCAGAAATT
GGCGGCATCACCATTGCTTGGAAGTTTGATTCTCAGGAAAGAATGTTTTGGAAT
CTGATGCCTTTTACCACCAGAGACTTCTCCATTCGGTCCCTAGCCGACCGCTTGG
GAGACTTGAATTACCTTATCTACGTGTTTCCTGATCGGCCAAAAGATGAAGTAT
ACTCCAAATACTACACACCAGTTCCCTGCGAGTCTGCTACTGCTAAAGCTGTTG
ATGGATACGTGAAGCCACAGATCAAGCAAGTGGTCCCTGAGTTTGTGAACGCA
TCTGCAGATGCCGGGGGCGGCAGCGCCACGTACATGGACCAGGCCCCCTCCCC
AGCTGTGTGTCCCCAGGCTCACTATAACATGTACCCACAGAACCCTGACTCAGT
CCTTGACACCGATGGGGACTTCGATCTGGAGGACACAATGGACGTAGCGCGGC
GTGTGGAGGAGCTCCTGGGCCGGCCAATGGACAGTCAGTGGATCCCGCACGCA
CAATCGTGACCCCGCGACCTCTCCATCTTCAGCTTCTTCATCTTCACCAGAGGAA
TCACTCTTGTGGATGTTTTAATTCCATGAATCGCTTCTCTTTTGAAACAATACTC
ATAATGTGAAGTGTTAATACTAGTTGTGACCTTAGTGTTTCTGTGCATGGTGGC
ACCAGCGAAGGGAGTGCGAGTATGTGTTTGTGTGTGTGTGTGTGTGTGTGTGTG
TGTGCGTGTTTGCACGTTATGGTGTTTCTCCCTCTCACTGTCTGAGAGTTTAGTT
GTAGCAGAGGGGCCACAGACAGAAGCTGTGGTGGTTTTTACTTTGTGCAAAAA
GGCAGTGAGTTTCGTGAAGCCTGGAAGTTGGCCATGTGTCTTAAGAGTGGCTGG
ACTTTGACATGTGGCTGTTTGAATAAGAGAAGGACAAAGGGAGGAGAAAGCAC
ATGTGCTCCAGTGAGTCTTCGTCACTCTGTCTGCCAAGCAATTGATATATAACC
GTGATTGTCTCTGCTTTTCTTCTGAAATGTAGATAACTGCTTTTTGACAAAGAGA
GCCTTCCCTCTCCCCCACCCCTGTGTTCTTGGGTAGGAATGGGAAAAGGGGCAA
CCTACAAAGATTGTTGGGGCAAGGGAAGTCACAAGCTTTCGGATGGGCGGTGG
CTTTTCACAAAACATTTAGCTCATCTTATTCTCTCTTTGTCCTCTCTCCCCTCCTG
CCCGCCCGCACCCTGGAATTGCCACTCAGTTCCTCTGGGTGTGCACATATGTTTG
GAGAAATAGAGGAGAGAAAAGAGGGCCACGTAACTGAGAGCTTACAGTGCCA
ATGCCGTTTGTGTTCTGGCCAGAGTGGAGTGCGCAGCCCTGACTCCCAGGCGCT
GAGATTGTTGCCTGGTTACCCAGGAAGCTGCTGTTCCGGCTGCCCAGCCTTTCTC
TGAGCCAGCGGATGCACAGTCCGTGGCCTTCTTCAGGCTTATTGATGATGCTTTT
TGCAAATGTTGAATCATGGTTCTGTTTCTAAGTTGGATCTTTTTTGTTTTCTCCTT
GCCACCCTAATTTGACATCAAAATTCTCTCTTGTGCATTGGGCCCTGGGTCATTC
AAACCCAGGTCACCTCATTCCCCTTCTCTGTTCACACCTAATGTCTTGAAGAGTA
GGTAGCAGCAGTGTGGGCTGAACCTAGGCCAGCTTGCTTAGCGGGTCACCCTGC
TGTGAAGTCCTGGCAGGTGTTGGTAATGTGTGGAAATGCAGTCAGCAAGTTTGC
TGGGGAGTTTGATAAAAGTATAAAACAAAACAAAAAAAGCCTCGGTATAATTT
TGTTCCACGACTTCTTCTGTAGCTTTACACCAGAAGGAAGGAATGGGCTACAGC
AGGTAGTGGAGGAAGAGGGGGGTGAGCAGGTGTATTAAAATAGCTTACGGGTA
AGGCCTAAAAGGTCACCCCTCGGCCCCCTCTCCAAAAGAAGGGCATGGGCACC
CCCAGGAGAGGATGGCCCCAAAAACCTTATTTTTATACATGAGAGTAAATAAA
CATATTTTTTTTACAAAAATAACTTCTGAATTTATCAGTGTTTTGCCGTTAAAAA
TATTCCTCTATAGTAAATTATTTATTGGAAGATGACTTTTTTAAAGCTGCCGTTT
GCCTTGGCTTGGTTTCATACACTGATTTATTTTTCTATGCCAGGCAGTAGAGTCT
CTCTGCCTCTGAGGAGCAGGCTACCCGCATCCCACTCAGCCCCTCCCTACCCCT
CAAGATTTGATGAAAATTCCAACCATGAGGATGGGTGCATCGGGGAAGGGTGA
GAAGGAGAGCCTGCCTGCTCAGGGATCCAGGCTCGTAGAGTCACTCCCTGCCCG
TCTCCCAGAGATGCTTCACCAGCACCTGCCTCTGAGACCTCGCTCTCTGTTCCAG
CAACCCTGGTTGGGGGGTCAGACTTGATACACTTTCAGGTTGGGAGTGGACCCA
CCCCAGGGCCTGCTGAGGACAGAGCAGCCAGGCCGTCCTGGCTCACTTTGCAGT
TGGCACTGGGTTGGGGAGGAAGAGAGCTGATGAGTGTGGCTTCCCTGAGCTGG
GGTTTCCCTGCTTGTCCAGTTGTGAGCTGTCCTCGGTGTTACCGAGGCTGTGCCT
AGAGAGTGGAGATTTTTGATGAAAGGTGTGCTCGCTCTCTGCGTTCTATCTTCTC
TCTCCTCCTTGTTCCTGCAAACCACAAGATAAAGGTAGTGGTGTGTCTCGACCC
CATCAGCCTCTCACCCACTCCCAGACACACACAAGTCCTCAAAAGTTTCAGCTC
CGTGTGTGAGATGTGCAGGTTTTTTCTAGGGGGTAGGGGGAGACTAAAATCGA
ATATAACTTAAAATGAAAGTATACTTTTTATAATTTTTCTTTTTAAAACTTGGTG
AAATTATTTCAGATACATATTTTAGTGTCAAGGCAGATTAGTTATTTAGCCACC
AAAAAAAAGTATTGTGTACAATTTGGGGCCTCAAATTTGACTCTGCCTCAAAAA
AAAGAAATATATCCTATGCAGAGTTACAGTCACAAAGTTGTGTATTTTATGTTA
CAATAAAGCCTTCCTCTGAAGGGA

In some embodiments, the engineered cytotoxic ILCs comprise a heterologous nucleic acid sequence that is at least 80%, 85%, 90%, 95%, 96%, 97%, 98%, or 99% identical to SEQ ID NO: 18, or SEQ ID NO: 24. Additionally or alternatively, in some embodiments, the expression levels and/or activity of STAT5B in the engineered cytotoxic ILC is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native cytotoxic ILC.

In some embodiments, the engineered cytotoxic ILC further comprises a first regulatory sequence operably linked to the nucleic acid encoding the STAT5B. In further embodiments, the first regulatory sequence directs the expression of the STAT5B. Additionally or alternatively, in some embodiments, the first regulatory sequence comprises, or consists essentially of, or yet further consists of a promoter, for example a constitutive promoter or a conditional promoter. In further embodiments, the conditional promoter is an immune cell specific promoter.

In one aspect, the engineered cytotoxic ILCs provided herein overexpress STAT5B and/or comprise a heterologous nucleic acid encoding the STAT5B gene. In certain embodiments, the engineered cytotoxic ILCs of the present disclosure target and kill a cancer cell expressing a target antigen more efficiently at a tissue site. The engineered cytotoxic ILCs disclosed herein can be generated by in vitro transduction of cytotoxic ILCs with a nucleic acid as disclosed herein.

### Chimeric Antigen Receptors (CARs)

Typical therapeutic anti-cancer monoclonal antibody (mAb), like those that bind to CD19, recognize cell surface proteins, which constitute only a tiny fraction of the cellular protein content. Most mutated or oncogenic tumor associated proteins are typically nuclear or cytoplasmic. In certain instances, these intracellular proteins can be degraded in the proteasome, processed and presented on the cell surface by MHC class I molecules as T cell epitopes that are recognized by T cell receptors (TCRs). The development of mAb that mimic TCR function, “TCR mimic (TCRm)” or “TCR-like”; (i.e., that recognize peptide antigens of key intracellular proteins in the context of MHC on the cell surface) greatly extends the potential repertoire of tumor targets addressable by potent mAb. TCRm Fab, or scFv, and mouse IgG specific for the melanoma Ags, NY-ESO-1, hTERT, MART 1, gp100, and PR1, among others, have been developed. The antigen binding portions of such antibodies can be incorporated into the CARs provided herein. HLA-A2 is the most common HLA haplotype in the USA and EU (about 40% of the population) (Marsh, S., Parham, P., Barber, L., The HLA FactsBook. 1 ed. The HLA FactsBook. Vol. 1. 2000: Academic Press. 416). Therefore, potent TCRm mAb and native TCRs against tumor antigens presented in the context of HLA-A2 are useful in the treatment of a large populations. Accordingly, in some embodiments, a receptor as disclosed herein binds to a target antigen. In further embodiments, the target antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target antigen is a tumor antigen presented in the context of an HLA-A2 molecule.

In some embodiments, the engineered cytotoxic ILCs provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as an ILC. In some embodiments, transfer of the coding sequence of the CAR is facilitated by nucleic acid vector, such as a retroviral vector.

There are currently three generations of CARs. In some embodiments, the engineered cytotoxic ILCs provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD35 chain, which is the primary transmitter of signals from endogenous TCRs. “First generation” CARs can provide de novo antigen recognition and cause activation of both CD4+ and CD8⁺ T cells through their CD3ζ chain signaling domain in a single fusion molecule, independent of HLA-mediated antigen presentation.

In some embodiments, the engineered cytotoxic ILCs provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the ILC. “Second generation” CARs comprise those that provide both co-stimulation (e.g., CD28 or 4-1BB) and activation (e.g., CD3ζ).

In some embodiments, the engineered cytotoxic ILCs provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the engineered cytotoxic ILCs provided herein comprise an extracellular antigen-binding domain, a transmembrane domain and an intracellular domain. Further, the activity of the engineered cytotoxic ILCs can be adjusted by selection of co-stimulatory molecules included in the chimeric antigen receptor.

Extracellular Antigen-Binding Domain of a CAR. In certain embodiments, the extracellular antigen-binding domain of a CAR specifically binds a target antigen. In certain embodiments, the extracellular antigen-binding domain is derived from a monoclonal antibody (mAb) that binds to a target antigen. In some embodiments, the extracellular antigen-binding domain comprises, or consists essentially of, or yet further consists of an scFv. In some embodiments, the extracellular antigen-binding domain comprises, or consists essentially of, or yet further consists of a Fab, which is optionally crosslinked. In some embodiments, the extracellular binding domain comprises, or consists essentially of, or yet further consists of a F(ab)₂. In some embodiments, any of the foregoing molecules are included in a fusion protein with a heterologous sequence to form the extracellular antigen-binding domain. In certain embodiments, the extracellular antigen-binding domain comprises, or consists essentially of, or yet further consists of a human scFv that binds specifically to a target antigen. In certain embodiments, the scFv is identified by screening scFv phage library with a target antigen-Fc fusion protein.

In certain embodiments, the extracellular antigen-binding domain of a presently disclosed CAR has a high binding specificity and high binding affinity to a target antigen. For example, in some embodiments, the extracellular antigen-binding domain of the CAR (embodied, for example, in a human scFv or an analog thereof) binds to a particular target antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed target-antigen-specific CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the target-antigen-specific CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed target-antigen-specific CAR is labeled with GFP.

In some embodiments, the extracellular antigen-binding domain of the expressed CAR binds to a target antigen. In some embodiments, the extracellular antigen-binding domain of the expressed CAR binds to a target antigen presented in the context of an MHC molecule. In some embodiments, the extracellular antigen-binding domain of the expressed CAR binds to a target antigen presented in the context of an HLA-A2 molecule. In some embodiments, the extracellular antigen-binding domain of the expressed CAR binds to a target antigen not in combination with an MHC protein.

In some embodiments, the extracellular antigen-binding domain of the expressed CAR binds to a target antigen that is expressed by a tumor cell. In some embodiments, the extracellular antigen-binding domain of the expressed CAR binds to a target antigen that is expressed on the surface of a tumor cell. In some embodiments, the extracellular antigen-binding domain of the expressed CAR binds to a target antigen that is expressed on the surface of a tumor cell in combination with an MHC protein. In some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to a target antigen that is expressed on the surface of a tumor cell not in combination with an MHC protein.

In certain embodiments, the extracellular antigen-binding domain (e.g., human scFv) comprises a heavy chain variable (V_{H}) region and a light chain variable (V_{L}) region, optionally linked with a linker sequence, for example a linker peptide (e.g., SEQ ID NO: 37), between the heavy chain variable (V_{H}) region and the light chain variable (V_{L}) region. In certain embodiments, the extracellular antigen-binding domain is a human scFv-Fc fusion protein or full length human IgG with V_{H} and V_{L} regions.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 37. In certain embodiments, the nucleotide sequence encoding the amino acid sequence of SEQ ID NO: 37 is set forth in SEQ ID NO: 38.

Exemplary amino acid sequences of heavy chain variable (V_{H}) region and a light chain variable (V_{L}) region include, but are not limited to:

CD19 V_{H} domain
(SEQ ID NO: 2)
EVKLQQSGAELVRPGSSVKISCKASGYAFSSYWMNWVKQRPGQGLEWIG
QIYPGDGDTNYNGKFKGQATLTADKSSSTAYMQLSGLTSEDSAVYFCAR
KTISSVVDFYFDYWGQGTTVTVSS
CD19 V_{L} domain
(SEQ ID NO: 3)
DIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTS
GGGTKLEIK
HER2 V_{H} domain
(SEQ ID NO: 41)
EVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCS
RWGGDGFYAMDYWGQGTLVTVSS
HER2 V_{L} domain
(SEQ ID NO: 42)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIK
GPC3 V_{H} domain
(SEQ ID NO: 43)
QVQLQQSGAELVRPGASVKLSCKASGYTFTDYEMHWVKQTPVHGLKW
IGALDPKTGDTAYSQKFKGKATLTADKSSSTAYMELRSLTSEDSAVYYC
TRFYSYTYWGQGTLVTVSA
GPC3 V_{L} domain
(SEQ ID NO: 44)
DVVMTQTPLSLPVSLGDQASISCRSSQSLVHSNGNTYLHWYLQKPGQSP
KLLIYKVSNRFSGVPDRFSGSGSGTDFTLKISRVEAEDLGVYFCSQNTH
VPPTFGSGTKLEIK

Exemplary nucleic acid sequences of heavy chain variable (V_{H}) region and a light chain variable (V_{L}) region include, but are not limited to:

CD19 V_{H} domain
(SEQ ID NO: 11)
GAGGTGAAGCTGCAGCAGTCTGGGGCTGAGCTGGTGAGGCCTGGGTC
CTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTG
GACAGATTTATCCTGGAGATGGTGATACTAACTACAATGGAAAGTTCAA
GGGTCAAGCCACACTGACTGCAGACAAATCCTCCAGCACAGCCTACATG
CAGCTCAGCGGCCTAACATCTGAGGACTCTGCGGTCTATTTCTGTGCAA
GAAAGACCATTAGTTCGGTAGTAGATTTCTACTTTGACTACTGGGGCCA
AGGGACCACGGTCACCGTCTCCTCA
CD19 V_{L} domain
(SEQ ID NO: 12)
GACATTGAGCTCACCCAGTCTCCAAAATTCATGTCCACATCAGTAGG
AGACAGGGTCAGCGTCACCTGCAAGGCCAGTCAGAATGTGGGTACTAAT
GTAGCCTGGTATCAACAGAAACCAGGACAATCTCCTAAACCACTGATTT
ACTCGGCAACCTACCGGAACAGTGGAGTCCCTGATCGCTTCACAGGCAG
TGGATCTGGGACAGATTTCACTCTCACCATCACTAACGTGCAGTCTAAA
GACTTGGCAGACTATTTCTGTCAACAATATAACAGGTATCCGTACACGT
CCGGAGGGGGGACCAAGCTGGAGATCAAA
HER2 V_{H} domain
(SEQ ID NO: 45)
GAGGTCCAGTTGGTTGAATCTGGTGGAGGTTTGGTCCAGCCAGGTGG
ATCTTTGAGATTGTCTTGTGCCGCTTCTGGTTTCAACATCAAGGACACC
TACATTCATTGGGTTAGACAAGCCCCTGGTAAGGGATTGGAGTGGGTTG
CCAGAATTTACCCAACAAACGGATACACAAGATACGCTGACTCTGTCAA
GGGAAGATTCACTATCTCTGCCGACACATCTAAGAACACTGCATACTTG
CAAATGAACTCTTTGAGAGCCGAAGACACAGCCGTCTACTACTGCTCTA
GATGGGGTGGTGACGGTTTTTACGCCATGGACTATTGGGGTCAAGGAAC
ATTGGTCACAGTCTCTTCT
HER2 V_{L} domain
(SEQ ID NO: 46)
GACATTCAGATGACCCAATCTCCATCTTCTTTGTCTGCCTCTGTCGGT
GATAGAGTTACCATCACCTGCAGAGCTTCTCAAGACGTCAATACCGCAG
TTGCCTGGTATCAACAGAAGCCAGGAAAGGCACCTAAGTTGTTGATCTA
CTCTGCTTCTTTTTTGTACTCTGGAGTCCCTTCTAGATTTTCTGGATCT
AGATCTGGTACCGATTTCACATTGACCATTTCTTCTTTGCAGCCTGAGG
ACTTTGCCACATATTACTGTCAGCAGCACTACACAACCCCTCCTACTTT
TGGTCAGGGAACTAAGGTCGAGATTAAG
GPC3 V_{H} domain
(SEQ ID NO: 47)
CAGGTCCAGCTGCAGCAGTCAGGAGCCGAACTGGTGCGGCCCGGCGC
AAGTGTCAAACTGTCATGCAAGGCCAGCGGGTATACCTTCACAGACTAC
GAGATGCACTGGGTGAAACAGACCCCTGTGCACGGCCTGAAGTGGATCG
GCGCTCTGGACCCAAAAACCGGGGATACAGCATATTCCCAGAAGTTTAA
AGGAAAGGCCACTCTGACCGCTGACAAGAGCTCCTCTACTGCCTACATG
GAGCTGAGGAGCCTGACATCCGAAGATAGCGCCGTGTACTATTGCACCC
GCTTCTACTCCTATACATACTGGGGCCAGGGGACTCTGGTGACCGTCTC
TGCA
GPC3 V_{L} domain
(SEQ ID NO: 48)
GACGTGGTCATGACACAGACTCCACTGTCCCTGCCCGTGAGCCTGGG
CGATCAGGCTAGCATTTCCTGTCGAAGTTCACAGAGTCTGGTGCACTCA
AACGGAAATACCTATCTGCATTGGTACCTGCAGAAGCCAGGCCAGTCTC
CCAAACTGCTGATCTATAAGGTGAGCAACCGGTTCTCCGGGGTCCCTGA
CAGATTTTCTGGAAGTGGCTCAGGGACAGATTTCACTCTGAAAATTAGC
AGAGTGGAGGCCGAAGATCTGGGCGTCTACTTTTGTAGCCAGAATACCC
ACGTCCCACCAACATTCGGAAGCGGCACTAAACTGGAAATCAAG

In certain embodiments, the extracellular antigen-binding domain (e.g., human scFv) comprises a heavy chain variable (V_{H}) region and a light chain variable (V_{L}) region comprising SEQ ID NO: 2 and SEQ ID NO: 3, SEQ ID NO: 41 and SEQ ID NO: 42, or SEQ ID NO: 43 and SEQ ID NO: 44, respectively.

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is covalently joined to the N-terminus of the extracellular antigen-binding domain. In certain embodiments, the signal peptide comprises a human CD8 signal polypeptide comprising amino acids having the sequence set forth in SEQ ID NO: 1 as provided below: MALPVTALLLPLALLLHA (SEQ ID NO: 1).

The nucleotide sequence encoding the amino acid sequence of SEQ ID NO: 1 is set forth in SEQ ID NO: 10, which is provided below:

(SEQ ID NO: 10)
ATGGCTCTCCCAGTGACTGCCCTACTGCTTCCCCTAGCGCTTCTCCTGC
ATGCA.

In certain embodiments, the signal peptide comprises a human CD8 signal polypeptide comprising amino acids having the sequence set forth in SEQ ID NO: 25 as provided below: MALPVTALLLPLALLLHAARP (SEQ ID NO: 25).

The nucleotide sequence encoding the amino acid sequence of SEQ ID NO: 25 is set forth in SEQ ID NO: 26, which is provided below:

(SEQ ID NO: 26)
ATGGCCCTGCCAGTAACGGCTCTGCTGCTGCCACTTGCTCTGCTCCTCC
ATGCAGCCAGGCCT.

In certain embodiments, the signal peptide comprises a mouse CD8 signal polypeptide comprising amino acids having the sequence set forth in SEQ ID NO: 27 as provided below: MASPLTRFLSLNLLLLGESII (SEQ ID NO: 27).

The nucleotide sequence encoding the amino acid sequence of SEQ ID NO: 27 is set forth in SEQ ID NO: 28, which is provided below:

(SEQ ID NO: 28)
ATGGCCAGCCCCCTGACCAGGTTCCTGAGCCTGAACCTGCTGCTGCTGG
GCGAGAGCATCATC.

In certain embodiments, the signal peptide comprises a mouse CD8 signal polypeptide comprising amino acids having the sequence set forth in SEQ ID NO: 29 as provided below: MASPLTRFLSLNLLLLGE (SEQ ID NO: 29).

The nucleotide sequence encoding the amino acid sequence of SEQ ID NO: 29 is set forth in SEQ ID NO: 30, which is provided below:

(SEQ ID NO: 30)
ATGGCCAGCCCCCTGACCAGGTTCCTGAGCCTGAACCTGCTGCTGCTGG
GCGAG.

In certain embodiments, the signal peptide comprises an IL2RB signal polypeptide comprising amino acids having the sequence set forth in SEQ ID NO: 49 as provided below:

(SEQ ID NO: 49)
MAAPALSWRLPLLILLLPLATSWASA.

The nucleotide sequence encoding the amino acid sequence of SEQ ID NO: 49 is set forth in SEQ ID NO: 50, which is provided below:

(SEQ ID NO: 50)
ATGGCGGCCCCTGCTCTGTCCTGGCGTCTGCCCCTCCTCATCCTCCTC
CTGCCCCTGGCTACCTCTTGGGCATCTGCA.

In certain embodiments, the signal peptide comprises an Ig signal polypeptide comprising amino acids having the sequence set forth in SEQ ID NO: 51 as provided below:

(SEQ ID NO: 51)
MDWIWRILFLVGAATGAHS

The nucleotide sequence encoding the amino acid sequence of SEQ ID NO: 51 is set forth in SEQ ID NO: 52, which is provided below:

(SEQ ID NO: 52)
ATGGATTGGATTTGGCGCATTCTGTTTCTGGTGGGAGCCGCAACCGG
AGCACATAGT

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a NKG2D polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 31), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 31 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 31. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 31. In certain embodiments, the CD28 polypeptide comprised in the transmembrane domain has an amino acid sequence of amino acids 153 to 179 of SEQ ID NO: 31.

SEQ ID NO: 31 is provided below:

(SEQ ID NO: 31)
MLRLLLALNLFPSIQVTGNKILVKQSPMLVAYDNALSCKYSYNLFSREF
RASLHKGLDSAVEVCWYGNYSQQLQVYSKTGFNCDGKLGNESVTFYLQN
LYQTDIYFCKIEVMYPPPYLDNEKSNGTIIHVKGKHLCPSPLFPGPSKP
FWVLVWGGVLACYSLLVTVAFIIFWVRSKRSRLLHSDYMNMTPRRPGPT
RKHYQPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 31 or amino acids 153 to 179 of SEQ ID NO: 31) comprises at least a portion of the sequence set forth in SEQ ID NO: 32 as provided below.

(SEQ ID NO: 32)
attgaagttatgtatcctcctccttacctagacaatgagaagagcaatg
gaaccattatccatgtgaaagggaaacacctttgtccaagtcccctatt
tcccggaccttctaagcccttttgggtgctggtggtggttggtggagtc
ctggcttgctatagcttgctagtaacagtggcctttattattttctggg
tgaggagtaagaggagcaggctcctgcacagtgactacatgaacatgac
tccccgccgccccgggcccacccgcaagcattaccagccctatgcccca
ccacgcgacttcgcagcctatcgctcc

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 33 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 33 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 33.

(SEQ ID NO: 33)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPWKSGDKPSLSARYV

In certain embodiments, the transmembrane domain comprises a CD8 polypeptide comprising amino acids having the sequence set forth in SEQ ID NO: 34 as provided below:

(SEQ ID NO: 34)
PTTTPAPRPPTPAPTIASQPLSLRPEACRPAAGGAVHTRGLDFACDIYI
WAPLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 34) comprises nucleic acids having the sequence set forth in SEQ ID NO: 35 as provided below.

(SEQ ID NO: 35)
CCCACCACGACGCCAGCGCCGCGACCACCAACCCCGGCGCCCACGATC
GCGTCGCAGCCCCTGTCCCTGCGCCCAGAGGCGTGCCGGCCAGCGGCG
GGGGGCGCAGTGCACACGAGGGGGCTGGACTTCGCCTGTGATATCTAC
ATCTGGGCGCCCCTGGCCGGGACTTGTGGGGTCCTTCTCCTGTCACTG
GTTATCACCCTTTACTGCAAC

In certain embodiments, the transmembrane domain comprises a NKG2D polypeptide. The NKG2D polypeptide can have an amino acid sequence that is at least about 85%, about 90%, about 95%, about 96%, about 97%, about 98%, about 99% or about 100%) homologous to SEQ ID NO: 36 (homology herein may be determined using standard software such as BLAST or FASTA) as provided below, or fragments thereof, and/or may optionally comprise up to one or up to two or up to three conservative amino acid substitutions. In certain embodiments, the NKG2D polypeptide can have an amino acid sequence that is a consecutive portion of SEQ ID NO: 36 which is at least 20, or at least 30, or at least 40, or at least 50, and up to 216 amino acids in length. Additionally or alternatively, in various embodiments, the NKG2D polypeptide has an amino acid sequence of amino acids 1 to 216, or 50 to 100 of SEQ ID NO: 36.

(SEQ ID NO: 36)
MGWIRGRRSRHSWEMSEFHNYNLDLKKSDFSTRWQKQRCPVVKSKCR
ENASPFFFCCFIAVAMGIRFIIMVTIWSAVFLNSLFNQEVQIPLTESY
CGPCPKNWICYKNNCYQFFDESKNWYESQASCMSQNASLLKVYSKEDQ
DLLKLVKSYHWMGLVHIPTNGSWQWEDGSILSPNLLTIIEMQKGDCAL
YASSFKGYIENCSTPNTYICMQRTV

In certain embodiments, the transmembrane domain comprises a NKG2D polypeptide comprising amino acids having the sequence set forth in SEQ ID NO: 4 as provided below:

(SEQ ID NO: 4)
PFFFCCFIAVAMGIRFIIMVT

In accordance with the presently disclosed subject matter, a “NKG2D nucleic acid molecule” refers to a polynucleotide encoding a NKG2D polypeptide. In certain embodiments, the NKG2D nucleic acid molecule encoding the NKG2D polypeptide comprised in the transmembrane domain of the presently disclosed CAR (SEQ ID NO: 4) comprises nucleic acids having the sequence set forth in SEQ ID NO: 13 as provided below.

(SEQ ID NO: 13)
CCATTTTTTTTCTGCTGCTTCATCGCTGTAGCCATGGGAATCCGTTT
CATTATTATGGTAACA

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the CH₂CH₃ region of immunoglobulin and portions of CD3, a portion of a CD28 polypeptide (e.g., SEQ ID NO: 31), a portion of a CD8 polypeptide (e.g., SEQ ID NO: 34), a portion of a NKG2D polypeptide (e.g., SEQ ID NO: 4), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., an ILC). CD3ζ comprises 3 ITAMs, and transmits an activation signal to the cell (e.g., a cell of the lymphoid lineage, e.g., an ILC) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 53), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 54 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 54. In certain embodiments, the CD32 polypeptide has an amino acid sequence of amino acids 52 to 164 of SEQ ID NO: 54.

SEQ ID NO: 54 is provided below:

(SEQ ID NO: 54)
MKWKALFTAAILQAQLPITEAQSFGLLDPKLCYLLDGILFIYGVILT
ALFLRVKFSRSADAPAYQQGQNQLYNELNLGRREEYDVLDKRRGRDP
EMGGKPQRRKNPQEGLYNELQKDKMAEAYSEIGMKGERRRGKGHDGL
YQGLSTATKDTYDALHMQALPPR

In certain embodiments, the CD3 polypeptide has the amino acid sequence set forth in SEQ ID NO: 55, which is provided below:

(SEQ ID NO: 55)
RVKFSRSAEPPAYQQGQNQLYNELNLGRREEYDVLDKRRGRDPEMGG
KPRRKNPQEGLYNELQKDKMAEAYSEIGMKGERRRGKGHDGLYQGLS
TATKDTYDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 55) comprised in the intracellular domain of the presently disclosed CAR comprises a nucleotide sequence as set forth in SEQ ID NO: 56 as provided below.

(SEQ ID NO: 56)
AGAGTGAAGTTCAGCAGGAGCGCAGAGCCCCCCGCGTACCAGCAGGG
CCAGAACCAGCTCTATAACGAGCTCAATCTAGGACGAAGAGAGGAGT
ACGATGTTTTGGACAAGAGACGTGGCCGGGACCCTGAGATGGGGGGA
AAGCCGAGAAGGAAGAACCCTCAGGAAGGCCTGTACAATGAACTGCA
GAAAGATAAGATGGCGGAGGCCTACAGTGAGATTGGGATGAAAGGCG
AGCGCCGGAGGGGCAAGGGGCACGATGGCCTTTACCAGGGTCTCAGT
ACAGCCACCAAGGACACCTACGACGCCCTTCACATGCAGGCCCTGCC
CCCTCGCG

In certain embodiments, the CD3ζ polypeptide has the amino acid sequence set forth in SEQ ID NO: 8, which is provided below:

(SEQ ID NO: 8)
RVKFSRSADAPAYQQGQNQLYNELNLGRREEYDVLDKRRGRDPEMGG
KPRRKNPQEGLYNELQKDKMAEAYSEIGMKGERRRGKGHDGLYQGLS
TATKDTYDALHMQALPPR

The CD3ζ nucleic acid molecule encoding the CD3ζ polypeptide (SEQ ID NO: 8) comprised in the intracellular domain of the presently disclosed CAR comprises a nucleotide sequence as set forth in SEQ ID NO: 17:

(SEQ ID NO: 17)
AGAGTGAAGTTCAGCAGGAGCGCAGACGCCCCCGCGTACCAGCAGG
GCCAGAACCAGCTCTATAACGAGCTCAATCTAGGACGAAGAGAGGAG
TACGATGTTTTGGACAAGAGACGTGGCCGGGACCCTGAGATGGGGGG
AAAGCCGAGAAGGAAGAACCCTCAGGAAGGCCTGTACAATGAACTGC
AGAAAGATAAGATGGCGGAGGCCTACAGTGAGATTGGGATGAAAGGC
GAGCGCCGGAGGGGCAAGGGGCACGATGGCCTTTACCAGGGTCTCAG
TACAGCCACCAAGGACACCTACGACGCCCTTCACATGCAGGCCCTGC
CCCCTCGC

In certain non-limiting embodiments, an intracellular domain of the CAR can comprise a truncated cytoplasmic domain of IL-2RβΔ. In certain embodiments, the IL2RbΔ polypeptide has an amino acid sequence of (SEQ ID NO: 7):

(SEQ ID NO: 7)
NCRNTGPWLKKVLKCNTPDPSKFFSQLSSEHGGDVQKWLSSPFPS
SSFSPGGLAPEISPLEVLERDKVTQLLPLNTDAYLSLQELQGQDP
THLV

The IL-2RβΔ nucleic acid molecule encoding the IL-2RβΔ polypeptide (SEQ ID NO: 7) comprised in the intracellular domain of the presently disclosed CAR comprises a nucleotide sequence as set forth in SEQ ID NO: 16:

(SEQ ID NO: 16)
AACTGCAGGAACACCGGGCCATGGCTGAAGAAGGTCCTGAAGTGT
AACACCCCAGACCCCTCGAAGTTCTTTTCCCAGCTGAGCTCAGAG
CATGGAGGAGACGTCCAGAAGTGGCTCTCTTCGCCCTTCCCCTCA
TCGTCCTTCAGCCCTGGCGGCCTGGCACCTGAGATCTCGCCACTA
GAAGTGCTGGAGAGGGACAAGGTGACGCAGCTGCTCCCCCTGAAC
ACTGATGCCTACTTGTCCCTCCAAGAACTCCAGGGTCAGGACCCA
ACTCACTTGGTG

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a NKG2C polypeptide, a NKG2D polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, a 2B4 polypeptide, a BTLA polypeptide, a NKG2C polypeptide, a NKG2D polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-LI. As one example, a 4-1BB ligand (i.e., 4-1BBL) may bind to 4-1BB (also known as “CD 137”) for providing an intracellular signal that in combination with a CAR signal induces an effector cell function of the CAR⁺ immune cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 57) or fragments thereof, and/or may optionally comprise up to one or up to two or up to three conservative amino acid substitutions.

SEQ ID NO: 57 is provided below:

(SEQ ID NO: 57)
MGNSCYNIVATLLLVLNFERTRSLQDPCSNCPAGTFCDNNRNQIC
SPCPPNSFSSAGGQRTCDICRQCKGVFRTRKECSSTSNAECDCTP
GFHCLGAGCSMCEQDCKQGQELTKKGCKDCCFGTFNDQKRGICRP
WTNCSLDGKSVLGTKERDWCGPSPADLSPGASSVTPPAPAREPGH
SPQIISFFLALTSTALLFLLFFLTLRFSWKRGRKKLLYIFKQPFM
RPVQTTQEEDGCSCRFPEEEEGGCEL

In certain embodiments, the 4-1BB co-stimulatory domain has the amino acid sequence set forth in SEQ ID NO: 58, which is provided below:

(SEQ ID NO: 58)
KRGRKKLLYIFKQPFMRPVQTTQEEDGCSCRFPEEEEGGCEL

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 58) comprised in the intracellular domain of the presently disclosed CAR comprises a nucleotide sequence as set forth in SEQ ID NO: 59 as provided below.

(SEQ ID NO: 59)
AAACGGGGCAGAAAGAAGCTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 60), or fragments thereof, and/or may optionally comprise up to one or up to two or up to three conservative amino acid substitutions.

SEQ ID NO: 60 is provided below:

(SEQ ID NO: 60)
MCVGARRLGRGPCAALLLLGLGLSTVTGLHCVGDTYPSNDRCCHE
CRPGNGMVSRCSRSQNTVCRPCGPGFYNDWSSKPCKPCTWCNLRS
GSERKQLCTATQDTVCRCRAGTQPLDSYKPGVDCAPCPPGHFSPG
DNQACKPWTNCTLAGKHTLQPASNSSDAICEDRDPPATQPQETQG
PPARPITVQPTEAWPRTSQGPSTRPVEVPGGRAVAAILGLGLVLG
LLGPLAILLALYLLRRDQRLPPDAHKPPGGGSFRTPIQEEQADAH
STLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 61) or fragments thereof, and/or may optionally comprise up to one or up to two or up to three conservative amino acid substitutions.

SEQ ID NO: 61 is provided below:

(SEQ ID NO: 61)
MKSGLWYFFLFCLRIKVLTGEINGSANYEMFIFHNGGVQILCKYP
DIVQQFKMQLLKGGQILCDLTKTKGSGNTVSIKSLKFCHSQLSNN
SVSFFLYNLDHSHANYYFCNLSIFDPPPFKVTLTGGYLHIYESQL
CCQLKFWLPIGCAAFVWCILGCILICWLTKKKYSSSVHDPNGEYM
FMRATAKKSRLTDVTL

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM (SEQ ID NO: 62) motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 63) (homology herein may be determined using standard software such as BLAST or FASTA) or fragments thereof, and/or may optionally comprise up to one or up to two or up to three conservative amino acid substitutions.

SEQ ID NO: 63 is provided below:

(SEQ ID NO: 63)
MACLGFQRHKAQLNLATRTWPCTLLFFLLFIPVFCKAMHVAQPAW
LASSRGIASFVCEYASPGKATEVRVTVLRQADSQVTEVCAATYMM
GNELTFLDDSICTGTSSGNQLTIQGLRAMDTGLYICKVELMYPPP
YYLGIGNGTQIYVIDPEPCPDSDFLLWILAAVSSGLFFYSFLLTA
VSLSKMLKKRSPLTTGVYVKMPPTEPECEKQFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 64) or fragments thereof, and/or may optionally comprise up to one or up to two or up to three conservative amino acid substitutions.

SEQ ID NO: 64 is provided below:

(SEQ ID NO: 64)
MQIPQAPWPVVWAVLQLGWRPGWFLDSPDRPWNPPTFSPALLWTE
GDNATFTCSFSNTSESFVLNWYRMSPSNQTDKLAAFPEDRSQPGQ
DCRFRVTQLPNGRDFHMSVVRARRNDSGTYLCGAISLAPKAQIKE
SLRAELRVTERRAEVPTAHPSPSPRPAGQFQTLVVGWGGLLGSLV
LLVWVLAVICSRAARGTIGARRTGQPLKEDPSAVPVFSVDYGELD
FQWREKTPEPPVPCVPEQTEYATIVFPSGMGTSSPARRGSADGPR
SAQPLRPEDGHCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 65) or fragments thereof, and/or may optionally comprise up to one or up to two or up to three conservative amino acid substitutions.

SEQ ID NO: 65 is provided below:

(SEQ ID NO: 65)
MWEAQFLGLLFLQPLWVAPVKPLQPGAEVPWWAQEGAPAQLPCSP
TIPLQDLSLLRRAGVTWQHQPDSGPPAAAPGHPLAPGPHPAAPSS
WGPRPRRYTVLSVGPGGLRSGRLPLQPRVQLDERGRQRGDFSLWL
RPARRADAGEYRAAVHLRDRALSCRLRLRLGQASMTASPPGSLRA
SDWVILNCSFSRPDRPASVHWFRNRGQGRVPVRESPHHHLAESFL
FLPQVSPMDSGPWGCILTYRDGFNVSIMYNLTVLGLEPPTPLTVY
AGAGSRVGLPCRLPAGVGTRSFLTAKWTPPGGGPDLLVTGDNGDF
TLRLEDVSQAQAGTYTCHIHLQEQQLNATVTLAIITVTPKSFGSP
GSLGKLLCEVTPVSGQERFVWSSLDTPSQRSFSGPWLEAQEAQLL
SQPWQCQLYQGERLLGAAVYFTELSSPGAQRSGRAPGALPAGHLL
LFLILGVLSLLLLVTGAFGFHLWRRQWRPRRFSALEQGIHPPQAQ
SKIEELEQEPEPEPEPEPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 66) or fragments thereof, and/or may optionally comprise up to one or up to two or up to three conservative amino acid substitutions.

SEQ ID NO: 66 is provided below:

(SEQ ID NO: 66)
MLGQWTLILLLLLKVYQGKGCQGSADHWSISGVPLQLQPNSIQTK
VDSIAWKKLLPSQNGFHHILKWENGSLPSNTSNDRFSFIVKNLSL
LIKAAQQQDSGLYCLEVTSISGKVQTATFQVFVFESLLPDKVEKP
RLQGQGKILDRGRCQVALSCLVSRDGNVSYAWYRGSKLIQTAGNL
TYLDEEVDINGTHTYTCNVSNPVSWESHTLNLTQDCQNAHQEFRF
WPFL VIIVILSALFLGTLACFCVWRRKRKEKQSETSPKEFLTIY
EDVKDLKTRRNHEQEQTFPGGGSTIYSMIQSQSSAPTSQEPAYTL
YSLIQPSRKSGSRKRNHSPSFNSTIYEVIGKSQPKAQNPARLSRK
ELENFDVYS

In some embodiments, the 2B4 polypeptide comprises the amino acid sequence of SEQ ID NO: 5:

(SEQ ID NO: 5)
WRRKRKEKQSETSPKEFLTIYEDVKDLKTRRNHEQEQTFPGGGST
IYSMIQSQSSAPTSQEPAYTLYSLIQPSRKSGSRKRNHSPSFNST
IYEVIGKSQPKAQNPARLSRKELENFDVYS

The 2B4 nucleic acid molecule encoding the 2B4 polypeptide (SEQ ID NO: 5) comprised in the intracellular domain of the presently disclosed CAR comprises a nucleotide sequence as set forth in SEQ ID NO: 14:

(SEQ ID NO: 14)
TGGAGGAGAAAGAGGAAGGAGAAGCAGTCAGAGACCAGTCCCAAG
GAATTTTTGACAATTTACGAAGATGTCAAGGATCTGAAAACCAGG
AGAAATCACGAGCAGGAGCAGACTTTTCCTGGAGGGGGGAGCACC
ATCTACTCTATGATCCAGTCCCAGTCTTCTGCTCCCACGTCACAA
GAACCTGCATATACATTATATTCATTAATTCAGCCTTCCAGGAAG
TCTGGATCCAGGAAGAGGAACCACAGCCCTTCCTTCAATAGCACT
ATCTATGAAGTGATTGGAAAGAGTCAACCTAAAGCCCAGAACCCT
GCTCGATTGAGCCGCAAAGAGCTGGAGAACTTTGATGTTTATTCC

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8+ cancer-specific T cells. BTLA has also been designated as CD272 (cluster of differentiation 272).

In accordance with the presently disclosed subject matter, a BTLA polypeptide can have an amino acid sequence that is at least about 85%, about 90%, about 95%, about 96%, about 97%, about 98%, about 99% or about 100% homologous to UniProtKB/Swiss-Prot Ref. No.: Q7Z6A9.3 (SEQ ID NO: 67) or fragments thereof, and/or may optionally comprise up to one or up to two or up to three conservative amino acid substitutions.

SEQ ID NO: 67 is provided below:

(SEQ ID NO: 67)
MKTLPAMLGTGKLFWVFFLIPYLDIWNIHGKESCDVQLYIKRQSE
HSILAGDPFELECPVKYCANRPHVTWCKLNGTTCVKLEDRQTSWK
EEKNISFFILHFEPVLPNDNGSYRCSANFQSNLIESHSTTLYVTD
VKSASERPSKDEMASRPWLLYRLLPLGGLPLLITTCFCLFCCLRR
HQGKQNELSDTAGREINLVDAHLKSEQTEASTRQNSQVLLSETGI
YDNDPDLCFRMQEGSEVYSNPCLEENKPGIVYASLNHSVIGPNSR
LARNVKEAPTEYASICVRS

In accordance with the presently disclosed subject matter, a “BTLA nucleic acid molecule” refers to a polynucleotide encoding a BTLA polypeptide.

In some embodiments, the co-stimulatory domain may comprise a DAP10 polypeptide including the amino acid sequence of LCARPRRSPAQEDGKVYINMPGRG (SEQ ID NO: 6). The nucleic acid molecule encoding the DAP10 polypeptide (SEQ ID NO: 6) comprised in the co-stimulatory domain of the presently disclosed CAR comprises a nucleotide sequence as set forth in SEQ ID NO: 15:

(SEQ ID NO: 15)
CTGTGCGCACGCCCACGCCGCAGCCCCGCCCAAGAAGATGGCAAA
GTCTACATCAACATGCCAGGCAGGGGC

Additionally or alternatively, in some embodiments, the heterologous nucleic acid encoding the IL-15 or STAT5B gene and/or any CAR disclosed herein is operably linked to an inducible promoter. In some embodiments, the heterologous nucleic acid encoding the IL-15 or STAT5B gene and/or any CAR disclosed herein is operably linked to a constitutive promoter.

In some embodiments, the inducible promoter is a synthetic Notch promoter that is activatable in a CAR⁺ ILC cell, where the intracellular domain of the CAR contains a transcriptional regulator that is released from the membrane when engagement of the CAR with the target antigen/polypeptide induces intramembrane proteolysis (see, e.g., Morsut et al., Cell 164(4): 780-791 (2016). Accordingly, further transcription of the target-antigen-specific CAR is induced upon binding of the engineered ILC with the antigen/polypeptide.

The presently disclosed subject matter also provides isolated nucleic acid molecules encoding the IL-15 or STAT5B gene and/or any CAR construct described herein or a functional portion thereof. In certain embodiments, the CAR construct comprises (a) an antigen binding fragment (e.g., an anti-target-antigen scFv or a fragment) that specifically binds to a target antigen, (b) a transmembrane domain comprising a CD8 polypeptide, CD28 polypeptide, or a NKG2D polypeptide, and (c) an intracellular domain comprising a truncated cytoplasmic domain of IL-2RβΔ, or a CD3ζ polypeptide, and optionally one or more of a co-stimulatory signaling region disclosed herein, a P2A self-cleaving peptide, and/or a reporter or selection marker provided herein. The at least one co-stimulatory signaling region can include a CD28 polypeptide, a CD3ζ (polypeptide, a 4-1BB polypeptide, an OX40 polypeptide, an ICOS polypeptide, a DAP-10 (HCST) polypeptide, a PD-1 polypeptide, a CTLA-4 polypeptide, a LAG-3 polypeptide, a 2B4 polypeptide, a BTLA polypeptide, a NKG2C polypeptide, a NKG2D polypeptide, a synthetic peptide (not based on a protein associated with the immune response), or a combination thereof. In some embodiments, the at least one co-stimulatory signaling region includes a DAP-10 polypeptide and a 2B4 polypeptide.

In certain embodiments, the isolated nucleic acid molecule encodes an IL-15 or STAT5B gene and any CAR construct disclosed herein (such as a CAR that specifically binds a target antigen) comprising an antigen binding fragment (e.g., a scFv) that specifically binds to a target antigen/polypeptide, fused to a synthetic Notch transmembrane domain and an intracellular cleavable transcription factor. In certain embodiments, the present disclosure provides an isolated nucleic acid molecule encoding an IL-15 or STAT5B gene and a receptor (such as a CAR that specifically binds a target antigen) that is inducible by release of the transcription factor of a synthetic Notch system.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the parent CAR. For example, functional portions encompass the portions, parts or fragments of a target-antigen-specific CAR that retains the ability to recognize a target cell, to treat cancer, to a similar, same, or even a higher extent as the parent CAR. In certain embodiments, an isolated nucleic acid molecule encoding a functional portion of a target-antigen-specific CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

The presently disclosed subject matter provides engineered immune cells expressing an IL-15 or STAT5B and a target-antigen-specific CAR or other ligand that comprises an extracellular antigen-binding domain, a transmembrane domain and an intracellular domain, where the extracellular antigen-binding domain specifically binds a target antigen/polypeptide. In certain embodiments, ILCs can be transduced with a presently disclosed CAR constructs such that the cells express the CAR. The presently disclosed subject matter also provides methods of using such cells for the treatment of cancer.

The engineered cytotoxic ILCs of the presently disclosed subject matter can express an IL-15 or STAT5B and/or an extracellular antigen binding domain (e.g., an anti-target-antigen scFv, an anti-target-antigen Fab that is optionally crosslinked, an anti-target-antigen F(ab)₂ or a fragment) that specifically binds to a target antigen, for the treatment of cancer.

In some embodiments, the higher the expression level of IL-15 or STAT5B and/or the CAR in an engineered cytotoxic ILC, the greater cytotoxicity and cytokine production the engineered ILC exhibits.

Additionally or alternatively, the cytotoxicity and cytokine production of a presently disclosed engineered cytotoxic ILC are proportional to the expression level of target antigen in a target tissue or a target cell. Additionally or alternatively, the cytotoxicity and cytokine production of a presently disclosed engineered cytotoxic ILC are proportional to the expression level of IL-15, STAT5B or CAR in the ILC. For example, the higher the expression level, the greater cytotoxicity and cytokine production the engineered cytotoxic ILC exhibits.

In certain embodiments, the antigen recognizing receptor is a chimeric co-stimulatory receptor (CCR). CCR is described in Krause, et al., J. Exp. Med. 188(4):619-626 (1998), and US20020018783, the contents of which are incorporated by reference in their entireties. CCRs mimic co-stimulatory signals, but unlike, CARs, do not provide a T-cell activation signal, e.g., CCRs lack a CD32 polypeptide. CCRs provide co-stimulation, e.g., a CD28-like signal, in the absence of the natural co-stimulatory ligand on the antigen-presenting cell. In certain embodiments, the CCR comprises (a) an extracellular antigen-binding domain that binds to an antigen different than the first target antigen, (b) a transmembrane domain, and (c) a co-stimulatory signaling region that comprises at least one co-stimulatory molecule, including, but not limited to, CD28, 4-1BB, OX40, ICOS, PD-1, CTLA-4, LAG-3, 2B4, NKG2C, NKG2D, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second target antigen is selected so that expression of both the first target antigen and the second target antigen is restricted to the targeted cells (e.g., cancerous cells). Similar to a CAR, the extracellular antigen-binding domain can be an scFv, a Fab, a F(ab)₂; or a fusion protein with a heterologous sequence to form the extracellular antigen-binding domain.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells. The truncated CAR functions as an adhesion molecule that enhances the avidity of a presently disclosed CAR, especially, one that has a low binding affinity to a target antigen, thereby improving the efficacy of the presently disclosed CAR or engineered cytotoxic ILC comprising the same. In certain embodiments, the truncated CAR comprises an extracellular antigen-binding domain that binds to a target antigen, and a transmembrane domain comprising a CD8 polypeptide. A presently disclosed ILC comprises or is transduced to express a presently disclosed CAR targeting a target antigen and a truncated CAR targeting a target antigen. In certain embodiments, the targeted cells are solid tumor cells.

### Polynucleotides, Polypeptides and Analogs

Also included in the presently disclosed subject matter are polynucleotides encoding IL-15, STAT5B, or any CAR disclosed herein (e.g., CAR including a truncated cytoplasmic domain of IL-2RβΔ) and their corresponding polypeptides or fragments that may be modified in ways that enhance their anti-tumor activity when expressed in an engineered cytotoxic ILC. The presently disclosed subject matter provides methods for optimizing an amino acid sequence or a nucleic acid sequence by producing an alteration in the sequence. Such alterations can comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program can be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications can occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or can result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein. Such analogs are administered according to methods of the presently disclosed subject matter. Such analogs can exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered cytotoxic ILC. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding IL-15, STAT5B, or any CAR disclosed herein (e.g., CAR including a truncated cytoplasmic domain of IL-2RβΔ) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

In some embodiments, a nucleic acid as disclosed herein further comprises a regulatory sequence directing the expression of IL-15, STAT5B, or any CAR disclosed herein (e.g., CAR including a truncated cytoplasmic domain of IL-2RβΔ). In further embodiments, the nucleic acid comprises a single regulatory sequence directing the expression of both the IL-15 or STAT5B gene, and any CAR disclosed herein (e.g., CAR including a truncated cytoplasmic domain of IL-2RβΔ). In other embodiments, the nucleic acid comprises a first regulatory sequence directing the expression of the IL-15 or STAT5B gene and a second regulatory sequence directing the expression of the CAR. In other embodiments, the first regulatory sequence is the same as the second regulatory sequence. In some embodiments, the first regulatory sequence is different from the second regulatory sequence.

### Vectors

Many expression vectors are available and known to those of skill in the art and can be used for nonendogenous expression of IL-15, STAT5B, or any CAR disclosed herein (e.g., CAR including a truncated cytoplasmic domain of IL-2RβΔ). The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 69) or a myc tag, hemagglutinin tag or a tag for purification, for example, a GST fusion, and a sequence for directing protein secretion and/or membrane association.

Heterologous expression of IL-15, STAT5B, or any CAR disclosed herein (e.g., CAR including a truncated cytoplasmic domain of IL-2RβΔ) can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25 (1983)); see also “Useful Proteins from Recombinant Bacteria”: in Scientific American 242:79-94 (1980)); plant expression vectors containing the nopaline synthetase promoter (Herrera-Estrella et al., Nature 505:209-213 (1984)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 510: 1 15-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 515:115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7:1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254:1372-1378 (1986)).

In addition to the promoter, the expression vector typically contains a transcription unit or expression cassette that contains all the additional elements required for the expression of an antibody, or antigen binding fragment thereof, in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

In some embodiments, the expression vector is a plasmid, a cosmid, a bacmid, a bacterial artificial chromosome (BAC), a yeast artificial chromosome (YAC), a viral vector, or a retroviral vector.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 poly A signal.

Genetic modification of engineered cytotoxic ILCs can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding IL-15, STAT5B, or any CAR disclosed herein (e.g., CAR including a truncated cytoplasmic domain of IL-2RβΔ) can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide ILCs expressing IL-15, STAT5B, or any CAR disclosed herein (e.g., CAR including a truncated cytoplasmic domain of IL-2RβΔ), a retroviral vector can be employed for transduction. However, any other suitable viral vector or non-viral delivery system can be used for genetic modification of cells. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller et al. (1985) Mol. Cell. Biol. 5:431-437); PA317 (Miller et al. (1986) Mol. Cell. Biol. 6:2895-2902); and CRIP (Danos et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al., Blood 80: 1418-1422 (1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secrete a cytokine (e.g., 4-1BBL and/or IL-12) in an engineered cytotoxic ILC. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71: 6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244:1275-1281 (1989); Eglitis et al., BioTechniques 6:608-614, (1988); Tolstoshev et al., Current Opinion in Biotechnology 1:55-61 (1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing IL-15, STAT5B, or any CAR disclosed herein (e.g., CAR including a truncated cytoplasmic domain of IL-2RβΔ) is a retroviral vector, e.g., an oncoretroviral vector. In some instances, the retroviral vector is a SFG retroviral vector or murine stem cell virus (MSCV) retroviral vector. In certain non-limiting embodiments, the vector expressing an IL-15, STAT5B, or CAR (e.g., CAR including a truncated cytoplasmic domain of IL-2RβΔ) nucleic acid sequence is a lentiviral vector. In certain non-limiting embodiments, the vector expressing an IL-15, STAT5B, or CAR (e.g., CAR including a truncated cytoplasmic domain of IL-2RβΔ) nucleic acid sequence is a transposon vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101: 512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

In some embodiments, a vector as disclosed herein further comprises a regulatory sequence directing the expression of IL-15, STAT5B, or any CAR disclosed herein. In further embodiments, the vector comprises a single regulatory sequence directing the expression of both of the IL-15 or STAT5B, and any CAR disclosed herein. In other embodiments, the vector comprises a first regulatory sequence directing the expression of the IL-15 or STAT5B and a second regulatory sequence directing the expression of any CAR disclosed herein. In other embodiments, the first regulatory sequence is the same as the second regulatory sequence. In some embodiments, the first regulatory sequence is different from the second regulatory sequence.

### Engineered Cytotoxic ILCs

The presently disclosed subject matter provides engineered cytotoxic ILCs that exhibit heterologous expression of IL-15, or STAT5B. Additionally or alternatively, in some embodiments, the engineered cytotoxic ILCs may further comprise an engineered receptor (e.g., a CAR) or other ligand that comprises an extracellular antigen-binding domain, a transmembrane domain and an intracellular domain, where the extracellular antigen-binding domain specifically binds a tumor antigen, including a tumor receptor or ligand. In another aspect, the present disclosure provides engineered cytotoxic ILCs comprising a CAR including a truncated cytoplasmic domain of IL-2RβΔ. In certain embodiments, cytotoxic ILCs can be transduced with a vector comprising nucleic acid sequences that encode IL-15, STAT5B, or any CAR disclosed herein (e.g., CAR including a truncated cytoplasmic domain of IL-2RβΔ).

Examples of tumor antigens include, but are not limited to, 5T4, alpha 5β1-integrin, 707-AP, AFP, ART-4, B7H4, BCMA, Bcr-abl, CA125, CA19-9, CDH1, CDH17, CAMEL, CAP-1, CASP-8, CD5, CD25, CDC27/m, CD37, CD52, CDK4/m, c-Met, CS-1, CT, Cyp-B, cyclin B1, DAGE, DAM, EBNA, ErbB3, ELF2M, EMMPRIN, ephrinB2, estrogen receptor, ETV6-AML1, FAP, ferritin, folate-binding protein, G250, GM2, HAGE, HLA-A*0201-R170I, HPV E6, HPV E7, HSP70-2M, HST-2, hTERT (or hTRT), iCE, IL-2R, IL-5, KIAA0205, LAGE, LDLR/FUT, MART-1/melan-A, MART-2/Ski, MC1R, mesothelin, MUC16, myc, MUM-2, MUM-3, NA88-A, NYESO-1, NY-Eso-B, proteinase-3, p190 minor bcr-abl, Pml/RARα, progesterone receptor, PSCA, RU1 or RU2, RORI, SART-1 or SART-3, survivin, TEL/AML1, TGFβ, TPI/m, TRP-1, TRP-2, TRP-2/INT2, tenascin, TSTA tyrosinase, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PIGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Ley) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, and peptide MHC complexes (with peptides derived from TP53, KRAS, MYC, EBNA1-6, PRAME, tyronsinase, MAGEA1-A6, pmel17, LMP2, or WT1).

The presently disclosed subject matter also provides methods of using such engineered cytotoxic ILCs for the treatment of a tumor. The engineered cytotoxic ILCs of the presently disclosed subject matter can express non-endogenous levels of IL-15, STAT5B, or any CAR disclosed herein (e.g., CAR including a truncated cytoplasmic domain of IL-2RβΔ) for the treatment of cancer, e.g., for treatment of tumor. Such engineered cytotoxic ILCs can be administered to a subject (e.g., a human subject) in need thereof for the treatment of cancer.

The presently disclosed engineered cytotoxic ILCs of the present technology may further include at least one recombinant or exogenous co-stimulatory ligand. For example, the presently disclosed engineered cytotoxic ILCs can be further transduced with at least one co-stimulatory ligand, such that the engineered cytotoxic ILCs co-expresses or is induced to co-express IL-15, STAT5B, or any CAR disclosed herein (e.g., CAR including a truncated cytoplasmic domain of IL-2RβΔ) and the at least one co-stimulatory ligand. Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-Tβ), CD257/B cell-activating factor (B AFF)/Bly s/THANK/Tall-1, glucocorticoid-induced TNF Receptor ligand (GITRL), and TF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins-they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered cytotoxic ILC comprises one recombinant co-stimulatory ligand (e.g., 4-1BBL). In certain embodiments, the engineered cytotoxic ILC comprises two recombinant co-stimulatory ligands (e.g., 4-1BBL and CD80). CARs comprising at least one co-stimulatory ligand are described in U.S. Pat. No. 8,389,282, which is incorporated by reference in its entirety.

Furthermore, the presently disclosed engineered cytotoxic ILCs can further comprise at least one exogenous cytokine. For example, a presently disclosed engineered cytotoxic ILC can be further transduced with at least one cytokine, such that the engineered cytotoxic ILCs secrete the at least one cytokine as well as express IL-15, STAT5B, or any CAR disclosed herein (e.g., CAR including a truncated cytoplasmic domain of IL-2RβΔ). Additionally or alternatively, in certain embodiments, the at least one cytokine is selected from the group consisting of IL-2, IL-4, IL-7, IL-12, IL-15, IL-18, IL-21 and IL-23.

The engineered cytotoxic ILCs can be generated from peripheral donor lymphocytes (see Examples described herein). The engineered cytotoxic ILCs can be autologous, non-autologous (e.g., allogeneic), or derived in vitro from engineered progenitor or stem cells.

In certain embodiments, the presently disclosed engineered cytotoxic ILC expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a heterologous nucleic acid encoding IL-15, STAT5B, or any CAR disclosed herein (e.g., CAR including a truncated cytoplasmic domain of IL-2RβΔ).

For example, the higher the non-endogenous levels of IL-15, STAT5B, or any CAR disclosed herein (e.g., CAR including a truncated cytoplasmic domain of IL-2RβΔ) in an engineered cytotoxic ILC, the greater cytotoxicity and/or cytokine production the engineered cytotoxic ILC exhibits.

Additionally, or alternatively, the cytotoxicity and cytokine production of a presently disclosed engineered cytotoxic ILC are proportional to the expression level of IL-15, STAT5B, or any CAR disclosed herein (e.g., CAR including a truncated cytoplasmic domain of IL-2RβΔ) in the ILC.

The unpurified source of ILCs can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the engineered cytotoxic ILCs comprise one or more additional modifications. For example, in some embodiments, the engineered cytotoxic ILCs comprise and express (is transduced to express) a chimeric co-stimulatory receptor (CCR). CCR is described in Krause et al. (1998) J. Exp. Med. 188(4):619-626, and US20020018783, the contents of which are incorporated by reference in their entireties. CCRs mimic co-stimulatory signals, but do not provide a T-cell activation signal, e.g., CCRs lack a CD32 polypeptide. CCRs provide co-stimulation, e.g., a CD28-like signal, in the absence of the natural co-stimulatory ligand on the antigen-presenting cell.

In some embodiments, the engineered cytotoxic ILCs are further modified to suppress expression of one or more genes. In some embodiments, the engineered cytotoxic ILCs are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered cytotoxic ILCs are modified to result in disruption or inhibition of PD-1, PDL-1, Tim-3 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Administration

The engineered cytotoxic ILCs of the presently disclosed subject matter can be provided systemically or directly to a subject for treating cancer. In certain embodiments, the engineered cytotoxic ILCs described herein are directly injected into an organ of interest. Additionally or alternatively, the engineered cytotoxic ILCs of the present technology are provided indirectly to the organ of interest, for example, by administration into the circulatory system (e.g., the tumor vasculature) or into the tissue of interest (e.g., solid tumor). Expansion and differentiation agents can be provided prior to, during or after administration of cells and compositions to increase production of the engineered cytotoxic ILCs either in vitro or in vivo.

Engineered cytotoxic ILCs of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered cytotoxic ILCs can comprise a purified population of cells. Those skilled in the art can readily determine the percentage of engineered cytotoxic ILCs in a cell population using various well-known methods, such as fluorescence activated cell sorting (FACS). The ranges of purity in cell populations comprising engineered cytotoxic ILCs can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered cytotoxic ILCs can be introduced by injection, catheter, or the like. If desired, factors can also be included, including, but not limited to, interleukins, e.g., IL-2, IL-4, IL-7, IL-12, IL-15, IL-18, IL-21, IL-23, as well as the other interleukins, the colony stimulating factors, such as G-, M- and GM-CSF, interferons, e.g., γ-interferon.

In certain embodiments, compositions of the presently disclosed subject matter comprise any and all embodiments of the engineered cytotoxic ILCs of the present technology with a pharmaceutically acceptable carrier. Administration can be autologous or non-autologous. For example, the engineered cytotoxic ILCs of the present technology and compositions comprising the same can be obtained from one subject, and administered to the same subject or a different, compatible subject. Peripheral blood derived cytotoxic ILCs of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising any and all embodiments of the engineered cytotoxic ILCs disclosed herein), it can be formulated in a unit dosage injectable form (solution, suspension, emulsion).

### Formulations

The engineered cytotoxic ILCs of the present technology and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising the engineered cytotoxic ILCs of the present technology, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered cytotoxic ILCs of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered cytotoxic ILCs as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered cytotoxic ILCs of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered cytotoxic ILCs of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered cytotoxic ILCs of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered cytotoxic ILCs are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Therapeutic Uses of the Engineered Cytotoxic ILCs of the Present Technology

For treatment, the amount of the engineered cytotoxic ILCs provided herein administered is an amount effective in producing the desired effect, for example, treatment of a cancer or one or more symptoms of a cancer. An effective amount can be provided in one or a series of administrations of the engineered cytotoxic ILCs provided herein. An effective amount can be provided in a bolus or by continuous perfusion. For adoptive immunotherapy using ILCs, cell doses in the range of about 10⁶ to about 10¹⁰ may be infused. Lower doses of the engineered cytotoxic ILCs may be administered, e.g., about 10⁴ to about 10⁸.

The engineered cytotoxic ILCs of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered cytotoxic ILCs and the compositions comprising thereof are intravenously administered to the subject in need. Methods for administering cells for adoptive cell therapies, including, for example, donor lymphocyte infusion and engineered immune cell therapies, and regimens for administration are known in the art and can be employed for administration of the engineered cytotoxic ILCs provided herein.

For example, the presently disclosed subject matter provides methods of reducing tumor burden in a subject. In one non-limiting example, the method of reducing tumor burden comprises administering an effective amount of the presently disclosed engineered cytotoxic ILCs to the subject and administering a suitable antibody targeted to the tumor, thereby inducing tumor cell death in the subject. In some embodiments, the engineered cytotoxic ILCs and the antibody are administered at different times. For example, in some embodiments, the engineered cytotoxic ILCs are administered and then the antibody is administered. In some embodiments, the antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours or more than 48 hours after the administration of the engineered cytotoxic ILCs of the present technology.

The presently disclosed subject matter provides various methods of using any and all embodiments of the engineered cytotoxic ILCs provided herein. For example, the presently disclosed subject matter provides methods of reducing tumor burden in a subject. In one non-limiting example, the method of reducing tumor burden comprises administering an effective amount of the presently disclosed engineered cytotoxic ILCs to the subject, thereby inducing tumor cell death in the subject.

The presently disclosed engineered cytotoxic ILCs can reduce the number of tumor cells, reduce tumor size, and/or eradicate the tumor in the subject. In certain embodiments, the method of reducing tumor burden comprises administering an effective amount of engineered cytotoxic ILCs of the present technology to the subject, thereby inducing tumor cell death in the subject. Non-limiting examples of suitable tumors include adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject with cancer (e.g., a tumor). In one non-limiting example, the method of increasing or lengthening survival of a subject with cancer (e.g., a tumor) comprises administering an effective amount of the presently disclosed engineered cytotoxic ILCs to the subject, thereby increasing or lengthening survival of the subject. The presently disclosed subject matter further provides methods for treating or preventing cancer (e.g., a tumor) in a subject, comprising administering the presently disclosed engineered cytotoxic ILCs to the subject. Also provided herein are methods for treating of inhibiting tumor growth or metastasis in a subject comprising contacting a tumor cell with an effective amount of any of the engineered cytotoxic ILCs provided herein.

Cancers whose growth may be inhibited using the engineered cytotoxic ILCs of the presently disclosed subject matter include cancers typically responsive to immunotherapy. Non-limiting examples of cancers for treatment include breast cancer, endometrial cancer, ovarian cancer, colon cancer, lung cancer, stomach cancer, prostate cancer, renal cancer, pancreatic cancer, brain cancer, acute lymphoblastic leukemia (ALL), chronic lymphocytic leukemia (CLL), acute myeloid leukemia (AML), and metastases thereof.

Additionally, the presently disclosed subject matter provides methods of increasing immune-activating cytokine production in response to a cancer cell in a subject in need thereof. In one non-limiting example, the method comprises administering the presently disclosed engineered cytotoxic ILCs to the subject. The immune-activating cytokine (which is also referred to herein as a cytokine) can be granulocyte macrophage colony stimulating factor (GM-CSF), IFNα, IFN-β, IFN-γ, TNFα, IL-2, IL-4, IL-7, IL-12, IL-15, IL-18, IL-21, IL-23, and combinations thereof.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

Another group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the engineered cytotoxic ILCs described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the engineered cytotoxic ILCs to avert or minimize the risks of immunological complications, or when healthy tissues express the same target antigens as the tumor cells, leading to outcomes similar to GvHD. Modification of the engineered cytotoxic ILCs can include engineering a suicide gene into the engineered cytotoxic ILCs. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable ILC elimination by administering anti-EGFR monoclonal antibody (e.g., cetuximab). The suicide gene can be included within the vector comprising nucleic acids encoding IL-15, STAT5B, or any CAR disclosed herein (e.g., CAR including a truncated cytoplasmic domain of IL-2RβΔ). A presently disclosed engineered cytotoxic ILC incorporated with a suicide gene can be pre-emptively eliminated at a given time point post ILC infusion, or eradicated at the earliest signs of toxicity.

In another aspect, the present disclosure provides a method of preparing immune cells for adoptive cell therapy (ACT) comprising: (a) isolating cytotoxic innate lymphoid cells (ILCs) from a donor subject, (b) transducing the cytotoxic ILCs with a nucleic acid encoding IL-15 or STAT5B or an expression vector comprising said nucleic acid, and (c) administering the transduced cytotoxic ILCs to a recipient subject. In certain embodiments, the nucleic acid encodes the amino acid sequence of SEQ ID NO: 19, SEQ ID NO: 20, SEQ ID NO: 9 or SEQ ID NO: 23. Additionally or alternatively, in some embodiments, the method further comprises transducing the cytotoxic ILCs with a nucleic acid encoding a chimeric antigen receptor (CAR) that binds to a tumor antigen. Also disclosed herein is a method of preparing immune cells for adoptive cell therapy (ACT) comprising: (a) isolating cytotoxic innate lymphoid cells (ILCs) from a donor subject, (b) transducing the cytotoxic ILCs with a nucleic acid encoding a chimeric antigen receptor (CAR) that binds to a tumor antigen or an expression vector comprising said nucleic acid, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains, and (c) administering the transduced cytotoxic ILCs to a recipient subject. In some embodiments of the ACT methods described herein, the donor subject and the recipient subject are the same or different.

### Combination Therapy

The engineered cytotoxic ILCs of the present technology may be employed in conjunction with other therapeutic agents useful in the treatment of cancers. For example, any and all embodiments of the engineered cytotoxic ILCs described herein may be separately, sequentially or simultaneously administered with at least one additional cancer therapy. Examples of additional cancer therapy include chemotherapeutic agents, immune checkpoint inhibitors, monoclonal antibodies that specifically target tumor antigens, immune activating agents (e.g., interferons, interleukins, cytokines), oncolytic virus therapy and cancer vaccines. In some embodiments, the additional cancer therapy is selected from among a chemotherapy, a radiation therapy, an immunotherapy, a monoclonal antibody, an anti-cancer nucleic acid, an anti-cancer protein, an anti-cancer virus or microorganism, a cytokine, or any combination thereof.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

In some embodiments, the methods further comprise sequentially, separately, or simultaneously administering an immunotherapy to the subject. In some embodiments, the immunotherapy regulates immune checkpoints. In further embodiments, the immunotherapy comprises, or consists essentially of, or yet further consists of an immune checkpoint inhibitor, such as a Cytotoxic T-Lymphocyte Associated Protein 4 (CTLA-4) inhibitor, or a Programmed Cell Death 1 (PD-1) inhibitor, or a Programmed Death Ligand 1 (PD-L1) inhibitor. In yet further embodiments, the immune checkpoint inhibitor comprises, or consists essentially of, or yet further consists of an antibody or an equivalent thereof recognizing and binding to an immune checkpoint protein, such as an antibody or an equivalent thereof recognizing and binding to CTLA-4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

In some embodiments, the methods further comprise sequentially, separately, or simultaneously administering a cytokine to the subject. In some embodiments, the cytokine is administered prior to, during, or subsequent to administration of the one or more cytotoxic ILCs. In some embodiments, the cytokine is selected from the group consisting of interferon α, interferon β, interferon γ, complement C5a, IL-2, TNFα, CD40L, IL-12, IL-23, IL-15, IL-18, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The methods for treating cancer may further comprise sequentially, separately, or simultaneously administering to the subject at least one chemotherapeutic agent, optionally selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicin, doxorubicin analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11.

### Kits

The presently disclosed subject matter provides kits for the treatment or prevention of a disease, such as cancer. In certain embodiments, the kit may be used in the manufacture of a therapeutic or prophylactic composition containing an effective amount of engineered cytotoxic ILCs. In some embodiments, the kits include a non-endogenous expression vector comprising a heterologous mammalian IL-15 or STAT5B nucleic acid. Additionally or alternatively, in some embodiments, the kit comprises a vector comprising any and all embodiments of the CARs disclosed herein, or other cell-surface ligand that binds to a target antigen, such as a tumor antigen. In certain embodiments, the CAR comprises (i) an extracellular antigen binding domain comprising a single chain variable fragment (scFv) that binds to the tumor antigen; (ii) a transmembrane domain; and (iii) an intracellular domain comprising a truncated cytoplasmic domain of IL-2RβΔ and one or more co-stimulatory domains.

In some embodiments, the vector comprising the heterologous mammalian IL-15 or STAT5B nucleic acid and the vector comprising the CAR or cell-surface ligand that binds to a target antigen are the same. In other embodiments, the vector comprising the heterologous mammalian IL-15 or STAT5B nucleic acid and the vector comprising the CAR or cell-surface ligand that binds to a target antigen are distinct.

In some embodiments, the kit comprises a sterile container which contains the kit components; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for preparing ILCs for adoptive cell therapy, and methods of using ILCs to treat cancer in a subject in need thereof. The instructions will generally include information about the use of the composition for the treatment or prevention of cancer. In other embodiments, the instructions include at least one of the following: description of the therapeutic agent; dosage schedule and administration for treatment or prevention of cancer or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

The at least one engineered cytotoxic ILC of the present technology may be provided to the subject in the form of a syringe or autoinjection pen containing a sterile, liquid formulation or lyophilized preparation (e.g., Kivitz et al., Clin. Ther. 28:1619-29 (2006))

A device capable of delivering the engineered cytotoxic ILCs of the present technology through an administrative route may be included in the kit. Examples of such devices include syringes (for parenteral administration) or inhalation devices.

The kit components may be packaged together or separated into two or more containers. In some embodiments, the containers may be vials that contain sterile, lyophilized formulations that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

### EXAMPLES

### Example 1: Materials and Methods (Related to Examples 1-9)

Study Design. The main goal of this study was to explore the heterogeneity of type 1 innate lymphocytes in mice using mouse genetics and molecular and cellular immunology approaches. The sample size for the flow cytometry and other experiments were based on previous studies and pilot experiments in the lab that allowed for conclusive data in measuring changes in cell populations. All experiments were performed at least two to three times independently, the number of individual mice or samples are always indicated in the figure legends, mice were age and sex-matched, and litter-mate or cage-mates were used where possible. The experiments were performed unblinded as mice of different genotypes were identified and kept track of, except for tumor measurements and scoring of histology slides.

Animal experimental models. Eomes^{fl}, Il17a^{iCre}, Il2rg^{-/-}, Il22^{iCre}, Il15^{tdT-iCre}, Rag1^{-/-}, Rag2^{-/-}, Rosa26^{LSL-DTA}, Rosa26^{FLPe}, and Tbx21^{fl} mice were purchased from Jackson Laboratories. Rosa26^{LSL-YFP}, IL-15^{-/-}, Prf1^{-/-}, MMTV-PyMT, Rosa26^{LSL-Stat5b-CA} and Tgfbr2^{fl} mouse strains were previously reported (3, 40-42), Gzmc^{tdTomato-T2A-iCre} mice were generated by insertion of a targeting construct into the Gzmc locus by CRISPR/Cas9-mediated homologous recombination in embryonic stem cells (ESCs) on the albino C57BL/6 background. The targeting construct was generated by inserting 2 kb homology arms upstream and downstream of the 3′UTR of Gzmc derived from BAC RP23 (BACPAC Resources Center) into Zero blunt plasmid backbone, and then inserting IRES, tandem dimer (td) Tomato, T2A self-cleaving peptide, iCre recombinase, and frt site-flanked PGK-Neomycin resistance gene (NEO)-BGHpA cassette in between homology arms as previously described (43). Guide RNA targeting the 3′ UTR of Gzmc (5′-TCCTGGACTCAGCTATGGGG-3′ (SEQ ID NO: 68)) was cloned into the pX335 vector. The targeting construct and pX335 vector were co-injected into ESCs. Neomycin-resistant ESC clones were subject for Southern Blot analysis after EcoRI restriction enzyme treatment to confirm proper insertion of the construct, and for karyotyping to confirm chromosome integrity. Clones with successful insertions and stable chromosomes were injected into pseudo-pregnant female C57BL/6 mice mixed with non-albino C57BL/6 ESCs. Of resulting pups, the construct successfully went germline, as noted by white fur chimerism and genotyped offspring. Mice harboring the construct were then crossed with Rosa26^{FLPe} mice to ensure removal of the NEO cassette. S1pr5^{eGFP-T2A-iCre} mice were generated in the same manner as Gzmc^{tdT-T2A-iCre} mice, except that the targeting vector contained eGFP in place of tdTomato and targeted the S1pr5 3′ UTR with associated homology arms, and BamHI was used in the Southern Blot analysis.

Immune cell isolation. Immune cells were isolated from spleen, salivary gland, liver, small intestine lamina propria, and PyMT tumor as described previously (3, 44). Briefly, spleen single-cell suspension was obtained by tissue disruption with glass slides. For liver, tissue was manually dissociated using a razor blade and digested with 1 mg/ml Collagenase D (Sigma Aldrich) for 30 minutes at 37° C. For small intestine, tissue was dissected and washed in PBS. Intestines were cut in small pieces and incubated in 1 mM dithiothreitol and 1 mM EDTA in PBS with gentle agitation for 30 minutes. The supernatant containing intraepithelial lymphocytes was removed. Remaining tissue was digested in 1 mg/ml Collagenase Type III (Worthington) and 4 μg/ml DNase I (Sigma-Aldrich) in completed RPMI with gentle agitation for 20 minutes. For salivary gland and PyMT tumor, tissue was manually dissociated using a razor blade and digested for 1 hour in 1 mg/ml Collagenase Type III (Worthington) and 4 μg/ml DNase I (Sigma-Aldrich) in HBSS at 37° C. with periodic vortex. After collagenase treatment, cells isolated from the liver, salivary gland, small intestine and tumor were filtered through a 70-μM cell strainer, layered in a 44% and 66% Percoll gradient (Sigma-Aldrich), and centrifuged at 1,900 g for 30 minutes without brake. Cells at the interface were collected and analyzed by flow cytometry. For bone marrow, bones were collected from limbs of mice, cleaned, sterilized, mashed in a mortar and pestle, and passed through 70 μm cell strainer. Lineage cells were depleted from bone marrow using MagniSort Lineage depletion kit (Invitrogen). For CLP and LSK, bones from one mouse were pooled per sample. For ILCp, bones from at least six mice were pooled per sample. Cells were then stained for sorting.

Flow cytometry. Cells were preincubated with 2.462 mAB (Bio X Cell) to block non-specific FcγR binding and Ghost Dye (Tonbo) to detect dead cells and were stained with panels of antibodies for 30 min on ice. Fluorochrome-conjugated or biotinylated antibodies against mouse CD103 (M290), CD11b (M1/70), CD27 (LG.3A10), CD4 (RM4-5), CD45 (30-F11), CD49a (Ha31/8), CD8β (H35-17.2), IL-17A (TC11-18H10.1), IL-5 (TRFK5), Ly6C (AL-21), and TCRγδ (GL3) were purchased from BD Biosciences. Antibodies against mouse αGalCer:CD1d (L363), CD127 (A7R34), CD19 (1D3), CD3ε (145-2C11), CD69 (H1.2F3), CD90.2/Thy1.2 (53-2.1), CXCR6 (SA051D1), granzyme C (SFC1D8), KLRG1 (2F1), NKp46 (29A1.4), and perforin (S16009A) were purchased from BioLegend. Antibodies against mouse CD200R1 (OX110), CD49b (Dx5), CD5 (53-7.3), Eomes (Dan11mag), IL-22 (1H8PWSR), NK1.1 (PK136), and T-bet (eBio4b10) and against Rabbit (anti-F(ab)2′) (polyclonal) were purchased from eBioscience, now Thermo Scientific. GFP (raised in rabbit) and human/mouse granzyme B (GB11) were purchased from Invitrogen. Mouse B220 (RA3-6B2), CD127 (A7R34), CD8a (53-6.7), F4/80 (BM8.1), IFN-γ (XMG1.2), Ly6G (1A8), and TCRβ (H57-597) were purchased from Tonbo Biosciences. CellTrace Violet was purchased from Thermo Fisher. For preservation of fluorescent proteins, cells were fixed in 4% PFA for 15 minutes on ice after surface staining. For intracellular antibodies, Tonbo transcription factor kit was used to fix and permeabilize cells for 30 minutes on ice, followed by intracellular staining for 30 minutes on ice. All samples were acquired and analyzed with LSRII flow cytometer (Becton Dickson), FACSDiva (Becton Dickson) and FlowJo (TreeStar) software.

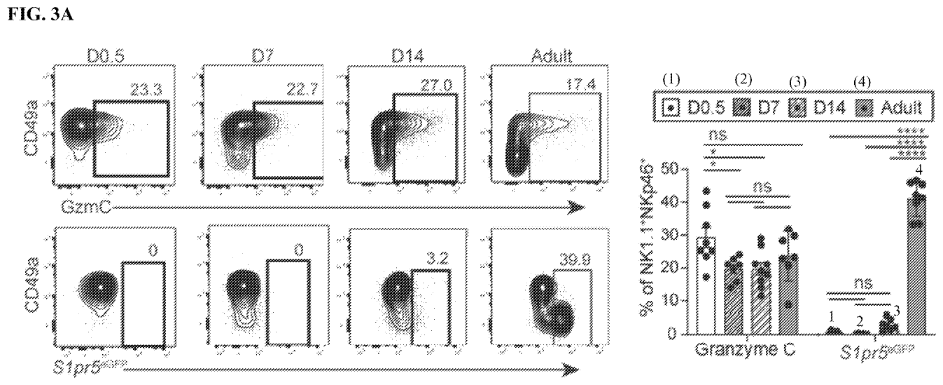

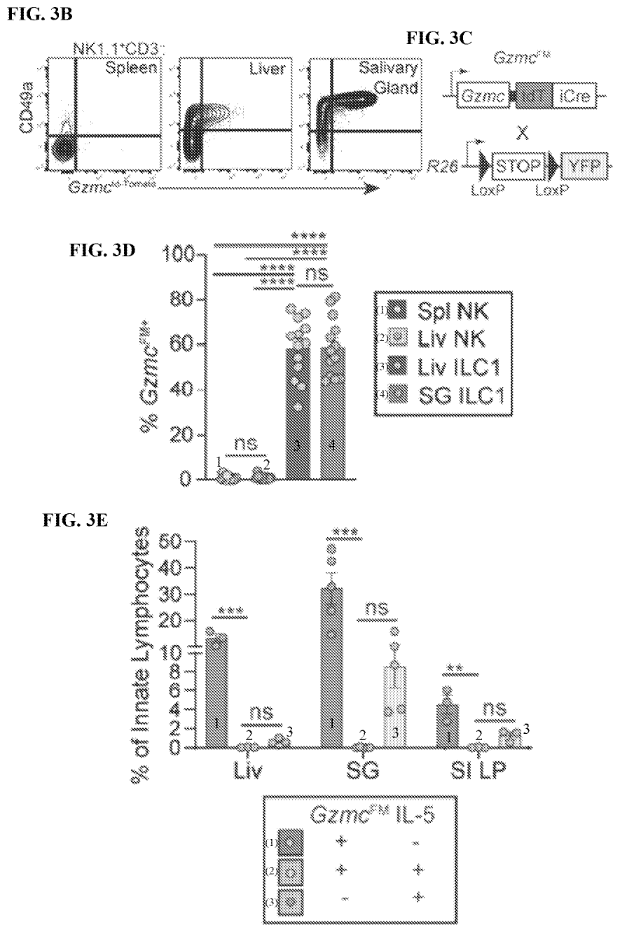

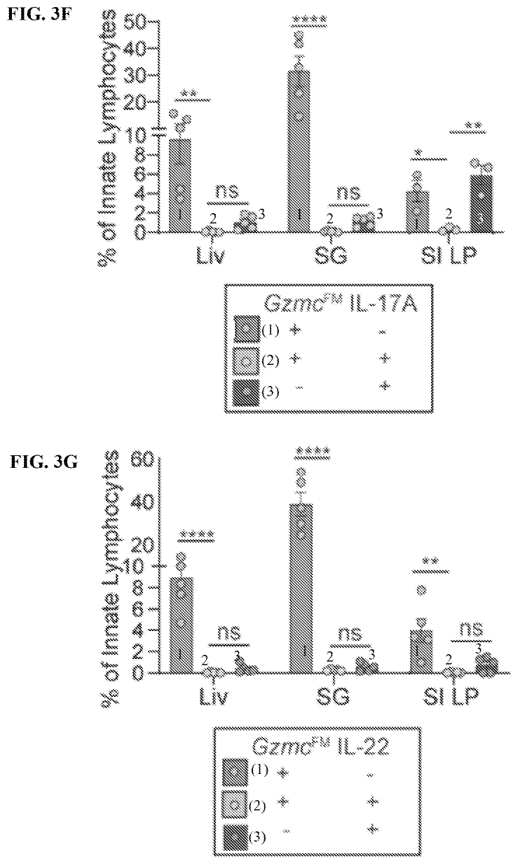

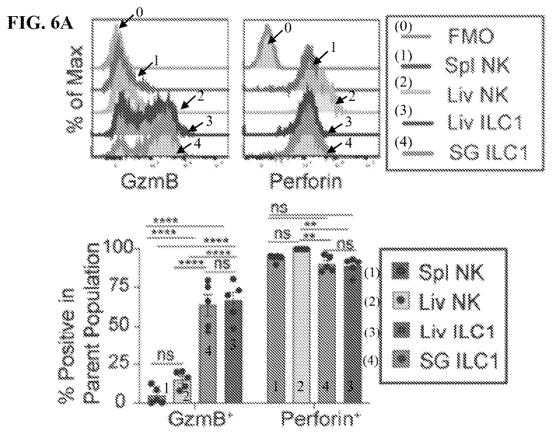

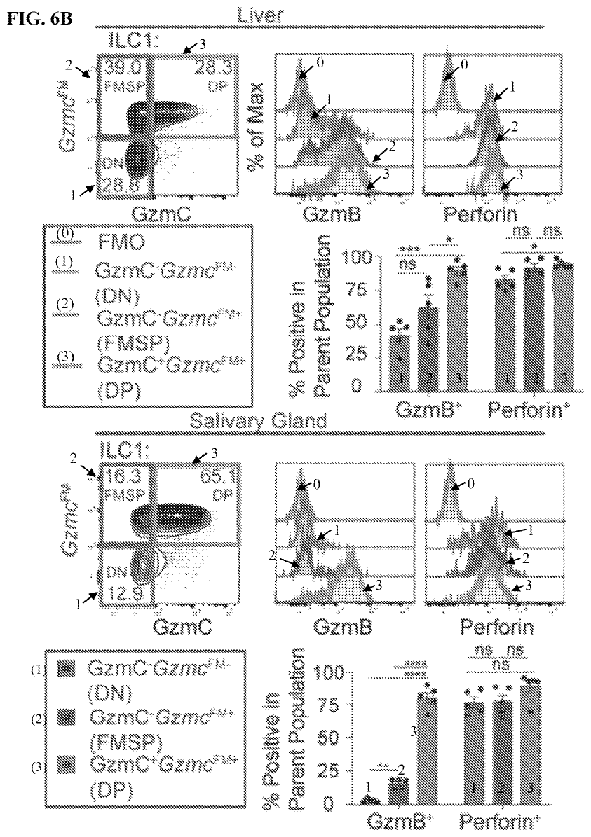

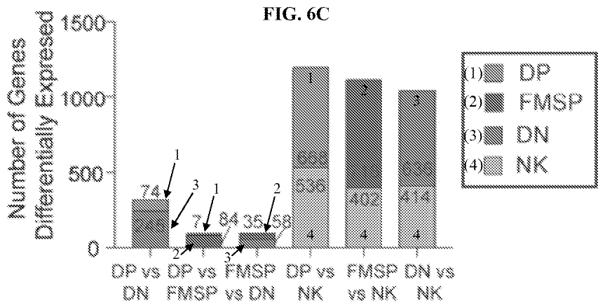

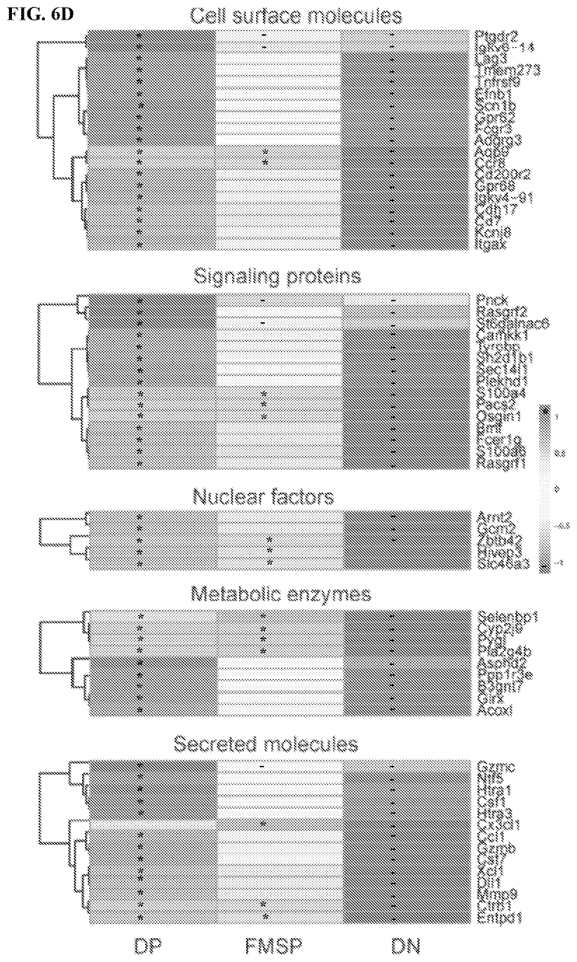

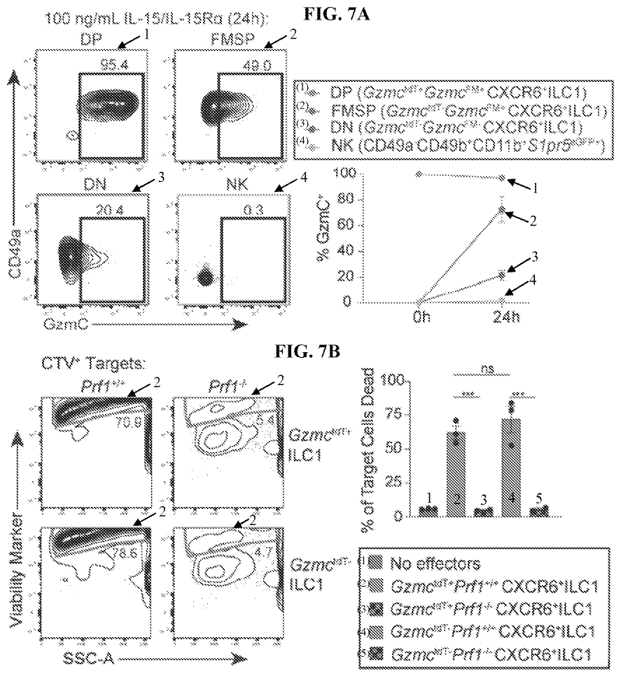

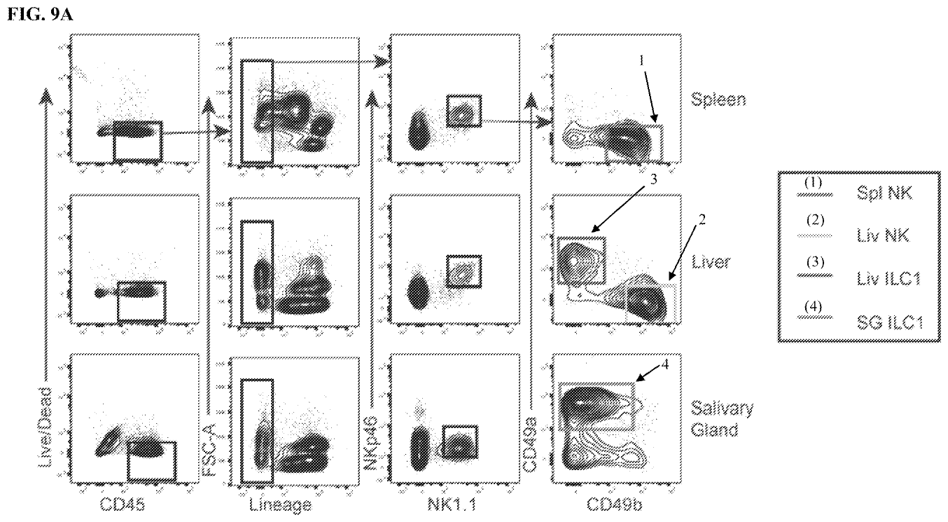

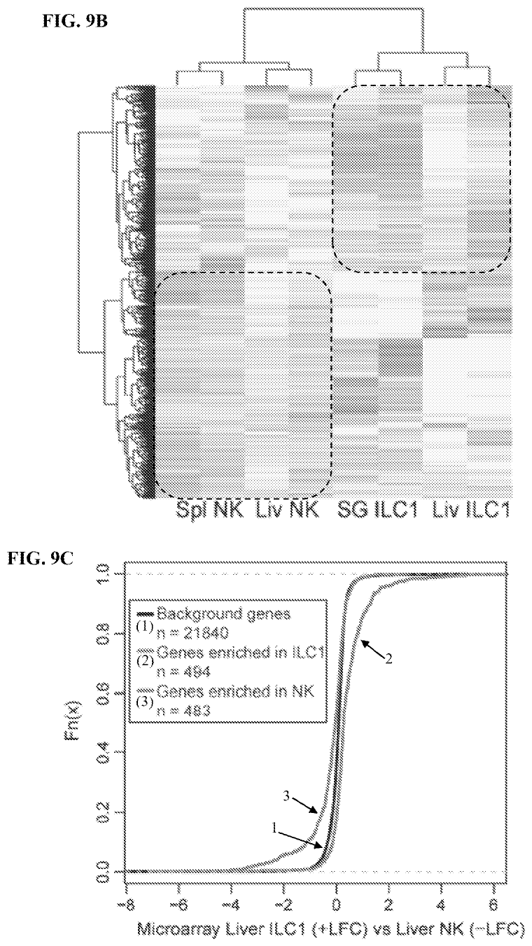

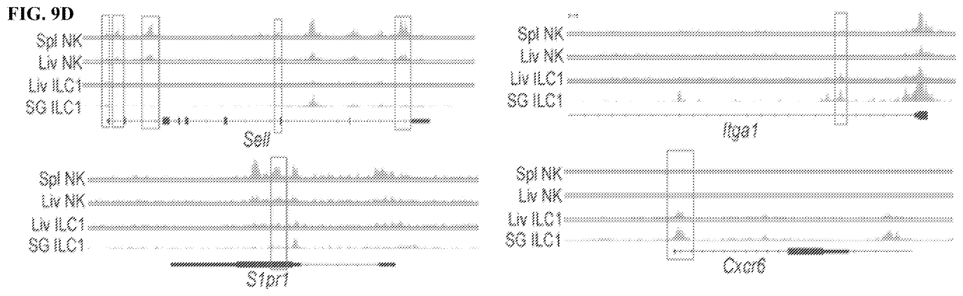

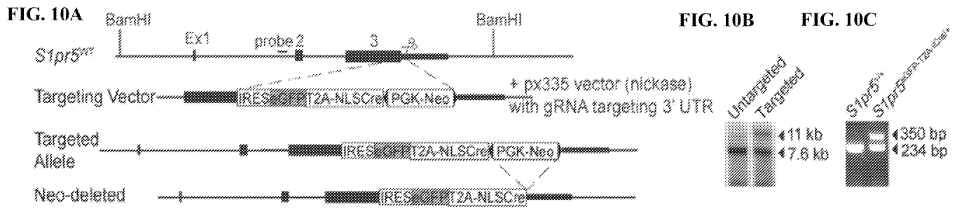

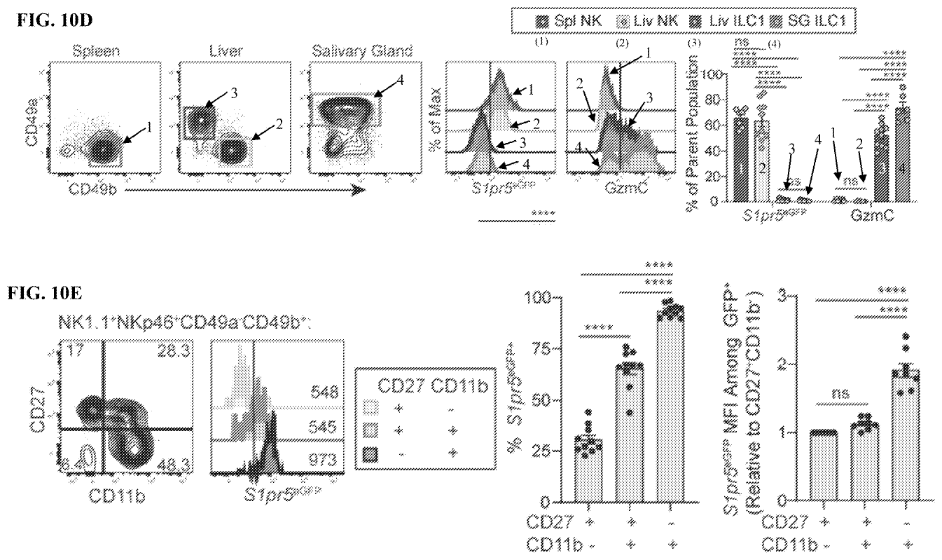

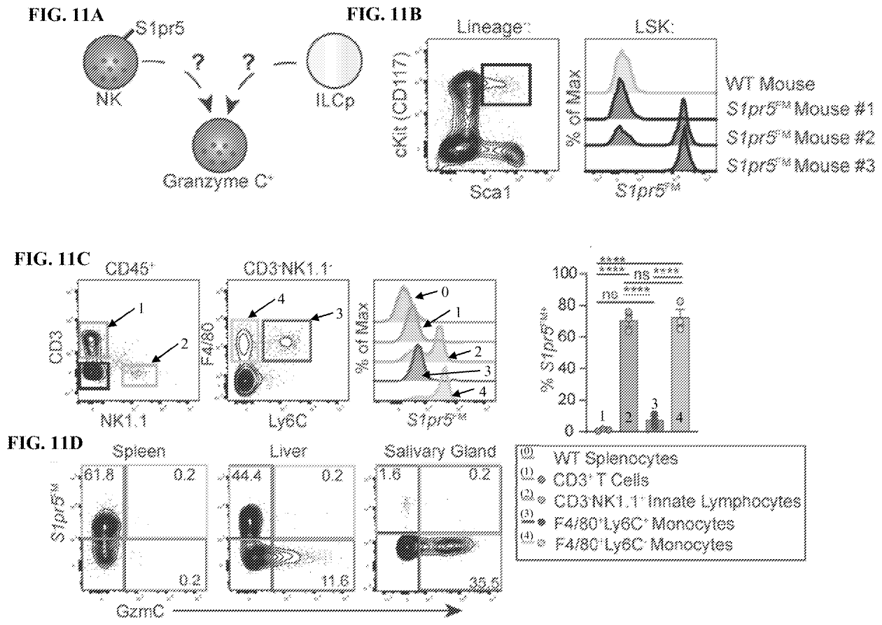

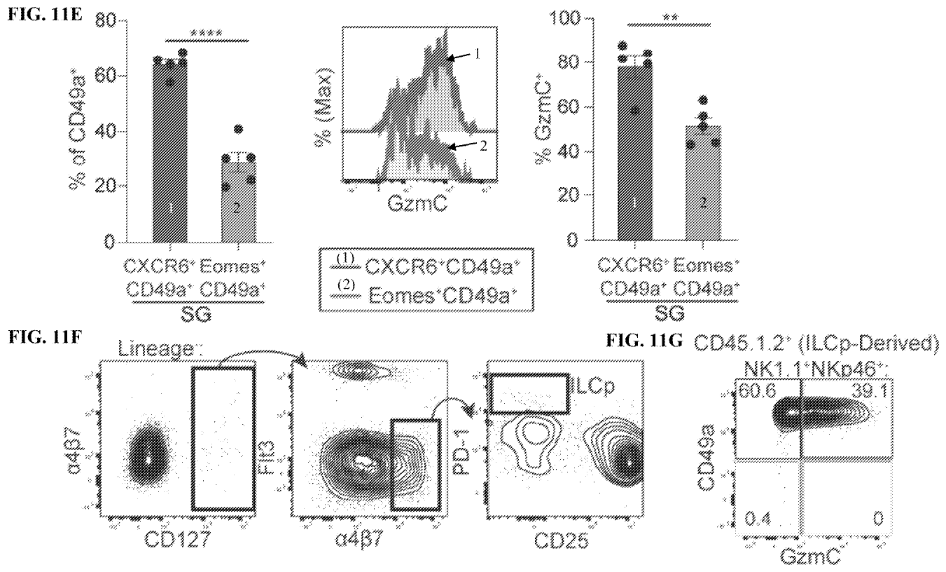

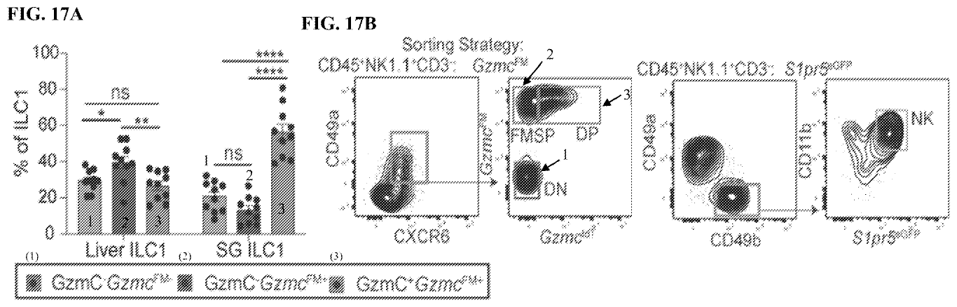

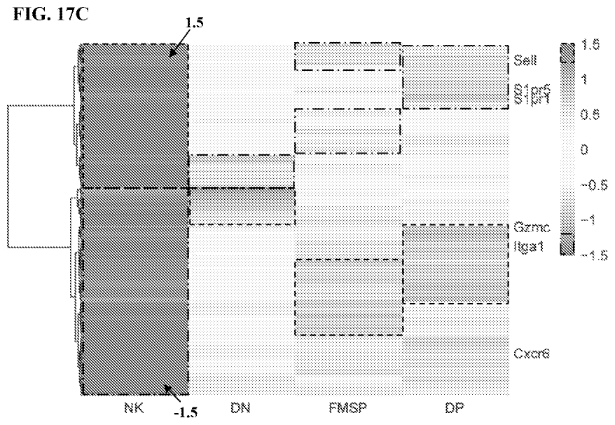

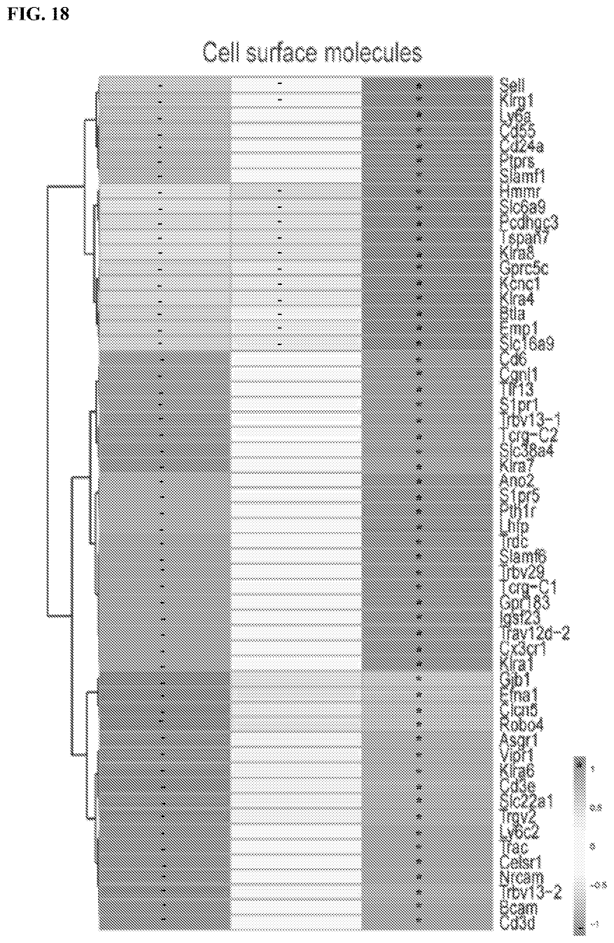

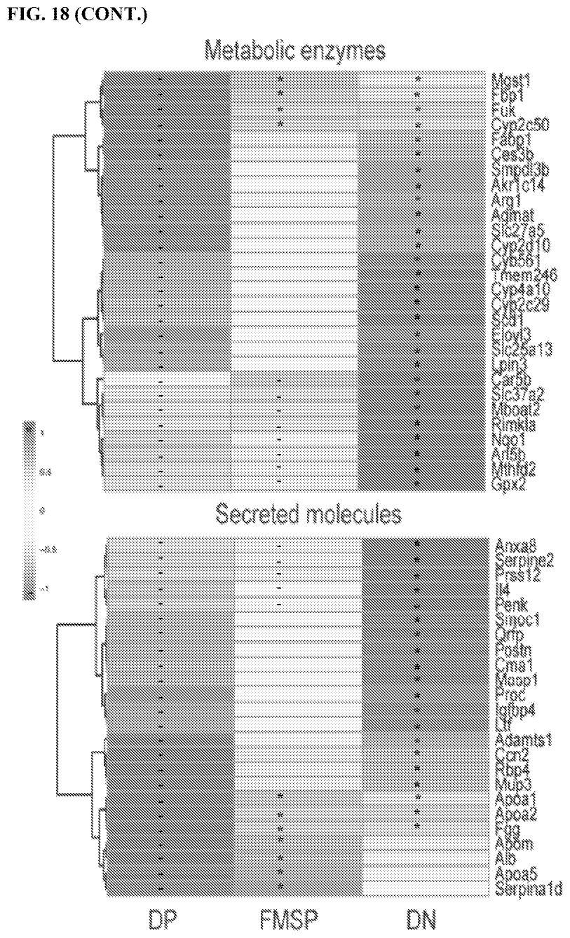

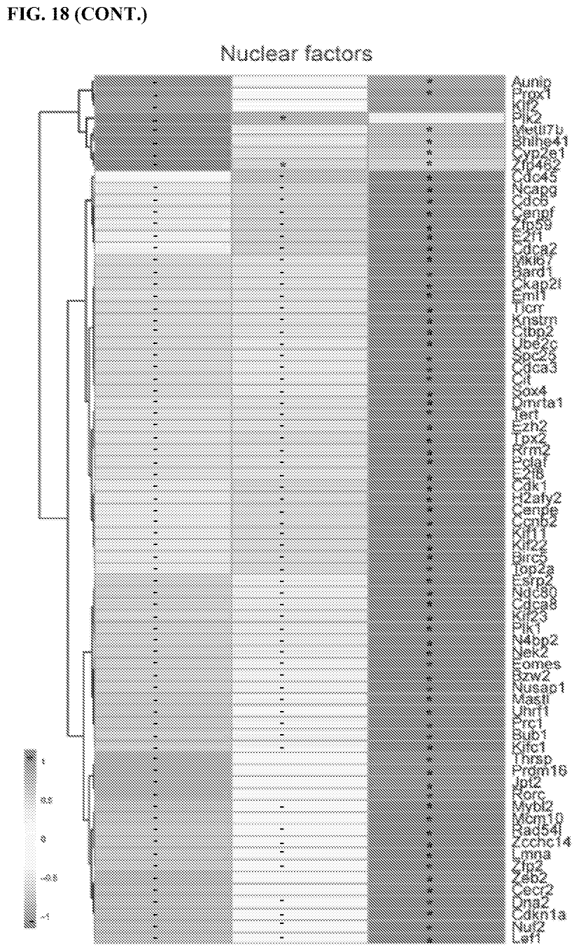

Cell sorting and transfer. After gating on morphology and singlets, CD45 Dead cells were gated as follows: related to FIG. 9-FIG. 10 for ATACsequencing: lineage (CD19, CD3, CD5, Ly6G, TCRB)-negative, NK1.1+NKp46*, for NK populations: CD49a-CD49b⁺ (spleen and liver), for ILC1 populations: CD49a⁺CD49b− (liver, salivary gland). Related to FIG. 3A and FIG. 11B, lineage (B220, CD11b, CD19, CD3e, Gr1, Ly6D, TER119)−, CD117⁺Sca1⁺, S1pr5^{FM-} (non-FM, YFP⁻, LSKs). Cell sorting was conducted on Aria II (Becton Dickson). Related to FIG. 11E, Lineage (same as LSK)-NK1.1⁻CD127⁺α4β7⁺Flt3⁻PD-1⁺CD25⁻ (ILCp). After sorting ILCp from pooled bone marrow of 6 mice (age 4-6 weeks), ILCp (around 4,000 cells) were transferred into one recipient Rag2^{-/-}Il2rg^{-/-} mouse. Recipient mice were analyzed around 4 weeks after transfer. Related to FIG. 6, FIG. 17 and FIG. 18 for RNA sequencing and FIG. 7A for IL-15 culture: NK1.1⁺CD3⁻CD49a⁺CXCR6⁺, then Gzmc^{tdT+}Gzmc^{FM+} (DP), Gzmc^{tdT-}Gzmc^{FM+} (FMSP), Gzmc^{tdT-}Gzmc^{FM-} (DP) from livers of Gzmc^{FM} mice, and NK1.1⁺CD3⁻CD49a⁻ CD49b⁺CD11b⁺S1pr5^{eGFP+} (NK) from livers of S1pr5^{eGFP} mice. Related to FIG. 7B for killing assay: NK1.1⁺CD3⁻CD49a⁺ CXCR6⁺, then Gzmc^{tdT+} or Gzmc^{tdT-} from livers of either Gzmc^{tdT-T2A-iCre/+} or Gzmc_{tdT-T2A-iCre/+}Prf1^{-/-} mice.

ATAC sample preparation and sequencing. 50,000 cells were sorted for each sample and frozen in FBS+10% DMSO. Profiling of chromatin was performed by ATACseq as described previously (45). Briefly, 50,000 viably frozen cells were washed in cold PBS and lysed. The transposition reaction was incubated at 42° C. for 45 minutes. The DNA was cleaned with the MinElute PCR Purification Kit (Qiagen catalog #28004) and material was amplified for 5 cycles. After evaluation by real-time PCR, 8-10 additional PCR cycles were done. The final product was cleaned by aMPure XP beads (Beckman Coulter catalog #A63882) at a 1× ratio. Libraries were sequenced on a Hiseq 2500 in High Output mode in a PE50 run, using the TruSeq SBS Kit v4 (Illumina). An average of 57 million paired reads were generated per sample.

Bone marrow chimera. Recipient mice (6-8 weeks old, CD45.1.2⁺) were irradiated with 900 Gyz. Eighteen hours after irradiation, bone marrow was harvested from CD45.2⁺ S1pr5^{eGPP-iCre/+}Rosa26^{LSL-YFP} mice, lineage depleted, stained with flow antibodies, sorted for non-fate mapped LSK populations (detailed above), and transferred intravenously into recipient mice. Mice were maintained on sulfatrim antibiotic diet and aged to allow bone marrow to graft. Mice were analyzed 4-5 months after transfer.

Tumor measurement. At 12 and 16 weeks of age, tumors were measured with a caliper. Tumor volume was calculated using the equation [(L×W²)×(π/6)], in which L denotes length and W denotes width. Individual tumor volumes were added together to calculate total tumor burden per mouse. Researchers were blinded to genotypes of mice during measurements,

RNA sample preparation and sequencing. All cells in a given population were sorted from one mouse per sample for DP, FMSP, DN and NK cells directly into 750 μL Trizol LS. After sort, total volume was brought up to 1 mL with sterile PBS, and samples were flash frozen. Four DP, four FMSP, three DN and four NK cell samples were processed and sequenced. For RNA extraction, phase separation in cells lysed in 1 mL TRIzol Reagent (ThermoFisher catalog #15596018) was induced with 200 μL chloroform and RNA was extracted from the aqueous phase using the miRNeasy Micro Kit (Qiagen catalog #217084) on the QIAcube Connect (Qiagen) according to the manufacturer's protocol with 350 μL input. Samples were eluted in 15 μL RNase-free water. For transcriptome sequencing, after RiboGreen quantification and quality control by Agilent BioAnalyzer, 282-300 pg total RNA with RNA integrity numbers ranging from 7.8 to 9.4 underwent amplification using the SMART-Seq v4 Ultra Low Input RNA Kit (Clonetech catalog #63488), with 12 cycles of amplification. Subsequently, 2.1-3 ng of amplified cDNA was used to prepare libraries with the KAPA Hyper Prep Kit (Kapa Biosystems KK8504) using 8 cycles of PCR. Samples were barcoded and run on a NovaSeq 6000 in a PE100 run, using the NovaSeq 6000 S4 Reagent Kit (200 Cycles) (Illumina). An average of 82 million paired reads were generated per sample and the percent of mRNA bases per sample ranged from 67% to 83%.

In vitro cell culture. Cells were first sorted as indicated above. Related to FIG. 7A, after sorting based on current and/or past expression of granzyme C, cells were cultured in T cell media (RPMI supplemented with 10% FCS, 1 mM sodium pyruvate, non-essential amino acids [Gibco], 10 mM Hepes, 55 uM 2-Mercaptoethanol, 100 U/mL Penicillin G and 0.1 mg/ml Streptomycin) supplemented with 100 ng/mL mouse IL-15/IL-15Rα (Invitrogen) for 24 hours at 37° C. Cells were then stained for flow cytometric analysis to measure expression of granzyme C.

Killing assay. Killing assay was performed as previously reported (3). Briefly, cells were sorted as described above, cultured in 100 ng/ml IL-15/IL-15Rα complex for one week, changing media every 3-4 days. Target RMA-S cells were maintained in T cell media. On day of experiment, RMA-S cells were stained with CellTrace Violet and mixed with sorted and expanded Gzmc^{tdT+} or Gzmc^{tdT-} ILC1s at a ratio of 10 effector:1 target cell. Cells were incubated for 16 hours at 37° C. in TCM supplemented with 100 ng/ML IL-15/IL-15Rα in 96-well U-bottom plate. After incubation, EDTA was added to separate cell conjugates. Cells were stained with Tonbo Ghost Dye to detect dead cells and were analyzed via flow cytometry for dead cells among CTV⁺ target cells. Experiment was repeated three times, each experiment including cells sorted from five mice.

Neonatal mouse necropsy phenotyping. Gzmc^{tdT-iCre}Rosa26^{LSL-STAT5b-CA}, Gzmc^{tdT-iCre}Rosa26^{LSL-STAT5b-CA}Perf^{-/-}, or wild-type littermate mice were euthanized at 14 days of age with CO₂. Following gross examination all organs were fixed in 10% neutral buffered formalin, followed by decalcification of bone in a formic acid solution (Surgipath Decalcifier I, Leica Biosystems). Tissues were then processed in ethanol and xylene and embedded in paraffin in a Leica ASP6025 tissue processor. Paraffin blocks were sectioned at 5 microns, stained with hematoxylin and eosin (H&E), and examined by a board-certified veterinary pathologist (AOM). The following tissues were processed and examined: heart, thymus, lungs, liver, gallbladder, kidneys, pancreas, stomach, duodenum, jejunum, ileum, cecum, colon, lymph nodes (submandibular, mesenteric), salivary glands, skin (trunk and head), urinary bladder, uterus, cervix, vagina, ovaries, oviducts, adrenal glands, spleen, thyroid gland, esophagus, trachea, spinal cord, vertebrae, sternum, femur, tibia, stifle join, skeletal muscle, nerves, skull, nasal cavity, oral cavity, teeth, ears, eyes, pituitary gland, and brain.

Immunohistochemistry. IHC was performed on a Leica Bond RX automated stainer using Bond reagents (Leica Biosystems, Buffalo Grove, IL), including a polymer detection system (DS9800, Novocastra Bond Polymer Refine Detection, Leica Biosystems). The chromogen was 3,3 diaminobenzidine tetrachloride (DAB), and sections were counterstained with hematoxylin. Details for each marker are shown in the table below.

| | | Primary | | Secondary | |
|---|---|---|---|---|---|
| | | antibody | Primary | antibody | Secondary |
| | | source, | antibody | source, | antibody |
| | Epitope | catalog | concen- | catalog | concen- |
| Marker | retrieval | number | tration | number | tration |
| Cleaved | Heat | Cell | 1:250 | Biotinylated | 1:100 |
| caspase | induced, | signaling, | | Anti-Rabbit | |
| 3 (CC3) | pH 6.0 | Cat. 9661 | | IgG (H + L), | |
| | | | | Vector, Cat. | |
| | | | | #BA1000 | |
| Mouse | Heat | R&D | 1:1000 | Biotinylated | Vector |
| NKp46 | induced, | Systems, | | Anti-Goat | Laboratories, |
| | pH 6.0 | Cat. | | IgG (H + L) | Cat. BA- |
| | | AF2225 | | | 5000 |

### Quantification and Statistical Analysis

ATACseq analysis. For all replicates in all biological conditions, we first used seqtk to trim 10 bp from either end of raw paired-end ATAC-seq reads. The trimmed fastqs were then aligned to the mm10 reference genome using bowtie2(46). Duplicates were removed using samtools (47, 48), and peaks were called using MACS2(49) with parameters “shift 100 --extsize 200 -p 0.2 -B-SPMR”. To define an atlas of peaks reproducible across replicates within each biological condition, we used the irreproducible discovery rate framework (IDR) at FDR P<0.1 for each pair of replicates within each condition, followed by merged peak calling across all replicates using MACS2. This defined a final atlas of 51412 peaks for downstream analysis. Peaks were annotated to the closest gene.

Differential accessibility analysis, CDF, differential gene expression. For differential accessibility analyses, we used DESeq2(50), and peaks were considered significantly differentially accessible at FDR P<0.05. We first performed two rounds of differential accessibility analysis-liver ILC1 vs liver NK cell and splenic NK cell, and then salivary gland ILC1 vs liver NK cell and splenic NK cell. Peaks commonly differentially accessible across both comparisons were considered ILC1 lineage peaks, and we repeated this procedure with chromatin accessibility profiles from NK cells to define NK cell lineage peaks. Differentially expressed genes between liver ILC1 and liver NK cell genes were obtained from (7); genes were considered significantly differentially expressed at FDR P<0.05. For CDF analyses, we overlapped all genes with significantly differentially accessible peaks from the comparison described above, and the Kolmogorov-Smirnov test was used to evaluate differences in log fold change distributions. Fold change versus fold change plot showing log fold change (LFC) of gene expression from microarray of liver ILC1 versus liver NK cell versus mean accessibility LFC for liver ILC1 versus liver NK cell was also generated.

RNA sequencing analysis. Paired-end reads in fastq format from 15 total samples (4 DP, 4 FMSP, 3 DN, and 4 NK) were quantified at the transcript level using the mm10 reference with Salmon (v1.6) (51) using default parameters, and then aggregated to gene-level counts using tximport (v1.2) (52). Differential expression analyses were conducted using DESeq2(50) separately for all six pairwise comparisons between the four cell types, and genes were included in the six lists of differentially expressed genes (data not shown) if they met all three of the following criteria: base mean expression >50, false discovery rate (FDR) P<0.05, and log 2 fold change either >1 or <−1. Genes were included in the ‘core’ list of NK cell and ILC1 genes (data not shown) if they were differentially expressed in the same direction in all three of the separate NK cell-ILC1 comparisons. Z-scores across groups of the log-regularized transformation values produced by DESeq2 were used to visualize the differentially expressed genes in heatmaps in FIG. 6D, FIG. 17C, and FIG. 18. Assignment of genes to the five classes in FIG. 6D and FIG. 18 (cell surface molecules, nuclear factors, metabolic enzymes, signaling proteins, and secreted molecules) was done manually.

Quantitative digital image analysis HALO. Expression of CC3 and NKp46 on IHC of mouse livers was evaluated quantitatively by automated image analysis. Whole-slide digital images were generated on a scanner (Panoramic Flash 250, 3DHistotech, 20×/0.8NA objective, Budapest, Hungary), at a 0.243094 μm/pixel resolution. Image analysis was performed on HALO software (Indica Labs, Albuquerque, NM), employing the Area Quantification module v.2.1.2.0. Region of interest (ROI) was annotated manually, including liver tissue and excluding folded tissue. The area quantification module was used to detect area of CC3 and NKp46 based on the optical density (OD) of DAB immunoreactivity after determining the OD threshold. Tissues from three mice per genotype were analyzed.

Statistical analysis. All data are displayed as mean+/−SEM, each dot represents one mouse. For pair-wise comparisons between two samples, unpaired student t test, two-tailed, was used. For three or more samples, one-way ANOVA with Tukey's multiple comparisons test was used. For survival analysis, Log-rank (Mantel Cox) test was performed. “ns”=not significant, *=p<0.05, **=p<0.01, ***=p<0.001, **=p<0.0001. All statistical analysis listed here was performed using GraphPad Prism software.

### Example 2: S1pr5 and Granzyme C Mark Subsets of Circulating and Tissue-Resident Group 1 Innate Lymphocytes, Respectively

The chromatin landscape is a key determinant of gene expression programs and can mark cell differentiation states. To explore chromatin accessibility among group 1 innate lymphocytes, we isolated two populations with an NK cell phenotype (NK1.1⁺NKp46⁺CD49a⁻CD49b⁺) from the liver and spleen, and two populations with an ILC1 phenotype (NK1.1⁺NKp46⁺CD49a⁺) from the liver and salivary gland for ATAC-sequencing (ATACseq) (FIG. 9A). Pairwise comparisons between each of the NK cell populations with each of the ILC1 populations yielded 1,005 and 821 peaks with increased accessibility among NK cells and ILC1s, respectively (FIG. 9B).

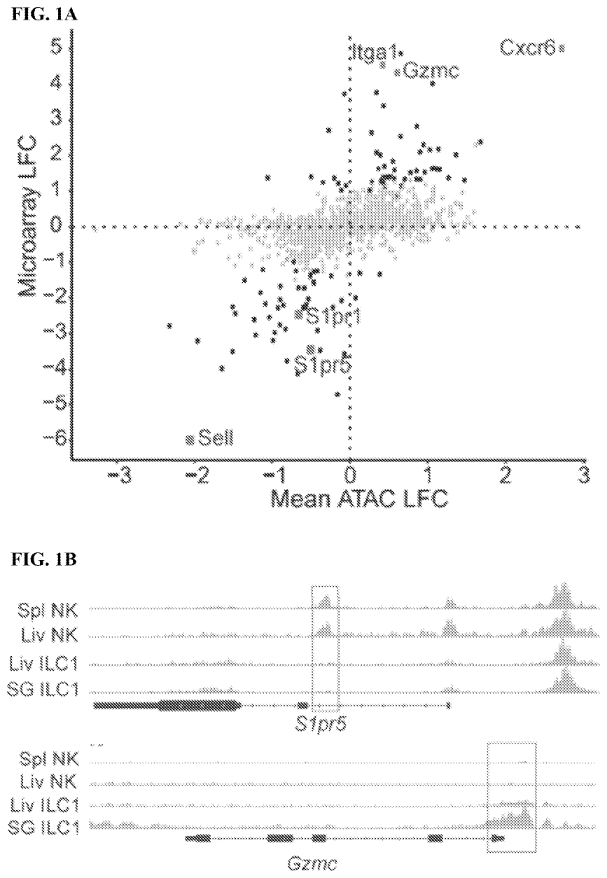

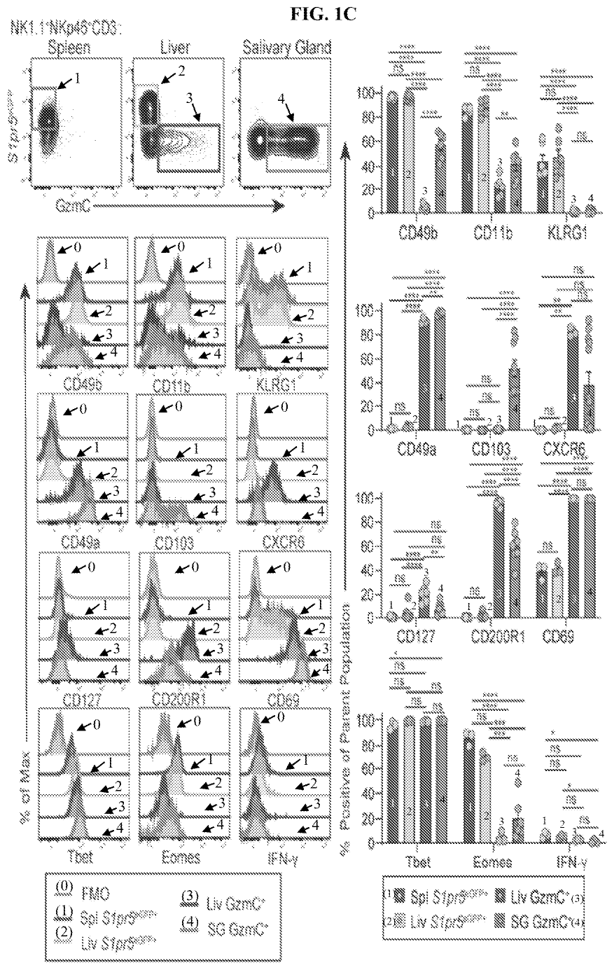

The differential peaks largely tracked with differential gene expression in NK cells and ILC1s (FIG. 9C)(7). Among the targets with positive association between locus accessibility and gene expression were those involved in cell trafficking and effector functions (FIG. 1A). Of note, NK cell-enriched genes included Sell, Slpr1, and S1pr5 (FIGS. 1A-1B and FIG. 9D), which promote cell exit from bone marrow and migration through blood vasculature and lymph(8). ILC1-enriched genes included the integrin Itga1 and chemokine receptor Cxcr6 (FIG. 9D), which promote cell retention in peripheral tissues(1). Unexpectedly, the Gzmc locus, which encodes the lytic granule-associated cytotoxic molecule granzyme C(9) was highly accessible, and the Gzmc transcript was expressed in cells with an ILC1 phenotype (FIGS. 1A-1B), raising the possibility that innate lymphocytes with cytotoxic potential reside among tissue-resident populations.

To mark and track NK cells and granzyme C-expressing innate lymphocytes at the single-cell level, we wished to create reporter and cell fate-mapper mouse strains. To this end, we selected S1pr5, given its specificity and importance in NK cell trafficking(10), whereas the genes S1pr1 and Sel1 were expressed more broadly in other lymphocyte populations. An expression cassette encoding enhanced green fluorescence protein (eGFP), T2A self-cleavage peptide, and improved Cre recombinase (iCre) was knocked into the 3′ untranslated region (UTR) of the S1pr5 locus (designated as S1pr5^{eGFP-T2A-iCre}) (FIGS. 10A-10C). As expected, S1pr5^{eGFP} and granzyme C protein were detected in NK and ILC1 phenotype cells, respectively (FIG. 10D). Neither marker was expressed by all cells within any of the populations analyzed for ATACseq, suggesting further heterogeneity or precursor states among group 1 innate lymphocytes (FIG. 10D).

When used in combination, S1pr5^{eGFP} and granzyme C exhibited a more exclusive expression pattern than that of CD49a and CD49b among NK1.1⁺NKp46⁺ innate lymphocytes (FIG. 1C and FIG. 9A). Overall, S1pr5^{eGFP}-positive cells had high expression of markers associated with mature NK cells, including CD49b, CD11b, and KLRG1 (FIG. 1C). S1pr5^{eGFP} expression increased as splenic NK cells matured, resulting in high levels of expression in virtually all CD27⁻CD11b⁺ mature NK cells (FIG. 10E), in agreement with previous findings(10). Granzyme C-positive cells expressed low levels of CD49b, CD11b, and KLRG1, but high levels of ILC1 markers to varying extents, including CD49a, CD103, CXCR6, CD127, CD200R1, and CD69 (FIG. 1C). Furthermore, the mature NK cell transcription factor Eomes exhibited higher, but not exclusive, expression in S1pr5^{eGFP}-positive cells than in granzyme C-positive cells, while the transcription factor T-bet and the type 1 inflammatory cytokine IFN-γ were less differentially expressed (FIG. 1C). Thus, relative to S1pr5^{eGFP}-positive cells, granzyme C-positive innate lymphocytes uniquely express markers of tissue residency, but exhibit overlapping expression of NK cell maturation markers.

### Example 3: Granzyme C-Expressing Innate Lymphocytes Differentiate from ILCps, but not from NK Cells, ILC2s, or ILC3s

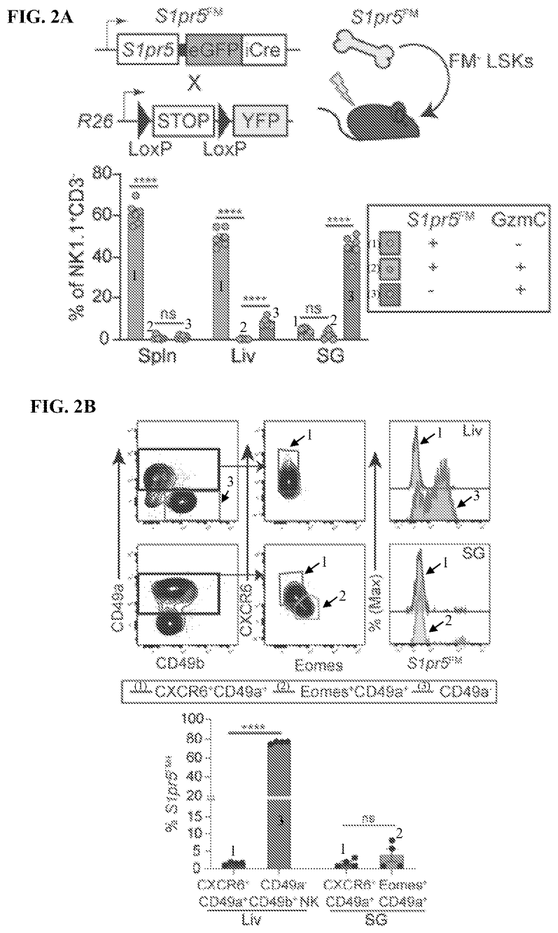

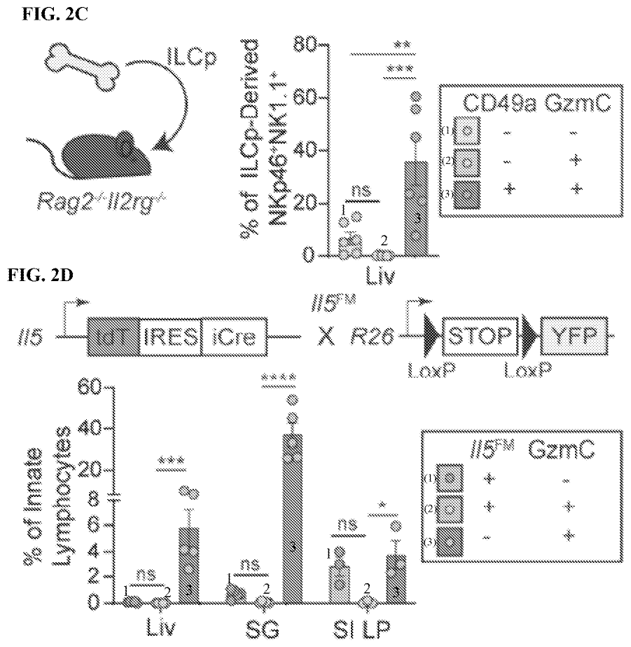

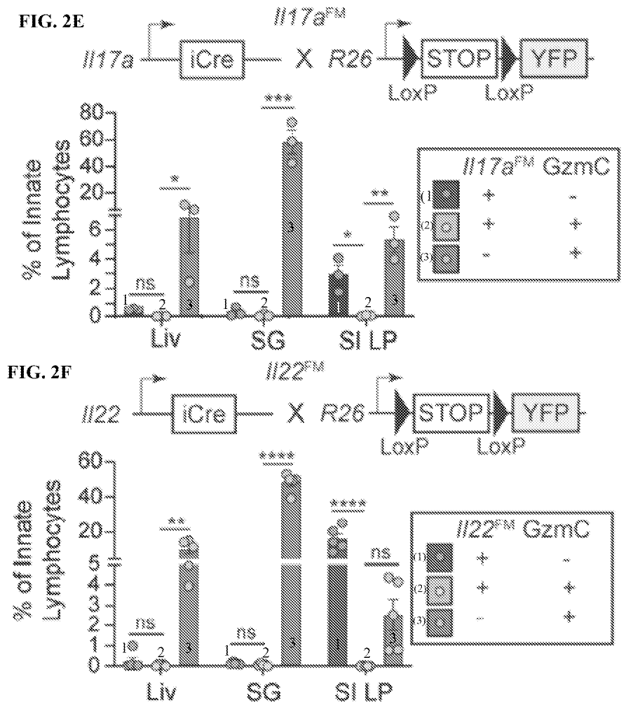

The shared properties of S1pr5-expressing NK cells and granzyme C-expressing ILC1-phenotype cells raised questions of their ontogenetic relationship. Previous adoptive cell transfer experiments have led to conflicting conclusions on the likelihood of NK cells giving rise to ILC1-like cells in tissues(11-13). To investigate whether granzyme C-positive innate lymphocytes might be converted from S1pr5-positive mature NK cells under physiological conditions (FIG. 11A), we crossed S1pr5^{eGFP-T2A-iCre} to the Rosa26^{LSL-YFP} allele, generating S1pr5 fate-mapper mice (S1pr5^{FM}) in which cells with a history of S1pr5 expression and their progeny are permanently marked by YFP expression. Stochastic Cre activity during embryogenesis led to varying degrees of fate-mapping among hematopoietic stem cells (HSCs) (FIG. 11B). To bypass this non-specific fate-mapping, we transferred non-fate-mapped HSCs to irradiated recipient mice to measure history of S1pr5 expression during hematopoiesis (FIG. 2A). Analysis of the blood of chimeric mice revealed that S1pr5^{FM} expression was restricted to cell populations that currently express S1pr5, namely circulating NK1.1⁺ innate lymphocytes and Ly6C⁻ patrolling monocytes, with minimal fate-mapping in Ly6C⁺ monocytes and no fate mapping in T cells (FIG. 11C). Investigation of granzyme C and S1pr5^{FM} expression among group 1 innate lymphocytes in the spleen, liver, and salivary gland revealed that the vast majority of granzyme C-expressing cells did not have a history of S1pr5 expression (FIG. 2A and FIG. 11D). The salivary gland ILC1-phenotype cells included subsets that expressed either CXCR6 or Eomes, both of which expressed granzyme C (FIG. 2B and FIG. 11E), thus explaining the heterogeneity of these markers in salivary gland granzyme C-expressing cells (FIG. 1C). Although Eomes was thought to mark all NK lineage cells, the vast majority of salivary gland Eomes⁺ CD49a⁺ cells did not express S1pr5^{FM}, as was true for the CXCR6⁺ ILC1 subsets in the liver and salivary gland, whereas almost all liver NK cells did (FIG. 2B). These findings demonstrate that S1pr5-positive mature NK cells largely do not give rise to granzyme C-positive innate lymphocytes, including both CXCR6⁺ and Eomes⁺ ILC1-phenotype cells.

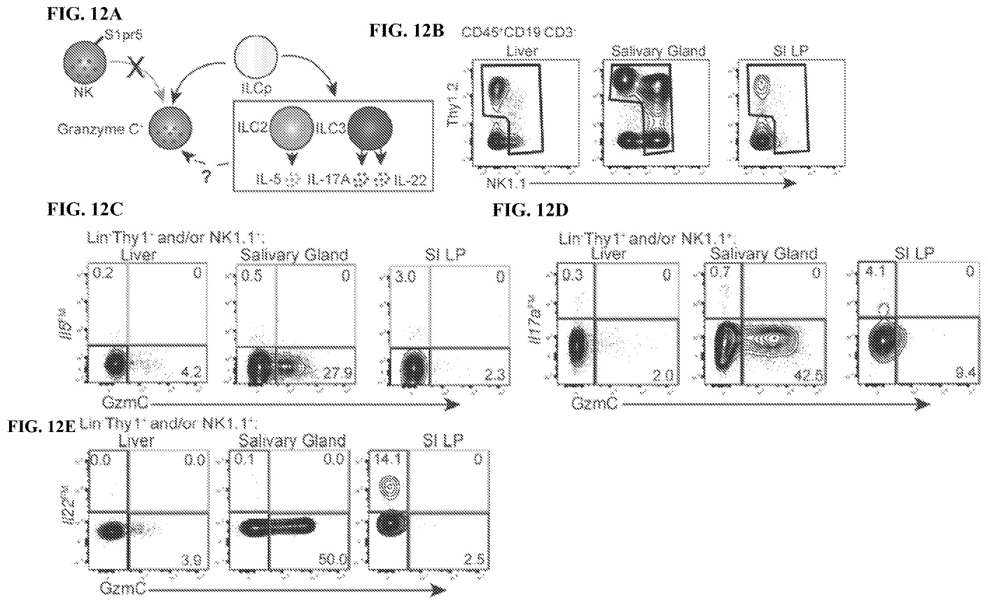

To explore whether granzyme C-expressing innate lymphocytes were differentiated from the ILC lineage, we transferred previously defined ILC progenitors (ILCp)(14, 15) into Rag2^{-/-}Il2rg^{-/-} lymphopenic hosts (FIG. 2C and FIG. 11F). Indeed, ILCp gave rise to a sizable population of granzyme C-positive cells that expressed the tissue residency marker CD49a (FIG. 2C and FIG. 11G), while NK cells were not detected among the progenies of ILCp (data not shown), unlike findings using a phenotypically distinct progenitor(16). Recent studies have revealed plasticity among ILC compartments, including the potential for group 2 or 3 ILCs to convert to an ILC1 phenotype cell(17-19). To determine whether IL-5-producing ILC2s, IL-17A- or IL-22-producing ILC3s could give rise to granzyme C-expressing innate lymphocytes, we crossed Il5^{iCre}, Il17a^{iCre}, and Il22^{iCre} each to Rosa26^{LSL-YFP} to generate IL-5 fate-mapper (Il5^{FM}), IL-17A fate-mapper (Il17a^{FM}) and IL-22 fate-mapper (Il22^{FM}) mice (FIGS. 2D-2F and FIG. 12A), and examined total innate lymphocytes (Lin⁻Thy⁺ and/or NK1.1⁺) across tissues (FIG. 12B). No co-expression of granzyme C with any cytokine fate-mapper was observed (FIGS. 2D-2F and FIGS. 12C-12E). These findings reveal that granzyme C-expressing innate lymphocytes can be differentiated from ILCp, but they are not converted from IL-5⁺ ILC2s, IL-17A⁺ or IL-22⁺ ILC3s at steady state.

### Example 4: Granzyme C-Expressing Cells are not Precursors for NK Cells, ILC2s, or ILC3s

Group 2 and 3 innate lymphocytes can seed tissues early in life (20, 21). As such, we investigated whether neonatal group 1 innate lymphocytes expressed granzyme C or S1pr5. There was a sizeable population of granzyme C-positive cells, which was associated with CD49a expression in livers of newborn mice and continuing through day seven (D7) and D14 of age (FIG. 3A). However, S1pr5 was not expressed through 14 days of age (FIG. 3A), supporting previous observations that ILC1-phenotype cells predominate in the liver of newborn mice while NK cells appear later in life(22).

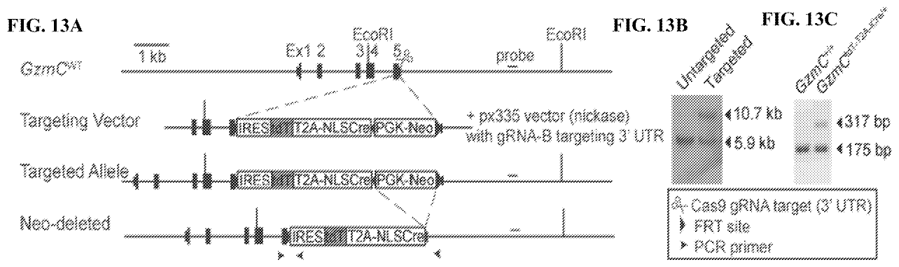

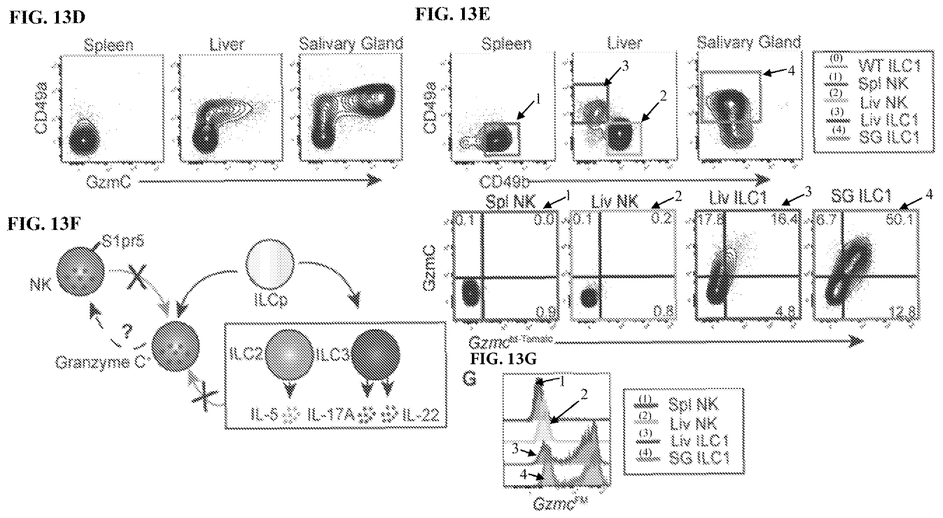

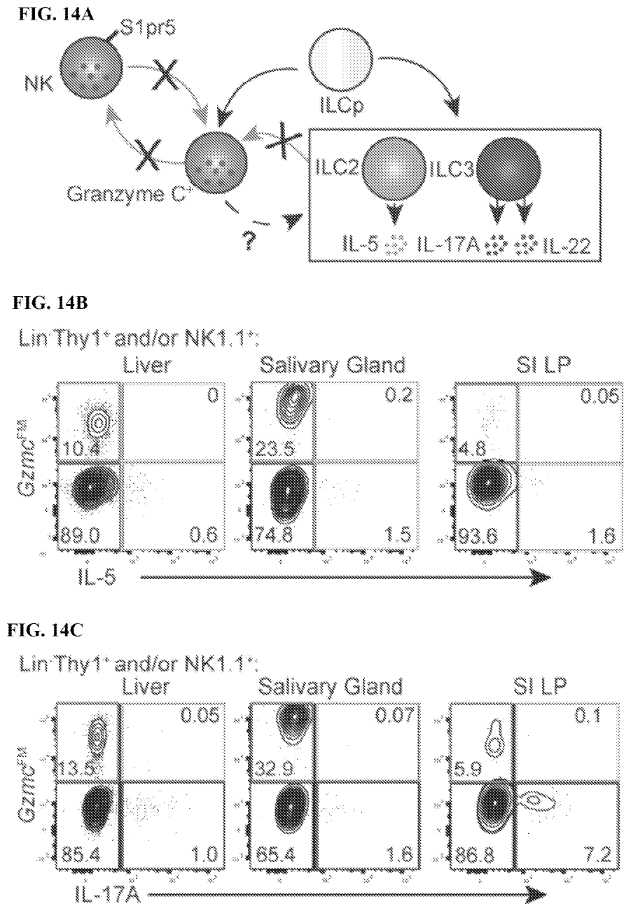

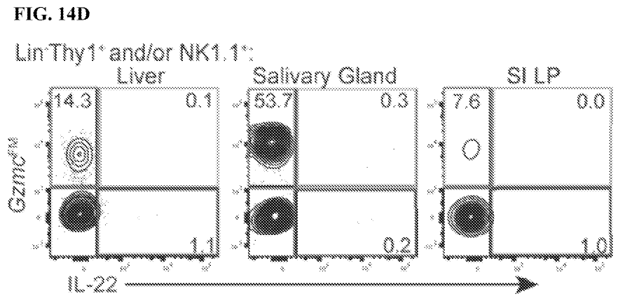

To track and target granzyme C-expressing cells, we generated a mouse line with an expression cassette encoding tandem dimer Tomato (td−Tomato), T2A self-cleaving peptide, and iCre knocked into the 3′ UTR of the Gzmc locus (designated as Gzmc^{tdT-T2A-iCre}) (FIGS. 13A-13C). Both granzyme C protein and td−Tomato reporter were associated with CD49a expression in the liver and salivary gland (FIG. 3B and FIG. 13D), and there was high correlation between Gzmc^{td-Tomato} and granzyme C protein itself (FIG. 13E). Given the early tissue seeding by granzyme C-expressing cells and previous observations that fetal liver “T-bet⁺Eomes⁻ immature NK cells” gave rise to mature NK cells in cell transfer experiments(23, 24), we explored the possibility that granzyme C-expressing cells represented a precursor state for mature NK cells under physiological conditions (FIG. 13F). To this end, we generated a granzyme C fate-mapper mouse (Gzmc^{FM}) by crossing Gzmc^{tdT-T2A-iCre} to the Rosa26^{LSL-YFP} allele (FIG. 3C). Analysis of the group 1 innate lymphocyte compartment revealed that a majority of ILC1-phenotype cells in the liver and salivary gland had a history of granzyme C expression (FIG. 3D and FIG. 13G). However, splenic and liver NK cells were not fate-mapped (FIG. 3D and FIG. 13G). To investigate whether granzyme C-expressing innate lymphocytes gave rise to other ILC populations, we measured production of IL-5, IL-17A, and IL-22 among innate lymphocytes in Gzmc^{FM} mice (FIG. 14A). Almost no cells that had been both fate-mapped and positive for either of these cytokines were detected across tissues (FIGS. 3E-3G and FIG. 14B-14D). Taken together, these findings reveal that granzyme C expression defines a distinct tissue-resident ILC subset that does not interconvert with circulating NK cells, IL-5⁺ ILC2s, or IL-17A⁺ or IL-22⁺ ILC3s at steady state.

### Example 5: Differential Regulation of Granzyme C-Expressing ILC1s Across Tissues by T-Bet, Eomes, and TGF-β

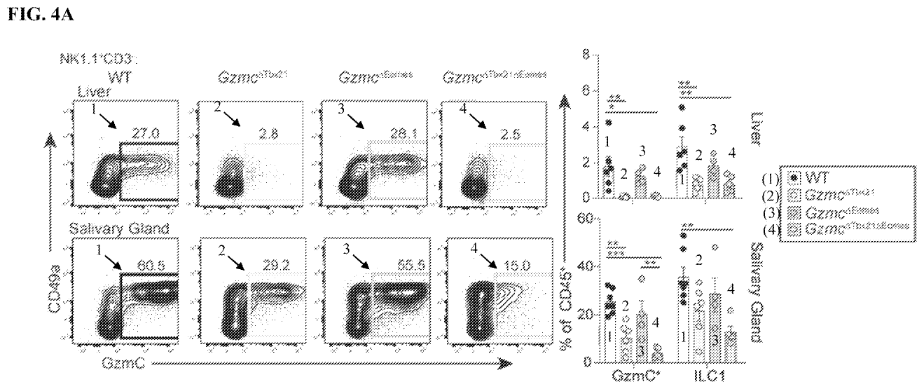

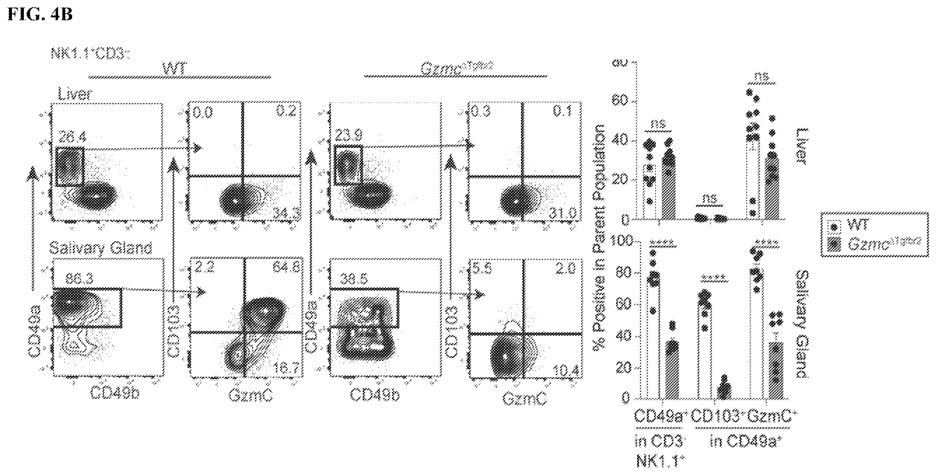

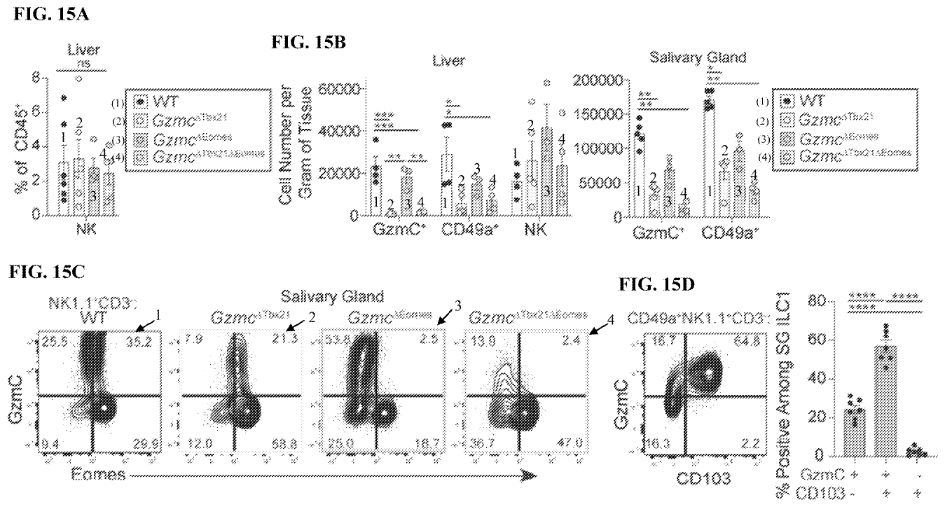

The transcription factors T-bet and Eomes control the differentiation of group 1 innate lymphocytes(11, 24). To investigate whether granzyme C-expressing ILC1s were dependent on these transcription factors for maintenance, we generated Gzmc^{ΔTbx21}, Gzmc^{ΔEomes}, and Gzmc^{ΔTbx21ΔEomes} mice by crossing Gzmc^{tdT-T2A-iCre} mice with mice carrying Tbx21^{fl/fl} and Eomes^{fl/fl} alleles. T-bet, but not Eomes, was required for the maintenance of all granzyme C-expressing ILC1s in the liver and thus a sizeable portion of the overall liver ILC1 population (FIG. 4A and FIGS. 15A-15B). In the salivary gland, where granzyme C-expressing ILC1s included CXCR6+ and Eomes⁺ subsets (FIG. 2B), T-bet promoted maintenance of both populations, while Eomes played an accessory role (FIG. 4A and FIGS. 15B-15C). In fact, Eomes itself was not required for granzyme C expression in most ILC1s, as Gzmc^{ΔEomes} mice maintained sizeable granzyme C-expressing populations (FIG. 4A and FIGS. 15B-15C). Nonetheless, in the absence of T-bet, Eomes supported granzyme C-expressing ILC1s in the salivary gland, while Gzmc^{ΔTbx21ΔEomes} mice exhibited the strongest loss of granzyme C-expressing cells (FIG. 4A and FIGS. 15B-15C).

Salivary gland ILC1s require TGF-β signaling for their differentiation, whereas liver ILC1s do not (25). We observed co-expression of the TGF-β target CD103 with granzyme C in salivary gland ILC1s (FIG. 15D), prompting us to explore whether mature granzyme C-expressing ILC1s required continued TGF-β signaling. To this end, we generated Gzmc^{ΔTgfbr2} mice by crossing Gzmc^{tdT-T2A-iCre} mice with mice carrying Tgfbr2^{fl/fl} alleles. Indeed, loss of TGF-β signaling impaired homeostasis of granzyme C-expressing ILC1s in the salivary gland, but not in the liver, leading to decreased CD49a expression among group 1 innate lymphocytes and decreased CD103 and granzyme C expression among CD49a⁺NK1.1⁺CD3⁻ cells (FIG. 4B). These observations demonstrate that TGF-β signaling is continually required for the maintenance of granzyme C-expressing ILC1s in the salivary gland.

### Example 6: Granzyme C-Expressing Innate Lymphocytes Expand in Tumor and Mediate Cancer Immunosurveillance

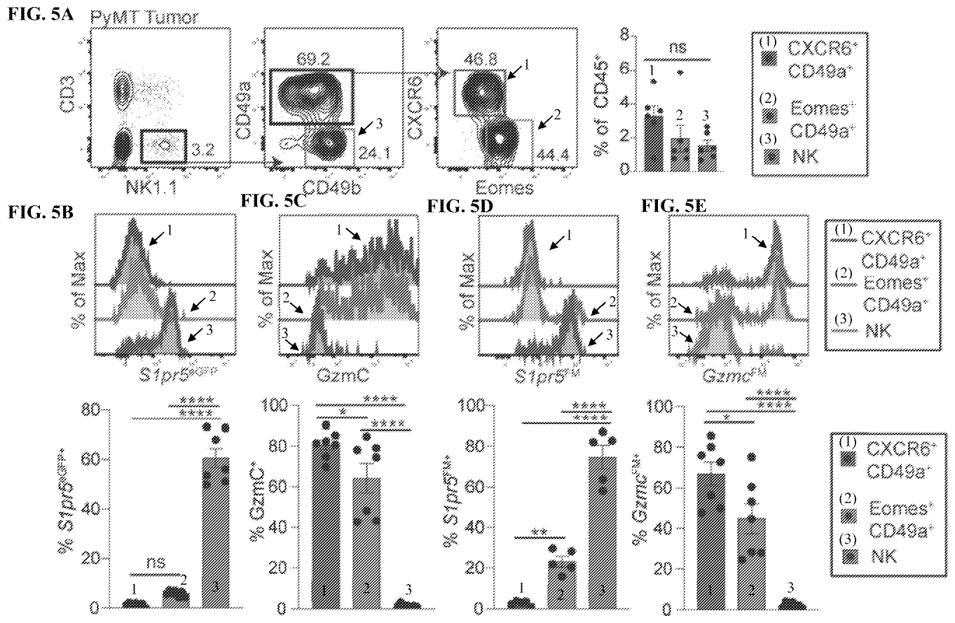

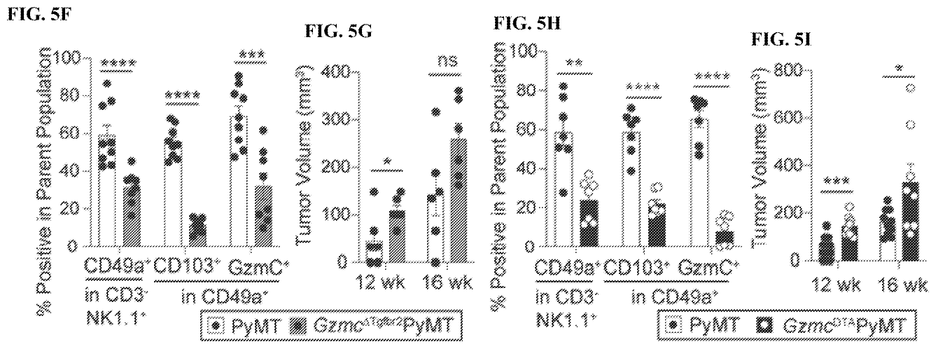

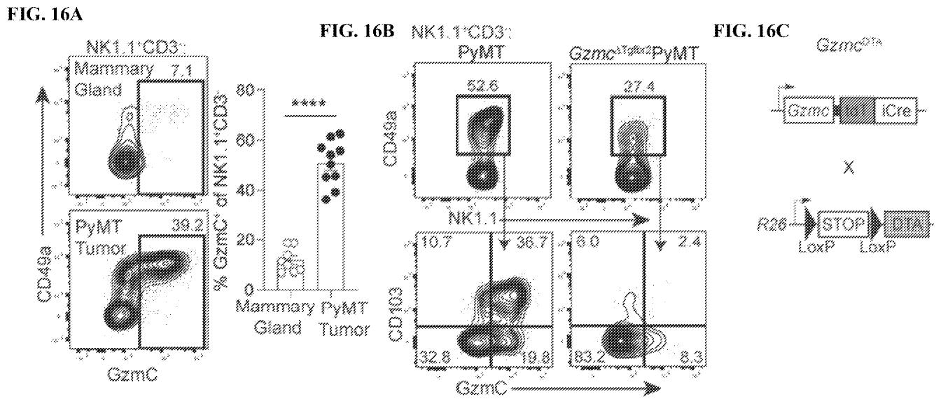

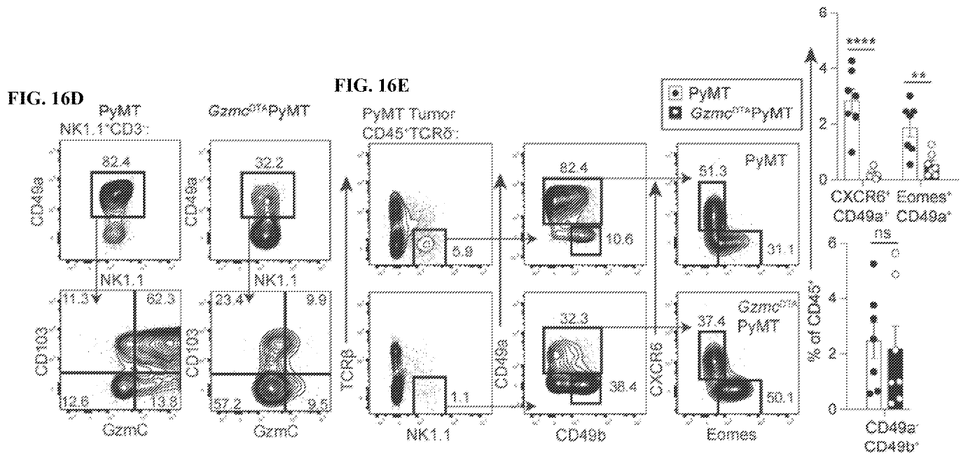

Beyond tissues at steady-state, we next explored the identity and origin of group 1 innate lymphocytes in the context of cancer using the MMTV-PyMT spontaneous model of breast cancer (3). Relative to healthy mammary glands, we observed an increase in granzyme C-expressing innate lymphocytes in PyMT tumors (FIG. 16A). NK1.1⁺CD3⁻ cells broadly included CD49a⁺CD49b⁺ NK cells and two subsets of CD49a⁺ cells differentiated by expression of CXCR6 and Eomes, all present at comparable frequencies (FIG. 5A). In the tumor, S1pr5eGFP was expressed in most NK cells, but minimally in Eomes⁺CD49a⁺ or CXCR6⁺CD49a⁺ cells (FIG. 5B), whereas granzyme C was highly expressed by both CXCR6⁺ and Eomes⁺ subsets of CD49a⁺ cells, with no expression in NK cells (FIG. 5C), similar to what was observed in steady state populations in the spleen, liver, and salivary gland (FIG. 1C). Conversion of NK cells to ILC1-like cells in tumors has been reported (13), and thus we generated S1pr5^{FM} bone marrow chimera mice in PyMT recipients to track history of S1pr5 expression among tumor-infiltrating group 1 innate lymphocytes. Virtually all CXCR6⁺CD49a⁺ cells and about 75% of Eomes⁺CD49a⁺ cells had no history of S1pr5 expression, whereas NK cells had high levels of S1pr5^{FM} expression (FIG. 5D). Conversely, Gzmc^{FM}PyMT mice revealed that cells with a history of granzyme C expression were restricted to CXCR6− and Eomes-expressing CD49a⁺ cells, with no contribution to NK cells (FIG. 5E). Thus, most granzyme C-expressing tissue-resident phenotype innate lymphocytes appear ontogenically distinct from circulating NK cells in PyMT tumors.

As in the salivary gland, ILC1-phenotype innate lymphocytes in mammary tumors can express CD103(3), suggesting that TGF-β signaling may control their differentiation and homeostasis. To this end, we generated Gzmc^{ΔTgfbr2}PyMT mice, which had decreased CD49a expression among group 1 innate lymphocytes as well as decreased CD103 and granzyme C expression among CD49a⁺ cells (FIG. 5F and FIG. 16B), similar to what was observed in the salivary gland (FIG. 4B). Notably, these mice displayed accelerated tumor growth (FIG. 5G), revealing that TGF-β signaling is required for the maintenance of granzyme C-expressing innate lymphocytes to support their anti-tumor function.

To further explore the cancer surveillance function of granzyme C-expressing innate lymphocytes, we generated Gzmc^{DTA}PyMT mice by crossing Gzmc^{tdT-T2A-iCre} mice with a conditional allele of Rosa26^{LSL-DTA} encoding the lethal diphtheria toxin A preceded by a floxed translation-STOP cassette to deplete granzyme C-expressing cells (FIG. 16C). Indeed, these mice had large reductions in CD49a expression in NK1.1⁺CD3⁻ cells as well as in CD103 and granzyme C expression among CD49a⁺ cells (FIG. 5H and FIG. 16D), and had depletion of both CXCR6⁺ and Eomes⁺ subsets of CD49a⁺ cells while leaving NK cell abundances unchanged (FIG. 16E). Of note, these mice displayed tumor acceleration (FIG. 5I), demonstrating a critical function for granzyme C-expressing innate lymphocytes in cancer immunosurveillance.

### Example 7: ILC1s Broadly Express Cytotoxic Molecules, with Granzyme C Expression Marking a Mature Effector State

Given that ILC1-like cells from PyMT tumors can directly kill cancer cells in a perforin-dependent manner(3), and granzyme C can induce cell death in target cells(9), we revisited ILC1s in the liver and salivary gland for expression of other cytotoxic molecules. Indeed, we observed granzyme B expression in subsets of liver and salivary gland ILC1s and to much higher levels than that of NK cells in the spleen and liver (FIG. 6A). Additionally, all ILC1s and NK cells expressed perforin with liver NK cells expressing relatively higher levels (FIG. 6A). Using past and current granzyme C expression as a way to subset ILC1s in the liver and salivary gland, we were able to identify ILC1s with current granzyme C expression (GzmC⁺Gzmc^{FM+}, double-positive [DP]), past but not current granzyme C expression (GzmC⁻Gzmc^{FM+}, fate mapped single-positive [FMSP]), and no history of expression (GzmC⁻Gzmc^{FM-}, double-negative [DN]) that were present in varying proportions in these tissues (FIG. 17A). Granzyme B expression was highest in the DP and lowest in the DN populations in both liver and salivary gland, whereas perforin was largely uniformly high across ILC1 subsets (FIG. 6B).

To further explore the heterogeneity among these ILC1 subsets, we performed RNA sequencing experiments on DP, FMSP, and DN ILC1s as well as S1pr5^{eGFP+} NK cells from the liver, carrying out six pairwise comparisons among the four populations (FIG. 6C, FIG. 17B). The largest differences were observed between each of the ILC1 subsets and the NK cell population (FIG. 6C). We identified core NK cell and ILC1 genes based on differentially expressed genes shared across the three ILC1-NK cell comparisons (FIG. 17C), including genes such as Sel1, S1pr5, S1pr1, Gzmc, Itga1, and Cxcr6 identified in our analysis using ATACseq and microarray performed on bulk liver ILC1s (FIG. 1 and FIGS. 9A-9B). Another core ILC1 gene included Zfp683, encoding the protein Hobit, (data not shown), which controls ILC1 lineage commitment(4, 26, 27). Among comparisons within ILC1 subsets, gene expression was most differential between DP and DN subsets, with 74 genes upregulated in DP and 245 genes upregulated in DN (FIG. 6D, FIG. 18). Genes enriched in DP population included those encoding secreted effector molecules such as Gzmb, Gzmc, Csf1, Ccl1, Cx3cl1, and Xcl1 (FIG. 6D). Interestingly, almost all of the genes upregulated in DP were expressed in FMSP at intermediate levels, suggesting FMSP may be an intermediate state between DN and DP (FIG. 6D). There were many more genes upregulated in the DN population, many of which also showed intermediate levels of expression in the FMSP population (FIG. 18). Among DN-enriched genes were those encoding regulators of cell cycle progression such as Cdc45, Cdc6, and Cdca2 (FIG. 18), suggesting that DN cells may be more proliferative and perhaps represent more of a precursor population. These findings suggest that the three ILC1 populations are relatively similar to one another, with granzyme C-expressing ILC1s in a mature effector state and granzyme C-nonproducing ILC1s including less differentiated cells.

### Example 8: IL-15 Induces Granzyme C Expression in ILC1s that Exhibit Lytic Granule-Mediated Cytotoxicity

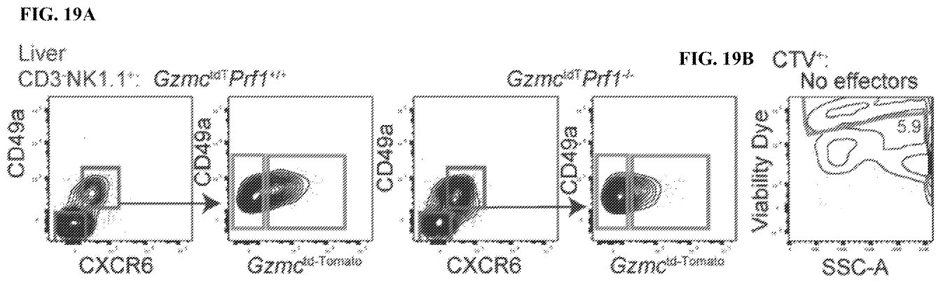

Differentiation and function of group 1 innate lymphocytes are promoted by the common γ chain cytokine interleukin-15 (IL-15) (28). The sizeable FMSP population in liver CXCR6⁺ ILC1s suggested that granzyme C expression could be modulated in vivo. To investigate how IL-15 might regulate granzyme C expression in group 1 innate lymphocytes, we cultured DP, FMSP, and DN ILC1s and S1pr5⁺ NK cells from the liver with IL-15/IL-15Rα complex (IL-15c). Notably, granzyme C protein expression was induced in both FMSP and DN ILC1s, but not NK cells (FIG. 7A), which was in agreement with the observation that Gzmc transcript was significantly higher in all three ILC1 subsets than in NK cells (FIG. 17C). These findings suggest that current granzyme C expression may not indicate a distinct subset of ILC1s but rather cells in a mature effector state, potentially induced by IL-15 stimulation. To further explore this possibility, we sorted Gzmc_{tdT+} and Gzmc^{tdT-} CXCR6⁺ ILC1s from the liver of mice sufficient or deficient in the cytolytic pore-forming protein perforin, expanded them in vitro with IL-15c, and tested their ability to kill RMA-S target cells (FIG. 19A). After exposure to IL-15c, which can induce granzyme C expression, both subsets of ILC1s were able to robustly kill target cells in a perforin-dependent manner, with no death above background in either of the perforin-deficient effector conditions (FIG. 7B and FIG. 19B). Thus, CXCR6⁺ ILCs have potent lytic granule-mediated cytotoxic capabilities.

### Example 9: IL-15 is Essential for Generation of Granzyme C-Expressing ILC1s with Enhanced Signaling Causing Perforin-Dependent Lethal Autoimmunity

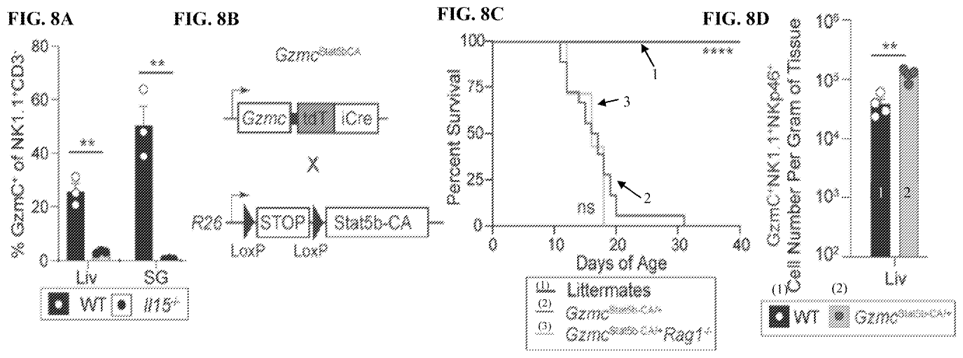

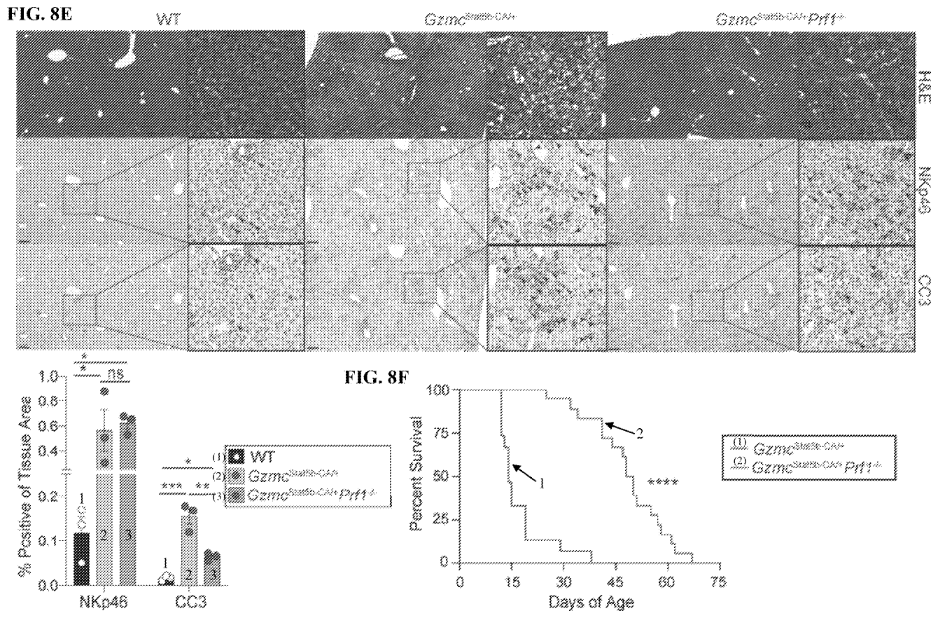

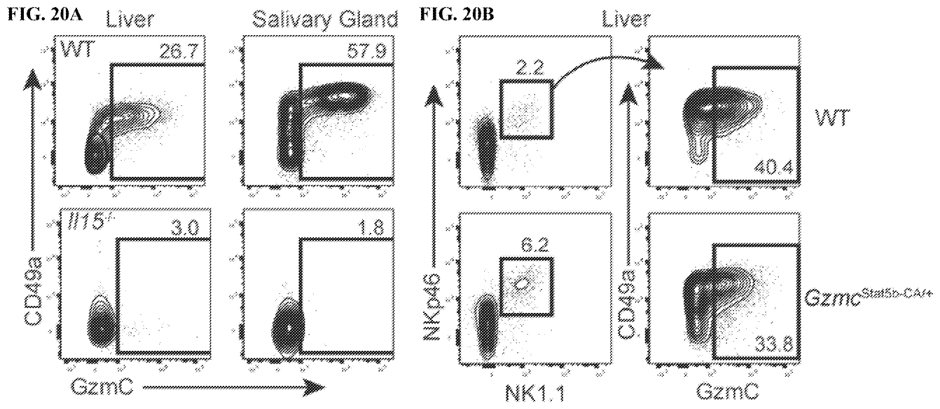

We next explored the requirement for IL-15 in vivo. Indeed, there was a total loss of granzyme C-expressing ILC1s in the liver and salivary gland of IL-15^{-/-} mice (FIG. 8A and FIG. 20A). IL-15 signaling is predominantly transduced through Jak kinases that activate the Stat5a/b family of transcription factors (29). Of note, tissue-associated IL-15 may act as an alarmin to modulate the function of tissue-resident group 1 innate lymphocytes, and its availability may be restricted at steady state (30). To investigate whether enhanced IL-15 signaling in granzyme C-expressing ILC1s would alter their phenotype in vivo, we crossed Gzmc^{tdT-T2A-iCre} mice with a conditional allele of Rosa26^{LSL-Stat5b-CA} encoding a constitutively active (CA) form of Stat5b preceded by a floxed translation-STOP (LSL) cassette (FIG. 8B). Strikingly, all Gzmc^{Stat5b-CA/+} mice died at a median age of 16.5 days, and this lethality did not require Rag1-dependent adaptive lymphocytes (FIG. 8C).

Granzyme C-expressing ILC1s were dramatically expanded in the liver of Gzmc^{Stat5b-CA} mice (FIG. 8D and FIG. 20B). Multi-organ immunopathology was also detected, exemplified by multifocal to coalescing hepatitis with hepatocellular necrosis in association with enhanced immunoreactivity of NKp46 and cleaved caspase 3 (CC3), which marked innate lymphocytes and apoptotic cells, respectively (FIG. 8E). To determine whether lytic granule-mediated cytotoxicity contributed to the cell death phenotype, we crossed Gzmc^{Stat5b-CA} mice onto a Prf1^{-/-} background. Indeed, while Gzmc^{Stat5b-CA/+} and Gzmc^{Stat5b-CA/+}Prf1^{-/-} had comparable infiltration of NKp46⁺ innate lymphocytes, CC3-marked apoptosis and hepatocellular damage were attenuated in the absence of perforin (FIG. 8E). Importantly, the neonatal lethal phenotype was substantially rescued, increasing the median survival time from 14 days in Gzmc^{Stat5b-CA/+} mice to 49 days in littermate Gzmc^{Stat5b-+Prf}1^{-/-} mice (FIG. 8F). Thus, ILC1s can carry out lethal autoimmunity in tissues including the liver via perforin-mediated cytotoxicity, and IL-15 may be an important regulator of this response.

Heterogenous populations of group 1 innate lymphocytes are widely distributed in circulation and peripheral tissues, and can be mobilized under immune challenging conditions. By generating two reporter and Cre mouse lines, this work allows for specific genetic fate-mapping to clarify the ontogeny and plasticity of multiple innate lymphocyte effector populations at both neonatal and adult time points. S1pr5-expressing NK cells and granzyme C-expressing ILC1s are largely distinct and represent two discrete terminally differentiated populations at steady state. These two populations appear at different points during development, with ILC1s present in peripheral organs at birth and S1pr5-expressing NK cells not detected until weeks later. Additionally, we did not observe interconversion between mature cytokine-producing ILC2s and ILC3s with granzyme C-expressing ILC1s, suggesting granzyme C-expressing ILC1s are terminally differentiated. However, interconversion of cells along the ILC1 and NKp46⁺ ILC3 lineage may remain possible in certain inflammatory conditions, as RORγt appears to be a suppressor of this cytotoxic ILC1 state(17, 19).

The expression of Eomes has been proposed as a defining marker for NK cells. Our data uncover more nuance in this definition. Given that S1pr5 is expressed by all CD27⁻ CD11b⁺ mature NK cells in the spleen, and given the lack of S1pr5-fate-mapping in a large majority of Eomes⁺ tissue-resident group 1 innate lymphocytes in the salivary gland or in the tumor, we can conclude that a plurality of these Eomes⁺ populations do not differentiate from mature circulating NK cells. This is in agreement with the lack of input from wild-type circulating cells to the empty ILC niche in salivary glands of Ncr1^{ΔTgfbr}2 parabionts(25). Given that Eomes is expressed before S1pr5, it remains possible the relevant precursor may be an NK cell-committed progenitor that retains the potential to gain tissue-residency, such as immature splenic NK (iNK)-phenotype cells, which have low S1pr5 expression and may explain the partial fate-mapping of Eomes⁺ population in the tumor. However, iNK-phenotype cells may also be a mixture of ILC-lineage precursors and NK cell-lineage immature cells(22), and until NK cell and ILC1 precursor populations can be fully clarified(31, 32), whether all Eomes⁺ cells are NK lineage cells remains an open question.

Previous work demonstrated “ILC1-like” cells from PyMT tumors can directly kill cancer cells in a perforin-dependent manner(3). Given that this population is a mixture of both CXCR6⁺ and Eomes⁺ tissue-resident cells, and these cells come from a disease state, we expanded on this finding by demonstrating perforin-dependent cytotoxicity of CXCR6⁺ ILC1s from the liver, both by those that initially express granzyme C and those that do not. Although these ILC1s also express TRAIL, which can induce cell death in a perforin-independent manner, we observed that their killing capabilities against RMA-S cells required perforin. Recent studies support our findings, as certain liver ILC1 subsets have the capacity to kill YAC-1 target cells(4, 5). Given that these experiments did not include addition of IL-15c and had a shorter coculture period, this may explain the differences in killing rate observed compared to our findings. These subsets include CD127⁻ mature ILC1, which are more enriched for granzyme B and C-expressing cells than the precursor CD127⁺ ILC1 population (4, 27). This maturation spectrum of ILC1s in the liver is in line with our RNAseq findings that DN ILC1s appear more proliferative and precursor-like, although we do not observe differential expression of CD127 among the three subsets we sequenced (data not shown). The fact that ILC1s can readily express cytolytic molecules and mediate cytotoxicity may help reconcile some observations in human, such as populations of ILC-like cells with cytotoxicity(33). Here we specifically disrupted granzyme C-expressing cells while leaving other IL-15-dependent and perforin-expressing immune cells such as NK cells intact and observed a similar acceleration in tumor growth, thus confirming an important role for these cells in cancer immunosurveillance.

Of note, TGF-β signaling promoted maintenance and anti-tumor functions of granzyme C-expressing tissue-resident innate lymphocytes. TGF-β may represent a tissue niche-associated signal that helps maintain proper localization of these cells in epithelial tissues, for instance through direct lymphocyte-epithelial cell contact via CD103 and E-cadherin interactions, which may provide additional survival and differentiation cues. A recent study of human head and neck tumors revealed a CD49a⁺CD103⁺ ILC1-like population with potent cytolytic activities against cancer cells(34). These cells can be differentiated from peripheral NK-phenotype cells, especially the CD94⁺NKp80⁺CD16⁻ immature subset, in a co-culture system with cancer cells and IL-15, and is dependent on TGF-β signaling(34). Nonetheless, TGF-β signaling-dependent conversion of NK-phenotype cells to ILC1-like cells appeared to suppress their cancer surveillance function in a murine fibrosarcoma model(13). Furthermore, excessive TGF-β signaling triggered by expression of a constitutively active form of TGF-β receptor in NK cells promotes fibrosarcoma development as a likely consequence of enhanced angiogenesis(13). These contrasting outcomes may be explained by the possibility that tumor types with epithelial cancers, but not mesenchymal fibrosarcoma, are subject to tissue-resident ILC1-mediated cancer immunosurveillance. In addition, inhibitory or stimulatory functions of TGF-β may be conditional on the strength and duration of tumor-associated signals such as IL-15.

Our studies also imply excessive cytotoxic ILC1 responses under conditions of enhanced Stat5 signaling can mediate immunopathology and autoimmunity.

When drawing comparisons between innate lymphocyte and T cell populations, innate-like T cells may be the more relevant analog to ILCs. These T cells include cytokine-producing CD1d-restricted invariant natural killer T (INKT) cells that do not kill despite their name, and bona fide killer innate-like T cells (ILTCks) that can be found in intestinal intraepithelial compartment and in cancer(3, 37). Innate-like T cells appear to differentiate via distinct thymic progenitors, do not require lymph node-mediated priming, and migrate directly to peripheral tissues where they are poised to mediate effector responses, similar to ILCs. Thus, iNKT cells may mirror helper ILC subsets, while ILTCks may mirror cytotoxic ILC1s. On the other hand, circulating conventional NK cells and lymphoid organ-homing LTi cells may better mirror conventional CD8⁺ and CD4⁺ T cells, respectively, although given that conventional T cell responses evolved after innate lymphocytes, they may include more sophisticated effector and regulatory modules that have no clear parallel among innate lymphocytes.

In conclusion, ILC1s are not cytokine-restricted helper populations but can express high levels of cytotoxic molecules, including granzyme B, granzyme C, and perforin, and they can mediate potent perforin-dependent cytotoxicity and contribute to autoimmunity and cancer immunosurveillance in mice.

### Example 10: Materials and Methods (Related to Examples 11-19)

Human subjects and tissue collection. All research activities were preapproved by the Institutional Ethics Review Board at Memorial Sloan Kettering Cancer Center and individuals were required to provide written informed consent to participate in the study. All histological diagnoses were confirmed by expert genitourinary pathologists. After institutional review board approval and patient consent, blood samples were collected in cellular preparation tubes (CPT) just prior to surgery. Tumor and adjacent normal kidney samples were directly obtained from the operating room during nephrectomy. Tissue samples were placed in separate labeled containers containing Roswell Park Memorial Institute (RPMI) medium and transported in regular ice to the laboratory. The CPTs were transported at room temperature. Overall transit time in all cases was less than 1 hour (from specimen extraction to cell dissociation) and the tissue samples were always kept in this medium. The cohort of ccRCC patients from which tissues were analyzed were 73.3% male, mean age 58.6, median age 58. The cohort of chRCC patients from which tissues were analyzed were 40% male, mean age 53.7, median age 58. For single-cell RNA sequencing, only tumor tissue was collected. The ccRCC patient was a 57-year-old female with stage IV disease and was treatment-naïve at the time of sample collection. The chRCC patient was an 84-year-old female with stage III disease and was treatment-naïve at the time of sample collection.

Mice. All in vivo mouse experimental procedures were performed under Sloan Kettering Institute (SKI) Institutional Animal Care and Utilization Committee (IACUC)-approved protocols. Mice were housed in designated specific pathogen-free animal facilities in ventilated cages with at most 5 animals per cage and provided food and water. Only female mice were used in this study. Littermates were used in all experiments, when possible, otherwise age-matched cage mates were used. CD11c-Cre, FSP1-Cre, Mrp8-Cre, Rosa26^{LSL-YFP}, Cdh1^{mCFP} and GCaMP5 calcium indicator (also known as PC-G5-tdT) mice are publicly available through Jackson Laboratories. IL-15^{2A-eGFP} mice were generously provided by R.M.K. IL-15^{fl} mice were generated by G.C. in the Ikuta lab and will be reported elsewhere. Briefly, the IL-15^{fl} allele was established by flanking exon 5 with two loxP sequences by standard homologous recombination in embryonic stem cells. Gzmc^{tdT-T2A-iCre} mice were generated by B.G.N. in the Li lab and will be reported elsewhere. Briefly, the Gzmc^{tdT-T2A-iCre} allele was generated by insertion of a targeting construct into the Gzmc locus by CRISPR/Cas9-mediated homologous recombination in embryonic stem cells.

Immune cell isolation from human and mouse tissues. Human blood was transferred from CPT tubes to 50 ml conical tubes containing red blood cell (Ammonium-Chloride-Potassium, ACK) lysis buffer and incubated for 10 minutes at room temperature. Tubes were spun at 2,400 RPM for 3 minutes, pellets were resuspended in FACS buffer (1×PBS with 1% FBS, 2 mM EDTA and 0.02% sodium azide) and stored on ice until staining procedure. Human tumor and adjacent normal kidney tissues, as well as mouse mammary glands and tumors, were prepared by mechanical disruption via mincing with a razor blade followed by treatment with 280 U/ml Collagenase Type 3 (Worthington Biochemical) and 4 μg/ml DNase I (Sigma) in HBSS medium at 37° C. for 1 hour with periodic short vortexing. Digested tissues were mashed through 70-μm filters and collected by centrifugation. Cell pellet was resuspended in 44% Percoll, layered on top of 66% Percoll (Sigma), and centrifuged at 1,900 g for 30 minutes without brake. Cells at the Percoll interface were collected, washed and resuspended in FACS buffer for downstream assays.

Tumor measurement. Tumors in PyMT mice were measured weekly using a caliper, beginning when a single tumor diameter reached approximately 3-4 mm. Tumor volume was calculated using the equation [(L×W²)×(π/6)] where “L”=length and “W”=width. Individual tumor volumes were added together to calculate total tumor burden per mouse.

Preparation of single-cell RNA sequencing libraries. A suspension of 10,000 FACS sorted live cells in 1×PBS (calcium and magnesium free) containing 0.04% BSA (Sigma) were used as input to the 10× chromium controller system (10× Genomics Inc., product code 120223). Cells were barcoded using the 10×™ GemCode™ Technology to separately index each cell's transcriptome by partitioning them into Gel Bead-in-EMulsions (GEMs). GEMs were generated by combining barcoded Single Cell 5′ Gel Beads, RT Master Mix with cells, and Partitioning Oil on a microfluidic chip. The GEM RT reaction was performed in thermocycler (53° C. for 45 minutes, 85° C. for 5 minutes, 4° C. hold overnight). After RT incubation, the GEMs were broken, and the first strand of cDNA was recovered using DynaBeads® MyOne™ Silane beads. 2-50 ng of amplified cDNA and the target enriched product respectively were used as input for library construction. Fragmentation, end repair and A tailing were performed to obtain final libraries containing the P5 and P7 priming sites used in Illumina® sequencing. High sensitivity DNA chips and the Agilent 2100 bioanalyzer (Agilent Technologies) were used for 5′ gene expression quality control and quantification. Quality control was performed twice before sequencing. The 5′ gene expression library was sequenced on NovaSeq 6000 S1 with sequencing depth of approximately 300-500 million reads per sample. For the mouse dataset, live, CD45⁺CD3⁻NK1.1⁺ innate lymphocytes from the pooled tumors of one PyMT mouse were sorted into 1×PBS containing 0.04% BSA. Library preparation, quality control and single-cell RNA sequencing were performed by the Integrated Genomics Operations Core of Sloan Kettering Institute at MSKCC.

Batch effect correction and single-cell count matrix processing. Pre-processing of single-cell RNA sequencing fastq files was conducted using Cell Ranger v3.0.2 (10× genomics). Single-cell RNA sequencing reads were aligned to the hg19 reference genome (ref-version 3.0.0). The count matrix used for downstream analysis was generated using the Cell Ranger count function with parameter—expect-cells=3000 (filter_matrix output). Cells with >20% of transcripts derived from mitochondrial genes were considered apoptotic and were thus excluded, and all mitochondrial genes were removed from the final count matrix. Ribosomal genes and the noncoding RNAs NEAT1 and MALAT1 were excluded (Freytag, S. et al. F1000Res 7, 1297 (2018)). Genes with mean raw count <3.0 were removed from the analysis, and cells with ambiguous phenotypes based on differentially expressed genes were excluded. This resulted in a final count matrix of 7402 cells and 14362 genes for downstream analysis. We used Seurat v2.3.4 to perform standard library size and log-normalization. The mean library size was 2240 transcripts per cell. Mouse single-cell RNA sequencing data (CD3⁻NK1.1⁺) was processed with the same pipeline; 873 cells and 10670 genes remained after QC.

To mitigate potential batch effects in the human data with two patients, we used the mutual nearest neighbors method (Haghverdi, L. et al. Nat Biotechnol 36, 421-427 (2018)). Log-normalized counts for each of the samples were used as input to the fastMNN( ) function from the R scran package (Lun, A. T. et al., F1000Res 5, 2122 (2016)) in Bioconductor with default parameters. The resulting batch-corrected PCA matrix was then input into Seurat using the SetDimReduction( ) function. The top 10 principal components were used as input for Louvain clustering using the FindClusters( ) function in Seurat at resolution 0.5 (Levine, J. H. et al., Cell 162, 184-197 (2015); Xu, C. & Su, Z. Bioinformatics 31, 1974-1980 (2015)). tSNE was used for cluster visualization. We computed differentially expressed genes using the Wilcox test in the FindMarkers ( ) function in Seurat, and genes with log fold change >0 and FDR P<0.05 were considered significantly differentially expressed. The definition of significantly differentially expressed genes was the same in the mouse dataset. Log-normalized counts were used for tSNE marker plots, violin plots and heatmaps.

Derivation of cell type signatures from single-cell RNA sequencing data. To interrogate TCGA bulk RNA sequencing data using single-cell RNA sequencing clusters, we sought to derive robust gene signatures from our single-cell RNA sequencing data. We generated two signatures-one for the innate lymphocyte cluster ILC1, and one for phenotypically exhausted T cells (clusters CD8_2A and CD8_2B combined). We first performed a pairwise differential expression analysis for each of these clusters—i.e., for the ILC1 cluster, we compared cluster ILC1 to each other cluster, and took the set of genes upregulated (FDR P<0.05, log fold change >0.3) in the ILC1 cluster in all 9 comparisons. We repeated this analysis for the combined clusters CD8_2A and CD8_2B. To ensure that signatures were comprised only of genes associated with immune cells and to enable robust application to TCGA dataset, we used the data from the Cancer Cell Line Encyclopedia to filter out genes expressed in any cancer cell line using a cutoff of 4 RPKM as previously described in Liu, M. et al., Nature 587, 115-120 (2020). Signatures were then applied to the bulk RNA sequencing data from the TCGA KICH (n=66 total; 65 with both RNA-seq and clinical data) and KIRC (N=534 with both RNA-seq and clinical data) cohorts using single-sample Gene Set Enrichment Analysis (ssGSEA) method using the R package GSVA, and patients were considered to be in the high group for each signature if the ssGSEA score exceeded the top quartile within each cancer type. We repeated the ILC1 signature associations with survival in the TCGA breast cancer (BRCA) cohort using all patients (N=1102) or in a subset of patients (N=231) with hotspot activating mutations (p.H1047R, p.E545K, p.E542K) in PIK3CA. Mutation data for the TCGA were obtained in the form of MutSigCV calls from the Broad Institute GDAC portal. The significance of the association of each signature with overall survival-computed using the Cox regression—was using a log-rank test. For the correlation of the ILC1 signature and IL-15 expression, the signature was applied to TCGA bulk RNA-seq data using ssGSEA, and the resulting score was plotted against the normalized expression of IL-15.

GSEA analysis. For the GSEA analysis comparing mouse innate lymphocytes to their human counterparts, we first assembled a list of all DEGs between the human ILC1 and NK cell clusters. We then computed DEGs as described above for the two clusters in the mouse dataset. We treated the human DEGs as a gene set, and the log fold changes from the mouse DEG analysis as a ranked list for input into a GSEA “preranked” analysis.

Validation of the ILC1 gene signature using bulk RNA sequencing data. Immune and non-immune cell populations were sorted from chRCC and ccRCC patient tumor samples. Library preparation, quality control and bulk RNA sequencing were performed by the Integrated Genomics Operations Core of Sloan Kettering Institute at MSKCC. We validated the ILC1 signature by applying it to bulk RNA sequencing data from the sorted populations using ssGSEA. We assessed discrimination performance by measuring the area under the receiver operating characteristic (ROC) and precision recall (PR) curves, when using the ILC1 signature to discriminate ILC1 populations (N=12) vs all others (N=57). The areas under the ROC and PR curves were calculated using the PRROC package in R.

Flow cytometry. For flow cytometry experiments (human and mouse), cells were pre-incubated with 2.4G2 mAb to block FcγR binding and then stained with panels of cell surface marker antibodies for 20 minutes on ice. Cells were washed 2× with FACS buffer and stained with LIVE/DEAD kit (Invitrogen) or Zombie Live/Dead kit (BioLegend) to exclude dead cells. Intracellular staining was carried out using the FoxP3/Transcription factor Fix/Perm Kit (Tonbo). All samples were acquired with a LSRII flow cytometer (Becton Dickinson) and analyzed with FlowJo software version 9.6.2 or 10.6.1 (Tree Star).

Antibodies and reagents for flow cytometry. Fluorochrome conjugated antibodies against human CD45 (clone H130), CD49a (SR84), CD16 (3G8), and CD14 (M5E2) were purchased from BD Biosciences. Antibodies against human CD3 (OKT3), PD-1 (EH12.2H7), Granzyme A (CB9), CD56 (HCD56), CD49a (TS2/T), and CD103 (Ber-ACT8) were purchased from Biolegend. Anti-human CD15 (MMA) was purchased from eBioscience, now Thermo Scientific, and anti-human NKG2A (REA110) was purchased from Miltenyi Biotec. Anti-human CD8α (RPA-T8) and biotinylated anti-human CD3 (UCHT1) were purchased from Tonbo Biosciences. Fluorochrome conjugated antibodies against mouse CD45 (clone 30-F11), CD49a (Ha31/8), CD103 (M290), Ly6G (1A8), F4/80 (T45-2342), CD11b (M1/70), MHC class II A-A/I-E (M5/114/15/2), and CD11c (N418) were purchased from BD Biosciences. Fluorochrome conjugated antibodies against mouse CD3ε (17A2), NK1.1 (PK136), CD19 (D1/CD19), XCR1 (ZET), CD49b (DX5), Ter119 (Ter-119), CD29 (HmB1-1), and EpCAM (G8.8) were purchased from BioLegend. Fluorochrome conjugated antibodies against mouse/human granzyme B (GB11) was purchased from Invitrogen. Fluorochrome conjugated antibodies against mouse CD27 (LG.7F9), CD31 (390), and CD24 (M1/69) were purchased from eBioscience.

FACS cell sorting. Immune cells isolated from either human or mouse tissues were resuspended in MACS buffer (1×PBS with 1% FBS and 100 U/ml Penicillin G and 0.1 mg/ml Streptomycin) with appropriate cell surface marker antibodies and incubated for 20 minutes on ice. Cells were washed 2× in MACS buffer and stained with LIVE/DEAD kit (Invitrogen) or Zombie Live/Dead kit (BioLegend) to exclude dead cells. Cells were sorted using the BD FACS Aria Cell Sorter. Samples were collected in RPMI with 10% FBS and processed immediately after sorting.

In vitro cell culture. Sorted cells were pelleted and resuspended in T cell medium (RPMI supplemented with 10% FBS, 1 mM sodium pyruvate, non-essential amino acids (Gibco), 10 mM Hepes, 55 μM 2-Mercaptoethanol, 100 U/ml Penicillin G and 0.1 mg/ml Streptomycin) with the indicated amount of IL-15/IL-15Rα complex (provided by Dr. Naikong Cheung, MSKCC). Cells were cultured for 7 days in U-bottom plates in a 37° C. humidified incubator.

Single-cell killing assay. Sorted innate lymphocyte populations were cultured for 7 days to recover from stress associated with cell sorting in T cell medium (RPMI supplemented with 10% FBS, 1 mM sodium pyruvate, non-essential amino acids (Gibco), 10 mM Hepes, 55 μM 2-Mercaptoethanol, 100 U/ml Penicillin G and 0.1 mg/ml Streptomycin). Cells were supplemented with either 10 ng/ml or 100 ng/ml human IL-15/IL-15Rα complex for the duration of culture. Polydimethylsiloxane grids containing 50×50×50 μm³ wells were applied to the bottom of an 8-well chamber plate which was then heated at 60° C. for 30 minutes, cooled and the appropriate medium was added to each chamber (T cell media supplemented with 10 ng/ml or 100 ng/ml human IL-15/IL-15Rα complex). 1 μg/ml propidium iodide (PI) was added to the medium to enable real-time labeling of dead cells. K562 cells were labeled with Cell-trace Violet (CTV) dye to facilitate their identification. Once labeled, K562 cells and effector innate lymphocytes were combined at a 1:1.5 ratio, mixed well, and added to the appropriate chamber well, which was then briefly spun down to place the cells within the wells. In general, individual wells contained 1 to 3 K562 cells. The chambers were imaged using a 20× objective lens (ZEN microscope) at 10-minute intervals for 12 hours. Brightfield, CTV and PI images were collected at each time point. Quantification was restricted to wells containing one effector cell in presence of at least one K562 cell in the same well. Only a single killing event per well was scored.

IL-15 ELISA. Mammary tissues were collected from 8-week-old wild-type and PyMT mice, pooled, weighed and immediately snap frozen in liquid nitrogen and stored at −80° C. Frozen tissues were thawed on ice, placed in a glass petri dish on ice, and minced using two scalpels. Tissue pieces were transferred to Eppendorf tubes. 1× lysis buffer [10 mM Tris-HCl (pH 8.0), 150 mM NaCl, 1% NP-40, 10% Glycerol, 5. 5 mM EDTA, Protease inhibitor cocktail (Roche)] was added with the following formula: 5× weight tissue in mg=volume of lysis buffer in μl. Samples were homogenized using an Eppendorf-fitting pestle and lysates were incubated for 1 hour at 4° C. while rotating. Lysates were then sonicated 7-minute regimen with 30-second intermittent on and off sonification periods at 4° C. using the Diagenode Bioruptor. Samples were centrifuged for 15 minutes at 14,000 rpm. Supernatants were collected, protein concentration was determined, and samples were stored at −80° C. Protein lysate was diluted 1:50 and tested for IL-15 using a Mouse IL-15 ELISA Kit according to the manufacturer's recommendations (Sigma). Values obtained were multiplied by the dilution factor, IL-15 quantity in ng was calculated for 1 mg of tissue.

IL-15 RT-PCR. Cells were directly sorted into TRIzol (Life Technologies). Total RNA was extracted with Direct-zol RNA MicroPrep kit (Zymo Research), and then reverse-transcribed to cDNA with Maxima First Strand cDNA synthesis Kit (Thermo Scientific). Quantitative PCR was performed with mouse IL-15-specific primers (forward, 5′-ACATCCATCTCGTGCTACTTGT-3′ (SEQ ID NO: 70); reverse, 5′-GCCTCTGTTTTAGGGAGACCT-3′ (SEQ ID NO: 71)). IL-15 expression data were normalized relative to Gapdh expression, and relative quantification levels were calculated.

Intravital confocal microscopy. At 12 weeks of age, intravital confocal microscopy of mammary tumors was performed. Briefly, mice were anaesthetized using a cocktail of ketamine and xylazine. Anaesthesia was maintained by continuous inhalation of 0.5% isoflurane and mice were kept to 37° C. and received oxygen (0.5 L/minute). The fur from the lower flank region was trimmed and the area sterilized using Betadine solution. The fourth left or right mammary gland was surgically exposed with a ventral skin incision and a skin flap. The mouse was transferred to a stage heated to 37° C. All imaging was done on an Olympus FVMPE-RS upright Multiphoton microscope fitted with a 25×1.05 NA Plan water-immersion objective and a Mai-Tai DeepSee Ti-Sapphire laser (Spectraphysics). Imaging was performed using λ=910 nm excitation and fluorescence emission was collected in three channels, using the following filter sets: a filter (480/40 nm) for CFP, a second filter (540/20 nm) for GFP and a third filter (605/70 nm) for tdTomato detection. Time-lapse images were acquired by scanning at 1024×1024 pixels with a z-step size of 3-4 μm and a total z-volume 50-100 μm, at 30 to 60 seconds intervals.

Immunofluorescence of formalin-fixed paraffin-embedded (FFPE) tissue. Tumor tissues were paraffin embedded and sliced by the Molecular Cytology Core Facility at MSKCC. Tumor slices were de-paraffinized and rehydrated as following: three 5-minute washes in HistoClear, two 10-minute washes in each of the following ethanol concentrations—100%, 90%, 70%, and 50% followed by two 5-minute washes in distilled water. Antigen retrieval was performed by bringing slides to a boil in 10 mM sodium citrate buffer (pH 6.0) and maintained at sub-boiling temperature for 10 minutes in the microwave, then cooled at room temperature for 30 minutes. Slides were washed twice for 5 minutes in distilled water. Endogenous peroxidase activity was quenched with 3% hydrogen peroxide in methanol for 30 minutes at room temperature then washed twice for 5 minutes in distilled water. Tissues were permeabilized by washing twice for 10 minutes in 1% animal serum in PBS with 0.4% Triton X-100 (PBS-T) followed by blocking for non-specific binding by incubating the tissue sections with 5% animal serum in PBS-T for 30 minutes at room temperature. Primary antibodies against CD103 (clone EPR4166, Abcam catalog number ab29202, concentration 1:100) and E-Cadherin-AF647 (clone EP700Y, Abcam catalog number ab 194982, concentration 1:100) were added in PBS-T and incubated overnight at 4° C. in a humidified chamber. Slides were washed twice in PBS-T and stained with secondary antibody (goat anti-rabbit AF488, Life Technologies catalog number A11034, 1:1000) for 1 hour at room temperature. Slides were washed twice in PBS-T and stained with DAPI then mounted with OmniMount (National Diagnostics catalog number HS-110). Slides were imaged with an upright widefield microscope with a 40× objective and images collected with Pannoramic Scanner Software 2.0. Images were pseudocolored and processed with FIJI software.

Immunofluorescence of frozen tissue. Tumors were harvested from E-Cadherin^{CFP}GzmC-iCre^{tdT}PyMT mice and fixed in Periodate-Lysine-Paraformaldehyde (PLP) for 16-24 hours, 30% sucrose for 24 hours, then frozen in OCT. Tissue was sectioned at 20 μm thickness, blocked for 30 minutes and stained with Hoechst and a fluorescently conjugated antibody against PyMT (Thermo Fisher Scientific, catalog number MA1-46061) overnight at 4° C. Slides were washed and stained with secondary antibody (goat anti-rat AF488, Life Technologies catalog number A-11006). Images were taken on confocal microscope using 4 color channels.

Statistical analysis. Spearman's correlation, log-rank and Wilcoxon tests were used to calculate statistical significance where appropriate. Two tailed unpaired t tests, ratio paired t tests, and one-way ANOVA were conducted using Prism 8 software. A value of P<0.05 was considered statistically significant.

### Example 11: ccRCC, but not chRCC, Tumors are Abundantly Infiltrated by PD1⁺CD8⁺ T Cells

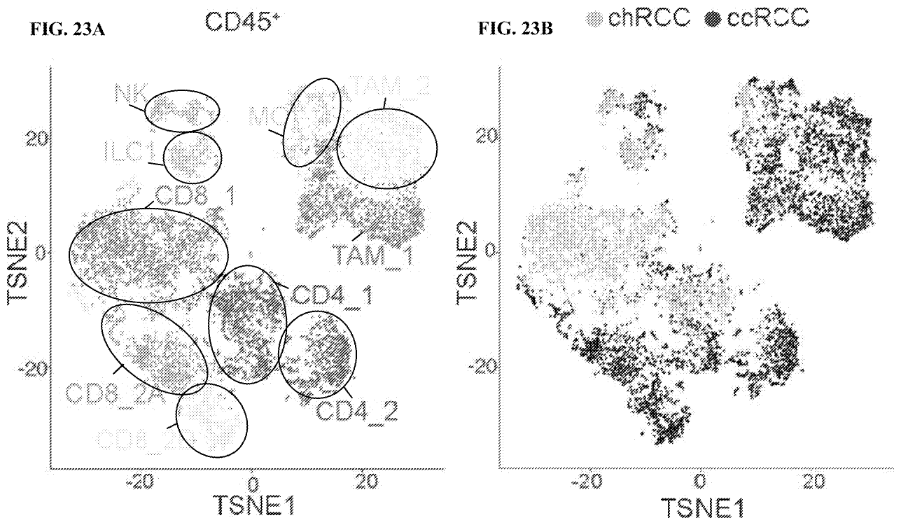

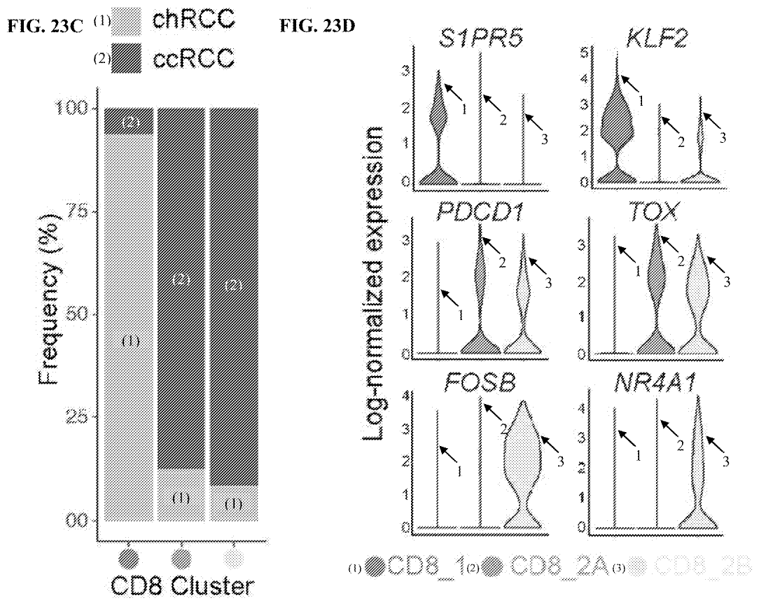

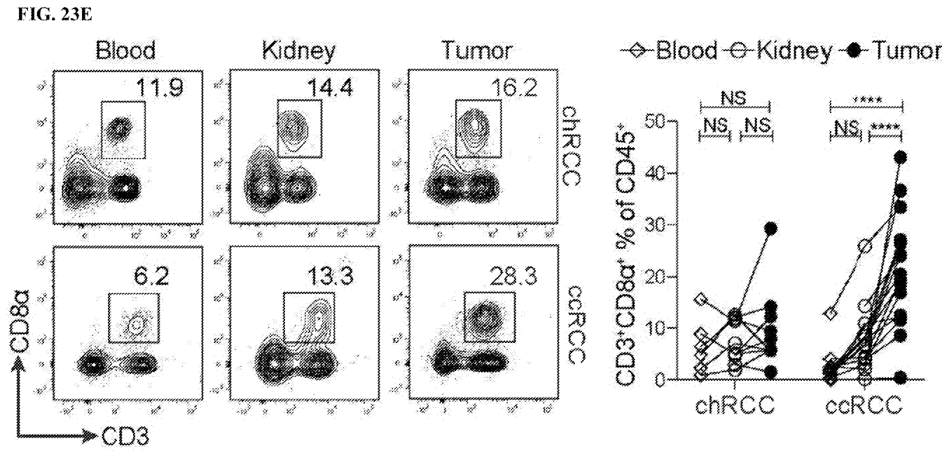

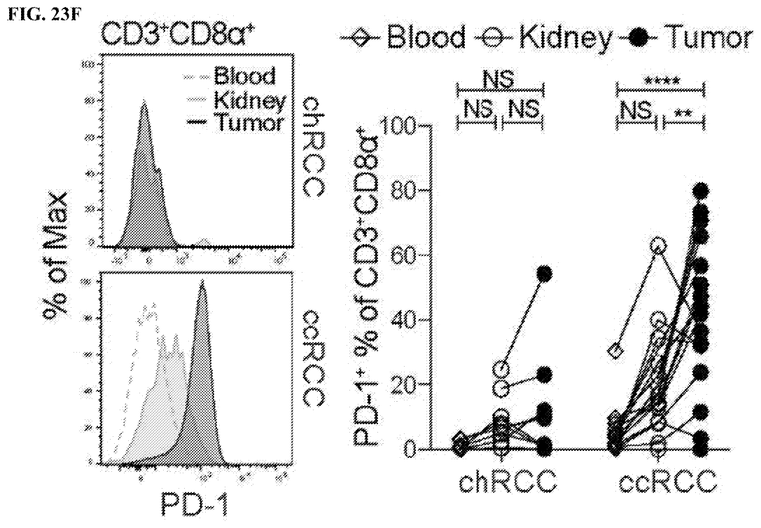

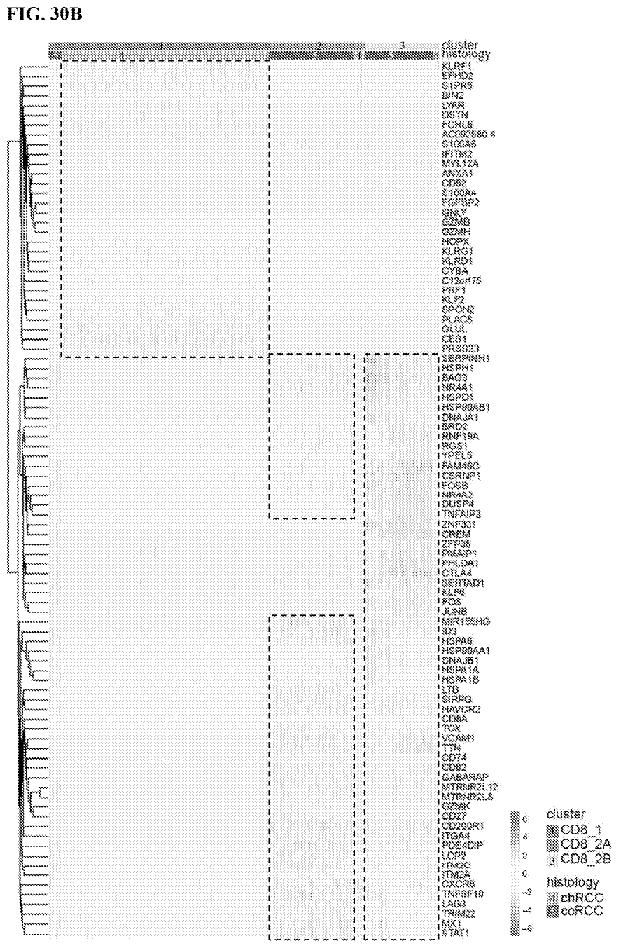

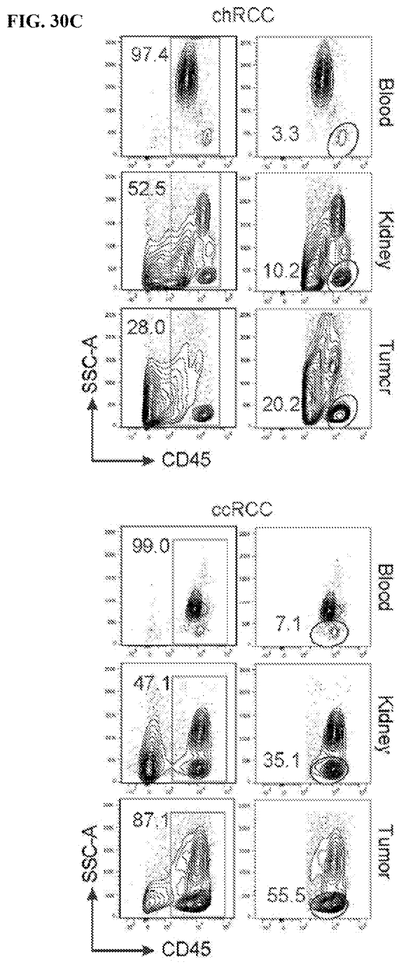

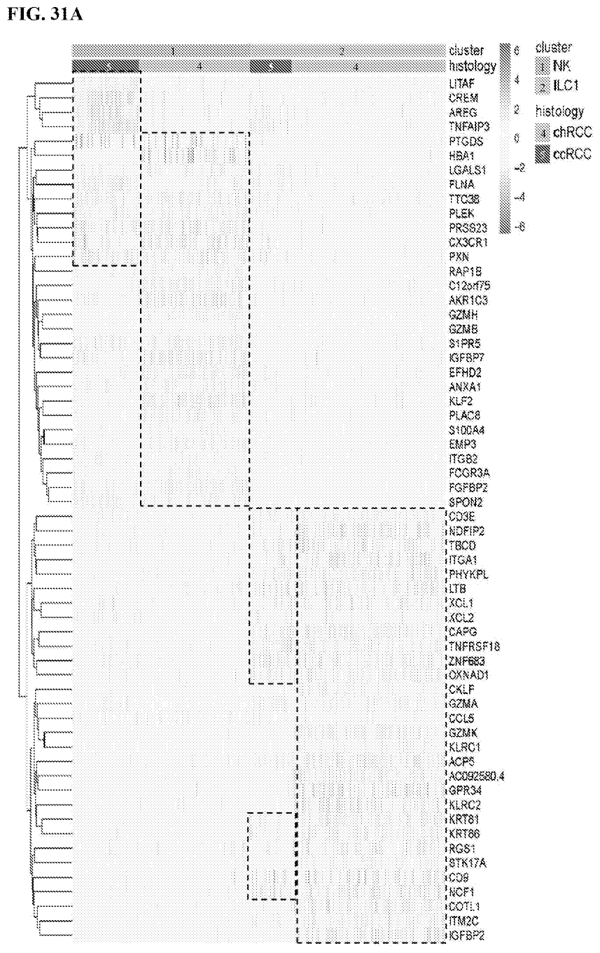

RCC is among the top 10 most common cancers in both men and women with histologically distinct subtypes manifesting differential responses to therapies. Notably, while immune checkpoint blockade therapy has significantly increased survival of ccRCC patients, chRCC patients are generally refractory to the treatment. To define the immunological basis of this differential outcome, we performed single-cell RNA sequencing on CD45⁺ cells isolated from late-stage tumors of drug treatment-naïve ccRCC and chRCC patients. Pooled data revealed 10 major clusters defined by lineage marker plots and differential gene analysis (FIG. 23A, FIG. 30A). Of note, the overall abundance of the different immune cell clusters was markedly different between the two cancer subtypes (FIG. 23B). Among the three CD8⁺ T cell clusters, cluster CD8_1 and clusters CD8_2A and 2B contained cells mostly from chRCC and ccRCC patients, respectively (FIG. 23C). Notably, cluster CD8_1 had a gene expression profile marked by high expression of SIPR5 and KLF2 (FIG. 23D, FIG. 30B), molecules implicated in mediating cell egress into the blood and lymphatic system. Clusters CD8_2A and 2B showed highly similar gene expression profiles and expressed T cell exhaustion markers PDCD1 and TOX (FIG. 23D, FIG. 30B), but clustered separately based on differences in the expression of immediate early genes including FOSB and NR4A1 (FIG. 23D, FIG. 30B).

We then sought to substantiate these findings and thus performed flow cytometric analyses on an extensive cohort of RCC patient tumor, tumor-adjacent normal kidney, and blood samples collected directly from the operating room. We observed that ccRCC tumors had significantly more CD8⁺ T cells compared to adjacent normal kidney and blood, reflecting either infiltration or expansion in the tumor, while chRCC patients had no significant difference in CD8⁺ T cell abundance across tissues (FIG. 23E and FIG. 30C). In addition, quantification of PD-1 expression revealed that higher percentages of CD8⁺ T cells from ccRCC tumors expressed PD-1 than their normal kidney and blood counterparts (FIG. 23F). With rare exception, chRCC CD8⁺ T cells were practically PD-1-negative across tissues suggesting that like the CD8⁺ T cells profiled by single-cell RNA sequencing in the chRCC patient, these cells were bystanders not engaged in active surveillance (FIG. 23F). Overall, these data suggest that contrary to ccRCC patients, chRCC patients generally do not have a conventional PD-1⁺CD8⁺ T cell response in tumors, revealing a potential mechanism for the failed response to anti-PD-1 therapy.

### Example 12: chRCC Tumors are Highly Enriched for CD56^{bright}CD49a⁺CD103⁺ ILCs

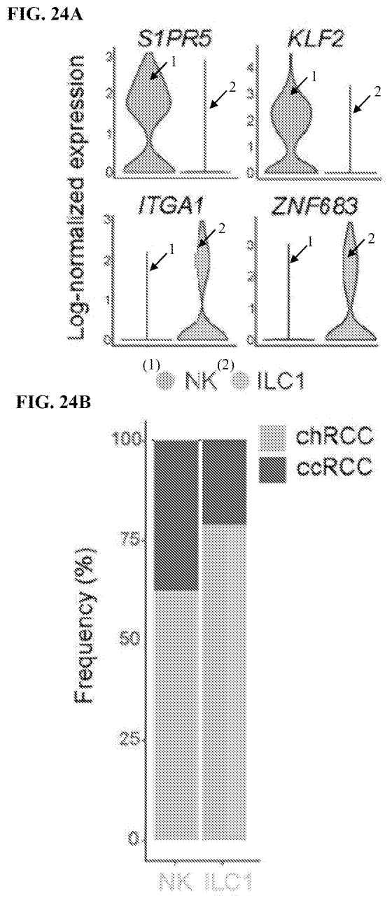

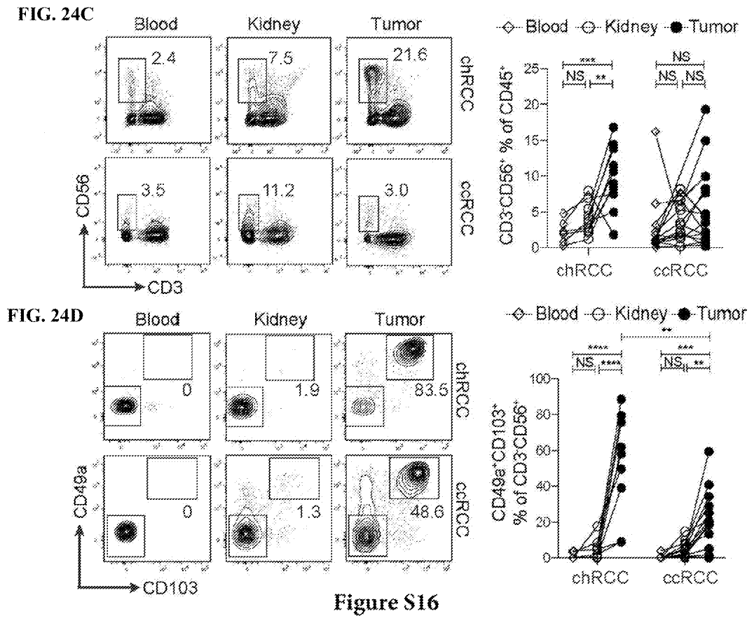

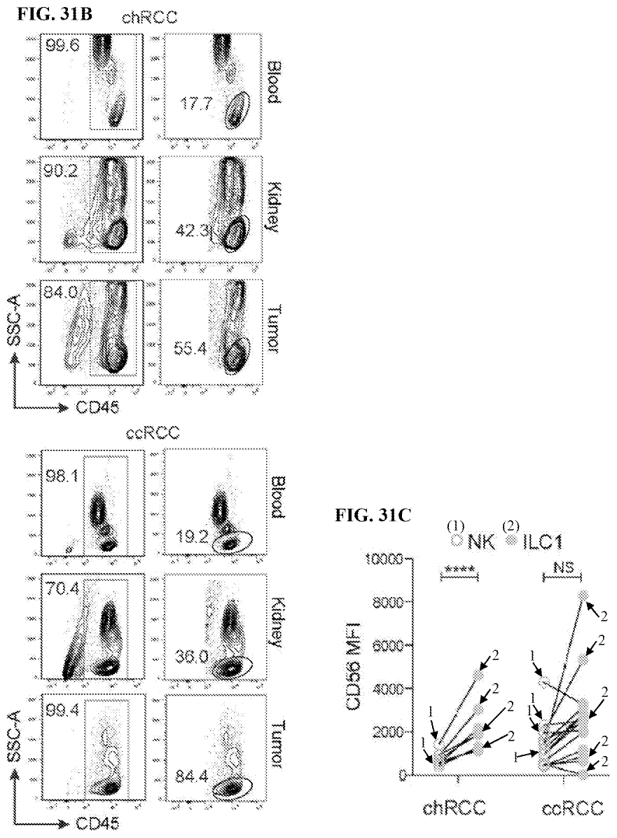

In addition to three clusters of CD8⁺ T cells, two clusters of innate lymphocytes defined by high expression of KLRB1 encoding the killer cell lectin-like receptor CD161 were detected by single-cell RNA sequencing (FIG. 23A and FIG. 31A). Their phenotypes were distinct in terms of expression of surface molecules and transcription factors with one cluster, like cluster CD8_1, expressing high levels of SIPR5 and KLF2 that define circulating NK cells (FIG. 24A, FIG. 31A). The other cluster expressed high levels of ITGA1 encoding the integrin CD49a, and ZNF683 which encodes the transcription factor Hobit that defines tissue-resident ILC1s (FIG. 24A, FIG. 31A). Both NK and ILC1 clusters were present in each histology, but cluster ILC1 was more abundant in chRCC than ccRCC (FIG. 24B). Both clusters displayed potential for cytotoxicity but had differences in granzyme expression, such as GZMH and GZMA expressed in cluster NK and cluster ILC1, respectively (FIG. 31A). These data suggest that two distinct populations of innate lymphocytes with cytotoxic potential exist in RCC tumors-one predominantly circulating with canonical NK cell features and the other tissue-resident with ILC1 properties.

To confirm these findings, we analyzed an extensive RCC patient cohort by flow cytometry. There was a significant increase in total CD3⁻CD56⁺ innate lymphocytes in chRCC, but not ccRCC, tumors compared to adjacent normal kidney and blood (FIG. 24C and FIG. 31B). We probed the CD3⁻CD56⁺ population for ILC1s and NK cells by expression of CD49a and CD103, another integrin molecule that promotes lymphocyte retention in epithelial cancer. Notably, the vast majority of CD3⁻CD56⁺ cells in the blood and adjacent normal kidney were CD49a⁺CD103⁻ NK-phenotype cells (FIG. 24D). In contrast, there was a substantial increase in CD49a⁺CD103⁺ ILC1s in tumor tissues of both histologies, however the magnitude of the response was significantly higher in chRCC compared to ccRCC (FIG. 24D). To further characterize these two innate lymphocyte populations in RCC tumors, we measured the level of CD56 expression and noted that the CD49a⁺CD103⁺ ILC1 population consistently expressed higher levels of CD56 than the CD49a⁺CD103⁻ NK cell subset in chRCC, but not ccRCC, tumors (FIG. 31C). Together, these findings reveal that chRCC tumors are characterized by robust expansion and activation of ILC1s.

### Example 13: ILC1 Response Predicts Better Overall Survival in chRCC

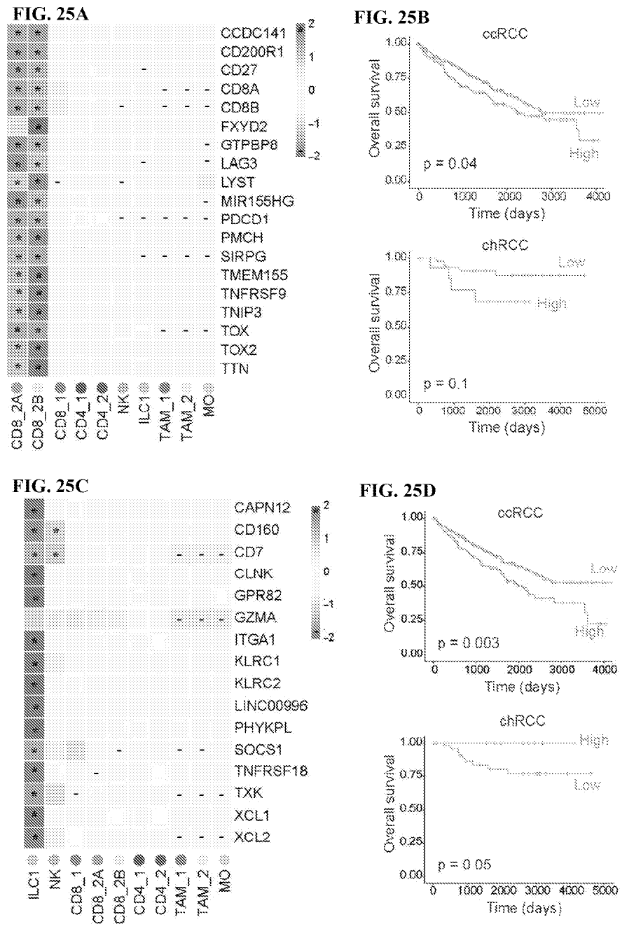

Thus far our data demonstrate that ccRCC tumors are populated by exhausted CD8+ T cells and ILC1s, while chRCC tumors are populated by only the latter. To explore the functional relevance of these tumor-elicited responses, we generated signatures of the CD8⁺ T cell and ILC1 clusters. Given the overall similarities of clusters CD8_2A and CD8_2B, we combined them into one cluster and performed pairwise differential gene expression (DEG) analysis relative to every other CD45⁺ cluster retrieved from single-cell RNA sequencing. The resulting gene set included genes significantly upregulated in CD8_2A and CD8_2B in every comparison, and included the exhaustion markers PDCD1, LAG3, and TOX (FIG. 25A). Application of the CD8_2 gene signature to the ccRCC and chRCC TCGA cohorts showed a negative association of enrichment of this signature and overall survival for both histologies (FIG. 25B), likely a reflection of an ineffectual CD8⁺ T cell response that tracks with disease progression.

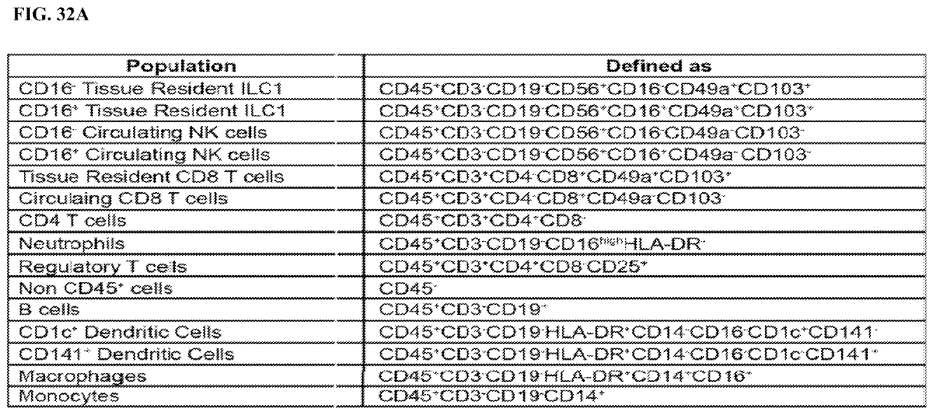

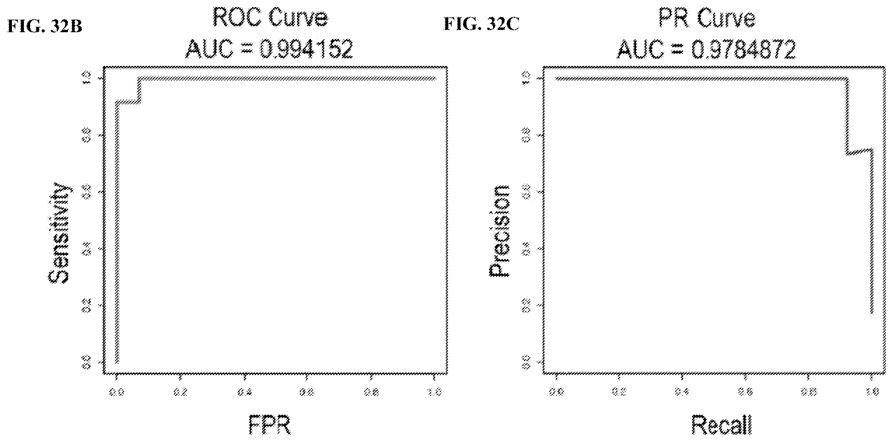

Similarly, we generated a gene signature for the ILC1 cluster by performing pairwise DEG analysis relative to every other cluster to obtain a set of significantly upregulated genes in the ILC1 cluster in every comparison (FIG. 25C). To further validate the ILC1 gene signature, we applied it to an independent cohort of 69 bulk RNA sequencing datasets of immune cell as well as non-immune cell populations isolated from ccRCC and chRCC patient tumors (FIG. 32A). For a total of 12 individual ILC1 samples and 57 non-ILC1 cell populations, the signature was highly discriminative for ILC1s as indicated by the high areas under both receiver operating characteristic (ROC) and precision recall (PR) curves (FIGS. 32B-32C). Interestingly, high expression of the ILC1 gene signature was associated with better and worse overall survival in chRCC and ccRCC patients, respectively (FIG. 25D). Of note, all chRCC patients that fell in the top quartile for ILC1 gene signature expression had a 100% survival rate regardless of tumor stage (FIG. 25D and data not shown), suggesting the potential for a prognostic marker as well as a mechanism of effective tumor control.

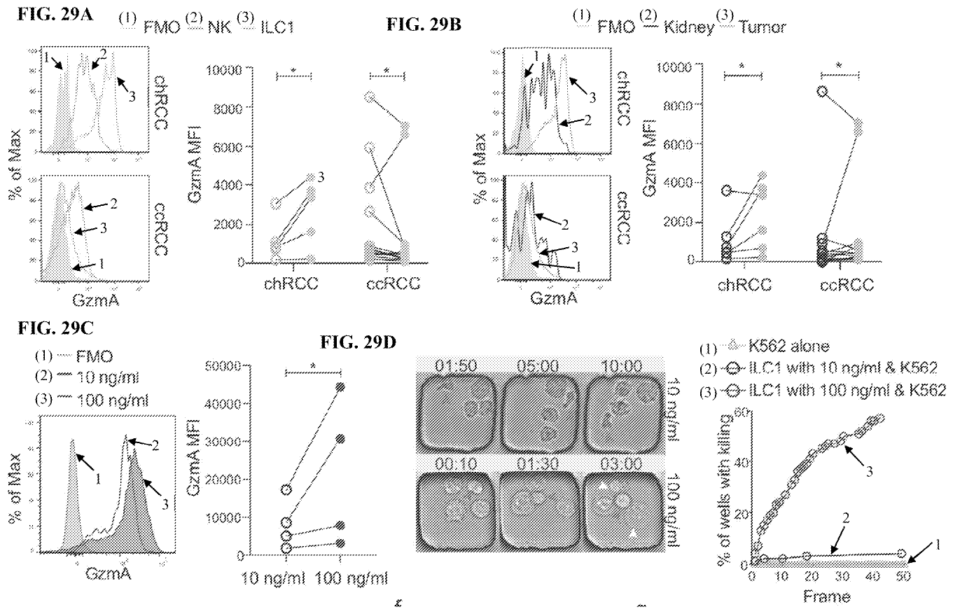

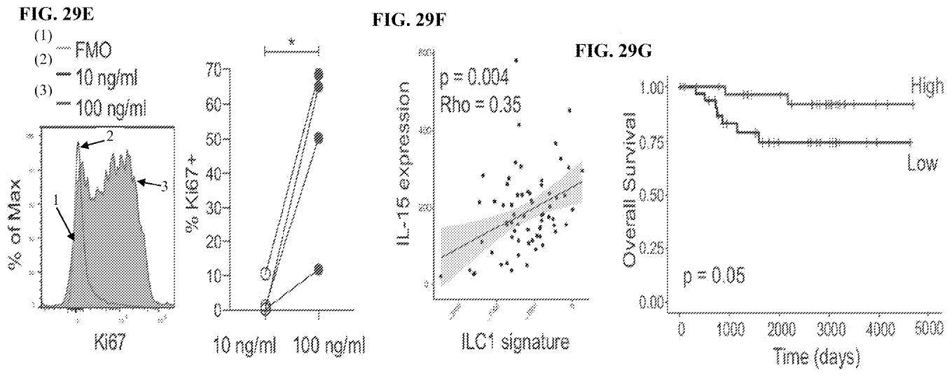

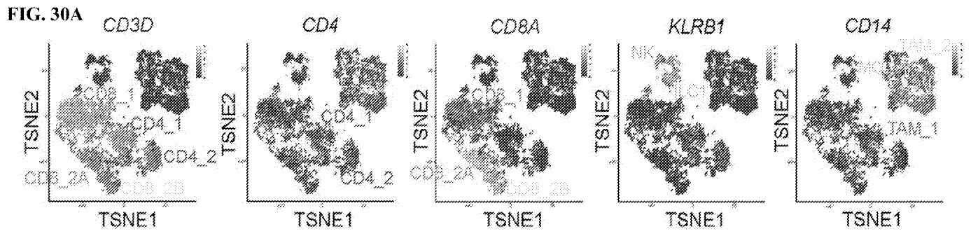

The opposing predictive values of the ILC1 gene signature on ccRCC and chRCC patients' survival suggested phenotypic and functional heterogeneity of ILC1s. A potentially functional ILC1 signature gene was GZMA (FIG. 25C), which encodes a cytotoxic molecule involved in lytic granule-mediated noncanonical apoptosis and pyroptosis. Examination of granzyme A protein expression among a cohort of chRCC and ccRCC patients revealed that ILC1s consistently expressed more granzyme A than matched NK cell counterparts, but only in chRCC tumors (FIG. 29A). In fact, ILC1s had lower granzyme A expression than NK cells in most ccRCC tumors (FIG. 29A). In addition, ILC1s from tumor tissue expressed higher levels of granzyme A than ILC1s from adjacent normal kidney, again more consistently in chRCC patients (FIG. 29B). Thus, despite induction of ILC1s in both chRCC and ccRCC tumors, they exhibit disparate patterns of granzyme A expression in association with histology-specific prognosis of the ILC1 gene signature.

### Example 14: IL-15 is a Crucial Regulator of ILC1 Phenotype and Function in chRCC

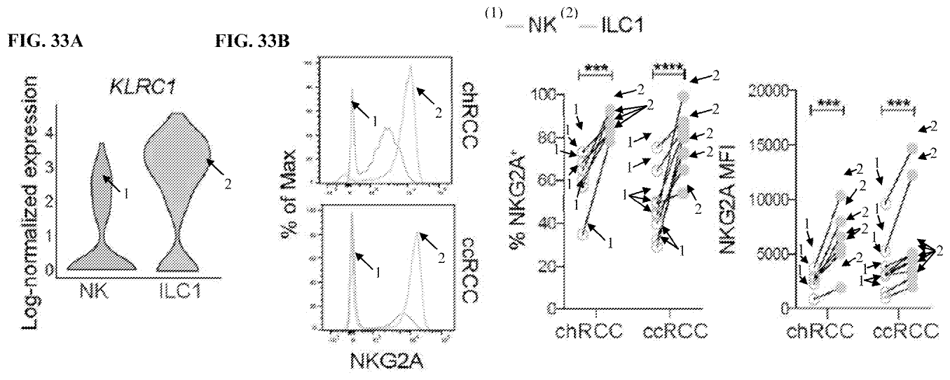

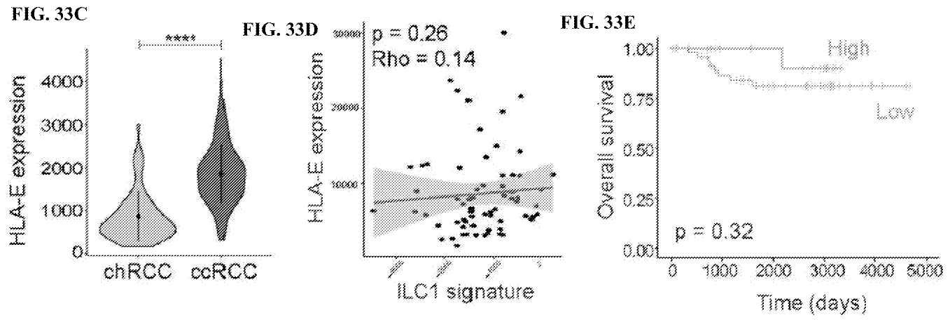

We wished to define how ILC1 responses were differentially regulated in RCC tumors. The ILC1 cluster was enriched for the KLRC1 transcript (FIG. 33A), which encodes for the inhibitory receptor NKG2A. Flow cytometry experiments revealed that compared to NK cells, ILC1s from both chRCC and ccRCC tumors had a higher fraction of cells expressing NKG2A as well as higher levels of NKG2A protein expression (FIG. 33B). To determine whether differential exposure of ILC1s to the NKG2A ligand HLA-E might affect ILC1 responses in RCC, we examined HLA-E transcript expression in the TCGA database, and observed lower HLA-E expression in chRCC tumors than ccRCC tumors (FIG. 33C). Nonetheless, HLA-E expression was not anti-correlated with ILC1 signature in chRCC tumors (FIG. 33D), nor did it negatively track with chRCC patient survival (FIG. 33E). These observations suggest that NKG2A sensing of HLA-E is unlikely a major determinant of the ILC1 response in RCC.

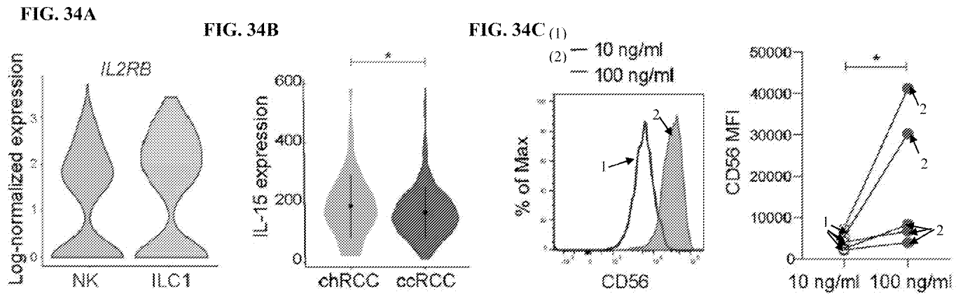

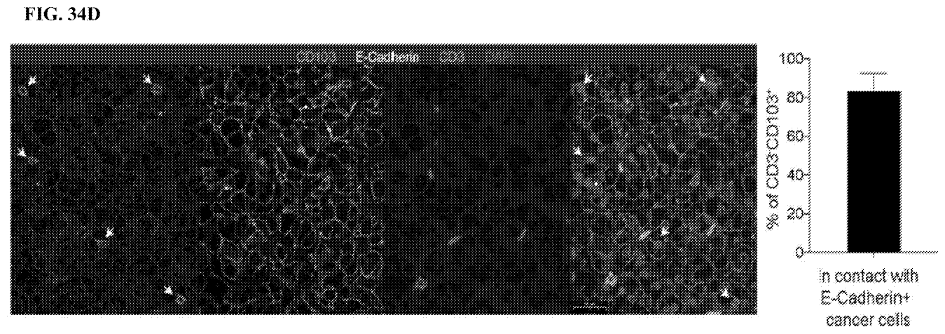

The cytokine IL-15 in complex with IL-15 receptor α chain (IL-15Rα) promotes the development, maintenance, and effector function of lymphocytes with cytotoxic potential. Previous studies have shown that brief priming with IL-15 markedly enhances the antitumor response of CD56^{bright} NK cells isolated from blood. Thus, we explored whether IL-15 played a role in the regulation of ILC1s. Indeed, both the NK and ILC1 clusters from single-cell RNA sequencing data showed high expression of IL2RB (FIG. 34A), encoding the shared signaling IL-2/IL-15 receptor β chain. Notably, chRCC tumors from the TCGA cohort had higher expression of IL-15 transcripts than ccRCC tumors (FIG. 34B), implying that the level of IL-15 in the tumor microenvironment may regulate tissue-resident innate lymphocyte responses in a dose-dependent manner.

To explore the potential role of IL-15 in control of ILC1 function, we isolated these cells from RCC tumors and cultured them with two different doses of an IL-15/IL-15Ra complex, 10 and 100 ng/ml, both of which maintained survival of the cells (data not shown). The higher dose of IL-15/IL-15Rα complex induced greater expression of granzyme A and CD56 (FIG. 29C and FIG. 34C), two proteins enriched in chRCC ILC1s (FIG. 29A and FIG. 31C). In addition, the higher dose of IL-15/IL-15Rα substantially enhanced the cytotoxicity of ILC1s against target cells in a single-cell killing assay (FIG. 29D). The higher dose of IL-15/IL-15Rα also resulted in increased expression of the cell proliferation marker Ki67 (FIG. 29E), in agreement with greater expansion of ILC1s in chRCC than ccRCC tumors (FIG. 24D). Of note, there was a positive correlation of level of IL-15 expression and enrichment of the ILC1 signature across the chRCC TCGA patient cohort (FIG. 29F). Moreover, patients with higher expression of IL-15 trended towards better overall survival than patients with lower expression (FIG. 29G). Collectively, these findings demonstrate that IL-15 regulates the cytotoxic program of tissue-resident ILC1s and enhances their ability to kill target cells, which may represent a critical mechanism for tumor control in chRCC patients.

### Example 15: ILC1s are Induced in Human and Murine Breast Cancers in Association with IL-15 Expression

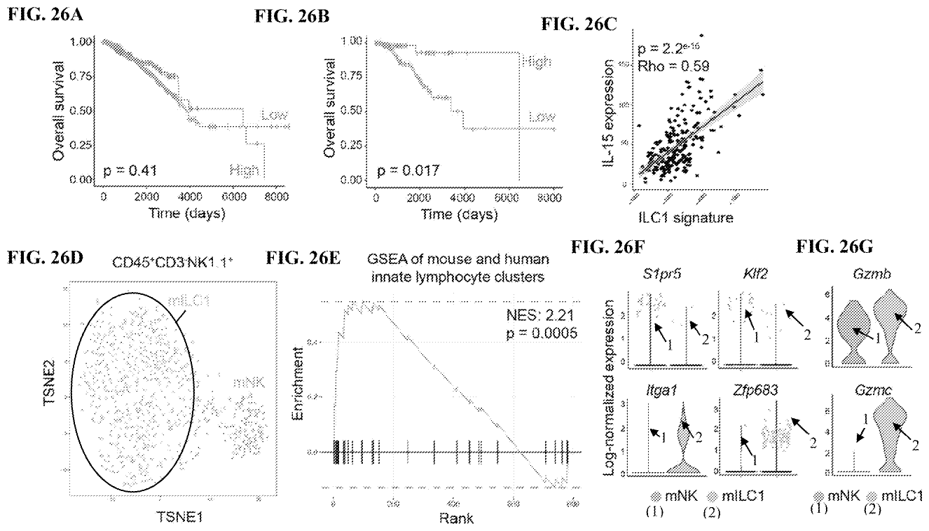

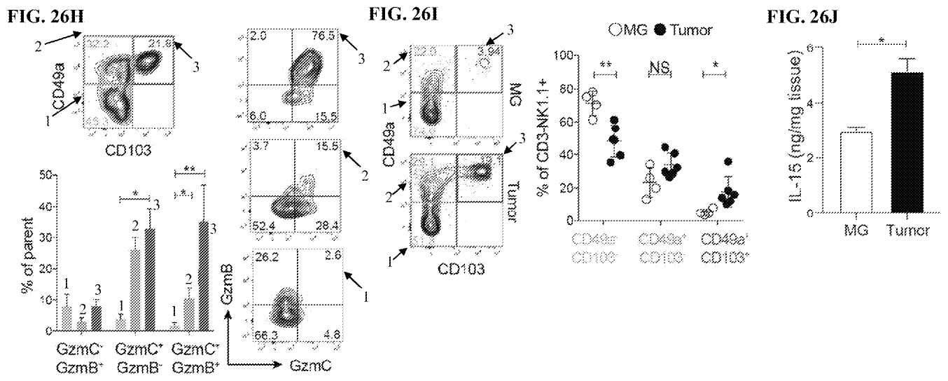

We next sought to explore whether IL-15 governs the ILC1-mediated cancer immunosurveillance in other epithelial malignancies. As the most common cancer worldwide, breast cancer composes of heterogenous subsets defined by immunohistochemical and genomic markers. Phenotypic profiling of mammary tumors revealed differential cell surface protein expression in tumor-infiltrating type 1 innate lymphocytes, including high expression of the ILC1-enriched NKG2A inhibitory receptor. Although these cells were broadly classified as “NK cells”, they could represent ILC1s as ILCs including Hobit-expressing ILC1s are abundant in mammary tumors revealed by a recent single-cell RNA sequencing study. To probe the function and regulation of ILC1s in breast cancer, we applied the ILC1 gene signature to the TCGA database and found that patients with higher ILC1 signature trended to have better survival at earlier time points (FIG. 26A), although it did not reach statistical significance. Subsetting breast cancer patients based on hormone receptors and HER2 expression did not substantially change the statistics (data not shown). However, the ILC1 signature predicted better survival of patients that harbor the hotspot PIK3CA mutations (p.H1047R, p.E545K, or p.E542K) (FIG. 26B), and the ILC1 signature value positively tracked with IL-15 expression (FIG. 26C). These observations suggest a particularly important function for IL-15-regulated ILC1s in surveillance of breast cancers driven by PIK3CA gain-of-function mutations.

We wished to further decipher the cellular mechanisms by which IL-15 regulates tissue-resident innate lymphocytes in the tumor microenvironment, and used a transgenic mouse model of breast cancer driven by the murine polyomavirus middle tumor antigen (PyMT) that activates PI3K, and thus models PIK3CA gain-of-function mutations. Our previous studies have revealed that tissue-resident cytotoxic ILC1-like innate lymphocytes expand in mammary tumors of PyMT mice. To profile tumor-associated innate lymphocytes in an unbiased manner, we performed single-cell RNA sequencing experiments of CD3⁻ NK1.1⁺ group 1 innate lymphocytes from PyMT tumors, and identified two major innate lymphocyte clusters, mouse NK (mNK) and mouse ILC1 (mILC1) (FIG. 26D). Gene set enrichment analysis (GSEA) revealed significant enrichment of differentially expressed DEGs between mNK and mILC1 clusters in the human NK and ILC1 DEG dataset (FIG. 26E), suggesting broad similarity between the murine and human clusters. Indeed, similar to their human counterparts, cluster mNK expressed higher levels of S1pr5 and Klf2, while cluster mILC1 had higher expression of the tissue residency marker Itga1 and Zfp683 (FIG. 26F). Yet, expression of granzymes diverged through evolution with Gzmc and Gzmb transcripts enriched to different degrees in cluster mILC1 (FIG. 26G). We confirmed by flow cytometry that CD49a⁺CD103⁺ ILC1s had highest co-expression of granzyme C and granzyme B, while CD49a⁺CD103⁻ NK cells expressed only low levels of granzyme B (FIG. 26H). Compared to healthy mammary glands, transformed mammary tissues were enriched for ILC1s within the CD3⁻NK1.1⁺ innate lymphocyte compartment in association with increased expression of IL-15 (FIGS. 26I-26J). These findings suggest that NK1.1⁺CD49a⁺CD103⁺ ILC1s represent the murine equivalent of human CD56⁺CD49a⁺CD103⁺ ILC1s with their induction similarly associated with increased expression of IL-15 in tumor.

### Example 16: ILC1s Function Independently of Dendritic Cell- and Macrophage-Expressed IL-15

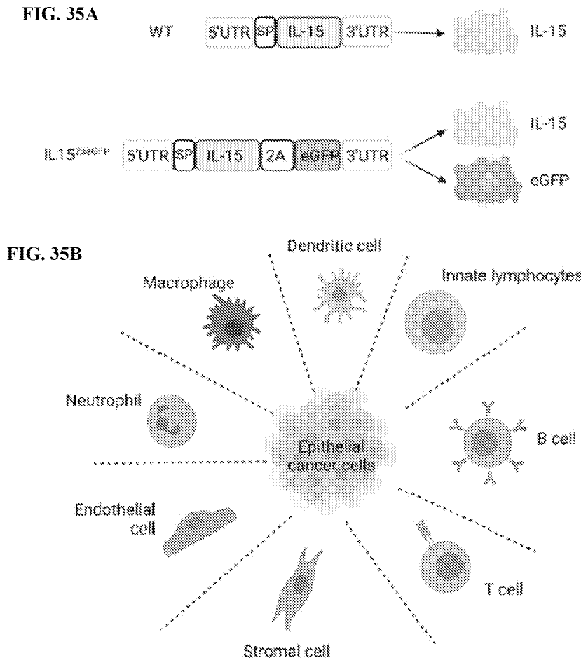

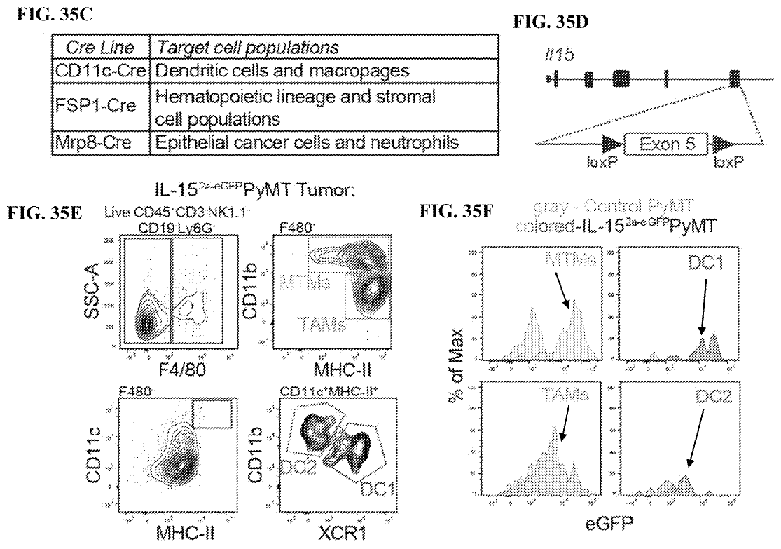

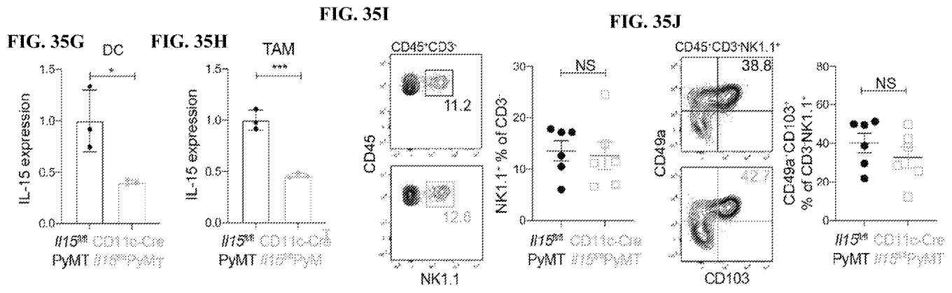

To investigate which cell types expressed IL-15 in tumor, we employed an IL-15 enhanced green fluorescent protein (eGFP) reporter mouse strain with eGFP transcription and translation under the control of the IL-15 gene locus (IL-15^{2A-eGFP}) (FIG. 35A). To interrogate the exact cellular sources of IL-15 that promotes tissue-resident innate lymphocyte responses in tumor, we utilized a mouse strain harboring an exon 5-floxed allele of the IL-15 gene (IL-15^{fl}) that could be inactivated by breeding with Cre recombinase transgenic mouse lines to target various immune cells, tumor stromal cell populations, and cancer cells (FIGS. 35B-35D).

Using a conditional knockout IL-15ra mouse strain, previous studies revealed that dendritic cells (DCs) and macrophages provide a critical source of IL-15 in support of homeostasis of circulating NK cells. Two prominent macrophage populations, tumor-associated macrophages (TAMs) and mammary tissue macrophages (MTMs), along with two conventional DC1 and DC2 subsets, were detected in PyMT tumors (FIG. 35E). eGFP reporter analysis showed that MTMs and XCR1+DCIs expressed higher levels of IL-15, followed by TAMs and CD11b⁺ DC2s in IL-15^{2A-eGFP}PyMT mice (FIG. 35F). To investigate the function of IL-15 produced by these cells, we crossed IL-15^{fl/fl}PyMT mice with CD11c-Cre transgenic mice that target DCs and TAMs (FIGS. 35G-35H). Surprisingly, IL-15 loss in DCs and macrophages had no significant effect on the abundance of CD3⁻NK1.1⁺ cells or the CD49a⁺CD103⁺ ILC1 subset in tumors of CD11c-CreIL-15^{fl/fl}PyMT mice (FIGS. 35I-35J) . Furthermore, there was no difference in granzyme B and granzyme C expression in ILC1s (FIGS. 35K-35L). In line with these findings, there was no difference in tumor burden in CD11c-CreIL-15^{fl/fl}PyMT mice compared to controls (FIG. 35M). Splenic NK cells, in particular the mature DX5⁺CD27⁻CD11b⁺ NK cell subset, were reduced in CD11c-CreIL-15^{fl/fl}PyMT mice (FIG. 35N-35O). These findings demonstrate that IL-15 derived from CD11c⁺ myeloid cell populations is dispensable for the induction of ILC1s in PyMT tumors.

### Example 17: ILC1s Function Independently of Hematopoietic and Stromal Cell Sources of IL-15

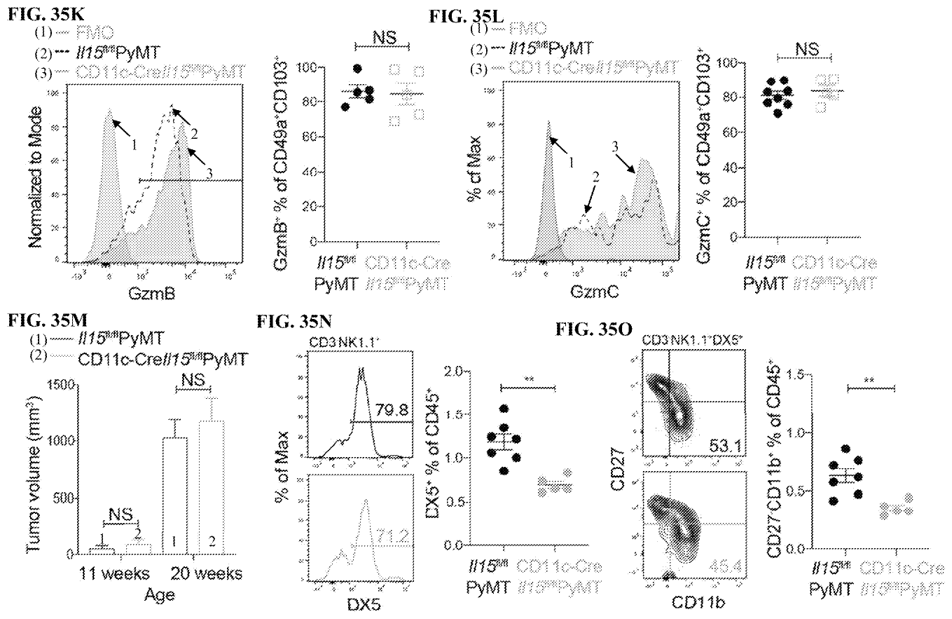

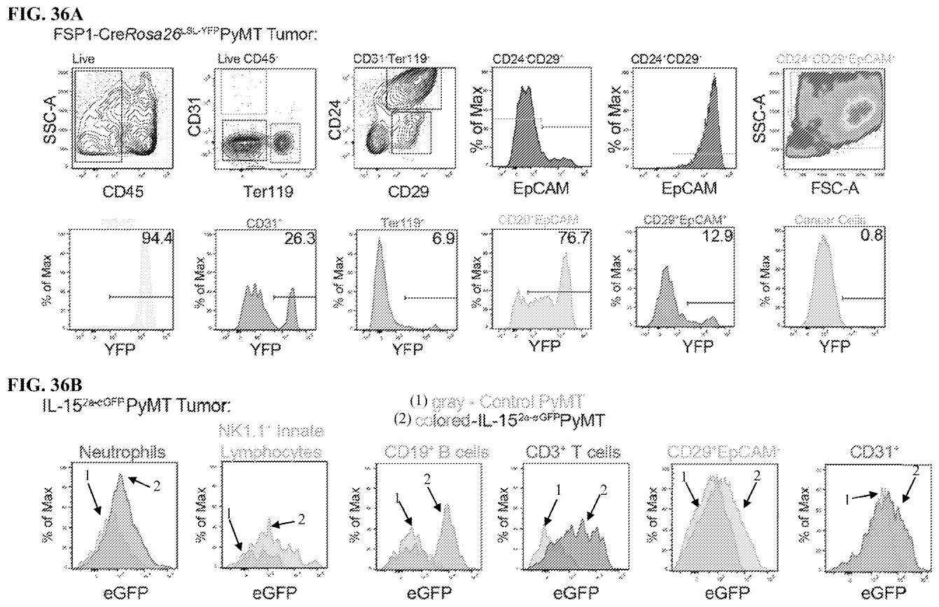

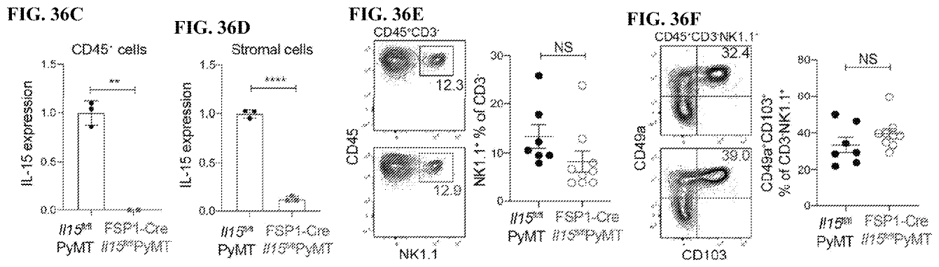

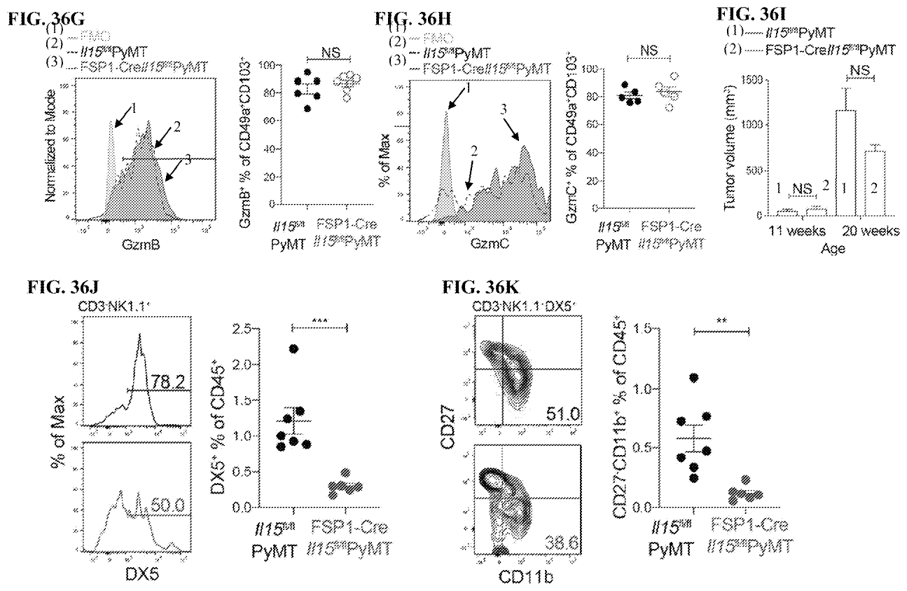

We wished to expand IL-15 depletion with the FSP1-Cre line that targets various mesenchymal and hematopoietic cell lineages in adult tissues. Using a Rosa26^{LSL-YFP} Cre activity reporter line bred to the FSP1-CrePyMT background, we observed YFP expression in approximately 94% CD45⁺ leukocytes, the majority of CD29⁺EpCAM stromal cells, and a subset of CD31⁺ endothelial cells, but minimally in Ter119⁺ erythrocytes or EpCAM⁺ cancer cells in PyMT tumors (FIG. 36A). eGFP reporter analysis of IL-15^{2A-eGFP}PyMT mice revealed that in addition to DCs and macrophages, Ly6G⁺ neutrophils, NK1.1⁺ innate lymphocytes, CD19⁺ B cells, CD3⁺ T cells, CD29⁺EpCAM stromal cells, and CD31⁺ endothelial cells in PyMT tumors expressed varying amounts of IL-15 (FIG. 36B).

To evaluate the function of IL-15 produced by FSP1-Cre-targeted cells, we bred FSP1-Cre mice to the IL-15^{fl/fl}PyMT background which led to effective deletion of IL-15 in virtually all CD45⁺ cells and CD29⁺EpCAM⁻ stromal cells (FIGS. 36C-36D). Unexpectedly, neither tumor CD3⁻NK1.1⁺ cells nor the CD49a⁺CD103⁺ ILC1 subset was substantially altered (FIGS. 36E-36F). Granzyme B and granzyme C expression were also comparable in ILC1s, which was associated with comparable tumor burdens in IL-15^{fl/fl}PyMT and FSP1-CreIL-15^{fl/fl}PyMT mice (FIGS. 36G-36I). As expected, splenic NK cells and specifically the mature DX5⁺CD27⁻CD11b⁺ subset were greatly diminished in FSP1-CreIL-15^{fl/fl}PyMT mice (FIGS. 36J-36K). Together, these findings suggest that hematopoietic and stromal sources of IL-15 are of no consequence to the induction and function of tissue-resident ILC1s in PyMT tumors.

### Example 18: ILC1s Interact with and Directly Sense Cancer Cells

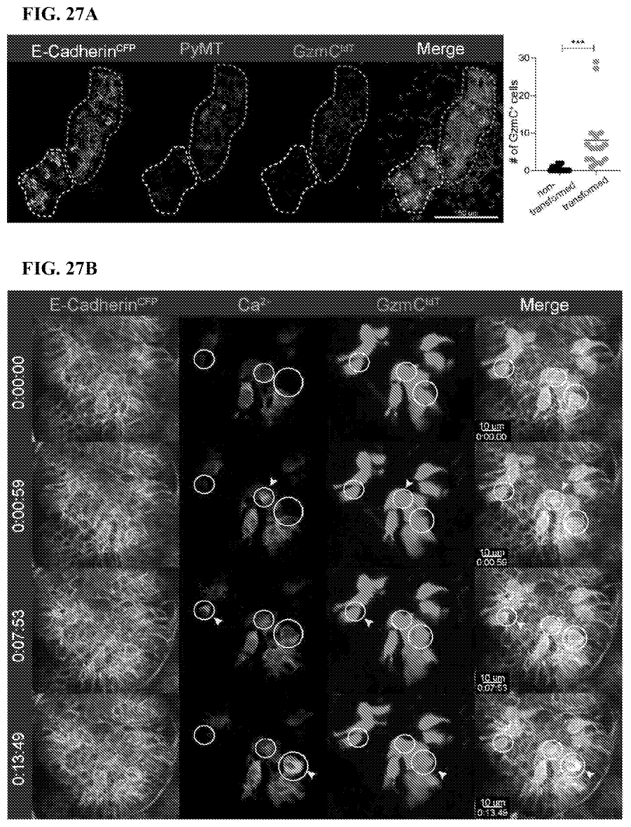

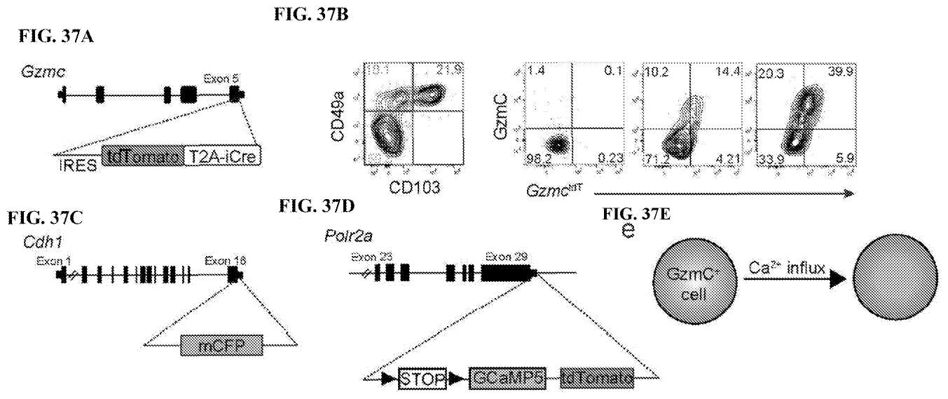

We wished to define the precise cellular niche in support of tissue-resident innate lymphocyte responses in tumor. Considering that granzyme C was selectively expressed in CD49a⁺CD103⁺ ILC1s (FIGS. 26G-26H), we generated an iCre recombinase reporter mouse line in which expression of iCre and a tandem dimer of Tomato (tdT) reporter was under the control of the Gzmc gene locus (FIG. 37A). We verified that tdT was expressed exclusively in the CD49a⁺CD103⁺ ILC1 subset of CD3⁻NK1.1⁺ innate lymphocytes and faithfully reported granzyme C protein expression (FIG. 37B). To interrogate the distribution of granzyme C-expressing cells in reference to the epithelial cell lineage, we crossed Gzmc^{tdT-T2A-iCre} mice to a E-Cadherin reporter mouse line that harbors a monomeric cyan fluorescent protein (mCFP) expression cassette under the control of the Cdh1 gene locus (FIG. 37C), which were further bred to the PyMT background. Mammary tissues were isolated from these mice and stained for the expression of PyMT oncoprotein to identify transformed mammary epithelial cells. Intriguingly, GzmC^{tdT}-expressing cells accumulated within PyMT-positive transformed areas of mammary tissues in close contact with E-Cadherin^{mCFP}-expressing cancer cells, and were rarely detected in PyMT-negative non-transformed epithelia (FIG. 27A). Notably, immunofluorescence staining also revealed that the vast majority of CD3 CD103⁺ ILC1s were in direct contact with E-Cadherin-expressing cancer cells in chRCC patients (FIG. 34D). Thus, the evolutionarily conserved tumor-elicited tissue-resident ILC1 response is characterized by close interactions between cancer cells and innate lymphocytes.

To further define the cell behavior of tissue-resident innate lymphocytes, we bred Gzmc^{tdT-T2A-iCre}Cdh1^{mCFP}PyMT mice to a mouse strain harboring a genetically encoded calcium indicator, GCaMP5 made of a circularly permutated green fluorescent protein fused to the calcium-binding protein calmodulin and a M13 peptide, along with a tdT reporter located 3′ of the housekeeping Polr2a gene preceded by a floxed-STOP cassette (FIG. 37D). Cre expression driven by the Gzmc^{tdT-T2A-iCre} allele would result in GCaMP5 and tdT expression in granzyme C-expressing cells, and activation signals causing calcium flux would result in the cells to flash green (FIG. 37E). To analyze these cells in real time, we performed live imaging experiments. GzmC^{tdT}-expressing cells appeared to be embedded within the tumor tissue in very close contact with E-Cadherin^{mCFP}-expressing cancer cells (FIG. 27B). Despite being stationary within the tumor tissue during the time window of imaging, the cells were active as demonstrated by frequent Ca²⁺ flux events (FIG. 27B). These observations suggest that tissue-resident ILC1s directly sense cancer cells.

### Example 19: Cancer Cell-Expressed IL-15 Dictates ILC1 Responses in Tumor

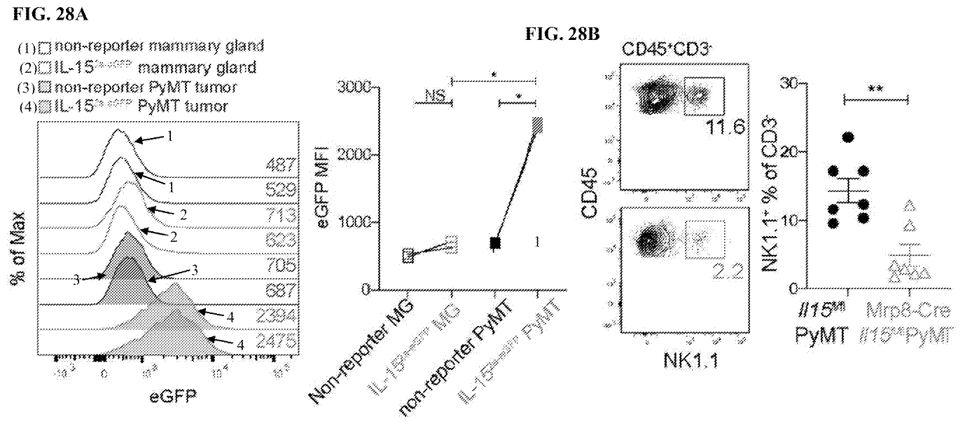

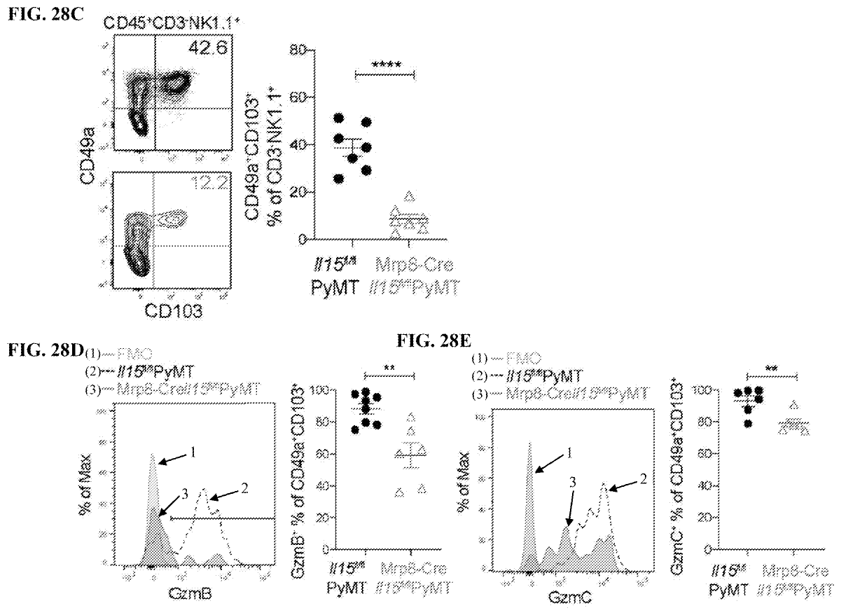

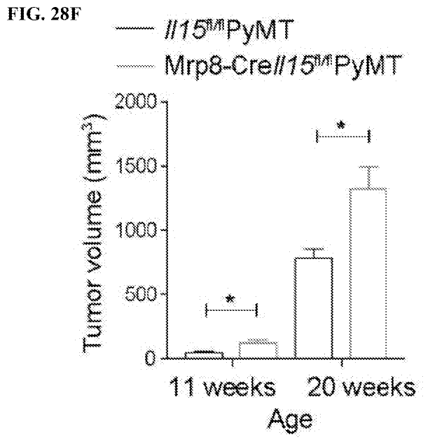

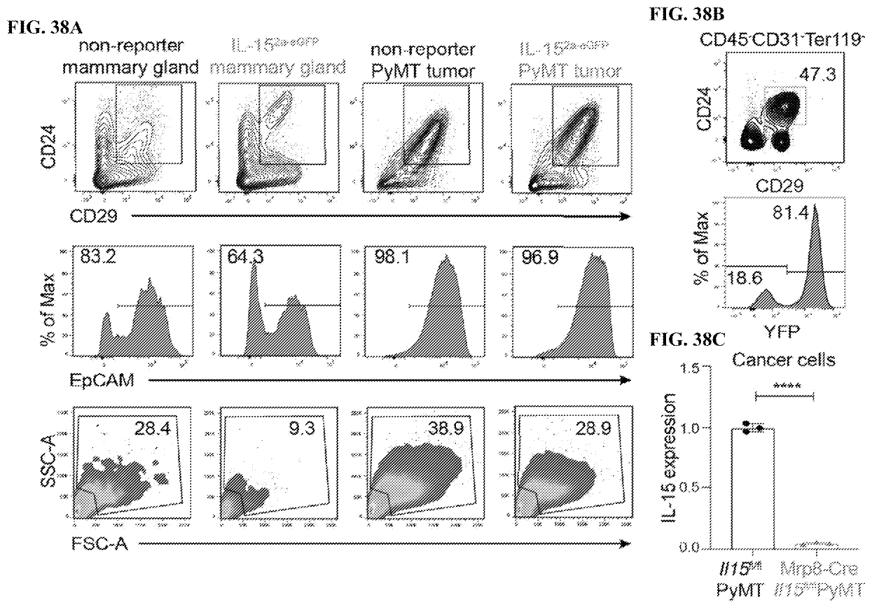

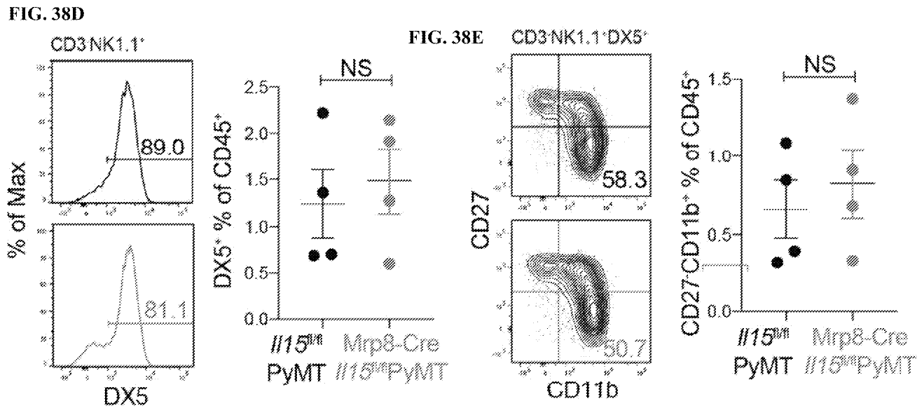

As ILC1s appear to interact with and directly sense cancer cells, we explored the possibility that cancer cells themselves might be the source of IL-15 driving the cancer immunosurveillance response. To determine whether IL-15 was induced following cell transformation, we analyzed eGFP reporter expression in CD24⁺CD29⁺EpCAM⁺ epithelial cells from mammary glands of IL-15^{2A-eGFP} mice or mammary tumors of IL-15^{2A-eGFP}PyMT mice (FIG. 38A). Of note, a higher level of eGFP reporter was detected in cancer cells compared to non-transformed mammary epithelia (FIG. 28A), implying that the enhanced IL-15 level in tumor tissue (FIG. 26J) was due to the increased IL-15 expression in cancer cells.

To target cancer cells, we utilized Mrp8-Cre transgenic mice that marked more than 80% of transformed mammary epithelial cells as read out by crossing the Mrp8-Cre line to a Rosa26^{LSL-YFP} on the PyMT background (FIG. 38B). To investigate the function of cancer cell-derived IL-15, we crossed Mrp8-Cre mice to the IL-15^{fl/fl}PyMT background, which led to effective deletion of IL-15 gene in cancer cells (FIG. 38C). Strikingly, although splenic NK cells were unaffected in Mrp8-CreIL-15^{fl/fl}PyMT mice (FIGS. 38D-38E), total CD3⁻NK1.1⁺ innate lymphocytes and the CD49a⁺CD103⁺ ILC1 subset were severely reduced in tumors from these mice (FIGS. 28B-28C). In addition, the remaining tissue-resident innate lymphocytes had reduced expression of granzyme B and granzyme C (FIGS. 28D-28E), suggesting decreased cytotoxic activity. Accompanying these cellular defects, Mrp8-CreIL-15^{fl/fl}PyMT mice displayed significant acceleration of tumor growth compared to controls (FIG. 28F). Similar observations were made in mice with IL-15 gene deleted in cancer cells with MMTV-Cre transgenic mice (data not shown). Collectively, these findings demonstrate that cancer cell-derived IL-15 regulates the expansion and effector function of tissue-resident cytotoxic ILC1s, and the lack of these effector cells results in impaired cancer immunosurveillance.

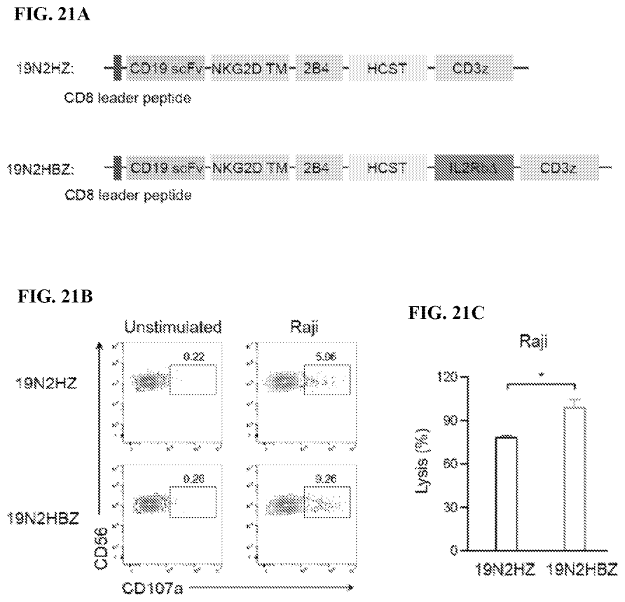

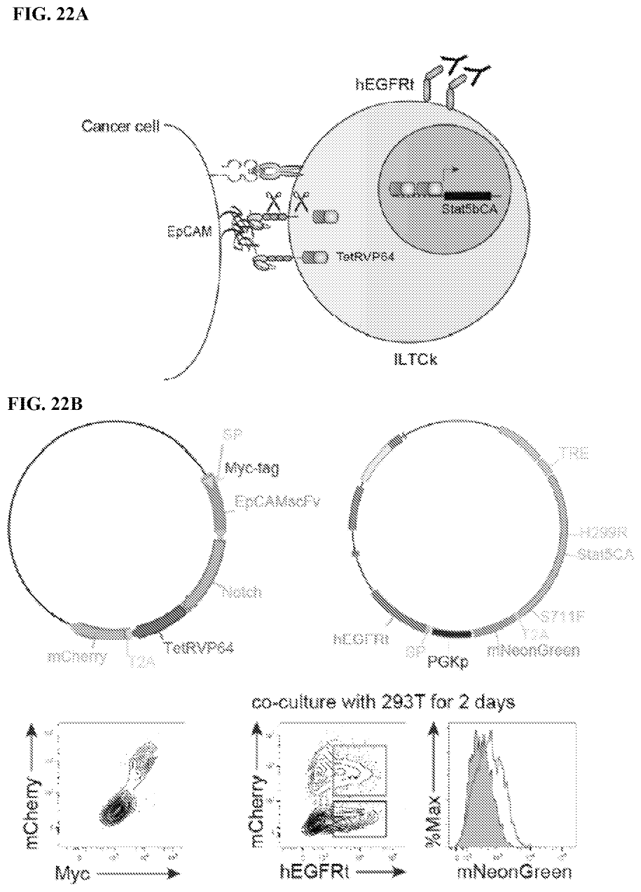

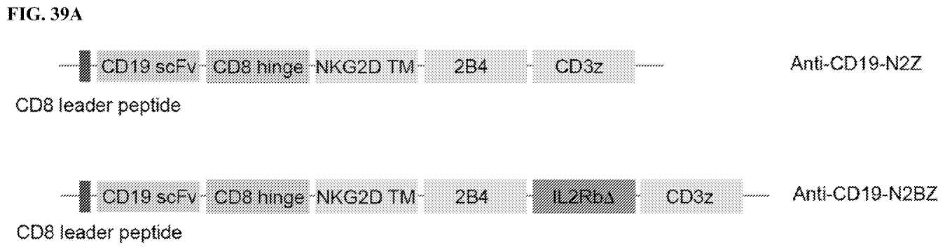

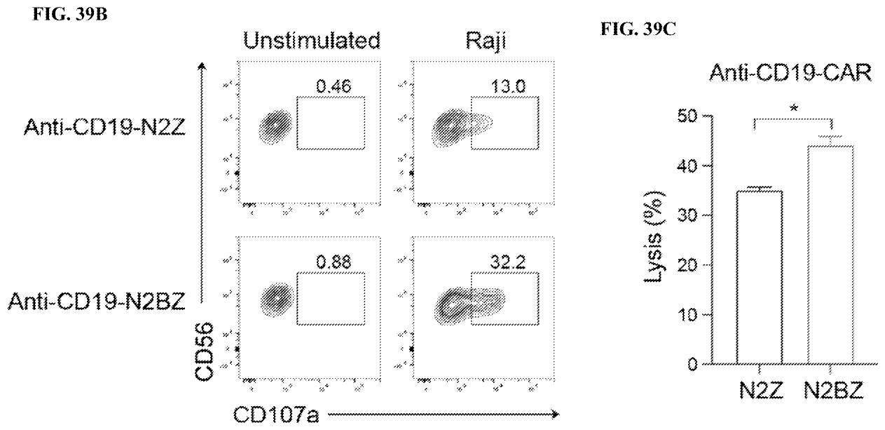

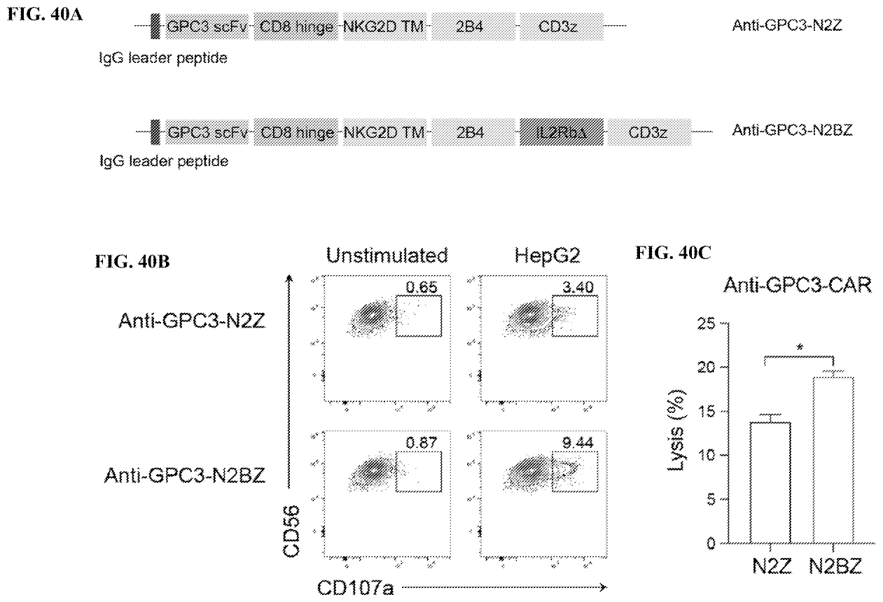

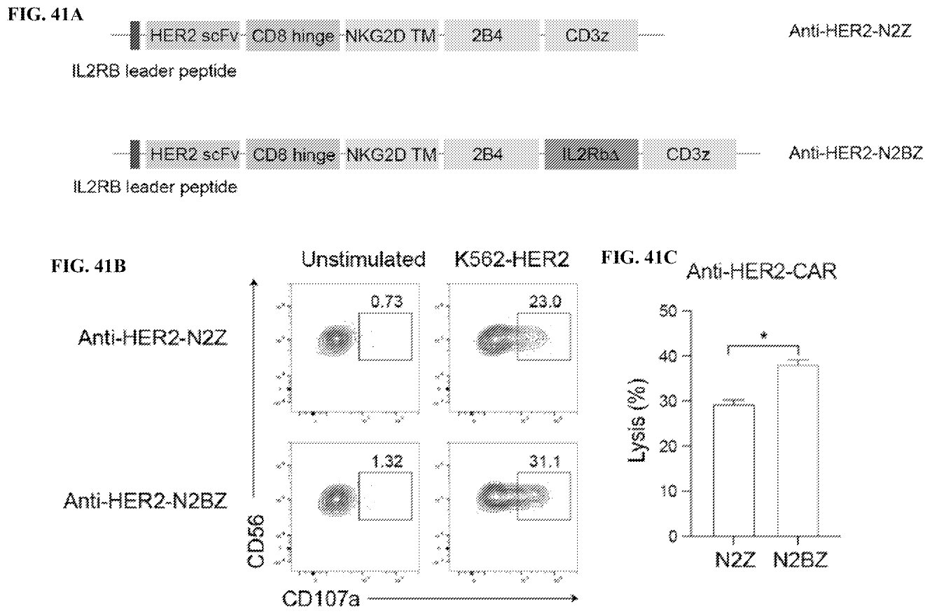

Human type 1 innate lymphoid cells armed with IL-2Rβ chain chimeric antigen receptor (CAR) exhibit enhanced cytotoxicity towards multiple cancer cell types (FIGS. 21A-21C, FIGS. 39A-39C, FIGS. 40A-40C, FIGS. 41A-41C). These results demonstrate that alternate strategies for inducing JAK-STAT activation in ILCs can be utilized to achieve anti-tumor effects. See FIGS. 22A-22B.

### REFERENCES

1. X. Fan, A. Y. Rudensky, Hallmarks of Tissue-Resident Lymphocytes. Cell 164, 1198-1211 (2016).
2. E. Vivier, D. Artis, M. Colonna, A. Diefenbach, J. P. Di Santo, G. Eberl, S. Koyasu, R. M. Locksley, A. N. J. Mckenzie, R. E. Mebius, F. Powrie, H. Spits, Innate Lymphoid Cells: 10 Years On. Cell 174, 1054-1066 (2018).
3. S. Dadi, S. Chhangawala, B. M. Whitlock, R. A. Franklin, C. T. Luo, S. A. Oh, A. Toure, Y. Pritykin, M. Huse, C. S. Leslie, M. O. Li, Cancer Immunosurveillance by Tissue-Resident Innate Lymphoid Cells and Innate-like T Cells. Cell 164, 365-377 (2016).
4. C. Friedrich, R. Taggenbrock, R. Doucet-Ladeveze, G. Golda, R. Moenius, P. Arampatzi, N. A. M. Kragten, K. Kreymborg, M. Gomez de Aguero, W. Kastenmuller, A. E. Saliba, D. Grun, K. van Gisbergen, G. Gasteiger, Effector differentiation downstream of lineage commitment in ILC1s is driven by Hobit across tissues. Nat Immunol 22, 1256-1267 (2021).
5. C. Di Censo, M. Marotel, I. Mattiola, L. Muller, G. Scarno, G. Pietropaolo, G. Peruzzi, M. Laffranchi, J. Mazej, M. S. Hasim, S. Asif, E. Russo, L. Tomaipitinca, H. Stabile, S. H. Lee, L. Vian, M. Gadina, A. Gismondi, H. Y. Shih, Y. Mikami, C. Capuano, G. Bernardini, M. Bonelli, S. Sozzani, A. Diefenbach, M. Ardolino, A. Santoni, G. Sciume, Granzyme A and CD160 expression delineates ILC1 with graded functions in the mouse liver. Eur J Immunol 51, 2568-2575 (2021).
6. H. Spits, J. H. Bernink, L. Lanier, NK cells and type 1 innate lymphoid cells: partners in host defense. Nat Immunol 17, 758-764 (2016).
7. M. L. Robinette, A. Fuchs, V. S. Cortez, J. S. Lee, Y. Wang, S. K. Durum, S. Gilfillan, M. Colonna, C. Immunological Genome, Transcriptional programs define molecular characteristics of innate lymphoid cell classes and subsets. Nat Immunol 16, 306-317 (2015).
8. C. Gregoire, L. Chasson, C. Luci, E. Tomasello, F. Geissmann, E. Vivier, T. Walzer, The trafficking of natural killer cells. Immunol Rev 220, 169-182 (2007).
9. H. Johnson, L. Scorrano, S. J. Korsmeyer, T. J. Ley, Cell death induced by granzyme C. Blood 101, 3093-3101 (2003).
10. T. Walzer, L. Chiossone, J. Chaix, A. Calver, C. Carozzo, L. Garrigue-Antar, Y. Jacques, M. Baratin, E. Tomasello, E. Vivier, Natural killer cell trafficking in vivo requires a dedicated sphingosine 1-phosphate receptor. Nat Immunol 8, 1337-1344 (2007).
11. C. Daussy, F. Faure, K. Mayol, S. Viel, G. Gasteiger, E. Charrier, J. Bienvenu, T. Henry, E. Debien, U. A. Hasan, J. Marvel, K. Yoh, S. Takahashi, I. Prinz, S. de Bernard, L. Buffat, T. Walzer, T-bet and Eomes instruct the development of two distinct natural killer cell lineages in the liver and in the bone marrow. J Exp Med 211, 563-577 (2014).
12. D. K. Sojka, B. Plougastel-Douglas, L. Yang, M. A. Pak-Wittel, M. N. Artyomov, Y. Ivanova, C. Zhong, J. M. Chase, P. B. Rothman, J. Yu, J. K. Riley, J. Zhu, Z. Tian, W. M. Yokoyama, Tissue-resident natural killer (NK) cells are cell lineages distinct from thymic and conventional splenic NK cells. Elife 3, e01659 (2014).
13. Y. Gao, F. Souza-Fonseca-Guimaraes, T. Bald, S. S. Ng, A. Young, S. F. Ngiow, J. Rautela, J. Straube, N. Waddell, S. J. Blake, J. Yan, L. Bartholin, J. S. Lee, E. Vivier, K. Takeda, M. Messaoudene, L. Zitvogel, M. W. L. Teng, G. T. Belz, C. R. Engwerda, N. D. Huntington, K. Nakamura, M. Holzel, M. J. Smyth, Tumor immunoevasion by the conversion of effector NK cells into type 1 innate lymphoid cells. Nat Immunol 18, 1004-1015 (2017).
14. M. G. Constantinides, B. D. McDonald, P. A. Verhoef, A. Bendelac, A committed precursor to innate lymphoid cells. Nature 508, 397-401 (2014).
15. Y. Yu, J. C. Tsang, C. Wang, S. Clare, J. Wang, X. Chen, C. Brandt, L. Kane, L. S. Campos, L. Lu, G. T. Belz, A. N. Mckenzie, S. A. Teichmann, G. Dougan, P. Liu, Single-cell RNA-seq identifies a PD-1 (hi) ILC progenitor and defines its development pathway. Nature 539, 102-106 (2016).
16. W. Xu, D. E. Cherrier, S. Chea, C. Vosshenrich, N. Serafini, M. Petit, P. Liu, R. Golub, J. P. Di Santo, An Id2(RFP)-Reporter Mouse Redefines Innate Lymphoid Cell Precursor Potentials. Immunity 50, 1054-1068 e1053 (2019).
17. J. H. Bernink, L. Krabbendam, K. Germar, E. de Jong, K. Gronke, M. Kofoed-Nielsen, J. M. Munneke, M. D. Hazenberg, J. Villaudy, C. J. Buskens, W. A. Bemelman, A. Diefenbach, B. Blom, H. Spits, Interleukin-12 and -23 Control Plasticity of CD127 (+) Group 1 and Group 3 Innate Lymphoid Cells in the Intestinal Lamina Propria. Immunity 43, 146-160 (2015).
18. J. S. Silver, J. Kearley, A. M. Copenhaver, C. Sanden, M. Mori, L. Yu, G. H. Pritchard, A. A. Berlin, C. A. Hunter, R. Bowler, J. S. Erjefalt, R. Kolbeck, A. A. Humbles, Inflammatory triggers associated with exacerbations of COPD orchestrate plasticity of group 2 innate lymphoid cells in the lungs. Nat Immunol 17, 626-635 (2016).
19. R. Fiancette, C. M. Finlay, C. Willis, S. L. Bevington, J. Soley, S. T. H. Ng, S. M. Baker, S. Andrews, M. R. Hepworth, D. R. Withers, Reciprocal transcription factor networks govern tissue-resident ILC3 subset function and identity. Nat Immunol 22, 1245-1255 (2021).
20. J. K. Bando, H. E. Liang, R. M. Locksley, Identification and distribution of developing innate lymphoid cells in the fetal mouse intestine. Nat Immunol 16, 153-160 (2015).
21. C. Schneider, J. Lee, S. Koga, R. R. Ricardo-Gonzalez, J. C. Nussbaum, L. K. Smith, S. A. Villeda, H. E. Liang, R. M. Locksley, Tissue-Resident Group 2 Innate Lymphoid Cells Differentiate by Layered Ontogeny and In Situ Perinatal Priming. Immunity 50, 1425-1438 e1425 (2019).
22. M. G. Constantinides, H. Gudjonson, B. D. McDonald, I. E. Ishizuka, P. A. Verhoef, A. R. Dinner, A. Bendelac, PLZF expression maps the early stages of ILC1 lineage development. Proc Natl Acad Sci USA 112, 5123-5128 (2015).
23. K. Takeda, E. Cretney, Y. Hayakawa, T. Ota, H. Akiba, K. Ogasawara, H. Yagita, K. Kinoshita, K. Okumura, M. J. Smyth, TRAIL identifies immature natural killer cells in newborn mice and adult mouse liver. Blood 105, 2082-2089 (2005).
24. S. M. Gordon, J. Chaix, L. J. Rupp, J. Wu, S. Madera, J. C. Sun, T. Lindsten, S. L. Reiner, The transcription factors T-bet and Eomes control key checkpoints of natural killer cell maturation. Immunity 36, 55-67 (2012).
25. V. S. Cortez, L. Cervantes-Barragan, M. L. Robinette, J. K. Bando, Y. Wang, T. L. Geiger, S. Gilfillan, A. Fuchs, E. Vivier, J. C. Sun, M. Cella, M. Colonna, Transforming Growth Factor-beta Signaling Guides the Differentiation of Innate Lymphoid Cells in Salivary Glands. Immunity 44, 1127-1139 (2016).
26. L. K. Mackay, M. Minnich, N. A. Kragten, Y. Liao, B. Nota, C. Seillet, A. Zaid, K. Man, S. Preston, D. Freestone, A. Braun, E. Wynne-Jones, F. M. Behr, R. Stark, D. G. Pellicci, D. I. Godfrey, G. T. Belz, M. Pellegrini, T. Gebhardt, M. Busslinger, W. Shi, F. R. Carbone, R. A. van Lier, A. Kallies, K. P. van Gisbergen, Hobit and Blimp1 instruct a universal transcriptional program of tissue residency in lymphocytes. Science 352, 459-463 (2016).
27. K. Yomogida, T. M. Bigley, T. Trsan, S. Gilfillan, M. Cella, W. M. Yokoyama, T. Egawa, M. Colonna, Hobit confers tissue-dependent programs to type 1 innate lymphoid cells. Proc Natl Acad Sci USA 118, (2021).
28. M. K. Kennedy, M. Glaccum, S. N. Brown, E. A. Butz, J. L. Viney, M. Embers, N. Matsuki, K. Charrier, L. Sedger, C. R. Willis, K. Brasel, P. J. Morrissey, K. Stocking, J. C. Schuh, S. Joyce, J. J. Peschon, Reversible defects in natural killer and memory CD8 T cell lineages in interleukin 15-deficient mice. J Exp Med 191, 771-780 (2000).
29. W. J. Leonard, J. X. Lin, J. J. O'Shea, The gammac Family of Cytokines: Basic Biology to Therapeutic Ramifications. Immunity 50, 832-850 (2019).
30. B. Jabri, V. Abadie, IL-15 functions as a danger signal to regulate tissue-resident T cells and tissue destruction. Nat Rev Immunol 15, 771-783 (2015).
31. A. Crinier, P. Milpied, B. Escaliere, C. Piperoglou, J. Galluso, A. Balsamo, L. Spinelli, I. Cervera-Marzal, M. Ebbo, M. Girard-Madoux, S. Jaeger, E. Bollon, S. Hamed, J. Hardwigsen, S. Ugolini, F. Vely, E. Narni-Mancinelli, E. Vivier, High-Dimensional Single-Cell Analysis Identifies Organ-Specific Signatures and Conserved NK Cell Subsets in Humans and Mice. Immunity 49, 971-986 e975 (2018).
32. A. P. McFarland, A. Yalin, S. Y. Wang, V. S. Cortez, T. Landsberger, R. Sudan, V. Peng, H. L. Miller, B. Ricci, E. David, R. Faccio, I. Amit, M. Colonna, Multi-tissue single-cell analysis deconstructs the complex programs of mouse natural killer and type 1 innate lymphoid cells in tissues and circulation. Immunity 54, 1320-1337 e1324 (2021).
33. L. Krabbendam, J. H. Bernink, H. Spits, Innate lymphoid cells: from helper to killer. Curr Opin Immunol 68, 28-33 (2021).
34. U. Y. Moreno-Nieves, J. K. Tay, S. Saumyaa, N. B. Horowitz, J. H. Shin, I. A. Mohammad, B. Luca, D. C. Mundy, G. S. Gulati, N. Bedi, S. Chang, C. Chen, M. J. Kaplan, E. L. Rosenthal, F. C. Holsinger, V. Divi, F. M. Baik, D. B. Sirjani, A. J. Gentles, A. M. Newman, A. G. Freud, J. B. Sunwoo, Landscape of innate lymphoid cells in human head and neck cancer reveals divergent NK cell states in the tumor microenvironment. Proc Natl Acad Sci USA 118, (2021). 35.

Y. T. Tang, D. Wang, H. Luo, M. Xiao, H. S. Zhou, D. Liu, S. P. Ling, N. Wang, X. L. Hu, Y. Luo, X. Mao, Q. L. Ao, J. Huang, W. Zhang, L. S. Sheng, L. J. Zhu, Z. Shang, L. L. Gao, P. L. Zhang, M. Zhou, K. G. Zhou, L. G. Qiu, Q. F. Liu, H. Y. Zhang, J. Y. Li, J. Jin, L. Fu, W. L. Zhao, J. P. Chen, X. Du, G. Huang, Q. F. Wang, J. F. Zhou, L. Huang, Aggressive NK-cell leukemia: clinical subtypes, molecular features, and treatment outcomes. Blood Cancer J 7, 660 (2017).
36. J. E. Melsen, G. Lugthart, A. C. Lankester, M. W. Schilham, Human Circulating and Tissue-Resident CD56 (bright) Natural Killer Cell Populations. Front Immunol 7, 262 (2016).
37. B. D. McDonald, B. Jabri, A. Bendelac, Diverse developmental pathways of intestinal intraepithelial lymphocytes. Nat Rev Immunol 18, 514-525 (2018).
38. J. Loh, D. T. Chu, A. K. O'Guin, W. M. Yokoyama, H. W. t. Virgin, Natural killer cells utilize both perforin and gamma interferon to regulate murine cytomegalovirus infection in the spleen and liver. J Virol 79, 661-667 (2005).
39. O. E. Weizman, N. M. Adams, I. S. Schuster, C. Krishna, Y. Pritykin, C. Lau, M. A. Degli-Esposti, C. S. Leslie, J. C. Sun, T. E. O'Sullivan, ILC1 Confer Early Host Protection at Initial Sites of Viral Infection. Cell 171, 795-808 e712 (2017).
40. T. Chinen, A. K. Kannan, A. G. Levine, X. Fan, U. Klein, Y. Zheng, G. Gasteiger, Y. Feng, J. D. Fontenot, A. Y. Rudensky, An essential role for the IL-2 receptor in Treg cell function. Nat Immunol 17, 1322-1333 (2016).
41. R. A. Franklin, W. Liao, A. Sarkar, M. V. Kim, M. R. Bivona, K. Liu, E. G. Pamer, M. O. Li, The cellular and molecular origin of tumor-associated macrophages. Science 344, 921-925 (2014).
42. W. Ouyang, S. A. Oh, Q. Ma, M. R. Bivona, J. Zhu, M. O. Li, TGF-beta cytokine signaling promotes CD8+ T cell development and low-affinity CD4+ T cell homeostasis by regulation of interleukin-7 receptor alpha expression. Immunity 39, 335-346 (2013).
43. A. G. Levine, A. Mendoza, S. Hemmers, B. Moltedo, R. E. Niec, M. Schizas, B. E. Hoyos, E. V. Putintseva, A. Chaudhry, S. Dikiy, S. Fujisawa, D. M. Chudakov, P. M. Treuting, A. Y. Rudensky, Stability and function of regulatory T cells expressing the transcription factor T-bet. Nature 546, 421-425 (2017).
44. M. H. Do, X. Wang, X. Zhang, C. Chou, B. G. Nixon, K. J. Capistrano, M. Peng, A. Efeyan, D. M. Sabatini, M. O. Li, Nutrient mTORC1 signaling underpins regulatory T cell control of immune tolerance. J Exp Med 217, (2020).
45. J. D. Buenrostro, P. G. Giresi, L. C. Zaba, H. Y. Chang, W. J. Greenleaf, Transposition of native chromatin for fast and sensitive epigenomic profiling of open chromatin, DNA-binding proteins and nucleosome position. Nat Methods 10, 1213-1218 (2013).
46. B. Langmead, S. L. Salzberg, Fast gapped-read alignment with Bowtie 2. Nat Methods 9, 357-359 (2012).
47. H. Li, B. Handsaker, A. Wysoker, T. Fennell, J. Ruan, N. Homer, G. Marth, G. Abecasis, R. Durbin, S. Genome Project Data Processing, The Sequence Alignment/Map format and SAMtools. Bioinformatics 25, 2078-2079 (2009).
48. H. Li, A statistical framework for SNP calling, mutation discovery, association mapping and population genetical parameter estimation from sequencing data. Bioinformatics 27, 2987-2993 (2011).
49. Y. Zhang, T. Liu, C. A. Meyer, J. Eeckhoute, D. S. Johnson, B. E. Bernstein, C. Nusbaum, R. M. Myers, M. Brown, W. Li, X. S. Liu, Model-based analysis of ChIP-Seq (MACS). Genome Biol 9, R137 (2008).
50. M. I. Love, W. Huber, S. Anders, Moderated estimation of fold change and dispersion for RNA-seq data with DESeq2. Genome Biol 15, 550 (2014).
51. R. Patro, G. Duggal, M. I. Love, R. A. Irizarry, C. Kingsford, Salmon provides fast and bias-aware quantification of transcript expression. Nat Methods 14, 417-419 (2017).
52. C. Soneson, M. I. Love, M. D. Robinson, Differential analyses for RNA-seq: transcript-level estimates improve gene-level inferences. F1000Res 4, 1521 (2015).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An engineered cytotoxic innate lymphoid cell (ILC) comprising a non-endogenous expression vector including a mammalian IL-15 nucleic acid sequence or a mammalian STAT5B nucleic acid sequence, wherein the IL-15 nucleic acid sequence or the STAT5B nucleic acid sequence is operably linked to an expression control sequence, optionally wherein the engineered ILC is derived from an autologous donor or an allogenic donor.

2. The engineered cytotoxic ILC of claim 1, wherein the expression control sequence comprises an inducible promoter, a constitutive promoter, a native IL-15 or STAT5B promoter, or a heterologous promoter; or
wherein the non-endogenous expression vector is a plasmid, a cosmid, a bacmid, a bacterial artificial chromosome (BAC), a yeast artificial chromosome (YAC), a viral vector, or a retroviral vector; or
wherein the IL-15 nucleic acid sequence encodes the amino acid sequence of SEQ ID NO: 19 or SEQ ID NO: 20; or
wherein the STAT5B nucleic acid sequence encodes the amino acid sequence of SEQ ID NO: 9 or SEQ ID NO: 23.

3. (canceled)

4. (canceled)

5. (canceled)

6. The engineered cytotoxic ILC of claim 1, further comprising a chimeric antigen receptor (CAR) that binds to a tumor antigen and/or a nucleic acid encoding the CAR.

7. The engineered cytotoxic ILC of claim 6, wherein the heterologous promoter is induced by binding of the CAR to the tumor antigen, optionally wherein binding of the CAR to the tumor antigen results in antigen-dependent JAK-STAT5 pathway activation; or
wherein the CAR comprises (i) an extracellular antigen binding domain; (ii) a transmembrane domain; and (iii) an intracellular domain comprising one or more co-stimulatory domains, wherein the extracellular antigen binding domain binds to the tumor antigen.

8. (canceled)

9. An engineered cytotoxic innate lymphoid cell (ILC) comprising a chimeric antigen receptor (CAR) that binds to a tumor antigen, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains, optionally wherein the truncated cytoplasmic domain of IL-2RβΔ comprises the amino acid sequence of SEQ ID NO: 7.

10. (canceled)

11. The engineered cytotoxic ILC of claim 7, wherein the extracellular antigen binding fragment comprises a single-chain variable fragment (scFv), optionally wherein the scFv is human; or
wherein the tumor antigen is selected from the group consisting of 5T4, alpha 5β1-integrin, 707-AP, AFP, ART-4, B7H4, BCMA, Bcr-abl, CA125, CA19-9, CDH1, CDH17, CAMEL, CAP-1, CASP-8, CD5, CD25, CDC27/m, CD37, CD52, CDK4/m, c-Met, CS-1, CT, Cyp-B, cyclin B1, DAGE, DAM, EBNA, ErbB3, ELF2M, EMMPRIN, ephrinB2, estrogen receptor, ETV6-AML1, FAP, ferritin, folate-binding protein, G250, GM2, HAGE, HLA-A*0201-R170I, HPV E6, HPV E7, HSP70-2M, HST-2, hTERT (or hTRT), iCE, IL-2R, IL-5, KIAA0205, LAGE, LDLR/FUT, MART-1/melan-A, MART-2/Ski, MC1R, mesothelin, MUC16, myc, MUM-2, MUM-3, NA88-A, NYESO-1, NY-Eso-B, proteinase-3, p190 minor bcr-abl, Pml/RARα, progesterone receptor, PSCA, RU1 or RU2, RORI, SART-1 or SART-3, survivin, TEL/AML1, TGFβ, TPI/m, TRP-1, TRP-2, TRP-2/INT2, tenascin, TSTA tyrosinase, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Ley) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, and peptide MHC complexes (with peptides derived from TP53, KRAS, MYC, EBNA1-6, PRAME, tyronsinase, MAGEA1-A6, pmel17, LMP2, or WT1).

12. (canceled)

13. (canceled)

14. The engineered cytotoxic ILC of claim 7, wherein the transmembrane domain comprises a CD8 transmembrane domain, a CD28 transmembrane domain, a NKG2D transmembrane domain, a CD3ζ transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, or a BTLA transmembrane domain.

15. The engineered cytotoxic ILC of any claim 7, wherein the one or more co-stimulatory domains are selected from the group consisting of a CD28 co-stimulatory domain, a 4-1BB co-stimulatory domain, an OX40 co-stimulatory domain, an ICOS co-stimulatory domain, a DAP-10 co-stimulatory domain, a PD-1 co-stimulatory domain, a CTLA-4 co-stimulatory domain, a LAG-3 co-stimulatory domain, a 2B4 co-stimulatory domain, a BTLA co-stimulatory domain, a NKG2C co-stimulatory domain, a NKG2D co-stimulatory domain, and any combination thereof.

16. The engineered cytotoxic ILC of claim 7, wherein the one or more co-stimulatory domains comprise a DAP-10 co-stimulatory domain and a 2B4 co-stimulatory domain.

17. (canceled)

18. A composition comprising an effective amount of the engineered cytotoxic ILC of claim 1 and a pharmaceutically acceptable carrier.

19. A method of preparing immune cells for adoptive cell therapy comprising: (a) isolating cytotoxic innate lymphoid cells (ILCs) from a donor subject, (b) transducing the cytotoxic ILCs with a nucleic acid encoding IL-15 or STAT5B or an expression vector comprising said nucleic acid, and (c) administering the transduced cytotoxic ILCs to a recipient subject, optionally wherein the nucleic acid encodes the amino acid sequence of SEQ ID NO: 19, SEQ ID NO: 20, SEQ ID NO: 9 or SEQ ID NO: 23.

20. (canceled)

21. The method of claim 19, further comprising
transducing the cytotoxic ILCs with a nucleic acid encoding a chimeric antigen receptor (CAR) that binds to a tumor antigen; or
transducing the cytotoxic ILCs with a nucleic acid encoding a chimeric antigen receptor (CAR) that binds to a tumor antigen or an expression vector comprising said nucleic acid, wherein the CAR comprises (i) an extracellular antigen binding domain that binds to the tumor antigen; (ii) a transmembrane domain; and (iii) an intracellular domain comprising a truncated cytoplasmic domain of IL-2RβΔ and one or more co-stimulatory domains.

22. (canceled)

23. The method of claim 19, wherein the donor subject and the recipient subject are the same or different.

24. A method for treating cancer or inhibiting tumor growth in a subject in need thereof comprising administering to the subject an effective amount of the engineered cytotoxic ILC of claim 1.

25. The method of claim 24, wherein the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

26. The method of claim 24, wherein the engineered cytotoxic ILC is administered pleurally, intravenously, subcutaneously, intranodally, intratumorally, intrathecally, intrapleurally or intraperitoneally.

27. The method of claim 24, further comprising sequentially, separately, or simultaneously administering to the subject an additional cancer therapy.

28. The method of claim 27, wherein the additional cancer therapy is selected from among chemotherapeutic agents, immune checkpoint inhibitors, monoclonal antibodies that specifically target tumor antigens, immune activating agents (e.g., interferons, interleukins, cytokines), oncolytic virus therapy and cancer vaccines.

29. A kit for preparing the engineered cytotoxic innate lymphoid cell (ILC) of claim 1 comprising an expression vector that includes a nucleic acid sequence encoding an amino acid sequence of SEQ ID NO: 19, SEQ ID NO: 20, SEQ ID NO: 9 or SEQ ID NO: 23 and instructions for transducing cytotoxic ILCs with the expression vector.

30. The kit of claim 29, further comprising a vector encoding an engineered CAR or other cell-surface ligand that binds to a tumor antigen.


SEQUENCE LISTING (71 sequences)

SEQ ID NO: 1 - protein, 18 aa, Homo sapiens
MALPVTALLLPLALLLHA

SEQ ID NO: 2 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 3 - protein, 107 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIK

SEQ ID NO: 4 - protein, 21 aa, Homo sapiens
PFFFCCFIAVAMGIRFIIMVT

SEQ ID NO: 5 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 6 - protein, 24 aa, Homo sapiens
LCARPRRSPAQEDGKVYINMPGRG

SEQ ID NO: 7 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 8 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 786 aa, Homo sapiens
MAMWIQAQQPQGDALHQMQALYGQHFPIEVRHYLSQWIESQAWDSIDLDNPQENIKATQLLEGLVQELQKKAEHQVGEDGFLLKIKLGHYATQLQSTYDRCPMELVRCIRHILYNEQRLVREANNGSSPAGSLADAMSQKHLQINQTFEELRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAERLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFDSQFSVGGNELVFQVKTLSLPVVVIVHGSQDNNATATVLWDNAFAEPGRVPFAVPDKVLWPQLCEALNMKFKAEVQSNRGLTKENLVFLAQKLFNISSNHLEDYNSMSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCEPATAKAADGYVKPQIKQVVPEFANAFTDAGSGATYMDQAPSPVVCPQAHYNMYPPNPDSVLDTDGDFDLEDMMDVARRVEELLGRPMDSQWIPHAQS

SEQ ID NO: 10 - DNA, 54 bp, Homo sapiens
  source 1..54: mol_type = genomic DNA
atggctctcccagtgactgccctactgcttcccctagcgcttctcctgcatgca

SEQ ID NO: 11 - DNA, 366 bp, synthetic construct
  source 1..366: mol_type = other DNA
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctca

SEQ ID NO: 12 - DNA, 321 bp, synthetic construct
  source 1..321: mol_type = other DNA
gacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaa

SEQ ID NO: 13 - DNA, 63 bp, Homo sapiens
  source 1..63: mol_type = genomic DNA
ccattttttttctgctgcttcatcgctgtagccatgggaatccgtttcattattatggtaaca

SEQ ID NO: 14 - DNA, 360 bp, Homo sapiens
  source 1..360: mol_type = genomic DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 15 - DNA, 72 bp, Homo sapiens
  source 1..72: mol_type = genomic DNA
ctgtgcgcacgcccacgccgcagccccgcccaagaagatggcaaagtctacatcaacatgccaggcaggggc

SEQ ID NO: 16 - DNA, 282 bp, Homo sapiens
  source 1..282: mol_type = genomic DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtg

SEQ ID NO: 17 - DNA, 336 bp, Homo sapiens
  source 1..336: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - DNA, 2358 bp, Homo sapiens
  source 1..2358: mol_type = genomic DNA
atggctatgtggatacaggctcagcagccccagggcgatgcccttcaccagatgcaggccttgtacggccagcatttccccatcgaggtgcgacattatttatcacagtggatcgaaagccaagcctgggactcaatagatcttgataatccacaggagaacattaaggccacccagctcctggagggcctggtgcaggagctgcagaagaaggcggagcaccaggtgggggaagatgggtttttgctgaagatcaagctggggcactatgccacacagctccagagcacgtacgaccgctgccccatggagctggttcgctgtatccggcacattctgtacaacgaacagaggctggttcgcgaagccaacaacggcagctctccagctggaagtcttgctgacgccatgtcccagaagcaccttcagatcaaccaaacgtttgaggagctgcgcctgatcacacaggacacggagaacgagctgaagaagctgcagcagacccaagagtacttcatcatccagtaccaggagagcctgcggatccaagctcagtttgcccagctgggacagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaatcggcagcagatccgcagggctgagcgcctgtgccagcagctgcccatcccaggccccgtggaggagatgctggctgaggtcaacgccaccatcacggacatcatctcagccctggtcaccagcacgttcatcatcgagaagcagcctcctcaggtcctgaagacccagaccaagtttgcggccactgtgcgcctgctggtgggggggaagctgaatgtgcacatgaaccccccgcaggtgaaggcgaccatcatcagcgagcagcaggccaagtccctgctcaagaatgagaacacccgcaatgattacagcggcgagatcctgaacaactgttgcgtcatggagtaccaccaggccactggcacgctcagcgcccacttcagaaacatgtccctgaaacgaatcaagagatctgaccgccgtggtgcagagtcagtaacggaagagaagttcacgatcctgtttgactcacagttcagcgtcggtggaaacgagctggtctttcaagtcaagaccttgtcgctcccggtggtggtgattgttcacggcagccaggacaacaatgccacagccactgtcctctgggacaacgcctttgcagagcctggcagggtgccatttgccgtgcctgacaaggtgctgtggccgcagctgtgtgaagcgctcaacatgaaattcaaggctgaagtacagagcaaccggggcttgaccaaggagaacctcgtgttcctggcacagaaactgttcaacatcagcagcaaccacctcgaggactacaacagcatgtccgtgtcctggtcccagttcaaccgggagaatttgccaggacggaattacactttctggcagtggtttgatggcgtgatggaagtattgaaaaaacatctcaagcctcactggaatgatggggctatcctgggtttcgtgaacaagcaacaggcccacgacctgctcatcaacaagccggacgggaccttcctgctgcgcttcagcgactcggaaatcgggggcatcaccattgcttggaagtttgactctcaggagagaatgttttggaatctgatgccttttaccactagagacttctctatccggtccctcgctgaccgcctgggggacctgaattacctcatatacgtgtttcctgatcggccaaaggatgaagtatattctaagtactacacaccggtcccctgtgagcccgcaactgcgaaagcagctgacggatacgtgaagccacagatcaagcaggtggtccccgagtttgcaaatgcattcacagatgctgggagtggcgccacctacatggatcaggctccttccccagtcgtgtgccctcaggctcactacaacatgtacccacccaacccggactccgtccttgataccgatggggacttcgatctggaagacatgatggacgtggcgcggcgcgtggaagagctcttaggccggcccatggacagtcagtggatccctcacgcacagtca

SEQ ID NO: 19 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 20 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - DNA, 2015 bp, Homo sapiens
  source 1..2015: mol_type = genomic DNA
cttttcgccaggggttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaa

SEQ ID NO: 22 - DNA, 2336 bp, Homo sapiens
  source 1..2336: mol_type = genomic DNA
cttttcgccaggggttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgttttcaactagtggccccactgtgtccggaattgatgggttcttggtctcactgacttcaagaatgaagccgcggaccctcgcggtgagtgttacagctcttaaggtggcgcatctggagtttgttccttctgatgttcggatgtgttcggagtttcttccttctggtgggttcgtggtctcgctggctcaggagtgaagctacagaccttcgcggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggatgcagctaatatacccagttggcccaaagcacctaacctatagttatataatctgactctcagttcagttttactctactaatgccttcatggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaa

SEQ ID NO: 23 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 24 - DNA, 5079 bp, Homo sapiens
  source 1..5079: mol_type = genomic DNA
ggcggccggagccgtcaccccgggcggggacccagcgcaggcaactccgcgcggcggcccggccgagggagggagcgagcgggcgggcgggcaagccagacagctgggccggagcagccgcgggcgcccgaggggccgagcgagattgtaaaccatggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgtgaccccgcgacctctccatcttcagcttcttcatcttcaccagaggaatcactcttgtggatgttttaattccatgaatcgcttctcttttgaaacaatactcataatgtgaagtgttaatactagttgtgaccttagtgtttctgtgcatggtggcaccagcgaagggagtgcgagtatgtgtttgtgtgtgtgtgtgtgtgtgtgtgtgtgtgcgtgtttgcacgttatggtgtttctccctctcactgtctgagagtttagttgtagcagaggggccacagacagaagctgtggtggtttttactttgtgcaaaaaggcagtgagtttcgtgaagcctggaagttggccatgtgtcttaagagtggctggactttgacatgtggctgtttgaataagagaaggacaaagggaggagaaagcacatgtgctccagtgagtcttcgtcactctgtctgccaagcaattgatatataaccgtgattgtctctgcttttcttctgaaatgtagataactgctttttgacaaagagagccttccctctcccccacccctgtgttcttgggtaggaatgggaaaaggggcaacctacaaagattgttggggcaagggaagtcacaagctttcggatgggcggtggcttttcacaaaacatttagctcatcttattctctctttgtcctctctcccctcctgcccgcccgcaccctggaattgccactcagttcctctgggtgtgcacatatgtttggagaaatagaggagagaaaagagggccacgtaactgagagcttacagtgccaatgccgtttgtgttctggccagagtggagtgcgcagccctgactcccaggcgctgagattgttgcctggttacccaggaagctgctgttccggctgcccagcctttctctgagccagcggatgcacagtccgtggccttcttcaggcttattgatgatgctttttgcaaatgttgaatcatggttctgtttctaagttggatcttttttgttttctccttgccaccctaatttgacatcaaaattctctcttgtgcattgggccctgggtcattcaaacccaggtcacctcattccccttctctgttcacacctaatgtcttgaagagtaggtagcagcagtgtgggctgaacctaggccagcttgcttagcgggtcaccctgctgtgaagtcctggcaggtgttggtaatgtgtggaaatgcagtcagcaagtttgctggggagtttgataaaagtataaaacaaaacaaaaaaagcctcggtataattttgttccacgacttcttctgtagctttacaccagaaggaaggaatgggctacagcaggtagtggaggaagaggggggtgagcaggtgtattaaaatagcttacgggtaaggcctaaaaggtcacccctcggccccctctccaaaagaagggcatgggcacccccaggagaggatggccccaaaaaccttatttttatacatgagagtaaataaacatattttttttacaaaaataacttctgaatttatcagtgttttgccgttaaaaatattcctctatagtaaattatttattggaagatgacttttttaaagctgccgtttgccttggcttggtttcatacactgatttatttttctatgccaggcagtagagtctctctgcctctgaggagcaggctacccgcatcccactcagcccctccctacccctcaagatttgatgaaaattccaaccatgaggatgggtgcatcggggaagggtgagaaggagagcctgcctgctcagggatccaggctcgtagagtcactccctgcccgtctcccagagatgcttcaccagcacctgcctctgagacctcgctctctgttccagcaaccctggttggggggtcagacttgatacactttcaggttgggagtggacccaccccagggcctgctgaggacagagcagccaggccgtcctggctcactttgcagttggcactgggttggggaggaagagagctgatgagtgtggcttccctgagctggggtttccctgcttgtccagttgtgagctgtcctcggtgttaccgaggctgtgcctagagagtggagatttttgatgaaaggtgtgctcgctctctgcgttctatcttctctctcctccttgttcctgcaaaccacaagataaaggtagtggtgtgtctcgaccccatcagcctctcacccactcccagacacacacaagtcctcaaaagtttcagctccgtgtgtgagatgtgcaggttttttctagggggtagggggagactaaaatcgaatataacttaaaatgaaagtatactttttataatttttctttttaaaacttggtgaaattatttcagatacatattttagtgtcaaggcagattagttatttagccaccaaaaaaaagtattgtgtacaatttggggcctcaaatttgactctgcctcaaaaaaaagaaatatatcctatgcagagttacagtcacaaagttgtgtattttatgttacaataaagccttcctctgaaggga

SEQ ID NO: 25 - protein, 21 aa, Homo sapiens
MALPVTALLLPLALLLHAARP

SEQ ID NO: 26 - DNA, 63 bp, Homo sapiens
  source 1..63: mol_type = genomic DNA
atggccctgccagtaacggctctgctgctgccacttgctctgctcctccatgcagccaggcct

SEQ ID NO: 27 - protein, 21 aa, Mus sp.
MASPLTRFLSLNLLLLGESII

SEQ ID NO: 28 - DNA, 63 bp, Mus sp.
  source 1..63: mol_type = genomic DNA
atggccagccccctgaccaggttcctgagcctgaacctgctgctgctgggcgagagcatcatc

SEQ ID NO: 29 - protein, 18 aa, Mus sp.
MASPLTRFLSLNLLLLGE

SEQ ID NO: 30 - DNA, 54 bp, Mus sp.
  source 1..54: mol_type = genomic DNA
atggccagccccctgaccaggttcctgagcctgaacctgctgctgctgggcgag

SEQ ID NO: 31 - protein, 214 aa, unidentified
MLRLLLALNLFPSIQVTGNKILVKQSPMLVAYDNALSCKYSYNLFSREFRASLHKGLDSAVEVCWYGNYSQQLQVYSKTGFNCDGKLGNESVTFYLQNLYQTDIYFCKIEVMYPPPYLDNEKSNGTIIHVKGKHLCPSPLFPGPSKPFWVLVWGGVLACYSLLVTVAFIIFWVRSKRSRLLHSDYMNMTPRRPGPTRKHYQPYAPPRDFAAYRS

SEQ ID NO: 32 - DNA, 321 bp, unidentified
  source 1..321: mol_type = unassigned DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 33 - protein, 234 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 34 - protein, 71 aa, synthetic construct
PTTTPAPRPPTPAPTIASQPLSLRPEACRPAAGGAVHTRGLDFACDIYIWAPLAGTCGVLLLSLVITLYCN

SEQ ID NO: 35 - DNA, 213 bp, synthetic construct
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 36 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 37 - protein, 15 aa, synthetic construct
GGGGSGGGGSGGGGS

SEQ ID NO: 38 - DNA, 45 bp, synthetic construct
  source 1..45: mol_type = other DNA
ggcggcggcggatctggaggtggtggctcaggtggcggaggctcc

SEQ ID NO: 39 - protein, 8 aa, synthetic construct
  VARIANT 2: note = V or I
  VARIANT 4: note = Any amino acid
DXEXNPGP

SEQ ID NO: 40 - protein, 75 aa, synthetic construct
  VARIANT 1..75: note = This sequence may encompass 1-15 GGGGS repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 41 - protein, 120 aa, synthetic construct [sequence omitted]

SEQ ID NO: 42 - protein, 107 aa, synthetic construct [sequence omitted]

SEQ ID NO: 43 - protein, 115 aa, synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 44 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 45 - DNA, 360 bp, synthetic construct
  source 1..360: mol_type = other DNA
gaggtccagttggttgaatctggtggaggtttggtccagccaggtggatctttgagattgtcttgtgccgcttctggtttcaacatcaaggacacctacattcattgggttagacaagcccctggtaagggattggagtgggttgccagaatttacccaacaaacggatacacaagatacgctgactctgtcaagggaagattcactatctctgccgacacatctaagaacactgcatacttgcaaatgaactctttgagagccgaagacacagccgtctactactgctctagatggggtggtgacggtttttacgccatggactattggggtcaaggaacattggtcacagtctcttct

SEQ ID NO: 46 - DNA, 321 bp, synthetic construct
  source 1..321: mol_type = other DNA
gacattcagatgacccaatctccatcttctttgtctgcctctgtcggtgatagagttaccatcacctgcagagcttctcaagacgtcaataccgcagttgcctggtatcaacagaagccaggaaaggcacctaagttgttgatctactctgcttcttttttgtactctggagtcccttctagattttctggatctagatctggtaccgatttcacattgaccatttcttctttgcagcctgaggactttgccacatattactgtcagcagcactacacaacccctcctacttttggtcagggaactaaggtcgagattaag

SEQ ID NO: 47 - DNA, 345 bp, synthetic construct
  source 1..345: mol_type = other DNA
caggtccagctgcagcagtcaggagccgaactggtgcggcccggcgcaagtgtcaaactgtcatgcaaggccagcgggtataccttcacagactacgagatgcactgggtgaaacagacccctgtgcacggcctgaagtggatcggcgctctggacccaaaaaccggggatacagcatattcccagaagtttaaaggaaaggccactctgaccgctgacaagagctcctctactgcctacatggagctgaggagcctgacatccgaagatagcgccgtgtactattgcacccgcttctactcctatacatactggggccaggggactctggtgaccgtctctgca

SEQ ID NO: 48 - DNA, 336 bp, synthetic construct
  source 1..336: mol_type = other DNA
gacgtggtcatgacacagactccactgtccctgcccgtgagcctgggcgatcaggctagcatttcctgtcgaagttcacagagtctggtgcactcaaacggaaatacctatctgcattggtacctgcagaagccaggccagtctcccaaactgctgatctataaggtgagcaaccggttctccggggtccctgacagattttctggaagtggctcagggacagatttcactctgaaaattagcagagtggaggccgaagatctgggcgtctacttttgtagccagaatacccacgtcccaccaacattcggaagcggcactaaactggaaatcaag

SEQ ID NO: 49 - protein, 26 aa, synthetic construct
MAAPALSWRLPLLILLLPLATSWASA

SEQ ID NO: 50 - DNA, 78 bp, synthetic construct
  source 1..78: mol_type = other DNA
atggcggcccctgctctgtcctggcgtctgcccctcctcatcctcctcctgcccctggctacctcttgggcatctgca

SEQ ID NO: 51 - protein, 19 aa, synthetic construct
MDWIWRILFLVGAATGAHS

SEQ ID NO: 52 - DNA, 57 bp, synthetic construct
  source 1..57: mol_type = other DNA
atggattggatttggcgcattctgtttctggtgggagccgcaaccggagcacatagt

SEQ ID NO: 53 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 164 aa, Homo sapiens; identical to SEQ ID NO: 53

SEQ ID NO: 55 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 337 bp, Homo sapiens
  source 1..337: mol_type = genomic DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 57 - protein, 251 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

SEQ ID NO: 58 - protein, 42 aa, synthetic construct
KRGRKKLLYIFKQPFMRPVQTTQEEDGCSCRFPEEEEGGCEL

SEQ ID NO: 59 - DNA, 126 bp, synthetic construct
  source 1..126: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 60 - protein, 276 aa, Homo sapiens
MCVGARRLGRGPCAALLLLGLGLSTVTGLHCVGDTYPSNDRCCHECRPGNGMVSRCSRSQNTVCRPCGPGFYNDWSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 61 - protein, 196 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVWCILGCILICWLTKKKYSSSVHDPNGEYMFMRATAKKSRLTDVTL

SEQ ID NO: 62 - protein, 4 aa, unidentified
YVKM

SEQ ID NO: 63 - protein, 220 aa, Homo sapiens
MACLGFQRHKAQLNLATRTWPCTLLFFLLFIPVFCKAMHVAQPAWLASSRGIASFVCEYASPGKATEVRVTVLRQADSQVTEVCAATYMMGNELTFLDDSICTGTSSGNQLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 64 - protein, 286 aa, Homo sapiens
MQIPQAPWPVVWAVLQLGWRPGWFLDSPDRPWNPPTFSPALLWTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGWGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 65 - protein, 524 aa, Homo sapiens
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 66 - protein, 368 aa, Homo sapiens
MLGQWTLILLLLLKVYQGKGCQGSADHWSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 67 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 68 - DNA, 20 bp, synthetic construct
  source 1..20: mol_type = other DNA
tcctggactcagctatgggg

SEQ ID NO: 69 - protein, 6 aa, synthetic construct
HHHHHH

SEQ ID NO: 70 - DNA, 22 bp, synthetic construct
  source 1..22: mol_type = other DNA
acatccatctcgtgctacttgt

SEQ ID NO: 71 - DNA, 21 bp, synthetic construct
  source 1..21: mol_type = other DNA
gcctctgttttagggagacct